(19) United States
(12) Patent Application Publication
(10) Pub. No.: US 2023/0406911 A1
(43) Pub. Date: Dec. 21, 2023

(54) COMPOSITIONS AND METHODS FOR PREVENTING, INHIBITING, DISRUPTING, OR TREATING A POLYMICROBIAL BIOFILM

(71) Applicant: RESEARCH INSTITUTE AT NATIONWIDE CHILDREN'S HOSPITAL, Columbus, OH (US)

(72) Inventors: Lauren O. Bakaletz, Hilliard, OH (US); Steven D. Goodman, Hilliard, OH (US)

(73) Assignee: RESEARCH INSTITUTE AT NATIONWIDE CHILDREN'S HOSPITAL, Columbus, OH (US)

(21) Appl. No.: 18/034,668

(22) PCT Filed: Nov. 1, 2021
(86) PCT No.: PCT/US2021/057626

§ 371 Date: Apr. 28, 2023

Related U.S. Application Data
(60) Provisional application No. 63/108,421, filed on Nov. 1, 2020.

(51) Int. Cl. C07K 16/12; A61P 31/04; A61K 45/06

(52) U.S. Cl. CPC C07K 16/12; A61P 31/04; A61K 45/06

(57) ABSTRACT

Provided are methods for one or more of the following: preventing, inhibiting, disrupting, dispersing or treating a polymicrobial biofilm comprising a Haemophilus bacteria in vitro and/or in vivo, such as in a subject in need thereof. The method comprises or consists essentially of, or yet further consists of contacting the polymicrobial biofilm with: (i) an anti DNA binding and bending protein (DNABII) antibody or a biologically active fragment thereof; and (ii) an anti majority subunit (PilA) of type IV pilus (T4P) antibody or a biologically active fragment thereof. Additionally, the methods may further comprise contacting the polymicrobial biofilm with an antibiotic optionally comprising a β-lactam antibiotic or a sulfonamide antibiotic. Also provided are compositions and kits suitable for use in the methods.

### CROSS-REFERENCE TO RELATED APPLICATIONS

This application claims the benefit under 35 U.S.C. § 119(e) of Provisional U.S. Ser. No. 63/108,421, filed Nov. 1, 2020 andp under 35 U.S.C. § 365(c) of U.S. Ser. No. 17/364,578, filed Jun. 30, 2021, the content of each of which is incorporated by reference into this application in its entirety.

### BACKGROUND

Centers for Disease Control and Prevention and the National Institutes of Health estimate that biofilms contribute to the pathogenesis of ˜80% of all bacterial infections (Dongari-Bagtzoglou, 2008). Biofilm-associated diseases such as otitis media (OM), cystic fibrosis, chronic obstructive pulmonary disease, chronic rhinosinusitis, chronic wound infections, periodontitis, cystitis and infections of medical implants and indwelling catheters, among many others, are typically chronic and/or recurrent due to the presence of bacteria within biofilms that are highly resistant to killing by host immune effectors and antibiotics (Costerton et al., 1999; Flemming and Wingender, 2010). Thus a need exits in the art to find effective therapies to treat these diseases. This disclosure satisfies this need and provides related advantages as well.

### SUMMARY OF THE DISCLOSURE

Many diseases of the upper and lower respiratory tracts are caused by nontypeable Haemophilus influenzae (NTHI) wherein a biofilm contributes significantly to each disease course (Autio et al., 2015; Cardines et al., 2012; Zhang et al., 2012). An example of one such disease wherein NTHI is the predominant pathogen is otitis media (OM) (Barkai et al., 2009; Cleary et al., 2018; Grevers et al., 2012; Vergison, 2008; Wiertsema et al., 2011), the most common bacterial disease in children (Hassan, 2013; Mittal et al., 2018). The role of biofilms in OM pathogenesis, chronicity and recurrence is widely accepted. Nonetheless, like most NTHI-induced diseases, OM is still commonly treated with broad-spectrum oral antibiotics, which do not reach sufficient levels in the middle ear (or other sites) to eradicate biofilms or even the planktonically growing bacteria within this anatomical niche (Belfield et al., 2015). Although their use is sometimes indicated or necessary, broad-spectrum antibiotics can also cause collateral damage in the form of skin rashes, diarrhea and life-long disruption of the gut microbiome, with accompanied immunological and/or developmental consequences (Gilbert et al., 2018; Kuehn et al., 2015; Lamont et al., 2020). Moreover, the all too common indiscriminate and often ineffective use of antibiotics contributes greatly to the globally burgeoning problem of development of multiple antibiotic-resistant bacteria (Beekmann et al., 2005; Leibovitz et al., 2010; Song et al., 2012).

Delivery of vaccines is the most cost-effective way to manage infectious diseases as these target prevention (Andre et al., 2008), and as such, remains a viable and truly ideal goal. However, for those children and adults with existing biofilm-associated chronic or recurrent infections, an effective therapeutic approach is greatly needed.

Applying the discoveries disclosed herein, provided herein is a method for one or more of: preventing, inhibiting, disrupting, dispersing or treating a polymicrobial biofilm that was caused by, will be caused by, or comprises a biofilm generating Haemophilus bacteria in a subject in need thereof. The method comprises, or consists essentially of, or yet further consists of administering to the subject: (i) an anti-DNABII antibody or a biologically active fragment thereof and an anti-PilA antibody or biologically active fragment thereof; or (ii) an anti-DNABII antibody or a biologically active fragment thereof and a PilA polypeptide or a biologically active fragment thereof. In one aspect, the anti-DNABII antibody comprises one or more of an anti-DNABII antibody, an anti-DNABII tip antibody, an anti-DNABII tip chimer antibody or biologically active fragment of each thereof. In one aspect, the anti-DNABII antibody comprises an anti-DNABII tip chimer antibody or biologically active fragment thereof. In certain embodiments, CDRs of an anti-tip chimer antibody are shown in SEQ ID NOs: 1-3, 7-9, 13 and 14 are illustrated below. The CDRs of an anti tail chimer are shown in SEQ ID NOs: 4-6 and 10-12. In one aspect the antibody is a polyclonal antibody. In one aspect the antibody is a monoclonal antibody. In another aspect, the antibody is a monoclonal antibody or a biologically active fragment thereof. In a further aspect, the anti-DNABII tip chimer antibody is a monoclonal antibody, a chimeric or a humanized antibody, e.g., as disclosed herein. In another aspect, the anti-PilA antibody specifically recognizes and binds the PilA peptide, examples of such include PilA surface antigens or recombinant soluble PilA (Genbank Accession No.: EFU96734.1) and Type IV Pilin (Genbank Accession No.: Yp-003864351.1). In some aspects, the polymicrobial biofilm is caused by Haemophilus, e.g. NTHI and one or more of Burkholderia cenocepacia, Staphylococcus aureus, Streptococcus pneumonia, or Moraxella catarrhalis. In some embodiments, the method further comprises administering to the subject an antibiotic. In further embodiments, the antibiotic comprises or consists essentially of, or yet further consists of a β-lactam antibiotic and/or a sulfonamide antibiotic.

Also provided is a method for one or more of: preventing, inhibiting, disrupting, dispersing or treating a disease related to a polymicrobial biofilm infection that was caused by, will be caused by, or comprises a Haemophilus bacteria in a subject in need thereof. The method comprises, or consists essentially of, or yet further consists of administering to the subject: i) an anti-DNABII antibody or a biologically active fragment thereof and an anti-PilA antibody or biologically active fragment thereof; or (ii) an anti-DNABII antibody or a biologically active fragment thereof and a PilA polypeptide or a biologically active fragment thereof. In one aspect the antibody is a polyclonal antibody or a monoclonal antibody. In one aspect, the anti-DNABII antibody comprises one or more of an anti-DNABII antibody, an anti-DNABII tip antibody, an anti-DNABII tip chimer antibody or biologically active fragment of each thereof. In one aspect, the anti-DNABII antibody comprises an anti-DNABII tip chimer antibody or biologically active fragment thereof. In certain embodiments, CDRs of an anti-tip chimer antibody are shown in SEQ ID NOs: 1-3, 7-9, 13 and 14 are illustrated below. In a further aspect, the anti-DNABII tip chimer antibody is a monoclonal antibody, a chimeric or a humanized antibody, e.g., as disclosed herein. In another aspect, the anti-PilA antibody specifically recognizes and binds the PilA peptide, examples of such include PilA surface antigens or recombinant soluble PilA (Genbank Accession No.: EFU96734.1) and Type IV Pilin (Genbank Accession No.: Yp-003864351.1). In some aspects, the polymicrobial biofilm is caused by Haemophilus, e.g. NTHI and one or more of Burkholderia cenocepacia, Staphylococcus aureus, Streptococcus pneumonia, or Moraxella catarrhalis. In some embodiments, the disease is selected from: cystic fibrosis (CF), lung CF, chronic obstructive pulmonary disease (COPD), chronic rhinosinusitis (CRS), periodontitis, periimplantitis, middle ear infection, otitis media, (OM), acute otitis media (AOM), otitis media with effusion (OME), post-tympanostomy tube otorrhea (PTTO), ventilator-associated pneumonia (VAP), community-acquired pneumonia (CAP), chest tube/catheter/indwelling device biofilm infections, tonsillitis, pharyngitis, laryngitis, epiglottitis, sinusitis, pneumonia, bronchitis, or other respiratory tract infection (RTI). In some embodiments, the method further comprises administering to the subject an antibiotic. In further embodiments, the antibiotic comprises, or consists essentially of, or yet further consists of a β-lactam antibiotic and/or a sulfonamide antibiotic.

In yet another aspect, provided is a method for one or more of: preventing, inhibiting, disrupting, dispersing or treating a polymicrobial biofilm that was caused by, will be caused by, or comprises a Haemophilus bacteria. The method comprises, or consists essentially of, or yet further consists of contacting the biofilm with (i) an anti DNA binding and bending protein (DNABII) antibody or a biologically active fragment thereof; and (ii) an anti majority subunit (PilA) of type IV pilus (T4P) antibody or a biologically active fragment thereof. In some aspects, the polymicrobial biofilm is caused by Haemophilus, e.g. NTHI and one or more of Burkholderia cenocepacia, Staphylococcus aureus, Streptococcus pneumonia, or Moraxella catarrhalis. In one aspect, the anti-DNABII antibody comprises one or more of an anti-DNABII antibody, an anti-DNABII tip antibody, an anti-DNABII tip chimer antibody or biologically active fragment of each thereof. In some aspects, the anti-DNABII antibody is a anti-DNABII tip chimer antibody or a biologically active fragment thereof. In certain embodiments, CDRs of an anti-tip chimer antibody are shown in SEQ ID NOs: 1-3, 7-9, 13 and 14 are illustrated below. In some embodiments, the method further comprises contacting the polymicrobial biofilm with an antibiotic. In further embodiments, the antibiotic comprises or consists essentially of, or yet further consists of a β-lactam antibiotic and/or a sulfonamide antibiotic. The contacting may be in vitro or in vivo.

In a further aspect, provided is a method for sensitizing a polymicrobial biofilm that was caused by, will be caused by, or comprises a Haemophilus bacteria for an antibiotic therapy or inducing bacteria that forms the polymicrobial biofilm to a newly released (NRel) state. The method comprises, or consists essentially of, or yet further consists of contacting the biofilm with (i) an anti DNA binding and bending protein (DNABII) antibody or a biologically active fragment thereof; and (ii) an anti majority subunit (PilA) of type IV pilus (T4P) antibody or a biologically active fragment thereof. The antibody can be a polyclonal antibody or monoclonal antibody or biologically active fragment thereof. In one aspect, the anti-DNABII antibody comprises one or more of an anti-DNABII antibody, an anti-DNABII tip antibody, an anti-DNABII tip chimer antibody or biologically active fragment of each thereof. In certain embodiments, CDRs of an anti-tip chimer antibody are shown in SEQ ID NOs: 1-3, 7-9, 13 and 14 are illustrated below. In one aspect, the anti-DNABII antibody comprises an anti-DNABII tip chimer antibody or biologically active fragment thereof. In a further aspect, the anti-DNABII tip chimer antibody is a monoclonal antibody, a chimeric or a humanized antibody, e.g., as disclosed herein. In another aspect, the anti-PilA antibody specifically recognizes and binds the PilA peptide, examples of such include PilA surface antigens or recombinant soluble PilA (Genbank Accession No.: EFU96734.1) and Type IV Pilin (Genbank Accession No.: Yp-003864351.1). In some embodiments, the method further comprises contacting the polymicrobial biofilm with an antibiotic. In further embodiments, the antibiotic comprises or consists essentially of, or yet further consists of a β-lactam antibiotic and/or a sulfonamide antibiotic. In some aspects, the polymicrobial biofilm is caused by Haemophilus, e.g. NTHI and one or more of Burkholderia cenocepacia, Staphylococcus aureus, Streptococcus pneumonia, or Moraxella catarrhalis. The contacting may be in vitro or in vivo.

In yet a further aspect, provided is a method for sensitizing a polyclonal microbial biofilm that was caused by, will be caused by, or comprises a Haemophilus bacteria in a subject in need thereof for an antibiotic therapy or inducing bacteria of the polymicrobial biofilm in a subject in need thereof to a newly released (NRel) state. The method comprises or consists essentially of, or yet further consists of administering to the subject: (i) an anti-DNABII antibody or a biologically active fragment thereof; and (ii) a PilA polypeptide or a biologically active fragment thereof. The antibody can be a polyclonal antibody or a monoclonal antibody. In one aspect, the anti-DNABII antibody comprises one or more of an anti-DNABII antibody, an anti-DNABII tip antibody, an anti-DNABII tip chimer antibody or biologically active fragment of each thereof. In one aspect, the anti-DNABII antibody comprises an anti-DNABII tip chimer antibody or biologically active fragment thereof. In certain embodiments, CDRs of an anti-tip chimer antibody are shown in SEQ ID NOs: 1-3, 7-9, 13 and 14 are illustrated below. In a further aspect, the anti-DNABII tip chimer antibody is a monoclonal antibody, a chimeric or a humanized antibody, e.g., as disclosed herein. In another aspect, the anti-PilA antibody specifically recognizes and binds the PilA peptide, examples of such include PilA surface antigens or recombinant soluble PilA (Genbank Accession No.: EFU96734.1) and Type IV Pilin (Genbank Accession No.: Yp-003864351.1). In some aspects, the polymicrobial biofilm is caused by Haemophilus, e.g. NTHI and one or more of Burkholderia cenocepacia, Staphylococcus aureus, Streptococcus pneumonia, or Moraxella catarrhalis. In some embodiments, the method further comprises administering to the subject an antibiotic. In further embodiments, the antibiotic comprises or consists essentially of, or yet further consists of a β-lactam antibiotic and/or a sulfonamide antibiotic.

Also provided are kits and compositions, such as those for use in a method as disclosed herein. In one aspect, provided is a kit comprising, or consisting essentially of, or yet further consisting of at least two of: (i) an anti-DNABII antibody or a biologically active fragment thereof, (ii) one or both of: an anti-PilA antibody or a biologically active fragment thereof, or a PilA polypeptide or a biologically active fragment thereof, or (iii) an antibiotic; and optionally instructions for use. The antibody can be a polyclonal antibody or a monoclonal antibody. In one aspect, the anti-DNABII antibody comprises one or more of an anti-DNABII antibody, an anti-DNABII tip antibody, an anti-DNABII tip chimer antibody or biologically active fragment of each thereof. In one aspect, the anti-DNABII antibody comprises an anti-DNABII tip chimer antibody or biologically active fragment thereof. In certain embodiments, CDRs of an anti-tip chimer antibody are shown in SEQ ID NOs: 1-3, 7-9, 13 and 14 are illustrated below. In a further aspect, the anti-DNABII tip chimer antibody is a monoclonal antibody, a chimeric or a humanized antibody, e.g., as disclosed herein. In another aspect, the anti-PilA antibody specifically recognizes and binds the PilA peptide, examples of such include PilA surface antigens or recombinant soluble PilA (Genbank Accession No.: EFU96734.1) and Type IV Pilin (Genbank Accession No.: Yp-003864351.1). In some embodiments, the antibiotic comprises or consists essentially of, or yet further consists of a β-lactam antibiotic and/or a sulfonamide antibiotic.

In another aspect, provided is a composition comprising, or consisting essentially of, or yet further consisting of at least two of the following: (i) an anti-DNABII antibody or a biologically active fragment thereof, (ii) one or both of: an anti-PilA antibody or a biologically active fragment thereof, or a PilA polypeptide or a biologically active fragment thereof, or (iii) an antibiotic; and a carrier optionally a pharmaceutically acceptable carrier. The antibody can be a polyclonal antibody or a monoclonal antibody. In one aspect, the anti-DNABII antibody comprises an anti-DNABII tip chimer antibody or biologically active fragment thereof. In certain embodiments, CDRs of an anti-tip chimer antibody are shown in SEQ ID NOs: 1-3, 7-9, 13 and 14 are illustrated below. In a further aspect, the anti-DNABII tip chimer antibody is a monoclonal antibody, a chimeric or a humanized antibody, e.g., as disclosed herein. In another aspect, the anti-PilA antibody specifically recognizes and binds the PilA peptide, examples of such include PilA surface antigens or recombinant soluble PilA (Genbank Accession No.: EFU96734.1) and Type IV Pilin (Genbank Accession No.: Yp-003864351.1). The antibodies can be detectably labeled. In some embodiments, the antibiotic comprises or consists essentially of, or yet further consists of a β-lactam antibiotic and/or a sulfonamide antibiotic.

In some embodiments, the antibody, polypeptide, biologically active fragment of each thereof, or any combination thereof may be administered to a subject in a composition as disclosed herein. In other embodiments, the antibody, polypeptide, biologically active fragment of each thereof, or any combination thereof may be administered to a subject as a polynucleotide encoding such antibody, polypeptide, biologically active fragment or a polynucleotide complementary thereto. In some embodiments, such polynucleotide may be suitable for expressing the antibody, polypeptide, biologically active fragment in the subject, for example via further comprising a regulatory sequence directing the expression. In further embodiments, a vector, such as a gene delivery vehicle, comprising a polynucleotide as disclosed herein may also be used for administration. The vector may be a viral or a non-viral vector as disclosed herein. In yet further embodiments, provided is a host cell comprising one or more of the polynucleotides and/or the vectors as disclosed herein. Accordingly, provided are kits and compositions comprising one or more of such polynucleotides and/or vectors and/or host cells in addition to or in replacement of the corresponding antibody, peptide or biological active fragment thereof expressed. Further provided are methods and compositions suitable for producing such polypeptides, antibodies, biologically active fragments, polynucleotides, vectors and host cells.

Additionally provided is a method for selecting a polymicrobial biofilm that was caused by, will be caused by, or comprises a Haemophilus bacteria suitable for one or more of the following: prevention, inhibition, disruption, dispersion, treatment or sensitization by a method as disclosed herein. In some aspects, the polymicrobial biofilm is caused by Haemophilus, e.g. NTHI and one or more of Burkholderia cenocepacia, Staphylococcus aureus, Streptococcus pneumonia, or Moraxella catarrhalis. The screening method comprises or consists essentially of, or yet further consists of (a) contacting the polymicrobial biofilm, or a polymicrobial biofilm isolated and grown therefrom, with any one of an anti-DNABII antibody or a biologically active fragment thereof, an anti-PilA antibody or biologically active fragment thereof, or a PilA polypeptide or a biologically active fragment thereof (b) and assaying released bacteria from the biofilm of (a) for expression of one or more of the following genes: deaD, artM, fis, folA, folP, emrA, emrB, or acrR and level of one or more of proteins: AsnC, CyaA, GlpC, NrfA, TehB, TrpB, TrpC, TrpD, TruA, TrxA; AbgA, AroE, AroK, ArtP, AtpH, BioB, BioF, CcmA, CitD, CitT, CmK, CoaD, DcD, DjlA, DksA, DnaE, DnaQ, DsbE, FabG, FdhX, FtnB, FtsE, FtsI, FumC, Fur, GlpA, GlpB, GmK, GpsA, GreA, Hfq, HisI, HugZ, InfA, InfB, IspD, IspF, Lic2A, LicC, LicD, LolA, LpsA, menC, MenC, NapA, NrfB, NrfC, NTHI0053, NTHI0249, NTHI0291, NTHI0319, NTHI0349, NTHI0436, NTHI0487, NTHI0490, NTHI0555, NTHI0732, NTHI0779, NTHI0820, NTHI1025, NTHI1085, NTHI1199, NTHI1437, NTHI1439, NTHI1503, NTHI1590, NTHI1748, OppF, PanF, PdxH, PepT, PhoB, PlsC, PolA, PpC, PpiB, PrfB, PurU, RadA, RelB, RibA, RplW, RpoE, RpoZ, RseA, SecF, Sxy, TesB, ThrB, TolA, UnG, UreG, UuaP, UvrB; ClpB, CydD, DeaD, DlD, DmsB, ExbD, GroES, HfeA, HfeB, HgpB, HisJ, HisJ, HitA, HktE, HxuC, LctP, LldD, LpxC, MurB, NdhA, NifS2, NTHI0043, NTHI0052, NTHI0175, NTHI0364, NTHI1208, NTHI1214, NTHI1369, NTHI1703, OmpP2, OrfG, Pal, PdgX, RbfA, RecN, RplO, RplU, RpmE, RpsE, RpsJ, RpsL, RpsQ, RpsT, SodA, Tbpl, TolB, TonB, TrpA, TrxA, or ZnuA. In some embodiments, one or more of the following indicates the biofilm is suitable for a method as disclosed herein: an altered gene expression of one or more of the following genes: deaD, artM, fis, folA, folP, emrA, emrB, or acrR; an altered level of one or more of the following proteins: AsnC, CyaA, GlpC, NrfA, TehB, TrpB, TrpC, TrpD, TruA, or TrxA; a low level of one or more of the following proteins: AbgA, AroE, AroK, ArtP, AtpH, BioB, BioF, CcmA, CitD, CitT, CmK, CoaD, DcD, DjlA, DksA, DnaE, DnaQ, DsbE, FabG, FdhX, FtnB, FtsE, FtsI, FumC, Fur, GlpA, GlpB, GmK, GpsA, GreA, Hfq, HisI, HugZ, InfA, InfB, IspD, IspF, Lic2A, LicC, LicD, LolA, LpsA, menC, MenC, NapA, NrfB, NrfC, NTHI0053, NTHI0249, NTHI0291, NTHI0319, NTHI0349, NTHI0436, NTHI0487, NTHI0490, NTHI0555, NTHI0732, NTHI0779, NTHI0820, NTHI1025, NTHI1085, NTHI1199, NTHI1437, NTHI1439, NTHI1503, NTHI1590, NTHI1748, OppF, PanF, PdxH, PepT, PhoB, PlsC, PolA, PpC, PpiB, PrfB, PurU, RadA, RelB, RibA, RplW, RpoE, RpoZ, RseA, SecF, Sxy, TesB, ThrB, TolA, UnG, UreG, UuaP, or UvrB; or a high level of one or more of the following proteins: ClpB, CydD, DeaD, DlD, DmsB, ExbD, GroES, HfeA, HfeB, HgpB, HisJ, HisJ, HitA, HktE, HxuC, LctP, LldD, LpxC, MurB, NdhA, NifS2, NTHI0043, NTHI0052, NTHI0175, NTHI0364, NTHI1208, NTHI1214, NTHI1369, NTHI1703, OmpP2, OrfG, Pal, PdgX, RbfA, RecN, RplO, RplU, RpmE, RpsE, RpsJ, RpsL, RpsQ, RpsT, SodA, Tbpl, TolB, TonB, TrpA, TrxA, or ZnuA. The antibody can be a polyclonal antibody or a monoclonal antibody. In one aspect, the anti-DNABII antibody comprises one or more of an anti-DNABII antibody, an anti-DNABII tip antibody, an anti-DNABII tip chimer antibody or biologically active fragment of each thereof. In one aspect, the anti-DNABII antibody comprises an anti-DNABII tip chimer antibody or biologically active fragment thereof. In a further aspect, the anti-DNABII tip chimer antibody is a monoclonal antibody, a chimeric or a humanized antibody, e.g., as disclosed herein. In certain embodiments, CDRs of an anti-tip chimer antibody are shown in SEQ ID NOs: 1-3, 7-9, 13 and 14 are illustrated below. In another aspect, the anti-PilA antibody specifically recognizes and binds the PilA peptide, examples of such include PilA surface antigens or recombinant soluble PilA (Genbank Accession No.: EFU96734.1) and Type IV Pilin (Genbank Accession No.: Yp-003864351.1). The antibodies can be detectably labeled. Optionally, the high, low, altered expression or level is compared to the same bacteria but grown planktonically. Further provided is a kit for use in the method comprising, or consisting essentially of, or yet further consisting of instruction for use and probes suitable for the assay. In some embodiments, one or more of the probes are labeled.

### BRIEF DESCRIPTION OF THE DRAWINGS

FIGS. 1A to 1B: show quantitation of NTHI released from biofilm-residence by either anti-rsPilA or anti-IHF. NTHI biofilms established for 16 h were incubated for an additional (FIG. 1A) 6 h with rabbit anti-rsPilA IgG or (FIG. 1B) 15 min with rabbit anti-IHF IgG or with each of three negative controls (sBHI, IgG isolated from naive serum or IgG isolated from anti-OMP P5 serum) followed by quantitation of NTHI recovered from supernatants above the biofilms. Anti-rsPilA and anti-IHF induced significant release of NTHI from biofilm residence into the Newly Released (NRel) state. Individual data points are shown, bars represent mean±SEM. ****, P<0.0001, One-way analysis of variance with the Holm-Sidak correction.

FIGS. 2A to 2E: show that anti-rsPilA NRel were released from a biofilm as individual cells whereas anti-IHF NRel were aggregated. FIG. 2A provides contour plots depicted side scatter and forward scatter profiles for two control samples, NTHI briefly sonicated to produce an individual cell suspension or NTHI colonies collected from an agar plate to represent bacterial aggregates, assessed by flow cytometry. Representative contour plots of (FIG. 2B) anti-rsPilA NRel and (FIG. 2C) anti-IHF NRel demonstrated unique scatter profiles between the two NRel populations. The distribution of anti-IHF NRel (right histogram) versus anti-rsPilA NRel (left histogram) by (FIG. 2D) forward scatter and (FIG. 2E) side scatter was also distinct. The percent increase in cumulative distribution of anti-IHF NRel compared to anti-rsPilA NRel shown in (FIG. 2D) and (FIG. 2E) was determined by Kolmogorov-Smirnov test (99% CI). These data represented an additional discriminative characteristic of NRel populations generated via exposure to either anti-rsPilA or anti-DNABII antibody-mediated release of NTHI from biofilm residence.

FIGS. 3A to 3D: show that release of NTHI from a biofilm by incubation with anti-rsPilA or anti-IHF antibodies generated NRel populations with distinct proteomic expression profiles compared to planktonically grown NTHI and, importantly, to each other. FIG. 3A provides principal component analysis (PCA) plot generated from the normalized spectral counts of each protein in anti-rsPilA NRel (dots in the middle circle), anti-IHF NRel (dots in the left circle) and planktonically grown NTHI (dots in the right circle). Triplicate samples of each population are encircled by 95% confidence ellipses. The proteomic expression profiles of anti-rsPilA and anti-IHF NRel were distinct from both planktonically grown NTHI, and from each other. FIG. 3B provides Venn diagram of the number of proteins with a significant (P<0.05) 1.5-fold increase (above the dashed line) or decrease (below the dashed line) specific to anti-rsPilA (left section), anti-IHF (right section), or shared (center section), compared to planktonic NTHI. FIG. 3C shows that the anti-rsPilA (top bar of each group) and anti-IHF (bottom bar of each group) NRel demonstrated distinct protein expression patterns with a significant (P<0.05) 1.5-fold increase or decrease represented by different COG categories when compared to planktonically grown NTHI. FIG. 3D provides direct comparison of differences in protein expression profiles of anti-IHF and anti-rsPilA NRel populations with a significant (P<0.05) 1.5-fold increase or decrease compared to each other, as shown by a volcano plot of anti-IHF NRel versus anti-rsPilA NRel. Negative significant fold decreases represent proteins with greater abundance in the anti-rsPilA NRel (dots in the top left section), while positive fold increases represent greater protein abundance in the anti-IHF NRel (dots in the top right section) compared to each other.

FIGS. 4A to 4C: show differences in relative gene expression support the observed distinct anti-IHF and anti-rsPilA NRel phenotypes, including antibiotic sensitivities. Results of qRT-PCR assay to examine the expression of genes by NRel relative to planktonic NTHI. FIG. 4A provides relative expression of deaD, artM, and fis, genes associated with lag phase of growth, was significantly greater in anti-IHF vs. anti-rsPilA NRel. FIG. 4B shows the enzymes targeted by TMP and SMX are encoded by folA and folP respectively, and thereby increased expression confers resistance. Relative expression of folA and folP by anti-rsPilA NRel was significantly less than by anti-IHF NRel. FIG. 4C shows that relative expression of emrA and emrB, which encode subunits of an efflux pump that transports TMP-SMX, was significantly less by anti-rsPilA vs. anti-IHF NRel. Expression of acrR, which represses the efflux pump that transports AMC, was significantly elevated in anti-IHF vs. anti-rsPilA NRel. These patterns of relative gene expression support the enhanced sensitivities of anti-rsPilA or anti-IHF NRel to TMP-SMX or AMC, respectively. *P<0.05, ***P<0.001, ****P<0.0001, Student's t-test.

FIGS. 5A to 5E: show that NRel NTHI populations were more sensitive to killing than their planktonic counterparts, and this sensitivity was distinct from each other. FIG. 5A provides a diagram of the four populations of NTHI tested herein. NRel were generated by incubation of NTHI biofilms with rabbit polyclonal IgG isolated from anti-rsPilA serum (6 h, left bottom) or from anti-IHF serum (15 min, right bottom). FIGS. 5B & FIG. 5C show that anti-rsPilA NRel were significantly more sensitive to killing by trimethoprim/sulfamethoxazole than planktonic NTHI (TMP-SMX at 0.94 μg and 4.7 μg per ml respectively, FIG. 5B), but only equally as sensitive to killing by amoxicillin/clavulanate (AMC at 2.5 μg and 1.25 μg per ml, respectively FIG. 5C). Biofilm-resident NTHI displayed minimal sensitivity to either TMP-SMX or AMC as expected. In contrast, as shown in FIGS. 5D & 5E, anti-IHF NRel were only equally as sensitive to killing by TMP-SMX as planktonic NTHI (0.09 and 0.45 μg/ml respectively, FIG. 5D), but significantly more sensitive to killing by AMC (0.30 and 0.15 μg/ml respectively, FIG. 5E). The uniquely heightened sensitivity of NTHI NRel to killing by either TMP-SMX or AMC was dependent upon the mechanism by which they were released from biofilm residence. Individual data points are shown, bars represent mean±SEM. ***P<0.001, ****P<0.0001, one-way analysis of variance with the Holm-Sidak correction.

FIGS. 6A to 6D: provide that enhanced sensitivity of anti-rsPilA or anti-IHF NRel NTHI to TMP-SMX or AMC, respectively, was independent of the timing of NTHI release from biofilm residence. Applicant exposed biofilms to rabbit polyclonal IgG isolated from either anti-rsPilA or anti-IHF serum for 2 h, then collected NRel NTHI and assayed for relative antibiotic sensitivity. (FIGS. 6A & FIG. 6B) show that anti-rsPilA NRel were significantly more sensitive to killing by TMP/SMX than planktonic NTHI (TMP-SMX at 0.09 μg and 0.45 μg per ml respectively, (FIG. 6A), but only equally as sensitive to killing by AMC (AMC at 0.3 μg and 0.15 μg per ml, respectively FIGURE). In contrast, as shown in (FIGS. 6C & FIG. 6D), anti-IHF NRel were only equally as sensitive to killing by TMP-SMX as planktonic NTHI (0.94 and 4.7 μg/ml respectively, FIG. 6C), but significantly more sensitive to killing by AMC (2.5 and 1.25 μg/ml respectively, FIG. 6D). These data showed that time-matched anti-rsPilA or anti-IHF NRel maintained the same distinct antibiotic sensitivity phenotype as shown when 15 min anti-IHF NRel were compared to 6 h anti-rsPilA NRel (see FIG. 5). Individual data points are shown, bars represent mean±SEM. ****P<0.0001, one-way analysis of variance with the Holm-Sidak correction.

FIG. 7: shows synergistic effects of an anti-rsPilA antibody and an anti-IHF (e.g., an anti-DNABII) antibody. Wells were incubated with NTHI+Streptococcus pneumoniae+Staphylococcus aureas in a 1:1:1 ratio and allowed to form a biofilm for 16 hours at 37° C. and 5% CO₂ prior to a 2 hour treatment to determine relative disruption compared to treatment with medium alone (see treatments marked in the figure). Bacteria were labeled with FM1-43 FX and relative fluorescence was calculated per well. A clear synergistic outcome was achieved via combinatorial treatment with anti-rsPilA plus anti-tip chimer monoclonal antibody, demonstrated here for the first time.

FIGS. 8A to 8C: show antibiotic sensitivity of NTHI newly released from a biofilm by anti-IHF_{NTHI}. Biofilms were grown at 37° C. for 16 hours, and then treated for 6 h with anti-IHF_{NTHI} (1:50 dilution) or sBHI only. Planktonic population was collected and assayed for antibiotic sensitivity. AC concentration used is 1/0.5 μg/ml and TS concentration used is 7.5/37.5 μg/ml. (FIG. 8A) plots data from anti-IHF diluted at 1/10 or no anti-IHF treatment. (FIG. 8B) plots data from anti-IHF diluted at 1/100 or no anti-IHF treatment. (FIG. 8C) provides percentage of killing. N=3.

FIGS. 9A to 9C: show synergistic activity against multi-species biofilms. (FIG. 9A) show NTHI+Burkholderia cenocepacia. The NTHI alone biofilms continued to perform as expected with the cocktails synergy clearly shown. For the 2-species biofilm, both antisera alone were effective but the cocktail induced a statistically significant synergistic release of both species from a 2-species biofilm. (FIG. 9B) shows activity against NTHI+Staphylococcus aureus biofilm. (FIG. 9C) shows NTHI+Streptococcus pneumonia.

FIGS. 10A to 10E: show synergistic activity of anti-rsPilA+anti-tip chimer to release by Moraxella catarrhalis (Mcat) from a dual species biofilm. (FIG. 10A) NRels released from either a 2-species biofilm formed by NTHI+Mcat or an NTHI biofilm after 2 hour exposure to 2.5 μg of either anti-rsPilA, anti-Tip or a combination of both antibodies compared to non-associated bacteria from a nontreated biofilm. (FIG. 10B) NRels released from either a 2-species biofilm formed by NTHI+Mcat or an NTHI biofilm after 6 hour exposure to 2.5 μg of either anti-rsPilA, anti-Tip or a combination of both antibodies compared to non-associated bacteria from a nontreated biofilm. (FIG. 10C) NRels released from either a 2-species biofilm formed by NTHI+Mcat or an NTHI biofilm after 16 hour exposure to 2.5 μg of either anti-rsPilA, anti-Tip or a combination of both antibodies compared to non-associated bacteria from a nontreated biofilm. (FIG. 10D) NRels released from either a 2-species biofilm formed by NTHI+Mcat or an NTHI biofilm after 6 hour exposure to 10 μg of either anti-rsPilA, anti-Tip or a combination of both antibodies compared to non-associated bacteria from a nontreated biofilm. (FIG. 10E) NRels released from either a 2-species biofilm formed by NTHI+Mcat or an NTHI biofilm after 16 hour exposure to 10 μg of either anti-rsPilA, anti-Tip or a combination of both antibodies compared to non-associated bacteria from a nontreated biofilm.

### DETAILED DESCRIPTION

### Definitions

All technical and patent publications cited herein are incorporated herein by reference in their entirety. Nothing herein is to be construed as an admission that the disclosure is not entitled to antedate such disclosure by virtue of prior disclosure. Throughout this disclosure, various technical publications are referenced by their first author and publication year or other identifying citation. The complete citations for the publications identified by first author and publication year can be found immediately preceding the claims.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods, devices, and materials are now described.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (Cold Spring Harbor Laboratory Press (2002)); Sohail (ed.) (2004) Gene Silencing by RNA Interference: Technology and Application (CRC Press).

All numerical designations, e.g., pH, temperature, time, concentration, and molecular weight, including ranges, are approximations which are varied (+) or (−) by increments of 0.1 or 1.0, where appropriate. It is to be understood, although not always explicitly stated that all numerical designations are preceded by the term “about.” It also is to be understood, although not always explicitly stated, that the reagents described herein are merely exemplary and that equivalents of such are known in the art.

The term “about,” as used herein when referring to a measurable value such as an amount or concentration and the like, is meant to encompass variations of 20%, 10%, 5%, 1%, 0.5%, or even 0.1% of the specified amount.

As used in the specification and claims, the singular form “a”, “an” and “the” include plural references unless the context clearly dictates otherwise. For example, the term “an antibody” includes a plurality of antibodies, including mixtures thereof.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this disclosure or process steps to produce a composition or achieve an intended result. Embodiments defined by each of these transition terms are within the scope of this disclosure.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

As used herein, comparative terms as used herein, such as high, low, increase, decrease, reduce, or any grammatical variation thereof, can refer to certain variation from the reference. In some embodiments, such variation can refer to an increase of about 10%, or about 20%, or about 30%, or about 40%, or about 50%, or about 60%, or about 70%, or about 80%, or about 90%, or about 1 fold, or about 2 folds, or about 3 folds, or about 4 folds, or about 5 folds, or about 6 folds, or about 7 folds, or about 8 folds, or about 9 folds, or about 10 folds, or about 20 folds, or about 30 folds, or about 40 folds, or about 50 folds, or about 60 folds, or about 70 folds, or about 80 folds, or about 90 folds, or about 100 folds or more higher than the reference. In some embodiments, such variation can refer to an decrease, such as about 1%, or about 2%, or about 3%, or about 4%, or about 5%, or about 6%, or about 7%, or about 8%, or about 0%, or about 10%, or about 20%, or about 30%, or about 40%, or about 50%, or about 60%, or about 70%, or about 75%, or about 80%, or about 85%, or about 90%, or about 95%, or about 96%, or about 97%, or about 98%, or about 99% of the reference.

As used herein, the term “polymicrobial biofilm” intends a biofilm caused by more than one species of biofilm causing bacteria. In some aspects, the polymicrobial biofilm is caused by Haemophilus, e.g. NTHI and another bacteria, e.g., one or more of Burkholderia cenocepacia, Staphylococcus aureus, Streptococcus pneumonia, or Moraxella catarrhalis.

In some embodiments, the term “disrupt” or a grammatical variation thereof intends interference with DNA/protein matrix that is a component of a microbial biofilm, such as targeting an antigen in the biofilm structural linchpin outside of the microbes, such as bacteria, thus breaking down the biofilm and resulting in reduction and/or elimination of the biofilm. In some embodiments, such disruption process is not contributed by the microbes, such as bacteria. In some embodiments as shown herein, an anti-DNABII antibody disrupts a biofilm. In further embodiments, such disruption is not microbe-species specific.

In certain embodiments, disrupting a biofilm refers to dispersing the biofilm, releasing microorganisms from the DNA/protein matrix of the biofilm, and optionally allowing killing the microorganisms by host immune effectors and/or antibiotics.

The term “disperse” or a grammatical variation refers to interference with a molecule on the surface of or inside a microbe in a biofilm, such as targeting an antigen on the surface of the microbe, thus breaking down the biofilm and resulting in reduction and/or elimination of the biofilm. In some embodiments as shown herein, an anti-PilA antibody disperses a biofilm. In further embodiments, the biofilm comprises and/or is generated by nontypeable Haemophilus influenzae.

“Inhibiting, preventing or disrupting” a biofilm intends the prophylactic or therapeutic reduction in the structure of a biofilm.

“Microbial DNA” intends single or double stranded DNA from a microorganism, such as a bacteria, that is incorporated into a biofilm. As used herein the term “eDNA” refers to extracellular DNA found as a component to pathogenic biofilms.

The terms “polynucleotide”, “nucleic acid” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form. The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching. In some embodiments, the polynucleotide as disclosed herein is a RNA. In some embodiments, the polynucleotide as disclosed herein is a DNA. In some embodiments, the polynucleotide as disclosed herein is a hybrid of DNA and RNA.

The term “isolated” as used herein with respect to nucleic acids, such as DNA or RNA, refers to molecules separated from other DNAs or RNAs, respectively that are present in the natural source of the macromolecule. The term “isolated nucleic acid” is meant to include nucleic acid fragments which are not naturally occurring as fragments and would not be found in the natural state. The term “isolated” is also used herein to refer to polypeptides, proteins and/or host cells that are isolated from other cellular proteins and is meant to encompass both purified and recombinant polypeptides. In other embodiments, the term “isolated” means separated from constituents, cellular and otherwise, in which the cell, tissue, polynucleotide, peptide, polypeptide, protein, antibody or fragment(s) thereof, which are normally associated in nature. For example, an isolated cell is a cell that is separated form tissue or cells of dissimilar phenotype or genotype. As is apparent to those of skill in the art, a non-naturally occurring polynucleotide, peptide, polypeptide, protein, antibody or fragment(s) thereof, does not require “isolation” to distinguish it from its naturally occurring counterpart.

In some embodiments, the term “engineered” or “recombinant” refers to having at least one modification not normally found in a naturally occurring protein, polypeptide, polynucleotide, strain, wild-type strain or the parental host strain of the referenced species. In some embodiments, the term “engineered” or “recombinant” refers to being synthetized by human intervention.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, fragment, polypeptide or nucleic acid, intends those having minimal homology while still maintaining desired structure or functionality. Unless specifically recited herein, it is contemplated that any polynucleotide, polypeptide or protein mentioned herein also includes equivalents thereof. In some embodiments, an equivalent to a reference nucleic acid, polynucleotide or oligonucleotide encodes the same sequence encoded by the reference. In some embodiments, an equivalent to a reference nucleic acid, polynucleotide or oligonucleotide hybridizes to the reference, a complement reference, a reverse reference, and/or a reverse-complement reference, optionally under conditions of high stringency.

Additionally or alternatively, an equivalent nucleic acid, polynucleotide or oligonucleotide is one having at least 70%, or at least 75%, or at least 80% sequence identity, or alternatively at least 85% sequence identity, or alternatively at least 90% sequence identity, or alternatively at least 92% sequence identity, or alternatively at least 95% sequence identity, or alternatively at least 97% sequence identity, or alternatively at least 98% sequence identity to the reference nucleic acid, polynucleotide, or oligonucleotide, or alternatively an equivalent nucleic acid hybridizes under conditions of high stringency to a reference polynucleotide or its complement. In one aspect, the equivalent must encode functional protein that optionally can be identified through one or more assays described herein. In another aspect, an equivalent has at least the 70%, or at least 75%, or at least 80% sequence identity, or alternatively at least 85% sequence identity, or alternatively at least 90% sequence identity, or alternatively at least 92% sequence identity, or alternatively at least 95% sequence identity, or alternatively at least 97% sequence identity, or alternatively at least 98% sequence identity to the reference nucleic acid, polynucleotide, or oligonucleotide, or alternatively an equivalent nucleic acid hybridizes under conditions of high stringency to a reference polynucleotide or its complement, with the proviso that one or more mutated polynucleotides identified herein having one or more non-naturally occurring glycosylation sites are not mutated from the corresponding mutated polynucleotides in the disclosed sequences.

In one aspect, an equivalent polynucleotide is one that hybridizes under stringent conditions to the polynucleotide or complement of the polynucleotide as described herein for use in the described methods. In another aspect, an equivalent antibody or antigen binding polypeptide intends one that binds to an antigen with at least 70%, or alternatively at least 75%, or alternatively at least 80%, or alternatively at least 85%, or alternatively at least 90%, or alternatively at least 95% affinity or higher affinity compared to a reference antibody or antigen binding fragment. In another aspect, the equivalent thereof competes with the binding of the reference antibody or antigen binding fragment to its antigen under a competitive ELISA assay. In another aspect, an equivalent intends at least about 80% homology or identity and alternatively, at least about 85%, or alternatively at least about 90%, or alternatively at least about 95%, or alternatively 98% percent homology or identity and exhibits substantially equivalent biological activity to the reference protein, polypeptide or nucleic acid. Examples of biologically equivalent polypeptides are provided in Table 9 of WO 2011/123396 which identifies conservative amino acid substitutions to the disclosed amino acid sequences.

A polynucleotide or polynucleotide region (or a polypeptide or polypeptide region) has a certain percentage (for example, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: www.ncbi.nlm.nih.gov/cgi-bin/BLAST.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

“Homology” or “identity” or “similarity” can also refer to two nucleic acid molecules that hybridize under stringent conditions. “Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme. When hybridization occurs in an antiparallel configuration between two single-stranded polynucleotides, the reaction is called “annealing” and those polynucleotides are described as “complementary.” A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

Hybridization reactions can be performed under conditions of different “stringency”. In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC. Other examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed. Hybridization reactions can also be performed under “physiological conditions” which is well known to one of skill in the art. A non-limiting example of a physiological condition is the temperature, ionic strength, pH and concentration of Mg²⁺ normally found in a cell.

The expression “amplification of polynucleotides” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or its flanking regions. Nucleic acid sequences generated by amplification may be sequenced directly. Alternatively, the amplified sequence(s) may be cloned prior to sequence analysis. A method for the direct cloning and sequence analysis of enzymatically amplified genomic segments is known in the art.

A “gene” refers to a polynucleotide containing at least one open reading frame (ORF) that is capable of encoding a particular polypeptide or protein after being transcribed and translated.

The term “express” refers to the production of a gene product. As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

A “gene product” or alternatively a “gene expression product” refers to the mRNA and/or the amino acid (e.g., peptide or polypeptide) generated when a gene is transcribed and translated.

“Under transcriptional control” is a term well understood in the art and indicates that transcription of a polynucleotide sequence, usually a DNA sequence, depends on its being operatively linked to an element which contributes to the initiation of, or promotes, transcription. “Operatively linked” intends the polynucleotides are arranged in a manner that allows them to function in a cell.

The term “a regulatory sequence” “an expression control element” or “promoter” as used herein, intends a polynucleotide that is operatively linked to a target polynucleotide to be transcribed and/or replicated, and facilitates the expression and/or replication of the target polynucleotide. A promoter is an example of an expression control element or a regulatory sequence. Promoters can be located 5′ or upstream of a gene or other polynucleotide, that provides a control point for regulated gene transcription. Polymerase II and III are examples of promoters.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

A “probe” when used in the context of polynucleotide manipulation refers to an oligonucleotide that is provided as a reagent to detect a target potentially present in a sample of interest by hybridizing with the target. Usually, a probe will comprise a detectable label or marker or a means by which a label or marker can be attached, either before or subsequent to the hybridization reaction. Alternatively, a “probe” can be a biological compound such as a polypeptide, antibody, or fragments thereof that is capable of binding to the target potentially present in a sample of interest.

A “primer” is a short polynucleotide, generally with a free 3′ —OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in MacPherson et al. (1991) PCR 1: A Practical Approach (IRL Press at Oxford University Press). All processes of producing replicate copies of a polynucleotide, such as PCR or gene cloning, are collectively referred to herein as “replication.” A primer can also be used as a probe in hybridization reactions, such as Southern or Northern blot analyses. Sambrook and Russell (2001), infra.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

A “C-terminal polypeptide” intends at least the 10, or alternatively at least the 15, or alternatively at least 20, or at least the 25 C-terminal amino acids or alternatively half of a polypeptide. In another aspect, for polypeptides containing 90 amino acids, the C-terminal polypeptide would comprise amino acids 46 through 90. In one aspect, the term intends the C-terminal 20 amino acids from the carboxyl terminus.

As used herein, an amino acid (aa) or nucleotide (nt) residue position in a sequence of interest “corresponding to” an identified position in a reference sequence refers to that the residue position is aligned to the identified position in a sequence alignment between the sequence of interest and the reference sequence. Various programs are available for performing such sequence alignments, such as Clustal Omega and BLAST.

The term “conservative amino acid substitution” refers to a substitution of a native amino acid residue with a normative residue, including naturally occurring and nonnaturally occurring amino acids, such that there is little or no effect on the polarity or charge of the amino acid residue at that position. For example, a conservative substitution results from the replacement of a non-polar residue in a polypeptide with any other non-polar residue. Further, any native residue in the polypeptide may also be substituted with alanine, according to the methods of “alanine scanning mutagenesis”. Naturally occurring amino acids are characterized based on their side chains as follows: basic: arginine, lysine, histidine; acidic: glutamic acid, aspartic acid; uncharged polar: glutamine, asparagine, serine, threonine, tyrosine; and non-polar: phenylalanine, tryptophan, cysteine, glycine, alanine, valine, proline, methionine, leucine, norleucine, isoleucine General rules for amino acid substitutions are set forth below.

Amino Acid Substitutions

| Original | Exemplary | Preferred |
|---|---|---|
| Residues | Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asn |
| Gly | Pro, Als | Ala |
| His | Asn, Gln, Lys, Arg | Arg |
| Ile | Leu, Val, Met, Ala, Phe, | Leu |
| Leu | Norleucine, Ile, Val, Met, | Leu |
| Lys | Arg, 1,4 Diaminobutyric | Arg |
| Met | Leu, Phe, Ile | Let |
| Phe | Leu, Val, Ile, Ala, Tyr | Arg |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, | Leu |

The term equivalent and biological equivalent are used interchangeably, for example when referring to an antibody, antibody fragment, protein or polypeptide as a reference. In some embodiments, an equivalent antibody, antibody fragment, protein or polypeptide is one having at least about 60%, at least about 70%, at least about 80%, at least about 85%, at least about 90%, at least about 91%, at least about 92%, at least about 93%, at least about 94%, or at least about 95%, at least about 96%, at least about 97%, at least about 98%, or at least about 99% sequence identity to the reference protein or polypeptide. In some embodiments, an equivalent antibody, antibody fragment, protein or polypeptide has at least about 60%, at least about 70%, at least about 80%, at least about 85%, at least about 90%, at least about 91%, at least about 92%, at least about 93%, at least about 94%, or at least about 95%, at least about 96%, at least about 97%, at least about 98%, or at least about 99% sequence identity to a polypeptide or protein as disclosed herein. In some embodiments, an equivalent antibody, antibody fragment, protein or polypeptide has at least about 60%, at least about 70%, at least about 80%, at least about 85%, at least about 90%, at least about 91%, at least about 92%, at least about 93%, at least about 94%, or at least about 95%, at least about 96%, at least about 97%, at least about 98%, or at least about 99% sequence identity to antibody, antibody fragment, polypeptide or protein encoded by an equivalent polynucleotide as noted herein. In addition or alternatively, the equivalent of a polynucleotide would encode an antibody, antibody fragment, protein or polypeptide of the same or similar function as the reference or parent polynucleotide.

In some embodiments, the equivalent is a functional protein that optionally can be identified through one or more assays described herein. In another aspect, an equivalent has at least about 60%, at least about 70%, at least about 80%, at least about 85%, at least about 90%, at least about 91%, at least about 92%, at least about 93%, at least about 94%, or at least about 95%, at least about 96%, at least about 97%, at least about 98%, or at least about 99% sequence identity to the reference protein or polypeptide.

In some embodiments, the equivalent or biologically equivalent antibody, antibody fragment, protein or polypeptide performs functions similar to a wildtype and/or at a similar level compared to a wildtype.

As used herein, the term “EC₅₀” refers to the concentration of an antibody or a fragment thereof which induces a response (for example, binding between the antibody or a fragment thereof and its target) halfway between the baseline and maximum after a specified exposure time.

Several parameters are used herein to described the binding and unbinding reaction of receptor (R, such as an antibody or a fragment thereof) and ligand (L, such as the target of the antibody or a fragment thereof) molecules, which is formalized as: R+L⇄RL. The reaction is characterized by the on-rate constant kₒₙ and the off-rate constant k_{off}, which have units of M⁻¹ s⁻¹ and s⁻¹, respectively. In equilibrium, the forward binding transition R+L→RL should be balanced by the backward unbinding transition RL→R+L. That is kₒₙ[R] [L]=k_{off}[RL] where [R], [L] and [RL] represent the concentration of unbound free receptors, the concentration of unbound free ligand and the concentration of receptor-ligand complexes. Further, the equilibrium dissociation constant “K_{D}” can be calculated as k_{off}/kₒₙ which is [R]×[L]/[RL], while the equilibrium association constant “K_{A}” can be calculated as kₒₙ/k_{off} which is [RL]/([R]×[L]).

As used herein, the term “tip” or “conformational tip domain” of a polypeptide refers to a polypeptide that comprises a primary amino acid sequence wherein the structure has an anti-parallel beta ribbon with a sharp turn that is typically mediated by a proline residue.

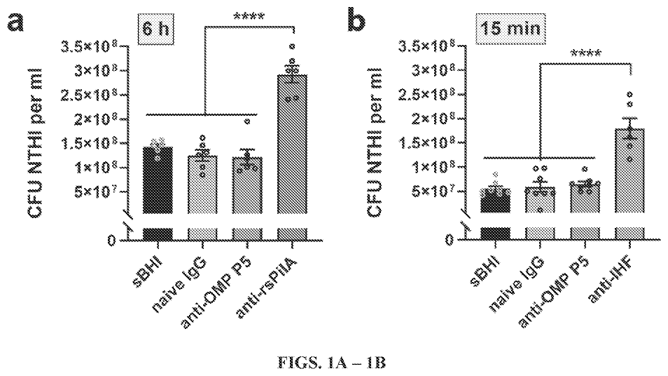

A “tip chimer” of a polypeptide intends a polypeptide comprising two tips of one or more polypeptides. The tip chimer may comprise two tips of the same polypeptide or a tip from two different polypeptides. A tip chimer of a DNABII polypeptide intends a DNABII polypeptide that, using IHFalpha and IHFbeta as examples, forms the two arms of the proteins. (see FIG. 1 of U.S. Pat. No. 11,104,723, incorporated by reference). Non-limiting examples of such include the tip fragment of IhfA, which is also referred to herein as A tip fragment: NFELRDKSSRPGRNPKTGDVV, SEQ ID NO: , and IhfB modified B4 which is also referred to herein as mB4 tip fragment: SLHHRQPRLGRNPKTGDSVNL (SEQ ID NO: ) or FSLHHRQPRLGRNPKTGDSV, SEQ ID NO: , or 2 IhfA tip fragments, or 2 IhfB modified B4 tip fragments. Additional examples are disclosed in PCT Publ. No. WO 2018/129078, incorporated herein by reference. In certain embodiments, the tip-chimeric peptide comprises the peptide designated IhfA5-mIhfB4_{NTHI} herein that comprises, or consists essentially of, or yet further consists of: a polypeptide sequence of RPGRNPX₁TGDVVPVSARRVV-X-FSLHHRQPRLGRNPX₁TGDSV (SEQ ID NO.), wherein “X” is an optional amino acid linker sequence, optionally comprising, or consisting essentially of, or yet further consisting of between 1 to 20 amino acids; and wherein “X₁” is any amino acid or alternatively “X₁” is selected from the amino acids Q, R, K, S, or T. In a further aspect, “X₁” is a K or Q. In a further embodiment, the tip-chimeric peptide IhfA5-mIhfB4_{NTHI} comprises, or consists essentially of, or yet further consists of: a polypeptide sequence of RPGRNPKTGDVVPVSARRVV-X-FSLHHRQPRLGRNPKTGDSV (SEQ ID NO: ), wherein “X” is an optional amino acid linker sequence optionally comprising, or consisting essentially of, or yet further consisting of between 1 to 20 amino acids. In yet a further embodiment, the tip-chimeric peptide IhfA5-mIhfB4_{NTHI} comprises or consists essentially of, or yet further consists of: a polypeptide sequence of RPGRNPKTGDVVPVSARRVVGPSLFSLHHRQPRLGRNPKTGDSV (SEQ ID NO: ), in some embodiments, which is referred to herein as the tip chimer), wherein GPSL serves as the amino acid linker sequence.

A “tail fragment” of a DNABII polypeptide intends a region of the protein that is both exposed to the bulk medium and not occluded by DNA or other polypeptides.

An immunodominant antigen intends a region of the protein that is recognized and binds with high affinity to an antibody.

As used herein, an “antibody” includes whole antibodies and any antigen-binding fragment or a single chain thereof, variant, or derivative thereof. Thus, the term “antibody” includes any protein or peptide containing molecule that comprises at least a portion of an immunoglobulin molecule. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein, any of which can be incorporated into an antibody of the present disclosure. The term “antibody” is further intended to encompass digestion fragments, specified portions, derivatives and variants thereof, including antibody mimetics or comprising portions of antibodies that mimic the structure and/or function of an antibody or specified fragment or portion thereof, including single chain antibodies and fragments thereof. Examples of binding fragments encompassed within the term “antigen binding portion” of an antibody include a Fab fragment, a monovalent fragment consisting of the V_{L}, V_{H}, C_{L} and CH, domains; a F(ab′)₂ fragment, a bivalent fragment comprising two Fab fragments linked by a disulfide bridge at the hinge region; a Fd fragment consisting of the V_{H} and C_{H}, domains; a Fv fragment consisting of the V_{L} and V_{H} domains of a single arm of an antibody, a dAb fragment (Ward et al. (1989) Nature 341:544-546), which consists of a V_{H} domain; and an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv)). Bird et al. (1988) Science 242:423-426 and Huston et al. (1988) Proc. Natl. Acad Sci. USA 85:5879-5883. Single chain antibodies are also intended to be encompassed within the term “fragment of an antibody” or “a biologically active fragment” of an antibody. Any of the above-noted antibody fragments are obtained using conventional techniques known to those of skill in the art, and the fragments are screened for binding specificity and neutralization activity in the same manner as are intact antibodies.

The terms “antibody,” “antibodies” and “immunoglobulin” also include immunoglobulins of any isotype, fragments of antibodies which retain specific binding to antigen, including, but not limited to, Fab, Fab′, F(ab)₂, Fv, scFv, dsFv, Fd fragments, dAb, V_{H}, V_{L}, VhH, and V-NAR domains; minibodies, diabodies, triabodies, tetrabodies and kappa bodies; multispecific antibody fragments formed from antibody fragments and one or more isolated. Examples of such include, but are not limited to a complementarity determining region (CDR) of a heavy or light chain or a ligand binding portion thereof, a heavy chain or light chain variable region (which is also referred to herein as a variable domain), a heavy chain or light chain constant region (which is also referred to herein as a constant domain), a framework (FR) region, or any portion thereof, at least one portion of a binding protein, chimeric antibodies, humanized antibodies, single-chain antibodies, and fusion proteins comprising an antigen-binding portion of an antibody and a non-antibody protein. The variable regions of the heavy and light chains of the immunoglobulin molecule contain a binding domain that interacts with an antigen. The constant regions of the antibodies (Abs) may mediate the binding of the immunoglobulin to host tissues.

The term “anti-” or “against” when used before a protein name, anti-DNABII, anti-IHF, anti-HU, anti-tip chimer, anti-PilA, anti-OMP P5, for example, refers to a monoclonal or polyclonal antibody or a biologically active fragment thereof that binds and/or has an affinity to a particular protein. For example, “anti-IHF” refers to an antibody that binds to the IHF protein. The specific antibody may have affinity or bind to proteins other than the protein it was raised against. For example, anti-IHF, while specifically raised against the IHF protein, may also bind other proteins that are related either through sequence homology or through structure homology.

Complementarity determining regions (CDRs) are part of the variable region of an antibody or a T cell receptor generated by B-cell s and T-cells respectively, wherein these molecules bind to their specific antigen (also called epitope). In certain embodiments, the terms “variable region” and “variable domain” are used interchangeably, referring to the polypeptide of a light or heavy chain of an antibody that varies greatly in its sequence of amino acid residues from one antibody to another, and that determines the conformation of the combining site which confers the specificity of the antibody for a particular antigen. In a further embodiment, the variable region is about 90 amino acids long to about 200 amino acids long, including but not limited to about 100 amino acids long, or alternatively about 110 amino acids long, or alternatively about 120 amino acids long, or alternatively about 130 amino acids long, or alternatively about 140 amino acids long, or alternatively about 150 amino acids long, or alternatively about 160 amino acids long, or alternatively about 170 amino acids long, or alternatively about 180 amino acids long, or alternatively about 190 amino acids long. In certain embodiments, a variable region of an amino acid sequence, as used herein, refers to that the first about 100 amino acids, or alternatively about 110 amino acids, or alternatively about 120 amino acids, or alternatively about 130 amino acids, or alternatively about 140 amino acids, or alternatively about 150 amino acids of the amino acid sequence (including or excluding a signal peptide if applicable) is the variable region.

A set of CDRs constitutes a paratope also called an antigen-binding site, which is a part of an antibody that recognizes and binds to an antigen. There are three CDRs (CDR1, CDR2 and CDR3), arranged non-consecutively, optionally from the amino terminus to the carboxyl terminus, on the amino acid sequence of a variable region of an antigen receptor, such as a heavy chain or a light chain. As used herein, CDRn refers to a CDRn in an immunoglobulin chain or derived from an immunoglobulin chain, wherein the number n is selected from 1-3. In one embodiment, CDRLn refers to a CDRn in a light chain or derived from a light chain, wherein the number n is selected from 1-3; while CDRHn refers to a CDRn in a heavy chain or derived from a heavy chain, wherein the number n is selected from 1-3. In certain embodiments, framework region (FR) refers to the part of a variable region which is not a CDR. In certain embodiments, FRn refers to a FR in a heavy chain or a light chain or derived from a heavy chain or a light chain, and wherein the number n is selected from 1-4. In certain embodiments, a variable region comprises or consists essentially of, or yet further consists of the following (optionally following the order as provided, and further optionally from the amino terminus to the carboxyl terminus): FR1, CDR1, FR2, CDR2, FR3, CDR3 and FR4.

Variable regions and/or CDRs of an antibody or a fragment thereof can be determined by one of skill in the art, for example, using publically or commercially available tools. Non-limiting examples of such tools include, IgBlast (accessible at www.ncbi.nlm.nih.gov/igblast/), Scaligner (available from drugdesigntech at www.scaligner.com/), IMGT rules and/or tools (see, for example, www.imgt.org/IMGTScientificChart/Nomenclature/IMGT-FRCDRdefinition.html, also accessible at www.imgt.org/), Chothia Canonical Assignment (accessible at www.bioinforg.uk/abs/chothia.html), Antigen receptor Numbering And Receptor Classification (ANARCI, accessible at opig.stats.ox.ac.uk/webapps/newsabdab/sabpred/anarci/), the Kabat numbering method/scheme (e.g., Kabat, E. A., et al., (1991) Sequences of Proteins of Immunological Interest, Fifth Edition, U.S. Department of Health and Human Services, NIH Publication No. 91-3242,) or the Paratome web server (accessible at www.ofranlab.org/paratome/, see Vered Kunik, et al, Nucleic Acids Research, Volume 40, Issue W1, Jul. 1, 2012, Pages W521-W524).

The antibodies can be polyclonal, monoclonal, multispecific (e.g., bispecific antibodies), and antibody fragments, so long as they exhibit the desired biological activity. Antibodies can be isolated from any suitable biological source, e.g., murine, rat, sheep and canine. As used herein and unless specifically noted, the term “antibody” includes biologically active fragments thereof, polyclonal, monoclonal, human, humanized, variant, derivatized, chimeric or other known modifications thereof.

The terms “polyclonal antibody” or “polyclonal antibody composition” as used herein refer to a preparation of antibodies that are derived from different B-cell lines. They are a mixture of immunoglobulin molecules secreted against a specific antigen, each recognizing a different epitope.

As used herein, “monoclonal antibody” refers to an antibody obtained from a substantially homogeneous antibody population. Monoclonal antibodies are highly specific, as each monoclonal antibody is directed against a single determinant on the antigen. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like.

Monoclonal antibodies may be generated using hybridoma techniques or recombinant DNA methods known in the art. A hybridoma is a cell that is produced in the laboratory from the fusion of an antibody-producing lymphocyte and a non-antibody producing cancer cell, usually a myeloma or lymphoma. A hybridoma proliferates and produces a continuous sample of a specific monoclonal antibody. Alternative techniques for generating or selecting antibodies include in vitro exposure of lymphocytes to antigens of interest, and screening of antibody display libraries in cells, phage, or similar systems.

The term “human antibody” as used herein, is intended to include antibodies having variable and constant regions derived from human germline immunoglobulin sequences. The human antibodies disclosed herein may include amino acid residues not encoded by human germline immunoglobulin sequences (e.g., mutations introduced by random or site-specific mutagenesis in vitro or by somatic mutation in vivo). However, the term “human antibody” as used herein, is not intended to include antibodies in which CDR sequences derived from the germline of another mammalian species, such as a mouse, have been grafted onto human framework sequences. Thus, as used herein, the term “human antibody” refers to an antibody in which substantially every part of the protein (e.g., CDR, framework, C_{L}, C_{H} domains (e.g., C_{H1}, C_{H2}, C_{H3}), hinge, (V_{L}, V_{H})) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

As used herein, a human antibody is “derived from” a particular germline sequence if the antibody is obtained from a system using human immunoglobulin sequences, e.g., by immunizing a transgenic mouse carrying human immunoglobulin genes or by screening a human immunoglobulin gene library. A human antibody that is “derived from” a human germline immunoglobulin sequence can be identified as such by comparing the amino acid sequence of the human antibody to the amino acid sequence of human germline immunoglobulins. A selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

As used herein, the term “humanized antibody” or “humanized immunoglobulin” refers to a human/non-human chimeric antibody that contains a minimal sequence derived from non-human immunoglobulin. For the most part, humanized antibodies are human immunoglobulins (recipient antibody) in which residues from a variable region or a fragment thereof (for example, 1, 2, 3, 4, 5, or all 6 CDRs) of the recipient are replaced by residues from a variable region or a fragment thereof (for example, 1, 2, 3, 4, 5, or all 6 CDRs) of a non-human species (donor antibody) such as mouse, rat, rabbit, or non-human primate having the desired specificity, affinity and capacity. Humanized antibodies may comprise residues that are not found in the recipient antibody or in the donor antibody. The humanized antibody can optionally also comprise at least a portion of an immunoglobulin constant region (Fc), typically that of a human immunoglobulin, a non-human antibody containing one or more amino acids in a framework region, a constant region or a CDR, that have been substituted with a correspondingly positioned amino acid from a human antibody. Without wishing to be bound by the theory, humanized antibodies produce a reduced immune response in a human host, as compared to a non-humanized version of the same antibody. The humanized antibodies may have conservative amino acid substitutions which have substantially no effect on antigen binding or other antibody functions. Conservative substitutions groupings include: glycine-alanine, valine-leucine-isoleucine, phenylalanine-tyrosine, lysine-arginine, alanine-valine, serine-threonine and asparagine-glutamine. Specifically, the humanized antibodies as disclosed herein specifically binds to a DNABII polypeptide or a fragment thereof (such as the tip chimeric peptide or the tail chimeric peptide) with certain range(s) of one or more of the following: EC₅₀, Kₒₙ, K_{off}, K_{A} and/or K_{D}, and inhibits or releases certain cytokine(s) upon treating a subject. In a further embodiment, the humanized antibody specifically binding to the tip region of a DNABII polypeptide (such as the tip chimeric peptide) disrupts biofilm both in vivo and in vitro. In addition, the process of humanization, while a rational design process, may produce unexpected changes (positive or negative) in e.g. binding affinity, antigen specificity, or physical properties such as solubility or aggregability; hence, properties of humanized antibodies are not inherently predictable from the properties of the starting non-human antibody.

In one embodiment, an antibody as used herein may be a recombinant antibody. The term “recombinant antibody”, as used herein, includes all antibodies that are prepared, expressed, created or isolated by recombinant means, such as antibodies isolated from an animal (e.g., a mouse) that is transgenic or transchromosomal for immunoglobulin genes or a hybridoma prepared therefrom, antibodies isolated from a host cell transformed to express the antibody, e.g., from a transfectoma, antibodies isolated from a recombinant, combinatorial antibody library, and antibodies prepared, expressed, created or isolated by any other means that involve splicing of immunoglobulin (Ig) gene sequences to other DNA sequences. In certain embodiments, however, such recombinant antibodies can be subjected to in vitro mutagenesis (or, when an animal transgenic for Ig sequences is used, in vivo somatic mutagenesis) and thus the amino acid sequences of the V_{H} and V_{L} regions of the recombinant antibodies are sequences that may not naturally exist within the antibody germline repertoire in vivo. Methods to making these antibodies are described herein.

In one embodiment, an antibody as used herein may be a chimeric antibody. As used herein, chimeric antibodies are antibodies whose light and heavy chain genes have been constructed, typically by genetic engineering, from antibody variable and constant region genes belonging to different species.

An antibody as used herein may be an “antibody derivative”. In some embodiments, an antibody derivative comprises a full-length antibody or a fragment of an antibody, wherein one or more of the amino acids are chemically modified by alkylation, pegylation, acylation, ester formation or amide formation or the like, e.g., for linking the antibody to a second molecule. This includes, but is not limited to, pegylated antibodies, cysteine-pegylated antibodies, and variants thereof. In some embodiments, the term “antibody derivative” is intended to encompass molecules that bind an epitope as defined above and which are modifications or derivatives of a native monoclonal antibody of this disclosure. Derivatives include, but are not limited to, for example, bispecific, multispecific, heterospecific, trispecific, tetraspecific, multispecific antibodies, diabodies, chimeric, recombinant and humanized.

An antibody as used herein may be an “antibody variant”, which intends to include antibodies produced in a species other than a mouse. It also includes antibodies containing post-translational modifications to the linear polypeptide sequence of the antibody or fragment. It further encompasses fully human antibodies.

The terms “antigen” and “antigenic” refer to molecules with the capacity to be recognized by an antibody or otherwise act as a member of an antibody-ligand pair. “Specific binding” or “binding” refers to the interaction of an antigen with the variable regions of immunoglobulin heavy and light chains. Antibody-antigen binding may occur in vivo or in vitro. The skilled artisan will understand that macromolecules, including proteins, nucleic acids, fatty acids, lipids, lipopolysaccharides and polysaccharides have the potential to act as an antigen. The skilled artisan will further understand that nucleic acids encoding a protein with the potential to act as an antibody ligand necessarily encode an antigen. The artisan will further understand that antigens are not limited to full-length molecules, but can also include partial molecules. The term “antigenic” is an adjectival reference to molecules having the properties of an antigen. The term encompasses substances which are immunogenic, i.e., immunogens, as well as substances which induce immunological unresponsiveness, or anergy, i.e., anergens.

As used herein, the term “specifically binding,” refers to the interaction between binding pairs (e.g., an antibody and an antigen, or a receptor and a ligand). In various instances, specifically binding can be embodied by an affinity constant of about 10⁻⁶ moles/liter, about 10⁻⁷ moles/liter, or about 10⁻⁸ moles/liter, or less.

An immunodominant antigen intends a region of the protein that is recognized and binds with high affinity to an antibody. In some embodiments, an immunoprotective antigen intends a region of the protein that is recognized and binds with high affinity to an antibody to interfere with protein function; the antibodies generated against an immunoprotective antigen are characterized by enhanced or optimal effect against a target indication as a result to the interference with protein function—in some embodiments, an improve capability to clear biofilms.

An “altered antigen” is one having a primary sequence that is different from that of the corresponding wild-type antigen. Altered antigens can be made by synthetic or recombinant methods and include, but are not limited to, antigenic peptides that are differentially modified during or after translation, e.g., by phosphorylation, glycosylation, cross-linking, acylation, proteolytic cleavage, linkage to an antibody molecule, membrane molecule or other ligand. See, e.g., Ferguson et al. (1988) Ann. Rev. Biochem. 57:285-320. A synthetic or altered antigen disclosed herein is intended to bind to the same CDR sets and/or TCR as the natural epitope.

“Immune response” broadly refers to the antigen-specific responses of lymphocytes to foreign substances. The terms “immunogen” and “immunogenic” refer to molecules with the capacity to elicit an immune response. All immunogens are antigens, however, not all antigens are immunogenic. An immune response disclosed herein can be humoral (via antibody activity) or cell-mediated (via T cell activation). The response may occur in vivo or in vitro. The skilled artisan will understand that a variety of macromolecules, including proteins, nucleic acids, fatty acids, lipids, lipopolysaccharides and polysaccharides have the potential to be immunogenic. The skilled artisan will further understand that nucleic acids encoding a molecule capable of eliciting an immune response necessarily encode an immunogen. The artisan will further understand that immunogens are not limited to full-length molecules, but may include partial molecules.

The term “passive immunity” refers to the transfer of immunity from one subject to another through the transfer of antibodies. Passive immunity may occur naturally, as when maternal antibodies are transferred to a fetus. Passive immunity may also occur artificially as when antibody compositions are administered to non-immune subjects. Antibody donors and recipients may be human or non-human subjects. Antibodies may be polyclonal or monoclonal, may be generated in vitro or in vivo, and may be purified, partially purified, or unpurified depending on the embodiment. In some embodiments described herein, passive immunity is conferred on a subject in need thereof through the administration of antibodies or antigen binding fragments that specifically recognize or bind to a particular antigen. In some embodiments, passive immunity is conferred through the administration of an isolated or recombinant polynucleotide encoding an antibody or antigen binding fragment that specifically recognizes or binds to a particular antigen.

The term “propagate” means to grow a cell or population of cells. The term “growing” also refers to the proliferation of cells in the presence of supporting media, nutrients, growth factors, support cells, or any chemical or biological compound necessary for obtaining the desired number of cells or cell type.

The term “culturing” refers to the in vitro propagation of cells or organisms on or in media of various kinds. It is understood that the descendants of a cell grown in culture may not be completely identical (i.e., morphologically, genetically, or phenotypically) to the parent cell.

A “cell” or “host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. In some embodiments, the cell is a eukaryotic cell. In other embodiments, the cell is a prokaryotic cell.

A population of cells intends a collection of more than one cell that is identical (clonal) or non-identical in phenotype and/or genotype. The population can be purified, highly purified, substantially homogenous or heterogeneous as described herein.

A “gene delivery vehicle” is defined as any molecule that can carry inserted polynucleotides into a host cell. Examples of gene delivery vehicles are liposomes, micelles biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A polynucleotide disclosed herein can be delivered to a cell or tissue or a subject using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmid vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene. This is a cheap and easy way of mass-producing a gene or the protein it then codes for.

A “yeast artificial chromosome” or “YAC” refers to a vector used to clone large DNA fragments (larger than 100 kb and up to 3000 kb). It is an artificially constructed chromosome and contains the telomeric, centromeric, and replication origin sequences needed for replication and preservation in yeast cells. Built using an initial circular plasmid, they are linearized by using restriction enzymes, and then DNA ligase can add a sequence or gene of interest within the linear molecule by the use of cohesive ends. Yeast expression vectors, such as YACs, YIps (yeast integrating plasmid), and YEps (yeast episomal plasmid), are extremely useful as one can get eukaryotic protein products with posttranslational modifications as yeasts are themselves eukaryotic cells, however YACs have been found to be more unstable than BACs, producing chimeric effects.

A “viral vector” is defined as a recombinantly produced virus or viral particle that comprises a polynucleotide to be delivered into a host cell, either in vivo, ex vivo or in vitro. Examples of viral vectors include retroviral vectors, lentiviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. In aspects where gene transfer is mediated by a retroviral vector, a vector construct refers to the polynucleotide comprising the retroviral genome or part thereof, and a therapeutic gene.

As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., PCT International Application Publication No. WO 95/27071. Ads do not require integration into the host cell genome. Recombinant Ad derived vectors, particularly those that reduce the potential for recombination and generation of wild-type virus, have also been constructed. See, PCT International Application Publication Nos. WO 95/00655 and WO 95/11984, Wild-type AAV has high infectivity and specificity integrating into the host cell's genome. See, Hermonat & Muzyczka (1984) Proc. Natl. Acad. Sci. USA 81:6466-6470 and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include DNA/liposome complexes, micelles and targeted viral protein-DNA complexes. Liposomes that also comprise a targeting antibody or fragment thereof can be used in the methods disclosed herein. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins disclosed herein are other non-limiting techniques

In aspects where gene transfer is mediated by a lentiviral vector, a vector construct refers to the polynucleotide comprising the lentiviral genome or part thereof, and a therapeutic gene. As used herein, “lentiviral mediated gene transfer” or “lentiviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. Retroviruses carry their genetic information in the form of RNA; however, once the virus infects a cell, the RNA is reverse-transcribed into the DNA form which integrates into the genomic DNA of the infected cell. The integrated DNA form is called a provirus. As used herein, lentiviral vector refers to a viral particle capable of introducing exogenous nucleic acid into a cell through a viral or viral-like entry mechanism. A “lentiviral vector” is a type of retroviral vector well-known in the art that has certain advantages in transducing nondividing cells as compared to other retroviral vectors. See, Trono D. (2002) Lentiviral vectors, New York: Spring-Verlag Berlin Heidelberg.

Lentiviral vectors of this disclosure are based on or derived from oncoretroviruses (the sub-group of retroviruses containing MLV), and lentiviruses (the sub-group of retroviruses containing HIV). Examples include ASLV, SNV and RSV all of which have been split into packaging and vector components for lentiviral vector particle production systems. The lentiviral vector particle according to the disclosure may be based on a genetically or otherwise (e.g. by specific choice of packaging cell system) altered version of a particular retrovirus.

That the vector particle according to the disclosure is “based on” a particular retrovirus means that the vector is derived from that particular retrovirus. The genome of the vector particle comprises components from that retrovirus as a backbone. The vector particle contains essential vector components compatible with the RNA genome, including reverse transcription and integration systems. Usually these will include gag and pol proteins derived from the particular retrovirus. Thus, the majority of the structural components of the vector particle will normally be derived from that retrovirus, although they may have been altered genetically or otherwise so as to provide desired useful properties. However, certain structural components and in particular the env proteins, may originate from a different virus. The vector host range and cell types infected or transduced can be altered by using different env genes in the vector particle production system to give the vector particle a different specificity.

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11 sequentially numbered, AAV serotypes are known in the art. Non-limiting exemplary serotypes useful in the methods disclosed herein include any of the 11 serotypes, e.g., AAV2, AAV8, AAV9, or variant or synthetic serotypes, e.g., AAV-DJ and AAV PHP.B. The AAV particle comprises, alternatively consists essentially of, or yet further consists of three major viral proteins: VP1, VP2 and VP3. In one embodiment, the AAV refers to of the serotype AAV1, AAV2, AAV4, AAV5, AAV6, AAV7, AAV8, AAV9, AAV10, AAV 11, AAV12, AAV13, AAV PHP.B, or AAV rh74. These vectors are commercially available or have been described in the patent or technical literature.

“Detectable label”, “label”, “detectable marker” or “marker” are used interchangeably, including, but not limited to radioisotopes, fluorochromes, chemiluminescent compounds, dyes, and proteins, including enzymes. Detectable labels can also be attached to a polynucleotide, polypeptide, antibody or composition described herein.

As used herein, the term “label” or a detectable label intends a directly or indirectly detectable compound or composition that is conjugated directly or indirectly to the composition to be detected, e.g., N-terminal histidine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of a luminescence signal. Suitable methods and luminophores for luminescently labeling assay components are known in the art and described for example in Haugland, Richard P. (1996) Handbook of Fluorescent Probes and Research Chemicals (6th ed). Examples of luminescent probes include, but are not limited to, aequorin and luciferases.

As used herein, the term “immunoconjugate” comprises an antibody or an antibody derivative associated with or linked to a second agent, such as a cytotoxic agent, a detectable agent, a radioactive agent, a targeting agent, a human antibody, a humanized antibody, a chimeric antibody, a synthetic antibody, a semisynthetic antibody, or a multispecific antibody.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6th ed.).

In another aspect, the fluorescent label is functionalized to facilitate covalent attachment to a cellular component present in or on the surface of the cell or tissue such as a cell surface marker. Suitable functional groups, include, but are not limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

As used herein, a purification label or maker refers to a label that may be used in purifying the molecule or component that the label is conjugated to, such as an epitope tag (including but not limited to a Myc tag, a human influenza hemagglutinin (HA) tag, a FLAG tag), an affinity tag (including but not limited to a glutathione-S transferase (GST), a poly-Histidine (His) tag, Calmodulin Binding Protein (CBP), or Maltose-binding protein (MBP)), or a fluorescent tag.

The terms or “acceptable,” “effective,” or “sufficient” when used to describe the selection of any components, ranges, dose forms, etc. disclosed herein intend that said component, range, dose form, etc. is suitable for the disclosed purpose.

In the context of this disclosure, a “ligand” is a polypeptide. In one aspect, the term “ligand” as used herein refers to any molecule that binds to a specific site on another molecule. In other words, the ligand confers the specificity of the protein in a reaction with an immune effector cell or an antibody to a protein or DNA to a protein. In one aspect it is the ligand site within the protein that combines directly with the complementary binding site on the immune effector cell.

As used herein, the terms “treating,” “treatment” and the like are used herein to mean obtaining a beneficial or desired pharmacologic and/or physiologic effect, and/or desired results, including clinical results. In some embodiments, the effect and/or results can be prophylactic in terms of one or more of: completely or partially preventing a disorder or sign or symptom thereof, and/or can be therapeutic in terms of a partial or complete cure for a disorder and/or adverse effect attributable to the disorder, and/or alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Examples of “treatment” include but are not limited to: preventing a disorder from occurring in a subject that may be predisposed to a disorder, but has not yet been diagnosed as having it; inhibiting a disorder, i.e., arresting its development; and/or relieving or ameliorating the symptoms of disorder. In one aspect, treatment is the arrestment of the development of symptoms of the disease or disorder. In some embodiments, they refer to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. In one aspect, treatment excludes prophylaxis or prevention.

In some embodiments, another beneficial or desired pharmacologic and/or physiologic effect achieved by the treatment may comprise, or consist essentially of, or yet further consist of one or more of the following: reducing dose of an antibiotic compared to a treatment using the antibiotic only, shorter course of using an antibiotic compared to a treatment using the antibiotic only, less frequent of using an antibiotic compared to a treatment using the antibiotic only, or less side effects (such as a milder side effect, free of the side effect, or less frequency of suffering from a side effect, shorter duration of suffering from a side effect) due to use of an antibiotic compared to a treatment using the antibiotic only. Such side effects may comprise, or consist essentially of, or yet further consist of one or more of: skin rashes, diarrhea and life-long disruption of the gut microbiome.

To prevent intends to prevent a disease or disorder or effect in vitro or in vivo in a system or subject that is predisposed to the disorder or effect. An example of such is preventing the formation of a biofilm in a system that is infected with a microorganism known to produce one.

In some embodiments, “treating a biofilm” or a grammatical variation thereof refers to breaking down the biofilm (such as via dispersal and/or disruption). Additionally or alternatively, “treating a biofilm” may also refer to reduction and/or elimination of microbial load (e.g., shown as numbers and/or concentrations) of a microbe forming the biofilm. In some embodiments, the microbial load is calculated based on microbes in any one or two or all three of the following forms: planktonic, biofilm or newly released. In some embodiments, the microbial load is calculated only based on microbes in the biofilm.

In one embodiment, the term “disease” or “disorder” as used herein refers to a disease such as those caused by and/or related to a polymicrobial biofilm, a status of being diagnosed with such disease, a status of being suspect of having such disease, or a status of at high risk of having such disease. In some embodiments, the disease is selected from: pneumonia, sinusitis, septicemia, endocarditis, epiglottitis, Septic arthritis, meningitis, postpartum and neonatal infections, postpartum and neonatal sepsis, acute and chronic salpingitis, pericardis, cellulitis, osteomyelitis, endocarditis, cholecystitis, intraabdominal infections, urinary tract infection, mastoiditis, aortic graft infection, conjunctitivitis, Brazilian pur puric fever, occult bacteremia and exacerbation of underlying lung diseases such as chronic bronchitis, bronchietasis and cystic fibrosis. Additionally or alternatively, the disease is selected from: cystic fibrosis (CF), lung CF, chronic obstructive pulmonary disease (COPD), chronic rhinosinusitis (CRS), periodontitis, periimplantitis, middle ear infection, otitis media, (OM), acute otitis media (AOM), otitis media with effusion (OME), post-tympanostomy tube otorrhea (PTTO), ventilator-associated pneumonia (VAP), community-acquired pneumonia (CAP), chest tube/catheter/indwelling device biofilm infections, tonsillitis, pharyngitis, laryngitis, epiglottitis, sinusitis, pneumonia, bronchitis, or other respiratory tract infection (RTI). In some embodiments, the disease is any pathological condition involving H. influenzae (typeable and nontypeable strains) such as OM, pneumonia, sinusitis, septicemia, endocarditis, epiglottitis, septic arthritis, meningitis, postpartum and neonatal infections, postpartum and neonatal sepsis, acute and chromic salpingitis, epiglottis, pericardis, cellulitis, osteomyelitis, endocarditis, cholecystitis, intraabdominal infections, urinary tract infection, mastoiditis, aortic graft infection, conjunctitivitis, Brazilian purpuric fever, occult bacteremia, chronic obstructive pulmonary disease and exacerbation of underlying lung diseases such as chronic bronchitis, bronchietasis and cystic fibrosis. In some embodiments, the disease is any disorder of a subject caused directly or indirectly by a bacterium biofilm as disclosed herein, such as NTHI. In some embodiments, the biofilm is a polymicrobial biofilm. In some aspects, the polymicrobial biofilm is caused by Haemophilus, e.g. NTHI and one or more of Burkholderia cenocepacia, Staphylococcus aureus, Streptococcus pneumonia, or Moraxella catarrhalis.

“Administration” or “delivery” of an antibody, vaccine, peptide, cell or vector or other agent and compositions containing same can be effected in one dose, continuously or intermittently throughout the course of treatment. Methods of determining the most effective means and dosage of administration are known to those of skill in the art and will vary with the composition used for therapy, the purpose of the therapy, the target infection or biofilm being treated, and the subject being treated. Single or multiple administrations can be carried out with the dose level and pattern being selected by the treating physician or in the case of animals, by the treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, intraperitoneal, infusion, nasal administration, inhalation, injection, and topical application.

The term administration shall include without limitation, administration by oral, parenteral (e.g., intramuscular, intraperitoneal, intravenous, intracerebroventricular (ICV), intrathecal, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The disclosure is not limited by the route of administration, the formulation or dosing schedule.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art.

An agent of the present disclosure can be administered for therapy by any suitable route of administration. It will also be appreciated that the optimal route will vary with the condition and age of the recipient, and the disease being treated.

As used herein, the term “contacting” means direct or indirect binding or interaction between two or more molecules. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

A “composition” is intended to mean a combination of active agent and another compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton). In some embodiments, “pharmaceutically acceptable carriers” refer to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

The term pharmaceutically acceptable carrier (or medium), which may be used interchangeably with the term biologically compatible carrier or medium, refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio. Pharmaceutically acceptable carriers suitable for use in the present disclosure include liquids, semi-solid (e.g., gels) and solid materials (e.g., cell scaffolds and matrices, tubes sheets and other such materials as known in the art and described in greater detail herein). These semi-solid and solid materials may be designed to resist degradation within the body (non-biodegradable) or they may be designed to degrade within the body (biodegradable, bioerodable). A biodegradable material may further be bioresorbable or bioabsorbable, i.e., it may be dissolved and absorbed into bodily fluids (water-soluble implants are one example), or degraded and ultimately eliminated from the body, either by conversion into other materials or breakdown and elimination through natural pathways.

As used herein, “solid phase support” or “solid support”, used interchangeably, is not limited to a specific type of support. Rather a large number of supports are available and are known to one of ordinary skill in the art. Solid phase supports include silica gels, resins, derivatized plastic films, glass beads, cotton, plastic beads, alumina gels. As used herein, “solid support” also includes synthetic antigen-presenting matrices, cells, and liposomes. A suitable solid phase support may be selected on the basis of desired end use and suitability for various protocols. For example, for peptide synthesis, solid phase support may refer to resins such as polystyrene (e.g., PAM-resin obtained from Bachem Inc., Peninsula Laboratories, etc.), POLYHIPE® resin (obtained from Aminotech, Canada), polyamide resin (obtained from Peninsula Laboratories), polystyrene resin grafted with polyethylene glycol (TentaGel®, Rapp Polymere, Tubingen, Germany) or polydimethylacrylamide resin (obtained from Milligen/Biosearch, Calif.).

An example of a solid phase support include glass, polystyrene, polypropylene, polyethylene, dextran, nylon, amylases, natural and modified celluloses, polyacrylamides, gabbros, and magnetite. The nature of the carrier can be either soluble to some extent or insoluble. The support material may have virtually any possible structural configuration so long as the coupled molecule is capable of binding to a polynucleotide, polypeptide or antibody. Thus, the support configuration may be spherical, as in a bead, or cylindrical, as in the inside surface of a test tube, or the external surface of a rod. Alternatively, the surface may be flat such as a sheet, test strip, etc. or alternatively polystyrene beads. Those skilled in the art will know many other suitable carriers for binding antibody or antigen, or will be able to ascertain the same by use of routine experimentation.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

A “subject” (which is used herein interchangeably with “individual” or “patient”) of diagnosis or treatment is a cell or an animal such as a mammal, or a human. Non-human animals subject to diagnosis or treatment and are those subject to infections or animal models, for example, simians, murines, such as, rats, mice, chinchilla, canine, such as dogs, leporids, such as rabbits, livestock, sport animals, and pets. The term “subject,” “host,” “individual,” and “patient” are as used interchangeably herein to refer to animals, typically mammalian animals. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, a pediatric patient, an adolescent, teen, a pre-pubescent subject, child, infant, a fetus or a mammal in utero). A mammal can be male or female. In some embodiments a subject is a human. In one aspect, the subject is a pre-symptomatic subject. In another aspect, the subject has minimal clinical symptoms of the disease. The subject can be a male or a female, adult, an infant or a pediatric subject. In an additional aspect, the subject is an adult. In some instances, the adult is an adult human, e.g., an adult human greater than 18 years of age.

As used herein, a biological sample, or a sample, can be obtained from a subject, cell line or cultured cell or tissue. Exemplary samples include, but are not limited to, cell sample, tissue sample, liquid samples such as blood and other liquid samples of biological origin (including, but not limited to, ocular fluids (aqueous and vitreous humor), peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood. In one embodiment, the biological sample is suspect of having a biofilm. In another embodiment, the biological sample comprise a biofilm. In some embodiments, the sample is from a subject's airway, such as mucus.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present disclosure for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks. In the context of an immunogenic composition, in some embodiments the effective amount is the amount sufficient to result in a protective response against a pathogen. In other embodiments, the effective amount of an immunogenic composition is the amount sufficient to result in antibody generation against the antigen. In some embodiments, the effective amount is the amount required to confer passive immunity on a subject in need thereof. With respect to immunogenic compositions, in some embodiments the effective amount will depend on the intended use, the degree of immunogenicity of a particular antigenic compound, and the health/responsiveness of the subject's immune system, in addition to the factors described above. The skilled artisan will be able to determine appropriate amounts depending on these and other factors.

An “immunogenic dose” of a composition is one that generates, after administration, a detectable humoral (antibody) and/or cellular (T cell) immune response in comparison to the immune response detectable before administration or in comparison to a standard immune response before administration. In some embodiments, the immune response results from the methods may be protective and/or therapeutic. In some embodiments, the antibody and/or T cell immune response protects the individual from a biofilm infection as disclosed herein, particularly infection of the middle ear and/or the nasopharynx or lower airway. In this use, the precise dose depends on the patient's state of health and weight, the mode of administration, the nature of the formulation, etc., but generally ranges from about 1.0.mu.g to about 5000.mu.g per 70 kilogram patient, more commonly from about 10 to about 500 .mu.g per 70 kg of body weight.

Humoral immune response may be measured by many well known methods, such as Single Radial Immunodiffusion Assay (SRID), Enzyme Immunoassay (EIA) and Hemagglutination Inhibition Assay (HAI). In particular, SRID utilizes a layer of a gel, such as agarose, containing the immunogen being tested. A well is cut in the gel and the serum being tested is placed in the well. Diffusion of the antibody out into the gel leads to the formation of a precipitation ring whose area is proportional to the concentration of the antibody in the serum being tested. EIA, also known as ELISA (Enzyme Linked Immunoassay), is used to determine total antibodies in the sample. The immunogen is adsorbed to the surface of a microtiter plate. The test serum is exposed to the plate followed by an enzyme linked immunoglobulin, such as IgG. The enzyme activity adherent to the plate is quantified by any convenient means such as spectrophotometry and is proportional to the concentration of antibody directed against the immunogen present in the test sample. HAI utilizes the capability of an immunogen such as viral proteins to agglutinate chicken red blood cells (or the like). The assay detects neutralizing antibodies, i.e., those antibodies able to inhibit hemagglutination. Dilutions of the test serum are incubated with a standard concentration of immunogen, followed by the addition of the red blood cells. The presence of neutralizing antibodies will inhibit the agglutination of the red blood cells by the immunogen. Tests to measure cellular immune response include determination of delayed-type hypersensitivity or measuring the proliferative response of lymphocytes to target immunogen.

In the case of an in vitro application, in some embodiments the effective amount will depend on the size and nature of the application in question. It will also depend on the nature and sensitivity of the in vitro target and the methods in use. The skilled artisan will be able to determine the effective amount based on these and other considerations. The effective amount may comprise one or more administrations of a composition depending on the embodiment.

### Modes for Carrying Out the Disclosure

In one aspect, provided is a method for one or more of: preventing, inhibiting, disrupting, dispersing or treating a polymicrobial biofilm that was caused by, will be caused by, or comprises a Haemophilus bacteria in a subject in need thereof. The method comprises, or consists essentially of, or yet further consists of administering to the subject: (i) an anti-DNABII antibody or a biologically active fragment thereof and an anti-PilA antibody or biologically active fragment thereof; or (ii) an anti-DNABII antibody or a biologically active fragment thereof and a PilA polypeptide or a biologically active fragment thereof. The antibody can be a polyclonal antibody or monoclonal antibody, derivative, variant fragment thereof. In one aspect, the anti-DNABII antibody comprises one or more of an anti-DNABII antibody, an anti-DNABII tip antibody, an anti-DNABII tip chimer antibody or biologically active fragment of each thereof. The antibody can be a polyclonal antibody or a monoclonal antibody. In one aspect, the anti-DNABII antibody comprises an anti-DNABII tip antibody or an anti-tip chimer antibody or biologically active fragment or each thereof. In a further aspect, the anti-DNABII tip chimer antibody is a monoclonal antibody, a chimeric or a humanized antibody, e.g., as disclosed herein. In another aspect, the anti-PilA antibody specifically recognizes and binds the PilA peptide, examples of such include PilA surface antigens or recombinant soluble PilA (Genbank Accession No.: EFU96734.1) and Type IV Pilin (Genbank Accession No.: Yp-003864351.1). The antibodies can be detectably labeled. In some aspects, the polymicrobial biofilm is caused by Haemophilus, e.g. NTHI and one or more of Burkholderia cenocepacia, Staphylococcus aureus, Streptococcus pneumonia, or Moraxella catarrhalis. In some aspects, the polymicrobial biofilm also is caused by a gram-positive or a gram-negative bacteria, e.g., an ESKAPE bacteria. In some embodiments, the method further comprises administering to the subject an antibiotic. In further embodiments, the antibiotic comprises, or consists essentially of, or yet further consists of a β-lactam antibiotic or a sulfonamide antibiotic. In one aspect, the subject is a mammal or a human patient.

In another aspect, provided is a method for one or more of: preventing, inhibiting, disrupting, dispersing or treating a disease related to a polymicrobial biofilm infection that was caused by, will be caused by, or comprises a Haemophilus bacteria in a subject in need thereof. The method comprises, or consists essentially of, or yet further consists of administering to the subject: (i) an anti-DNABII antibody or a biologically active fragment thereof and an anti-PilA antibody or biologically active fragment thereof, or (ii) an anti-DNABII antibody or a biologically active fragment thereof and a PilA polypeptide or a biologically active fragment thereof. The antibody can be a polyclonal antibody or monoclonal antibody, derivative, variant fragment thereof. In one aspect, the anti-DNABII antibody comprises one or more of an anti-DNABII antibody, an anti-DNABII tip antibody, an anti-DNABII tip chimer antibody or biologically active fragment of each thereof. In one aspect, the anti-DNABII antibody comprises an anti-DNABII tip or tip chimer antibody or biologically active fragment of each thereof. In a further aspect, the anti-DNABII tip chimer antibody is a monoclonal antibody, a chimeric or a humanized antibody, e.g., as disclosed herein. In another aspect, the anti-PilA antibody specifically recognizes and binds the PilA peptide, examples of such include PilA surface antigens or recombinant soluble PilA (Genbank Accession No.: EFU96734.1) and Type IV Pilin (Genbank Accession No.: Yp-003864351.1). The antibodies can be detectably labeled. In some aspects, the polymicrobial biofilm is caused by Haemophilus, e.g. NTHI and one or more of Burkholderia cenocepacia, Staphylococcus aureus, Streptococcus pneumonia, or Moraxella catarrhalis. In some aspects, the polymicrobial biofilm also is caused by a gram-positive or a gram-negative bacteria, e.g., an ESKAPE bacteria. In some embodiments, the method further comprises administering to the subject an antibiotic. In further embodiments, the antibiotic comprises, or consists essentially of, or yet further consists of a β-lactam antibiotic or a sulfonamide antibiotic. In some embodiments, the disease is as disclosed herein, for example, selected from: cystic fibrosis (CF), lung CF, chronic obstructive pulmonary disease (COPD), chronic rhinosinusitis (CRS), periodontitis, periimplantitis, middle ear infection, otitis media, (OM), acute otitis media (AOM), otitis media with effusion (OME), post-tympanostomy tube otorrhea (PTTO), ventilator-associated pneumonia (VAP), community-acquired pneumonia (CAP), chest tube/catheter/indwelling device biofilm infections, tonsillitis, pharyngitis, laryngitis, epiglottitis, sinusitis, pneumonia, bronchitis, or other respiratory tract infection (RTI). In one aspect, the subject is a mammal or a human patient.

In yet another aspect, provided is a method for sensitizing a polyclonal microbial biofilm that was caused by, will be caused by, or comprises a Haemophilus bacteria in a subject in need thereof for an antibiotic therapy or inducing bacteria of the polymicrobial biofilm in a subject in need thereof to a newly released (NRel) state. The method comprises, or consists essentially of, or yet further consists of administering to the subject: (i) an anti-DNABII antibody or a biologically active fragment thereof and an anti-PilA antibody or biologically active fragment thereof, or (ii) an anti-DNABII antibody or a biologically active fragment thereof and a PilA polypeptide or a biologically active fragment thereof. The antibody can be a polyclonal antibody or monoclonal antibody, derivative, variant fragment thereof. In one aspect, the anti-DNABII antibody comprises one or more of an anti-DNABII antibody, an anti-DNABII tip antibody, an anti-DNABII tip chimer antibody or biologically active fragment of each thereof. In one aspect, the anti-DNABII antibody comprises an anti-DNABII tip chimer antibody or biologically active fragment thereof. The antibody can be a polyclonal antibody or a monoclonal antibody. In one aspect, the anti-DNABII antibody comprises an anti-DNABII tip chimer antibody or biologically active fragment thereof. In a further aspect, the anti-DNABII tip chimer antibody is a monoclonal antibody, a chimeric or a humanized antibody, e.g., as disclosed herein. In another aspect, the anti-PilA antibody specifically recognizes and binds the PilA peptide, examples of such include PilA surface antigens or recombinant soluble PilA (Genbank Accession No.: EFU96734.1) and Type IV Pilin (Genbank Accession No.: Yp-003864351.1). The antibodies can be detectably labeled. In some aspects, the polymicrobial biofilm is caused by Haemophilus, e.g. NTHI and one or more of Burkholderia cenocepacia, Staphylococcus aureus, Streptococcus pneumonia, or Moraxella catarrhalis. In some aspects, the polymicrobial biofilm also is caused by a gram-positive or a gram-negative bacteria, e.g., an ESKAPE bacteria. In some embodiments, the method further comprises administering to the subject an antibiotic. In further embodiments, the antibiotic comprises, or consists essentially of, or yet further consists of a β-lactam antibiotic or a sulfonamide antibiotic. In one aspect, the subject is a mammal or a human patient.

In one aspect, provided is a method for one or more of: preventing, inhibiting, disrupting, dispersing or treating a polymicrobial biofilm that was caused by, will be caused by, or comprises a Haemophilus bacteria. The method comprises, or consists essentially of, or yet further consists of administering to the subject: (i) an anti-DNABII antibody or a biologically active fragment thereof and an anti-PilA antibody or biologically active fragment thereof; or (ii) an anti-DNABII antibody or a biologically active fragment thereof and a PilA polypeptide or a biologically active fragment thereof. The antibody can be a polyclonal antibody or monoclonal antibody, derivative, variant fragment thereof. In one aspect, the anti-DNABII antibody comprises an anti-DNABII tip chimer antibody or biologically active fragment thereof. The antibody can be a polyclonal antibody or a monoclonal antibody. In one aspect, the anti-DNABII antibody comprises an anti-DNABII tip chimer antibody or biologically active fragment thereof. In one aspect, the anti-DNABII antibody comprises an anti-DNABII tip chimer antibody or biologically active fragment thereof. In a further aspect, the anti-DNABII tip chimer antibody is a monoclonal antibody, a chimeric or a humanized antibody, e.g., as disclosed herein. In another aspect, the anti-PilA antibody specifically recognizes and binds the PilA peptide, examples of such include PilA surface antigens or recombinant soluble PilA (Genbank Accession No.: EFU96734.1) and Type IV Pilin (Genbank Accession No.: Yp-003864351.1). The antibodies can be detectably labeled. In some aspects, the polymicrobial biofilm is caused by Haemophilus, e.g. NTHI and one or more of Burkholderia cenocepacia, Staphylococcus aureus, Streptococcus pneumonia, or Moraxella catarrhalis. In some aspects, the polymicrobial biofilm also is caused by a gram-positive or a gram-negative bacteria, e.g., an ESKAPE bacteria. In some embodiments, the method further comprises contacting the biofilm with an antibiotic. In further embodiments, the antibiotic comprises, or consists essentially of, or yet further consists of a β-lactam antibiotic or a sulfonamide antibiotic. In some embodiments, the contacting is in vitro or in vivo.

In another aspect, provided is a method for sensitizing a polymicrobial biofilm for an antibiotic therapy or inducing bacteria that forms the polymicrobial biofilm that was caused by, will be caused by, or comprises a Haemophilus bacteria to a newly released (NRel) state. The method comprises, or consists essentially of, or yet further consists of administering to the subject: (i) an anti-DNABII antibody or a biologically active fragment thereof and an anti-PilA antibody or biologically active fragment thereof, or (ii) an anti-DNABII antibody or a biologically active fragment thereof and a PilA polypeptide or a biologically active fragment thereof. The antibody can be a polyclonal antibody or monoclonal antibody, derivative, variant fragment thereof. In one aspect, the anti-DNABII antibody comprises an anti-DNABII tip chimer antibody or biologically active fragment thereof. In one aspect, the anti-DNABII antibody comprises an anti-DNABII tip chimer antibody or biologically active fragment thereof. The antibody can be a polyclonal antibody or a monoclonal antibody. In one aspect, the anti-DNABII antibody comprises an anti-DNABII tip chimer antibody or biologically active fragment thereof. In a further aspect, the anti-DNABII tip chimer antibody is a monoclonal antibody, a chimeric or a humanized antibody, e.g., as disclosed herein. In another aspect, the anti-PilA antibody specifically recognizes and binds the PilA peptide, examples of such include PilA surface antigens or recombinant soluble PilA (Genbank Accession No.: EFU96734.1) and Type IV Pilin (Genbank Accession No.: Yp-003864351.1). The antibodies can be detectably labeled. In some aspects, the polymicrobial biofilm is caused by Haemophilus, e.g. NTHI and one or more of Burkholderia cenocepacia, Staphylococcus aureus, Streptococcus pneumonia, or Moraxella catarrhalis. In some aspects, the polymicrobial biofilm also is caused by a gram-positive or a gram-negative bacteria, e.g., an ESKAPE bacteria. In some embodiments, the method further comprises contacting the biofilm with an antibiotic. In further embodiments, the antibiotic comprises, or consists essentially of, or yet further consists of a β-lactam antibiotic or a sulfonamide antibiotic. In some embodiments, the contacting is in vitro or in vivo.

In some embodiments of any aspect as disclosed herein, the antibiotic is contacted with the biofilm or administrated to the subject concurrently with or after (i) an anti-DNABII antibody or a biologically active fragment thereof and an anti-PilA antibody or biologically active fragment thereof, or (ii) an anti-DNABII antibody or a biologically active fragment thereof and a PilA polypeptide or a biologically active fragment thereof. The antibody can be a polyclonal antibody or monoclonal antibody, derivative, variant fragment thereof. In one aspect, the anti-DNABII antibody comprises an anti-DNABII tip chimer antibody or biologically active fragment thereof. In one aspect, the anti-DNABII antibody comprises an anti-DNABII tip chimer antibody or biologically active fragment thereof. The antibody can be a polyclonal antibody or a monoclonal antibody. In one aspect, the anti-DNABII antibody comprises an anti-DNABII tip chimer antibody or biologically active fragment thereof. In a further aspect, the anti-DNABII tip chimer antibody is a monoclonal antibody, a chimeric or a humanized antibody, e.g., as disclosed herein. In another aspect, the anti-PilA antibody specifically recognizes and binds the PilA peptide, examples of such include PilA surface antigens or recombinant soluble PilA (Genbank Accession No.: EFU96734.1) and Type IV Pilin (Genbank Accession No.: Yp-003864351.1). In some embodiments of any aspect as disclosed herein, the method further comprises identifying the bacteria in a sample of the subject or the polymicrobial biofilm prior to the contacting or administering step. In some aspects, the polymicrobial biofilm also is caused by a gram-positive or a gram-negative bacteria, e.g., an ESKAPE bacteria. In yet further embodiments, the bacteria identified as comprising, or consisting essentially of, or yet further consisting of more than one Haemophilus bacteria. In some embodiments, the bacteria identified as comprising, or consisting essentially of, or yet further consisting of a Haemophilus bacteria and another non-Haemophilus bacteria. In some aspects, the bacteria is Haemophilus, e.g. NTHI and one or more of Burkholderia cenocepacia, Staphylococcus aureus, Streptococcus pneumonia, or Moraxella catarrhalis.

In some embodiments of any aspect as disclosed herein, via applying a method as disclosed herein, the polymicrobial biofilm that was caused by, will be caused by, or comprises a Haemophilus bacteria is one or more of: prevented, inhibited, disrupted, dispersed or treated, in a synergistic manner compared to contacting or administering one of (i) an anti-DNABII antibody or a biologically active fragment thereof and an anti-PilA antibody or biologically active fragment thereof, or (ii) an anti-DNABII antibody or a biologically active fragment thereof and a PilA polypeptide or a biologically active fragment thereof, or an antibiotic alone. In some aspects, the polymicrobial biofilm is caused by Haemophilus, e.g. NTHI and one or more of Burkholderia cenocepacia, Staphylococcus aureus, Streptococcus pneumonia, or Moraxella catarrhalis. In some aspects, the polymicrobial biofilm also is caused by a gram-positive or a gram-negative bacteria, e.g., an ESKAPE bacteria.

In some embodiments of any aspect as disclosed herein, the newly released (NRel) state of the bacteria released from the biofilm is different from the same bacteria but grown planktonically. In some embodiments, the NRel bacteria comprises one or more of the following: an altered gene expression of one or more of the following genes: deaD, artM, fis, folA, folP, emrA, emrB, or acrR; an altered level of one or more of the following proteins: AsnC, CyaA, GlpC, NrfA, TehB, TrpB, TrpC, TrpD, TruA, or TrxA; a low level of one or more of the following proteins: AbgA, AroE, AroK, ArtP, AtpH, BioB, BioF, CcmA, CitD, CitT, CmK, CoaD, DcD, DjlA, DksA, DnaE, DnaQ, DsbE, FabG, FdhX, FtnB, FtsE, FtsI, FumC, Fur, GlpA, GlpB, GmK, GpsA, GreA, Hfq, HisI, HugZ, InfA, InfB, IspD, IspF, Lic2A, LicC, LicD, LolA, LpsA, menC, MenC, NapA, NrfB, NrfC, NTHI0053, NTHI0249, NTHI0291, NTHI0319, NTHI0349, NTHI0436, NTHI0487, NTHI0490, NTHI0555, NTHI0732, NTHI0779, NTHI0820, NTHI1025, NTHI1085, NTHI1199, NTHI1437, NTHI1439, NTHI1503, NTHI1590, NTHI1748, OppF, PanF, PdxH, PepT, PhoB, PlsC, PolA, PpC, PpiB, PrfB, PurU, RadA, RelB, RibA, RplW, RpoE, RpoZ, RseA, SecF, Sxy, TesB, ThrB, TolA, UnG, UreG, UuaP, or UvrB; a high level of one or more of the following proteins: ClpB, CydD, DeaD, DlD, DmsB, ExbD, GroES, HfeA, HfeB, HgpB, HisJ, HisJ, HitA, HktE, HxuC, LctP, LldD, LpxC, MurB, NdhA, NifS2, NTHI0043, NTHI0052, NTHI0175, NTHI0364, NTHI1208, NTHI1214, NTHI1369, NTHI1703, OmpP2, OrfG, Pal, PdgX, RbfA, RecN, RplO, RplU, RpmE, RpsE, RpsJ, RpsL, RpsQ, RpsT, SodA, Tbpl, TolB, TonB, TrpA, TrxA, or ZnuA. In further embodiments, the high (also referred to herein as increased), low (also referred to herein as decreased), altered expression or level is compared to the same bacteria but grown planktonically.

In one aspect, provided is a method for selecting a polymicrobial biofilm suitable for one or more of the following: prevention, inhibition, disruption, dispersion, treatment or sensitization by a method as disclosed herein. The method comprises, or alternatively consists essentially of, or yet further consists of (a) contacting the polymicrobial biofilm, or a polymicrobial biofilm isolated and grown therefrom, with one or both of (i) an anti-DNABII antibody or a biologically active fragment thereof and an anti-PilA antibody or biologically active fragment thereof, or (ii) an anti-DNABII antibody or a biologically active fragment thereof and a PilA polypeptide or a biologically active fragment thereof, as disclosed herein; and (b) assaying released bacteria from the biofilm of (a) for expression of one or more of a gene as disclosed herein and/or a protein as disclosed herein. In some embodiments, the gene is selected from one or more of the following: deaD, artM, fis, folA, folP, emrA, emrB, or acrR. Additionally or alternatively, the protein is selected from one or more of the following: AsnC, CyaA, GlpC, NrfA, TehB, TrpB, TrpC, TrpD, TruA, TrxA; AbgA, AroE, AroK, ArtP, AtpH, BioB, BioF, CcmA, CitD, CitT, CmK, CoaD, DcD, DjlA, DksA, DnaE, DnaQ, DsbE, FabG, FdhX, FtnB, FtsE, FtsI, FumC, Fur, GlpA, GlpB, GmK, GpsA, GreA, Hfq, HisI, HugZ, InfA, InfB, IspD, IspF, Lic2A, LicC, LicD, LolA, LpsA, menC, MenC, NapA, NrfB, NrfC, NTHI0053, NTHI0249, NTHI0291, NTHI0319, NTHI0349, NTHI0436, NTHI0487, NTHI0490, NTHI0555, NTHI0732, NTHI0779, NTHI0820, NTHI1025, NTHI1085, NTHI1199, NTHI1437, NTHI1439, NTHI1503, NTHI1590, NTHI1748, OppF, PanF, PdxH, PepT, PhoB, PlsC, PolA, PpC, PpiB, PrfB, PurU, RadA, RelB, RibA, RplW, RpoE, RpoZ, RseA, SecF, Sxy, TesB, ThrB, TolA, UnG, UreG, UuaP, UvrB; ClpB, CydD, DeaD, DlD, DmsB, ExbD, GroES, HfeA, HfeB, HgpB, HisJ, HisJ, HitA, HktE, HxuC, LctP, LldD, LpxC, MurB, NdhA, NifS2, NTHI0043, NTHI0052, NTHI0175, NTHI0364, NTHI1208, NTHI1214, NTHI1369, NTHI1703, OmpP2, OrfG, Pal, PdgX, RbfA, RecN, RplO, RplU, RpmE, RpsE, RpsJ, RpsL, RpsQ, RpsT, SodA, Tbpl, TolB, TonB, TrpA, TrxA, or ZnuA. The antibody can be a polyclonal antibody or monoclonal antibody, derivative, variant fragment thereof. In one aspect, the anti-DNABII antibody comprises an anti-DNABII tip chimer antibody or biologically active fragment thereof. In one aspect, the anti-DNABII antibody comprises an anti-DNABII tip chimer antibody or biologically active fragment thereof. The antibody can be a polyclonal antibody or a monoclonal antibody. In one aspect, the anti-DNABII antibody comprises an anti-DNABII tip chimer antibody or biologically active fragment thereof. In a further aspect, the anti-DNABII tip chimer antibody is a monoclonal antibody, a chimeric or a humanized antibody, e.g., as disclosed herein. In another aspect, the anti-PilA antibody specifically recognizes and binds the PilA peptide, examples of such include PilA surface antigens or recombinant soluble PilA (Genbank Accession No.: EFU96734.1) and Type IV Pilin (Genbank Accession No.: Yp-003864351.1). The antibodies can be detectably labeled.

In some embodiments, the method further comprises contacting the selected biofilm with (i) an anti-DNABII antibody or a biologically active fragment thereof and an anti-PilA antibody or biologically active fragment thereof, or (ii) an anti-DNABII antibody or a biologically active fragment thereof and a PilA polypeptide or a biologically active fragment thereof; and (iii) an optional antibiotic optionally comprising a β-lactam antibiotic or a sulfonamide antibiotic. In some embodiments, the selected polymicrobial biofilm is in a subject. In further embodiments, the method further comprises administering the subject with: (i) an anti-DNABII antibody or a biologically active fragment thereof, (ii) a PilA polypeptide or a biologically active fragment thereof, and (iii) an optional antibiotic optionally comprising a β-lactam antibiotic or a sulfonamide antibiotic. The antibody can be a polyclonal antibody or monoclonal antibody, derivative, variant fragment thereof. In one aspect, the anti-DNABII antibody comprises an anti-DNABII tip chimer antibody or biologically active fragment thereof. In one aspect, the anti-DNABII antibody comprises an anti-DNABII tip chimer antibody or biologically active fragment thereof. The antibody can be a polyclonal antibody or a monoclonal antibody. In one aspect, the anti-DNABII antibody comprises an anti-DNABII tip chimer antibody or biologically active fragment thereof. In a further aspect, the anti-DNABII tip chimer antibody is a monoclonal antibody, a chimeric or a humanized antibody, e.g., as disclosed herein. In another aspect, the anti-PilA antibody specifically recognizes and binds the PilA peptide, examples of such include PilA surface antigens or recombinant soluble PilA (Genbank Accession No.: EFU96734.1) and Type IV Pilin (Genbank Accession No.: Yp-003864351.1). The antibodies can be detectably labeled.

In some embodiments of any aspect as disclosed herein, one or more of the following indicates the biofilm is suitable for a method as disclosed herein, and thus is selected:
an altered gene expression of one or more of the following genes: deaD, artM, fis, folA, folP, emrA, emrB, or acrR;
an altered level of one or more of the following proteins: AsnC, CyaA, GlpC, NrfA, TehB, TrpB, TrpC, TrpD, TruA, or TrxA;
a low level of one or more of the following proteins: AbgA, AroE, AroK, ArtP, AtpH, BioB, BioF, CcmA, CitD, CitT, CmK, CoaD, DcD, DjlA, DksA, DnaE, DnaQ, DsbE, FabG, FdhX, FtnB, FtsE, FtsI, FumC, Fur, GlpA, GlpB, GmK, GpsA, GreA, Hfq, HisI, HugZ, InfA, InfB, IspD, IspF, Lic2A, LicC, LicD, LolA, LpsA, menC, MenC, NapA, NrfB, NrfC, NTHI0053, NTHI0249, NTHI0291, NTHI0319, NTHI0349, NTHI0436, NTHI0487, NTHI0490, NTHI0555, NTHI0732, NTHI0779, NTHI0820, NTHI1025, NTHI1085, NTHI1199, NTHI1437, NTHI1439, NTHI1503, NTHI1590, NTHI1748, OppF, PanF, PdxH, PepT, PhoB, PlsC, PolA, PpC, PpiB, PrfB, PurU, RadA, RelB, RibA, RplW, RpoE, RpoZ, RseA, SecF, Sxy, TesB, ThrB, TolA, UnG, UreG, UuaP, or UvrB; or
a high level of one or more of the following proteins: ClpB, CydD, DeaD, DlD, DmsB, ExbD, GroES, HfeA, HfeB, HgpB, HisJ, HisJ, HitA, HktE, HxuC, LctP, LldD, LpxC, MurB, NdhA, NifS2, NTHI0043, NTHI0052, NTHI0175, NTHI0364, NTHI1208, NTHI1214, NTHI1369, NTHI1703, OmpP2, OrfG, Pal, PdgX, RbfA, RecN, RplO, RplU, RpmE, RpsE, RpsJ, RpsL, RpsQ, RpsT, SodA, Tbpl, TolB, TonB, TrpA, TrxA, or ZnuA.

In some embodiments of any aspect as disclosed herein, an altered expression refers to both increased and decreased expression.

In some embodiments of any aspect as disclosed herein, a high expression refers to the expression increased by about 10%, or about 20%, or about 30%, or about 40%, or about 50%, or about 60%, or about 70%, or about 80%, or about 90%, or about 1 fold, or about 2 folds, or about 3 folds, or about 4 folds, or about 5 folds, or about 6 folds, or about 7 folds, or about 8 folds, or about 9 folds, or about 10 folds, or about 20 folds, or about 30 folds, or about 40 folds, or about 50 folds, or about 60 folds, or about 70 folds, or about 80 folds, or about 90 folds, or about 100 folds or more higher than the reference.

In some embodiments of any aspect as disclosed herein, a low expression refers to the expression decreased by about 1%, or about 2%, or about 3%, or about 4%, or about 5%, or about 6%, or about 7%, or about 8%, or about 0%, or about 10%, or about 20%, or about 30%, or about 40%, or about 50%, or about 60%, or about 70%, or about 75%, or about 80%, or about 85%, or about 90%, or about 95%, or about 96%, or about 97%, or about 98%, or about 99% of the reference.

In some embodiments of any aspect as disclosed herein, the reference is the corresponding expression of the same bacteria but grown planktonically.

In one aspect, provided is a kit, such as for use in a method as disclosed herein. In some embodiments, the kit comprises, or alternatively consists essentially of, or yet further consists of one or more of the following: (i) an anti-DNABII antibody or a biologically active fragment thereof and an anti-PilA antibody or biologically active fragment thereof; or (ii) an anti-DNABII antibody or a biologically active fragment thereof and a PilA polypeptide or a biologically active fragment thereof, a polynucleotide encoding each element thereof, a vector comprising the polynucleotide or a host cell comprising the polynucleotide and/or the vector and optionally expressing the antibody or fragment thereof, or (iii) an antibiotic optionally comprising a β-lactam antibiotic or a sulfonamide antibiotic, and optionally instructions for use. The antibody can be a polyclonal antibody or monoclonal antibody, derivative, variant fragment thereof. In one aspect, the anti-DNABII antibody comprises an anti-DNABII tip chimer antibody or biologically active fragment thereof. In one aspect, the anti-DNABII antibody comprises an anti-DNABII tip chimer antibody or biologically active fragment thereof. The antibody can be a polyclonal antibody or a monoclonal antibody. In one aspect, the anti-DNABII antibody comprises an anti-DNABII tip chimer antibody or biologically active fragment thereof. In a further aspect, the anti-DNABII tip chimer antibody is a monoclonal antibody, a chimeric or a humanized antibody, e.g., as disclosed herein. In another aspect, the anti-PilA antibody specifically recognizes and binds the PilA peptide, examples of such include PilA surface antigens or recombinant soluble PilA (Genbank Accession No.: EFU96734.1) and Type IV Pilin (Genbank Accession No.: Yp-003864351.1). The antibodies can be detectably labeled.

In another aspect, provided is a composition comprising, or alternatively consisting essentially of, or yet further consisting of one or more of: (i) an anti-DNABII antibody, a biologically active fragment thereof, a polynucleotide encoding the same, a vector comprising the polynucleotide or a host cell comprising the polynucleotide and/or the vector and optionally expressing the antibody or fragment thereof, (ii) an anti-PilA antibody or a biologically active fragment thereof, a PilA polypeptide or a biologically active fragment thereof, a polynucleotide encoding the same, a vector comprising the polynucleotide or a host cell comprising the polynucleotide and/or the vector and optionally expressing the antibody or fragment thereof, or (iii) an antibiotic optionally comprising a β-lactam antibiotic or a sulfonamide antibiotic, and a carrier optionally a pharmaceutically acceptable carrier. The antibody can be a polyclonal antibody or monoclonal antibody, derivative, variant fragment thereof. In one aspect, the anti-DNABII antibody comprises an anti-DNABII tip chimer antibody or biologically active fragment thereof. The antibody can be a polyclonal antibody or a monoclonal antibody. In one aspect, the anti-DNABII antibody comprises an anti-DNABII tip chimer antibody or biologically active fragment thereof. In a further aspect, the anti-DNABII tip chimer antibody is a monoclonal antibody, a chimeric or a humanized antibody, e.g., as disclosed herein. In another aspect, the anti-PilA antibody specifically recognizes and binds the PilA peptide, examples of such include PilA surface antigens or recombinant soluble PilA (Genbank Accession No.: EFU96734.1) and Type IV Pilin (Genbank Accession No.: Yp-003864351.1).

Also provided are kits and compositions, such as those for use in a method as disclosed herein. In one aspect, provided is a kit comprising, or consisting essentially of, or yet further consisting of at least two of: (i) an anti-DNABII antibody or a biologically active fragment thereof, (ii) one or both of: an anti-PilA antibody or a biologically active fragment thereof, or a PilA polypeptide or a biologically active fragment thereof, or (iii) an antibiotic; and optionally instructions for use. In one aspect, the anti-DNABII antibody comprises an anti-DNABII tip chimer antibody or biologically active fragment thereof. In one aspect, the anti-DNABII antibody comprises an anti-tip chimer antibody. In some embodiments, the antibiotic comprises or consists essentially of, or yet further consists of a β-lactam antibiotic and/or a sulfonamide antibiotic. The antibody can be a polyclonal antibody or monoclonal antibody, derivative, variant fragment thereof. In one aspect, the anti-DNABII antibody comprises an anti-DNABII tip chimer antibody or biologically active fragment thereof. The antibody can be a polyclonal antibody or a monoclonal antibody. In one aspect, the anti-DNABII antibody comprises an anti-DNABII tip chimer antibody or biologically active fragment thereof. In one aspect, the anti-DNABII antibody comprises an anti-DNABII tip chimer antibody or biologically active fragment thereof. In a further aspect, the anti-DNABII tip chimer antibody is a monoclonal antibody, a chimeric or a humanized antibody, e.g., as disclosed herein. In another aspect, the anti-PilA antibody specifically recognizes and binds the PilA peptide, examples of such include PilA surface antigens or recombinant soluble PilA (Genbank Accession No.: EFU96734.1) and Type IV Pilin (Genbank Accession No.: Yp-003864351.1). The antibodies can be detectably labeled.

In another aspect, provided is a composition comprising, or consisting essentially of, or yet further consisting of at least two of the following: (i) an anti-DNABII antibody or a biologically active fragment thereof, (ii) one or both of: an anti-PilA antibody or a biologically active fragment thereof, or a PilA polypeptide or a biologically active fragment thereof, or (iii) an antibiotic; and a carrier optionally a pharmaceutically acceptable carrier. The antibody can be a polyclonal antibody or monoclonal antibody, derivative, variant fragment thereof. In one aspect, the anti-DNABII antibody comprises an anti-DNABII tip chimer antibody or biologically active fragment thereof. In one aspect, the anti-DNABII antibody comprises an anti-DNABII tip chimer antibody or biologically active fragment thereof. The antibody can be a polyclonal antibody or a monoclonal antibody. In one aspect, the anti-DNABII antibody comprises an anti-DNABII tip chimer antibody or biologically active fragment thereof. In a further aspect, the anti-DNABII tip chimer antibody is a monoclonal antibody, a chimeric or a humanized antibody, e.g., as disclosed herein. In another aspect, the anti-PilA antibody specifically recognizes and binds the PilA peptide, examples of such include PilA surface antigens or recombinant soluble PilA (Genbank Accession No.: EFU96734.1) and Type IV Pilin (Genbank Accession No.: Yp-003864351.1). The antibodies can be detectably labeled. In some embodiments, the antibiotic comprises or consists essentially of, or yet further consists of a β-lactam antibiotic and/or a sulfonamide antibiotic.

In some embodiments, the antibody, polypeptide, biologically active fragment of each thereof, or any combination thereof may be administered to a subject in a composition as disclosed herein. In other embodiments, the antibody, polypeptide, biologically active fragment of each thereof, or any combination thereof may be administered to a subject as a polynucleotide encoding such antibody, polypeptide, biologically active fragment or a polynucleotide complementary thereto. In some embodiments, such polynucleotide may be suitable for expressing the antibody, polypeptide, biologically active fragment in the subject, for example via further comprising a regulatory sequence directing the expression. In further embodiments, a vector, such as a gene delivery vehicle, comprising a polynucleotide as disclosed herein may also be used for administration. The vector may be a viral or a non-viral vector as disclosed herein. In yet further embodiments, provided is a host cell comprising one or more of the polynucleotides and/or the vectors as disclosed herein. Accordingly, provided are kits and compositions comprising one or more of such polynucleotides and/or vectors and/or host cells in addition to or in replacement of the corresponding antibody, peptide or biological active fragment thereof expressed. Further provided are methods and compositions suitable for producing such polypeptides, antibodies, biologically active fragments, polynucleotides, vectors and host cells.

### Biofilms

A “biofilm” intends an organized community of microorganisms that at times adhere to the surface of a structure, which may be organic or inorganic, together with the polymers such as DNA that they secrete, release and/or become available in the extracellular milieu due to bacterial lysis. The biofilms are very resistant to microbiotics and antimicrobial agents. They live on gingival tissues, teeth and restorations, causing caries and periodontal disease, also known as periodontal plaque disease. They also cause chronic middle ear infections. Biofilms can also form on the surface of dental implants, stents, catheter lines and contact lenses. They grow on pacemakers, heart valve replacements, artificial joints and other surgical implants. The Centers for Disease Control estimate that over 65% of nosocomial (hospital-acquired) infections are caused by biofilms. They cause chronic vaginal infections and lead to life-threatening systemic infections in people with hobbled immune systems. Biofilms also are involved in numerous diseases. In one embodiment, the biofilm comprises a DNABII polypeptide or protein. In a further embodiment, the biofilm comprises an IHF and/or an HU. In yet a further embodiment, the biofilm comprises an IHFA and/or an IHFB.

A bacterium may also present planktonically, i.e., floating as single cells and/or adhering to a surface but not forming a biofilm. In some embodiments, planktonic bacteria are generated artificially, for example, in a laboratory via sonication. In some embodiments, as used herein planktonic bacteria do not comprise those bacteria released from a biofilm due to treatment of a composition as disclosed herein, since Applicant discovered that such newly released (NRel) bacteria perform differently compared to a planktonic bacteria generated by sonication. More characterizations of the NRel bacteria and their differences compared to a planktonic one are disclosed herein, for example, in Tables 2 and 3.

Biofilms also are involved in numerous diseases, including but not limited to those caused by Aggregatibacter actinomycetemcomitans, Borrelia burgdorferi (e.g., B31), Bordetella pertussis (e.g., Tohama I), Burkholderia pseudomallei (e.g., 668), Burkholderia cenocepacia (e.g., HI2424), Escherichia coli (e.g., K12 MG1655), Enterococcus faecalis (e.g., V583), Haemophilus influenzae (e.g., Rd KW20), Helicobacter pylori (e.g., 26695), Klebsiella pneumoniae, Moraxella catarrhalis (e.g., RH4), Mycobacterium smegmatis (e.g., MC2), Mycobacterium tuberculosis (e.g., CDC1551), Neisseria gonorrhoeae (e.g., FA1090), Neisseria meningitidis (e.g., MC58), Pseudomonas aeruginosa, Porphyromonas gingivalis (e.g., W83), Prevotella intermedia (e.g., 17), Prevotella melaninogenica (e.g., ATCC® 25845), Staphylococcus aureus (e.g., MW2), Staphylococcus epidermidis (e.g., RP62A), Streptococcus agalactiae (e.g., 2603V/R), Streptococcus bovis, Streptococcus gallolyticus (e.g., UCN34), Streptococcus gordonii (e.g., NCTC 7868 (Challis)), Streptococcus mutans (e.g., UA159), Streptococcus pneumoniae (e.g., R6), Streptococcus pyogenes (e.g., MGAS10270), Streptococcus sobrinus (e.g., 6715), Salmonella enterica (e.g., typhi, CT18), Treponema denticola (e.g., ATCC® 35405), Treponema palladum (e.g., Nichols), Vibrio cholera (e.g., El Tor, N16961). Additional organisms known to associate with and/or form biofilms include but are not limited to Campylobacter spp., Candida spp., Legionella pneumophila, and Listeria monocytogenes. For instance, cystic fibrosis patients have Pseudomonas infections that often result in antibiotic resistant biofilms. Other diseases associated with biofilms include, but are not limited to, lung infections of cystic fibrosis patients, otitis media, post-tympanostomy tube ottorhea, chronic suppurative otitis media, native valve infectious endocarditis, osteomyelitis, rhinosinositis, prostatitis, urinary tract infection, wounds, dental caries and periodontitis. Foodborne pathogens, such as but not limited to some of the above listed organisms (e.g., Listeria monocytogenes, Escherichia coli, Salmonella enterica) may also form biofilms on the food which they contaminate. Disease causing biofilms in animals (e.g., Escherichia coli, Salmonella, and Shigella species) may also cause downstream food contamination and/or disease in human hosts. Further, biofilms need not be of one homogeneous microbial population and may incorporate other pathogens and even host cells.

In addition to being associated with disease—both nosocomial and otherwise—and food contamination, biofilms are often causes of industrial contamination, most notably in relation to process waters and surfaces in contact therewith. Complications involving organisms that form biofilm as industrial contaminants include but are not limited to biocorrosion, biofouling, and equipment damage as a result of biofilm formation. Non-limiting exemplary organisms associated with biofilms in industrial settings include those disclosed in Ferrera et al. (2015) Biofouling 31(2):173-180 and Desulfovibrio species. Additional details regarding biofilms may be found in, for example, Donlan (2002) Emerging Infectious Diseases 8(9):881-890.

Haemophilus is a genus of Gram-negative, pleomorphic, coccobacilli bacteria belonging to the family Pasteurellaceae. These organisms inhabit the mucous membranes of the upper respiratory tract, mouth, vagina, and intestinal tract. The genus includes commensal organisms along with some significant pathogenic species such as Haemophilus influenza and Haemophilus ducreyi, the causative agent of chancroid. All members are either aerobic or facultatively anaerobic.

The term “Haemophilus influenzae” refers to pathogenic bacteria that can cause many different infections such as, for example, ear infections, eye infections, OM and sinusitis. Clinical isolates of H. influenza are classified either as serotypes “a” through “f” or as non-typeable (NTHI) depending on the presence or absence, respectively, of type-specific polysaccharide capsules on the bacteria. Many different strains of Haemophilus influenzae have been isolated and have an IhfA, ihfB and hupA genes or protein. Some non-limiting examples of different strains of Haemophilus influenzae include Rd KW20, 86-028NP, R2866, PittGG, PittEE, R2846, and 2019. A prototype NTHI isolate is the low passage isolate 86-028NP which was recovered from a child with chronic OM. 86-028NP was deposited with the American Type Culture Collection, 10801 University Blvd., Manassas, Va. 20110, on Oct. 16, 2001 and assigned accession no. PTA-4764.

In some embodiments of any aspect as disclosed herein, a biofilm as discussed herein is a polymicrobial biofilm, i.e., comprising, or alternatively consisting essentially of, or yet further consisting of more than one bacteria. In some embodiments, the biofilm comprises a Haemophilus bacterium. In further embodiments, the polymicrobial biofilm comprises, or alternatively consists essentially of, or yet further consists of more than one Haemophilus bacteria. Additionally or alternatively, the polymicrobial biofilm comprises, or alternatively consists essentially of, or yet further consists of one or more than one Haemophilus bacteria and a bacteria which is not Haemophilus (i.e., a non-Haemophilus bacterium). In some embodiments, a Haemophilus bacterium comprises or alternatively consists essentially of, or yet further consists of Haemophilus influenza (e.g., one or more of Nontypeable Haemophilus influenzae (NTHI), Rd KW20, 86-028NP, R2866, PittGG, PittEE, R2846, and 2019). In some aspects, the polymicrobial biofilm is caused by Haemophilus and one or more of Burkholderia cenocepacia, Staphylococcus aureus, Streptococcus pneumonia, or Moraxella catarrhalis.

In some embodiments, a Haemophilus bacterium comprises or alternatively consists essentially of, or yet further consists of Nontypeable Haemophilus influenzae (NTHI). In one embodiment, a Haemophilus bacterium comprises or alternatively consists essentially of, or yet further consists of one strain of Nontypeable Haemophilus influenzae (NTHI), such as 86-028NP, and optionally one or more bacteria other than NTHI (i.e., non-NTHI bacteria). Other NTHI strains can be identified by one of skill in the art, such as HI1716 or NTHI 2019. See for example, Greiner et al. 2004. In another embodiment, a Haemophilus bacterium comprises or alternatively consists essentially of, or yet further consists of more than one strains of Nontypeable Haemophilus influenzae (NTHI), and optionally one or more bacteria other than NTHI. In some aspects, the polymicrobial biofilm is caused by NTHI and one or more of Burkholderia cenocepacia, Staphylococcus aureus, Streptococcus pneumonia, or Moraxella catarrhalis.

In some embodiments of any aspect as disclosed herein, the non-Haemophilus bacterium and/or the non-NTHI bacterium is selected from one or more of the following: Moraxella catarrhalis, Staphylococcus aureus, Pseudomonas aeruginosa, Streptococcus pneumoniae, Burkholderia cenocepacia, an ESKAPEE pathogen (selected from Enterococcus faecium, Klebsiella pneumoniae, Acinetobacter baumannii, Pseudomonas aeruginosa, and Enterobacter spp.), Aggregatibacter actinomycetemcomitans, Borrelia burgdorferi (e.g., B31), Bordetella pertussis (e.g., Tohama I), Burkholderia pseudomallei (e.g., 668), Burkholderia cenocepacia (e.g., HI2424), Escherichia coli (e.g., K12 MG1655), Enterococcus faecalis (e.g., V583), Helicobacter pylori (e.g., 26695), Klebsiella pneumoniae, Moraxella catarrhalis (e.g., RH4), Mycobacterium smegmatis (e.g., MC2), Mycobacterium tuberculosis (e.g., CDC1551), Neisseria gonorrhoeae (e.g., FA1090), Neisseria meningitidis (e.g., MC58), Pseudomonas aeruginosa, Porphyromonas gingivalis (e.g., W83), Prevotella intermedia (e.g., 17), Prevotella melaninogenica (e.g., ATCC @25845), Staphylococcus aureus (e.g., MW2), Staphylococcus epidermidis (e.g., RP62A), Streptococcus agalactiae (e.g., 2603V/R), Streptococcus bovis, Streptococcus gallolyticus (e.g., UCN34), Streptococcus gordonii (e.g., NCTC 7868 (Challis)), Streptococcus mutans (e.g., UA159), Streptococcus pneumoniae (e.g., R6), Streptococcus pyogenes (e.g., MGAS10270), Streptococcus sobrinus (e.g., 6715), Salmonella enterica (e.g., typhi, CT18), Treponema denticola (e.g., ATCC® 35405), Treponema palladum (e.g., Nichols), Vibrio cholera (e.g., El Tor, N16961), Campylobacter spp., Candida spp., Legionella pneumophila, Vibrio vulruficus, Vibrio cholera, E. coli, Legionella pneumophila Salmonella, Shigella, Listeria, Aggregatibacter, Neisseria, Burkholderia cenocepacia (B. cenocepacia), B. multivorans, B. mallei, B. cepaci, B. pseudomallei, or Listeria monocytogenes.

In some embodiments of any aspect as disclosed herein, the polymicrobial biofilm comprises, or alternatively consists essentially of, or yet further consists of NTHI, Streptococcus pneumonia, Staphylococcus aureus, or Moraxella catarrhalis.

In some embodiments of any aspect as disclosed herein, the polymicrobial biofilm is recurrent, or recalcitrant to an antibiotic monotherapy. As used herein, a biofilm recalcitrant to an antibiotic monotherapy refers to that an antibiotic monotherapy, i.e., only using an antibiotic in treating the biofilm, does not result in a desirable effects and/or does not prevent the biofilm from recurring.

Biofilms contribute significantly to the chronicity and recurrence of bacterial diseases due to the fact that biofilm-resident bacteria are highly recalcitrant to killing by host immune effectors and antibiotics. Thus, antibody-mediated release of bacteria from biofilm residence into the surrounding milieu supports a powerful strategy to resolve otherwise difficult-to-treat biofilm-associated diseases. In the prior work, Applicant revealed that antibodies directed against two unique determinants of nontypeable Haemophilus influenzae (NTHI) (e.g. the Type IV pilus (T4P) of NTHI or a bacterial DNABII DNA-binding protein, a species-independent target that provides structural integrity to bacterial biofilms) release biofilm-resident bacteria via discrete mechanisms. Herein, Applicant showed that the phenotype of the resultant newly released (or NRel) NTHI is dependent upon the specific mechanism of release. Applicant used flow cytometry, proteomic profiles, and targeted transcriptomics to show that the two NRel populations were significantly different not only from planktonically grown NTHI, but importantly, from each other despite genetic identity. Moreover, each NRel population had a distinct, significantly increased susceptibility to killing by either a sulfonamide or β-lactam antibiotic compared to planktonic NTHI, an observation consistent with their individual proteomes and further supported by relative differences in targeted gene expression. The distinct phenotypes of NTHI released from biofilms by antibodies directed against specific epitopes of T4P or DNABII binding proteins provide new opportunities to develop targeted therapeutic strategies for biofilm eradication and disease resolution. It is also known that anti-rsPilA specifically targets an NTHI antigen that is not expressed by Moraxella catarrhalis but unexpectedly, that whereas anti-rsPilA has no effect on a pure M. catarrhalis biofilm, when M. catarrhalis is allowed to build a biofilm with NTHI a unique structure forms whereby M. catarrhalis builds these ‘barrel’-looking structures amidst a lawn of NTHI biofilm and then NTHI uses its type IV pilus to twitch up and over the M. catarrhalis ‘barrels’ to completely cover them up. It was shown that treatment of a NTHI+Mcat polymicrobial biofilm with anti-rsPilA, NTHI disperses itself.

In some embodiments, the biofilm is derived from a gram negative or a gram positive biofilm producing bacteria. Non-limiting examples of conditions are selected from the group of: chronic non-healing wounds, including venous ulcers and diabetic foot ulcers, ear infections, sinus infections, urinary tract infections, gastrointestinal tract ailments, pulmonary infections, respiratory tract infections, cystic fibrosis, chronic obstructive pulmonary disease, catheter-associated infections, indwelling devices associated infections, infections associated with implanted prostheses, osteomyelitis, cellulitis, abscesses, and periodontal disease.

### PilA and Vaccines

In Applicant's long-standing efforts to develop a vaccine for diseases of the respiratory tract caused by NTHI, Applicant focused on two unique, biofilm-associated determinants. The first target is the NTHI T4P, a critical adhesin with multiple roles in adherence, colonization, biofilm formation, twitching motility and competence (Bakaletz et al., 2005; Carruthers et al., 2012; Das et al., 2017; Jurcisek et al., 2007; Mokrzan et al., 2019; Mokrzan et al., 2016; Toone et al., 2020). Antibodies against the majority subunit of NTHI T4P (PilA), and specifically a recombinant and soluble form of PilA (‘rsPilA’), induce dispersal of pre-existing NTHI as well as polymicrobial biofilms in vitro, and also those present within the middle ear in a chinchilla model of NTHI-induced OM wherein biofilm dispersal leads to rapid disease resolution (Mokrzan et al., 2018; Novotny et al., 2009; Novotny et al., 2013b; Novotny et al., 2016; Novotny et al., 2015; Ysebaert et al., 2019). The mechanism for this outcome requires expression of both T4P and LuxS, the latter mediates quorum sensing in NTHI (Armbruster et al., 2009; Daines et al., 2005; Surette et al., 1999). Armbruster et al. showed that luxS-induced production of the quorum-sensing molecule autoinducer 2 (AI-2) leads to increased biofilm formation in vitro and persistence in vivo in a chinchilla model of OM (Armbruster et al., 2009), and further revealed that NTHI takes up AI-2 from its environment via RbsB (Armbruster et al., 2011). The role of luxS-mediated AI-2 signaling in biofilm maturation and prevention of biofilm dispersal was further demonstrated by Pang et al., who used an NTHI construct wherein luxS expression was inducible (Pang et al., 2018).

The studies revealed an additional role for luxS quorum signaling specifically during biofilm dispersal induced by anti-rsPilA antibodies, which requires both NTHI T4P expression and luxS-induced production of AI-2 (Novotny et al., 2015). NTHI are released in a ‘top down’ process, with maximal dispersal into the supernatant within 6 h of incubation (Mokrzan et al., 2018; Novotny et al., 2015). Armbruster et al. also showed that Moraxella catarrhalis, which does not express AI-2, nonetheless “eavesdrops” on the AI-2 signal produced by NTHI within a polymicrobial biofilm formed by these two species, which leads to increased M. catarrhalis biofilm formation (Armbruster et al., 2010). Intriguingly, when Applicant incubated a pre-formed dual-species NTHI plus M. catarrhalis biofilm with antibody directed against rsPilA (to target an antigen expressed exclusively by NTHI), both NTHI and M. catarrhalis were dispersed from the biofilm (Mokrzan et al., 2018). The mechanism for M. catarrhalis dispersal revealed another example wherein M. catarrhalis had eavesdropped on the AI-2 produced by NTHI in response to exposure to anti-rsPilA (Mokrzan et al., 2018).

PilA is short for Type IV pilus major subunit protein. Type IV pili are typically 5-7 nm in diameter, several micrometers in length and comprised of a single protein Sub perturn (Bardy et al., Microbiology, 149,295-304, 2003; Wall and Kaiser, Mol. Microbiol., 32,1-10, 1999). Type IV pilin Subunits are usually 145-160 amino acids in length and may be glycosylated or phosphorylated. There are two classes of pilin subunits, type IVa and type IVb, which are distinguished from one another by the average length of the leader peptide and the mature subunit, which N-methylated amino acid occupies the N-terminal position of the mature protein, and the average length of the D-region (for disulfide region). Most of the respiratory pathogens express class IVa pilins, whereas the enteropathogens more typically express class IVb pilins. Type IVa pili are distinguished by the presence of a highly conserved, hydrophobic N-terminal methylated phenylala 1C.

In some embodiments, a biologically active fragment of a PilA has at least about 50%, or at least about 60%, or at least about 70%, or at least about 80%, or at least about 85%, or at least about 90%, or at least about 95%, or at least about 96%, or at least about 97%, or at least about 98%, or at least about 99%, or at least about 100%, or at least 1.5 folds, or at least 2 folds, or at least 3 folds, or at least 4 folds, or at least 5 folds, or at least 6 folds, or at least 7 folds, or at least 8 folds, or at least 9 folds, or at least 10 folds, or at least 20 folds, or at least 50 folds, or at least 100 folds, or more of the biological activity of the wild type PilA. In some embodiments, a biologically active fragment of PilA comprises, or consists essentially of, or yet further consists of a fragment of PilA. In some embodiments, the biological activity comprises, or consists essentially of, or yet further consists of inducing an antibody or other immune response in a subject against PilA. Such biological activity can be measured by one of skill in the art via, for example, quantifying concentration and/or PilA affinity of antibodies in a sample of the subject immunized with the biologically active fragment. Additionally or alternatively, the biological activity here comprises, or consists essentially of, or yet further consists of one or more of: preventing, inhibiting, disrupting, dispersing or treating a biofilm in a subject.

In some embodiments of any aspect as disclosed herein, the PilA is a chimeric protein as disclosed herein. In some embodiments of any aspect as disclosed herein, the PilA or a biologically active fragment thereof comprises, or alternatively consists essentially of, or yet further consists of a chimeric proteins comprising a fragment of the Type IV pilus major subunit protein (PilA) of nontypeable H. influenzae (NTHI) and a fragment of NTHI OMP P5 protein (also called P5-fimbrin, fimbrin or OMP P5-homologous adhesin). In further embodiments, the PilA or a biologically active fragment thereof comprises, or alternatively consists essentially of, or yet further consists of a chimeric proteins comprising PilA modified to present the B-cell epitope of the LB1 peptide. Also provided herein as the PilA or a biologically active fragment thereof are vaccine compositions comprising one or more chimeric proteins as disclosed herein and methods of eliciting an immune response using the chimeric proteins as disclosed herein. Such chimeric protein is disclosed in U.S. Pat. No. 7,811,591, which is incorporated herein by reference in its entirety.

In some embodiments of any aspect as disclosed herein, the PilA or a biologically active fragment thereof comprises, or consists essentially of, or yet further consists of a chimeric protein as described herein.

The LB1 peptide is a 40 amino acid synthetic chimeric P5-fimbrin derived peptide (Arg Ser Asp Tyr Lys Phe Tyr Glu Asp Ala Asn Gly Thir Arg Asp His Lys Lys Gly Pro Ser Leu Lys Leu Leu Ser Leu Ile Lys Gly Val Ile Val His Arg Leu Glu Gly Val Glu, SEQ ID NO: ) that induces an immunogenic response to NTHI and is advantageous because it does not require tedious purification techniques. The LB1 peptide comprises an N-terminal 19 amino acid peptide that is a B-cell epitope (Arg Ser Asp Tyr Lys Phe Tyr Glu Asp Ala Asn Gly Thr Arg Asp His Lys Lys Gly, SEQ ID NO: ). The B-cell epitope was derived from the predicted surface-exposed loop 3 of an outer membrane protein (fimbrin) of NTHi denoted as OMP P5 (also called P5-fimbrin or OMP P5-homologous adhesin). The LB1 peptide further comprises a short 5-mer linker peptide and a 16-residue T cell promiscuous epitope. The T cell epitope was derived from a fusion protein of the measles virus. The T cell promiscuous epitope induces a very strong T cell response in individuals exposed to this epitope.

In some embodiments, the PilA or a biologically active fragment thereof comprises, or consists essentially of, or yet further consists of a fragment of the LB1 peptide inserted into a safer and selective carrier protein that does not reduce the effectiveness of inducing a B-cell response. Preferably, the fragment of the LB1 peptide is inserted into a carrier that itself also confers protection against NTHi-induced diseases. One such carrier that may induce protection against NTHi induced diseases is the protein that comprises the NTHi Type IV pilus (twitching pilus) protein, also known as PilA protein (comprising, or alternatively consisting essentially of, or yet further consisting of a sequence of Met Lys Leu Thr Thr Gln Gln Thr Leu Lys Lys Gly Phe Thr Leu Ile Glu Leu Met Ile Val Ile Ala Ile Ile Ala Ile Leu Ala Thr Ile Ala Ile Pro Ser Tyr Gln Asn Tyr Thr Lys Lys Ala Ala Val Ser Glu Leu Leu Gln Ala Ser Ala Pro Tyr Lys Ala Asp Val Glu Leu Cys Val Tyr Ser Thr Asn Glu Thr Thr Asn Cys Thr Gly Gly Lys Asn Gly Ile Ala Ala Asp Ile Thr Thr Ala Lys Gly Tyr Val Lys Ser Val Thr Thr Ser Asn Gly Ala Ile Thr Val Lys Gly Asp Gly Thr Leu Ala Asn Met Glu Tyr Ile Leu Gln Ala Thr Gly Asn Ala Ala Thr Gly Val Thr Trp Thr Thr Thr Cys Lys Gly Thr Asp Ala Ser Leu Phe Pro Ala Asn Phe Cys (SEQ ID NO: 51). The PilA protein is encoded by the pilA gene (comprising, or alternatively consisting essentially of, or yet further consisting of a sequence of atg aaa cta aca aca cag caa acc ttg aaa aaa ggg ttt aca tta ata gag cta atg att gtg att gca att att get att tta gcc act atc gca att ccc tct tat caa aat tat act aaa aaa gca gcg gta tct gaa tta ctg caa gcg tca gcg cct tat aag get gat gtg gaa tta tgt gta tat agc aca aat gaa aca aca aac tgt acg ggt gga aaa aat ggt att gca gca gat ata acc aca gca aaa ggc tat gta aaa tca gtg aca aca agc aac ggt gca ata aca gta aaa ggg gat ggc aca ttg gca aat atg gaa tat att ttg caa get aca ggt aat get gca aca ggt gta act tgg aca aca act tgc aaa gga acg gat gcc tct tta ttt cca gca aat ttt tgc gga agt gtc aca caa, SEQ ID NO: ).

In some embodiments, the PilA or a biologically active fragment thereof comprises, or consists essentially of, or yet further consists of a fragment of the LB1 peptide in order to present the peptide to induce an immunogenic response. Such fragment of the LB1 peptide can be 12 to 35 amino acids, or 15 to 30 amino acids, or 18 to 19 amino acids long and is a subunit of the fimbrin protein. In some embodiments, the fragment of the LB1 peptide comprises, or consists essentially of, or yet further consists of the N-terminal amino acid sequence RSDYKFYEDANGTRDHKKG (SEQ ID NO: ).

In another embodiment, the PilA or a biologically active fragment thereof comprises, or consists essentially of, or yet further consists of a PilA protein modified to present a 24 amino acid peptide. The 24 amino acid peptide may comprise the B-cell epitope of the LB1 peptide modified as set out in the amino acid sequence of LVRSDYKFYEDANGTRDHKKGRHT (SEQ ID NO: ) in which a leucine and valine are added to the N terminus of the B-cell epitope of LB1 and an arginine, histidine and threonine are at the C terminus of the B-cell epitope of LB1. These modifications to the B-cell epitope are contemplated to assist in protein folding and/or antigen presentation. Any modifications to the B-cell epitope of LB1 may be used that will assist in protein folding and/or antigen presentation.

The amino acid sequence of the surface exposed loop 3 of NTHi OMP P5 can vary between NTHi strains. The PilA or a biologically active fragment thereof comprises, or consists essentially of, or yet further consists of a fragment of the PilA protein modified to present the B cell epitope of any variant amino acid sequence of loop 3 of the NTHi OMP P5. In particular, The PilA or a biologically active fragment thereof comprises, or consists essentially of, or yet further consists of a PilA protein modified to present one of the following variant NTHi OMP P5 amino acids sequences: RSDYKLYNKNSSSNSTLKNLGE (SEQ ID NO: ), RSDYKLYNKNSSTLKDLGE (SEQ ID NO: ) and RSDYKFYDNKRID (SEQ ID NO: ). The variant peptides also may be presented with a leucine and valine added to the N terminus and an arginine, histidine and threonine added to the C terminus or any other modification to assist in protein folding and/or antigen presentation.

The chimeric proteins of the invention comprise the modified PilA amino acids wherein the native PilA amino acids have been substituted with a portion of the LB1 peptide. In addition, the chimeric proteins of the invention comprise a modified PilA amino acid sequence wherein a portion of the LB1 peptide is inserted within and in addition to the native PilA amino acids. The chimeric proteins of the invention have the ability to induce the formation of antibodies directed against two proteins and therefore are more effective and more specific vaccine candidates.

In one embodiment, the chimeric proteins comprise the mature amino acid sequence (residues 13-149) of the NTHi PilA protein (SEQ ID NO: 51) wherein a portion of the LB1 peptide is inserted between the cysteine residues at positions 62 and 72 of SEQ ID NO: 51 and may substitute the native amino acids, such as the chimeric protein having the amino acid sequence of Gly Ser His Met Thr Lys Lys Ala Ala Val Ser Glu Leu Leu Gln Ala Ser Ala Pro Tyr Lys Ala Asp Val Glu Leu Cys Leu Val Arg Ser Asp Tyr Lys Phe Tyr Glu Asp Ala Asn Gly Thr Arg Asp His Lys Lys Gly Arg His Thr Cys Thr Gly Gly Lys Asn Gly Ile Ala Ala Asp Ile Thr Thr Ala Lys Gly Tyr Val Lys Ser Val Thr Thr Ser Asn Gly Ala Ile Thr Val Lys Gly Asp Gly Thr Leu Ala Asn Met Glu Tyr Ile Leu Gln Ala Thr Gly Asn Ala Ala Thr Gly Val Thr Trp Thr Thr Thr Cys Lys Gly Thr Asp Ala Ser Leu Phe Pro Ala Asn Phe Cys Gly Ser Val Thr Gln, SEQ ID NO:. This chimeric protein comprises residues 40-149 of SEQ ID NO: and has the B-cell epitope of LB1 (Leu Val Arg Ser Asp Tyr Lys Phe Tyr Glu Asp Ala Asn Gly Thr Arg Asp His Lys Lys Gly Arg His Thr, SEQ ID NO: 52) inserted between residues 62 and 72 of SEQ ID NO: 51. In another embodiment, the portion of the LB1 peptide is inserted between the cysteine residues at positions 131 and 144 of SEQ ID NO: 51 and may substitute the native amino acids such as the protein having the amino acid sequence of Gly Ser His Met Thr Lys Lys Ala Ala Val Ser Glu Leu Leu Gln Ala Ser Ala Pro Tyr Lys Ala Asp Val Glu Leu Cys Val Tyr Ser Thr Asn Glu Thr Thr Asn Cys Thr Gly Gly Lys Asn Gly Ile Ala Ala Asp Ile Thr Thr Ala Lys Gly Tyr Val Lys Ser Val Thr Thr Ser Asn Gly Ala Ile Thr Val Lys Gly Asp Gly Thr Leu Ala Asn Met Glu Tyr Ile Leu Gln Ala Thr Gly Asn Ala Ala Thr Gly Val Thr Trp Thr Thr Thr Cys Leu Val Arg Ser Asp Tyr Lys Phe Tyr Glu Asp Ala Asn Gly Thr Arg Asp His Lys Lys Gly Arg His Thr Cys Gly Ser Val Thr Gln, SEQ ID NO:. This chimeric protein comprises residues 40-149 of SEQ ID NO: 51 and has the B-cell epitope of LB1 (SEQ ID NO: 52) inserted between residues 131 and 144 of SEQ ID NO: 51.

In another embodiment, the chimeric proteins comprise the mature amino acid sequence (residues 13-149) of the NTHi PilA protein (SEQ ID NO: 51) wherein the portion of the LB1 peptide is inserted at the C-terminus of the PilA protein. For example, the chimeric protein of Gly Ser His Met Thr Lys Lys Ala Ala Val Ser Glu Leu Leu Gln Ala Ser Ala Pro Tyr Lys Ala Asp Val Glu Leu Cys Val Tyr Ser Thr Asn Glu Thr Thr Asn Cys Thr Gly Gly Lys Asn Gly Ile Ala Ala Asp Ile Thr Thr Ala Lys Gly Tyr Val Lys Ser Val Thr Thr Ser Asn Gly Ala Ile Thr Val Lys Gly Asp Gly Thr Leu Ala Asn Met Glu Tyr Ile Leu Gln Ala Thr Gly Asn Ala Ala Thr Gly Val Thr Trp Thr Thr Thr Cys Lys Gly Thr Asp Ala Ser Leu Phe Pro Ala Asn Phe Cys Gly Ser Val Thr Gln Leu Val Arg Ser Asp Tyr Lys Phe Tyr Glu Asp Ala Asn Gly Thr Arg Asp His Lys Lys Gly Arg His Thr, SEQ ID NO: , comprises residues 40-149 of SEQ ID NO: 51 and the B-cell epitope of LB1 (SEQ ID NO: 52) is inserted following residue 149 of SEQ ID NO: 51.

In another embodiment, the chimeric proteins comprise the mature amino acid sequence (residues 13-149) of NTHi PilA protein (SEQ ID NO: 51) wherein the portion of the LB1 peptide is inserted at the N-terminus of the PilA protein. For example, the chimeric protein of Gly Ser His Met Leu Val Arg Ser Asp Tyr Lys Phe Tyr Glu Asp Ala Asn Gly Thr Arg Asp His Lys Lys Gly Arg His Thr Gly Pro Ser Leu Lys Leu Thr Lys Lys Ala Ala Val Ser Glu Leu Leu Gln Ala Ser Ala Pro Tyr Lys Ala Asp Val Glu Leu Cys Val Tyr Ser Thr Asn Glu Thr Thr Asn Cys Thr Gly Gly Lys Asn Gly Ile Ala Ala Asp Ile Thr Thr Ala Lys Gly Tyr Val Lys Ser Val Thr Thr Ser Asn Gly Ala Ile Thr Val Lys Gly Asp Gly Thr Leu Ala Asn Met Glu Tyr Ile Leu Gln Ala Thr Gly Asn Ala Ala Thr Gly Val Thr Trp Thr Thr Thr Cys Lys Gly Thr Asp Ala Ser Leu Phe Pro Ala Asn Phe Cys Gly Ser Val Thr Gln,(SEQ ID NO: ), comprises residues 40-149 of SEQ ID NO: 2 and the B-cell epitope of LB1 (SEQ ID NO: 52) is inserted before residue 40 of SEQ ID NO: 51.

In a further embodiment, chimeric proteins comprises a portion of the NTHi PilA protein and one or more of the LB1 peptides described herein. The chimeric proteins of the invention include those which present the same LB1 peptide more than once within a portion of the NTHi PilA protein and those which present two or more different LB1 peptides within a portion of the NTHi PilA protein.

The disclosure further provides for chimeric proteins comprising a portion of the NTHi PilA protein and any antigenic protein that will elicit an immune response.

The chimeric proteins may comprise the full length or a portion of the major subunit of the NTHi Type IV Pilus which is encoded by the gene pilA. The PilA protein of the NTHi isolate 86-028NP (for example, SEQ ID NO: 51) is encoded by the nucleic acid sequences described in U.S. Pat. No. 7,501,131, incorporated by reference herein in its entirety. Also provided are polynucleotides encoding PilA polypeptides from NTHi clinical isolates 1728MEE, 1729MEE, 3224A, 10548MEE, 1060MEE, 1885MEE, 1714MEE, 1236MEE, 1128MEE and 214NP. The amino acid sequences of these PilA polypeptides are set out in

(SEQ ID NO: 54)
Met Lys Leu Thr Thr Gln Gln Thr Leu Lys Lys Gly Phe Thr Leu Ile Glu Leu Met Ile Val Ile
Ala Ile Ile Ala Ile Leu Ala Thr Ile Ala Ile Pro Ser Tyr Gln Asn Tyr Thr Lys Lys Ala Ala Val
Ser Glu Leu Leu Gln Ala Ser Ala Pro Tyr Lys Ala Asp Val Glu Leu Cys Val Tyr Ser Thr
Asn Glu Thr Thr Asn Cys Thr Gly Gly Lys Asn Gly Ile Ala Ala Asp Ile Thr Thr Ala Lys Gly
Tyr Val Lys Ser Val Thr Thr Ser Asn Gly Ala Ile Thr Val Lys Gly Asp Gly Thr Leu Ala Asn
Met Glu Tyr Ile Leu Gln Ala Thr Gly Asn Ala Ala Thr Gly Val Thr Trp Thr Thr Thr Cys Lys
Gly Thr Asp Ala Ser Leu Phe Pro Ala Asn Phe Cys Gly Ser Val Thr Gln;
(SE ID NO: 55)
Met Lys Leu Thr Thr Gln Gln Thr Leu Lys Lys Gly Phe Thr Leu Ile Glu Leu Met Ile Val Ile
Ala Ile Ile Ala Ile Leu Ala Thr Ile Ala Ile Pro Ser Tyr Gln Asn Tyr Thr Lys Lys Ala Ala Val
Ser Glu Leu Leu Gln Ala Ser Ala Pro Tyr Lys Ala Asp Val Glu Leu Cys Val Tyr Ser Thr
Asn Glu Thr Thr Asn Cys Thr Gly Gly Lys Asn Gly Ile Ala Ala Asp Ile Thr Thr Ala Lys Gly
Tyr Val Lys Ser Val Thr Thr Ser Asn Gly Ala Ile Thr Val Lys Gly Asp Gly Thr Leu Ala Asn
Met Glu Tyr Ile Leu Gln Ala Thr Gly Asn Ala Ala Thr Gly Val Thr Trp Thr Thr Thr Cys Lys
Gly Thr Asp Ala Ser Leu Phe Pro Ala Asn Phe Cys Gly Ser Val Thr Gln,;
(SEQ ID NO: 56)
Met Lys Leu Thr Thr Gln Gln Thr Leu Lys Lys Gly Phe Thr Leu Ile Glu Leu Met Ile Val Ile
Ala Ile Ile Ala Ile Leu Ala Thr Ile Ala Ile Pro Ser Tyr Gln Asn Tyr Thr Lys Lys Ala Ala Val
Ser Glu Leu Leu Gln Ala Ser Ala Pro Tyr Lys Ala Asp Val Glu Leu Cys Val Tyr Ser Thr
Asn Glu Thr Thr Asn Cys Thr Gly Gly Lys Asn Gly Ile Ala Ala Asp Ile Thr Thr Ala Lys Gly
Tyr Val Lys Ser Val Thr Thr Ser Asn Gly Ala Ile Thr Val Lys Gly Asp Gly Thr Leu Ala Asn
Met Glu Tyr Ile Leu Gln Ala Thr Gly Asn Ala Ala Thr Gly Val Thr Trp Thr Thr Thr Cys Lys
Gly Thr Asp Ala Ser Leu Phe Pro Ala Asn Phe Cys Gly Ser Val Thr Gln;
(SEQ ID NO: 57)
Met Lys Leu Thr Thr Gln Gln Thr Leu Lys Lys Gly Phe Thr Leu Ile Glu Leu Met Ile Val Ile
Ala Ile Ile Ala Ile Leu Ala Thr Ile Ala Ile Pro Ser Tyr Gln Asn Tyr Thr Lys Lys Ala Ala Val
Ser Glu Leu Leu Gln Ala Ser Ala Pro Tyr Lys Ala Asp Val Glu Leu Cys Val Tyr Ser Thr
Asn Glu Thr Thr Asn Cys Thr Gly Gly Lys Asn Gly Ile Ala Ala Asp Ile Thr Thr Ala Lys Gly
Tyr Val Lys Ser Val Thr Thr Ser Asn Gly Ala Ile Thr Val Lys Gly Asp Gly Thr Leu Ala Asn
Met Glu Tyr Ile Leu Gln Ala Thr Gly Asn Ala Ala Thr Gly Val Thr Trp Thr Thr Thr Cys Lys
Gly Thr Asp Ala Ser Leu Phe Pro Ala Asn Phe Cys Gly Ser Val Thr Gln,,
(SEQ ID NO: 58)
Met Lys Leu Thr Thr Gln Gln Thr Leu Lys Lys Gly Phe Thr Leu Ile Glu Leu Met Ile Val Ile
Ala Ile Ile Ala Ile Leu Ala Thr Ile Ala Ile Pro Ser Tyr Gln Asn Tyr Thr Lys Lys Ala Ser Val
Ser Glu Leu Leu Gln Ala Ser Ala Pro Tyr Lys Ala Asp Val Glu Leu Cys Val Tyr Ser Thr
Asn Glu Thr Thr Asn Cys Thr Gly Gly Lys Asn Gly Ile Ala Ala Asp Ile Thr Thr Ala Lys Gly
Tyr Val Ala Ser Val Lys Thr Gln Ser Gly Gly Ile Thr Val Lys Gly Asn Gly Thr Leu Ala Asn
Met Glu Tyr Ile Leu Gln Ala Lys Gly Asn Ala Thr Ala Gly Val Thr Trp Thr Thr Thr Cys Lys
Gly Thr Asp Ala Ser Leu Phe Pro Ala Asn Phe Cys Arg Ser Val Thr Lys,,
(SEQ ID NO: 59)
Met Lys Leu Thr Thr Gln Gln Thr Leu Lys Lys Gly Phe Thr Leu Ile Glu Leu Met Ile Val Ile
Ala Ile Ile Ala Ile Leu Ala Thr Ile Ala Ile Pro Ser Tyr Lys Asn Tyr Thr Lys Lys Ala Ala Val
Ser Glu Leu Leu Gln Ala Ser Ala Pro Tyr Lys Ala Asp Val Glu Leu Cys Val Tyr Ser Thr
Asn Glu Ile Thr Asn Cys Met Gly Gly Lys Asn Gly Ile Ala Ala Asp Ile Thr Thr Ala Lys Gly
Tyr Val Ala Ser Val Lys Thr Gln Ser Gly Gly Ile Thr Val Lys Gly Asp Gly Thr Leu Ala Asn
Met Glu Tyr Ile Leu Gln Ala Thr Gly Asn Ala Ala Ala Gly Val Thr Trp Thr Thr Thr Cys Lys
Gly Thr Asp Ala Ser Leu Phe Pro Ala Asn Phe Cys Gly Ser Ile Thr Gln,
(SEQ ID NO: 60)
Met Lys Leu Thr Thr Leu Gln Thr Leu Lys Lys Gly Phe Thr Leu Ile Glu Leu Met Ile Val Ile
Ala Ile Ile Ala Ile Leu Ala Thr Ile Ala Ile Pro Ser Tyr Gln Asn Tyr Thr Lys Lys Ala Ala Val
Ser Glu Leu Leu Gln Ala Ser Ala Pro Tyr Lys Ala Asp Val Glu Leu Cys Val Tyr Ser Thr Gly
Lys Pro Ser Thr Cys Ser Gly Gly Ser Asn Gly Ile Ala Ala Asp Ile Thr Thr Ala Lys Gly Tyr
Val Ala Ser Val Lys Thr Gln Ser Gly Gly Ile Thr Val Lys Gly Asn Gly Thr Leu Ala Asn Met
Glu Tyr Ile Leu Gln Ala Thr Gly Asn Ala Ala Thr Gly Val Thr Trp Thr Thr Thr Cys Lys Gly
Thr Asp Ala Ser Leu Phe Pro Ala Asn Phe Cys Gly Ser Val Thr Gln,,
(SEQ ID NO: 61)
Met Lys Leu Thr Thr Leu Gln Thr Leu Lys Lys Gly Phe Thr Leu Ile Glu Leu Met Ile Val Ile Ala
Ile Ile Ala Ile Leu Ala Thr Ile Ala Ile Pro Ser Tyr Gln Asn Tyr Thr Lys Lys Ala Ala Val Ser
Glu Leu Leu Gln Ala Ser Ala Pro Tyr Lys Ala Asp Val Glu Leu Cys Val Tyr Ser Thr Gly
Lys Leu Ser Thr Cys Ser Gly Gly Ser Asn Gly Ile Ala Ala Asp Ile Thr Thr Ala Lys Gly Tyr
Val Ala Ser Val Lys Thr Gln Ser Gly Gly Ile Thr Val Lys Gly Asn Gly Thr Leu Ala Asn Met
Glu Tyr Ile Leu Gln Ala Lys Gly Asn Ala Thr Ala Gly Val Thr Trp Thr Thr Thr Cys Lys Gly
Thr Asp Ala Ser Leu Phe Pro Ala Asn Phe Cys Gly Ser Val Thr Lys,;
(SEQ ID NO: 62)
Met Lys Leu Thr Thr Gln Gln Thr Leu Lys Lys Gly Phe Thr Leu Ile Glu Leu Met Ile Val Ile Ala
Ile Ile Ala Ile Leu Ala Thr Ile Ala Ile Pro Ser Tyr Gln Asn Tyr Thr Lys Lys Ala Ser Val Ser
Glu Leu Leu Gln Ala Ser Ala Pro Tyr Lys Ser Asp Val Glu Leu Cys Val Tyr Ser Thr Gly Lys
Pro Ser Thr Cys Ser Gly Gly Ser Asn Gly Ile Ala Ala Asp Ile Thr Thr Ala Lys Gly Tyr Val
Ala Ser Val Lys Thr Gln Ser Gly Gly Ile Thr Val Lys Gly Asn Gly Thr Leu Ala Asn Met Glu
Tyr Ile Leu Gln Ala Lys Gly Asn Ala Thr Ala Gly Val Thr Trp Thr Thr Thr Cys Lys Gly Thr
Asp Ala Ser Leu Phe Pro Ala Asn Phe Cys Arg Ser Val Thr Lys,;
and
(SEQ ID NO: 63)
Met Lys Leu Thr Thr Gln Gln Thr Leu Lys Lys Gly Phe Thr Leu Ile Glu Leu Met Ile Val Ile Ala
Ile Ile Ala Ile Leu Ala Thr Ile Ala Ile Pro Ser Tyr Gln Asn Tyr Thr Lys Lys Ala Ala Val Ser
Glu Leu Leu Gln Ala Ser Ala Pro Tyr Lys Ala Asp Val Glu Leu Cys Val Tyr Ser Thr Gly
Lys Pro Ser Ser Cys Ser Gly Gly Ser Asn Gly Ile Ala Ala Asp Ile Thr Thr Ala Lys Gly Tyr
Val Lys Ser Val Thr Thr Ser Asn Gly Ala Ile Thr Val Lys Gly Asp Gly Thr Leu Ala Asn Met
Glu Tyr Ile Leu Gln Ala Ser Gly Asn Ala Ala Thr Gly Val Thr Trp Thr Thr Thr Cys Lys Gly
Thr Asp Ala Ser Leu Phe Pro Ala Asn Phe Cys Gly Ser Val Thr Gln

respectively. The possibility of alternative codon usage is specifically contemplated in polynucleotides encoding the polypeptides. In one embodiment, the polypeptides are respectively encoded by the nucleotide sequences set out in the SEQ ID NOS: 33, 35, 37, 39, 41, 43, 45, 47, 49 and 51 as disclosed in U.S. Pat. No. 7,811,591 (each of the sequences is incorporated herein by reference in its entirety).

In some embodiments, a chimeric protein comprises a portion of NTHi PilA protein. In one embodiment the polypeptides comprise the NTHi 86-028NP amino acid sequences respectively set out in SEQ ID NO: 51. Polypeptides of the invention also include PilA polypeptides set out in SEQ ID NOS: 54, 55, 56, 57, 58, 59, 60, 61, 62 and 63. In additional embodiments, the PilA polypeptides of the invention are those of other non-typeable H. influenzae strains and from H. influenzae strains a, b, c, e and f.

Polypeptides of PilA specifically include peptide fragments (i.e., peptides) or fragments of the PilA polypeptide that retain one or more biological or immunogenic properties of a full length polypeptide of the invention. In one embodiment, PilA peptide fragments provided by the invention are designated TfpQ2, TfpQ3, TfpQ4 and OLP3 and respectively comprise amino acids 35 through 68 of SEQ ID NO: 51, amino acids 69 through 102 of SEQ ID NO: 51, amino acids 103 through 137 of SEQ ID NO: 51, and amino acids 21 through 35 of SEQ ID NO: 51. Another PilA peptide fragment provided by the disclosure comprises amino acids 40 through 149 of SEQ ID NO: 51.

In some embodiments, a chimeric protein comprises a portion of a PilA polypeptide with one or more conservative amino acid substitutions that do not affect the biological and/or immunogenic activity of the PilA polypeptide. Alternatively, the PilA polypeptides are contemplated to have conservative amino acids substitutions which may or may not alter biological activity.

In some embodiments, a chimeric protein comprises a portion of a variants of the NTHi PilA polypeptides of the present invention (e.g., a polypeptide exhibiting at least about 65%, at least about 70%, at least about 75%, at least about 80%, at least about 85%, 86%, 87%, 88%, 89%, at least about 90%, 91%, 92%, 93%, 94%, typically at least about 95%, 96%, 97%, more typically at least about 98%, or most typically at least about 99% amino acid identity to a polypeptide of SEQ ID NOS: 51, 54, 55, 56, 57, 58, 59, 60, 61, 62 and 63 that retain biological and/or immunogenic activity. In addition, modifications in the sequence are easily made by substitution, addition or omission of appropriate residues. For example, a cysteine residue may be added at the carboxy terminus to provide a sulfhydryl group for convenient linkage to a carrier protein, or spacer elements, such as an additional glycine residue, may be incorporated into the sequence between the linking amino acid at the C-terminus and the remainder of the peptide.

In some embodiments of any aspect as disclosed herein, the PilA is a recombinant and soluble PilA (rsPilA). Recombinant PilA protein (rPilA) may be generated to serve as a more readily renewable product. To do this, the published protocol of Keizer et al. (J. Biol. Chem., 276: 24186-14193, 2001), who studied a pilin which also had four Cys residues as it will be critical that rPilA similarly be properly folded so as to possess functional qualities of the native pilin subunit, is utilized. Briefly, a truncated pilin is engineered wherein the first 28 residues are removed from the N-terminus to prevent aggregation, and this truncated pilin will be further engineered to be transported to the periplasm by means of the incorporation of an OmpA leader sequence in the construct. Using this strategy Keizer et al. generated a recombinant soluble monomeric P. aeruginosa pilin protein that was able to bind to its receptor (asialo GM1) in in vitro assays and decrease morbidity and mortality in mice when the peptide was delivered 15 minutes prior to heterologous challenge. This soluble, monomeric, truncated form of NTHi PilA will be useful in the studies described herein.

In some embodiments, the chimeric proteins may be synthesize, purified and sequenced using standard techniques. For example, the chimeric proteins may be assembled semi-manually by stepwise Fmoc-tert-butyl solid-phase synthesis and purified by HPLC. The composition and amino acid sequence of recombinant and synthetic chimeric proteins may be confirmed by amino acid analysis and/or mass spectral analysis.

### Anti-PilA Antibodies

In some embodiments, the anti-PilA antibody a polyclonal antibody or a monoclonal antibody. In one aspect, the polyclonal antibody is in a sera isolated from a subject immunized with a PilA polypeptide or a biologically active fragment thereof. In some embodiments, such sera may be purified and processed, such as digested to produce Fab fragments of the antibodies.

In one aspect, the antibody specifically recognizes and bind the PilA peptide, examples of such include surface antigens or recombinant soluble PilA (Genbank Accession No.: EFU96734.1) and Type IV Pilin (Genbank Accession No.: Yp-003864351.1). In one aspect the antibody is a polyclonal antibody. In another aspect, the antibody is a monoclonal antibody or a biologically active fragment thereof. Methods to generate monoclonal antibodies, derivatives, fragments and variants thereof are known in the art.

In some embodiments, a biologically active fragment of an anti-PilA antibody has at least about 50%, or at least about 60%, or at least about 70%, or at least about 80%, or at least about 85%, or at least about 90%, or at least about 95%, or at least about 96%, or at least about 97%, or at least about 98%, or at least about 99%, or at least about 100%, or at least 1.5 folds, or at least 2 folds, or at least 3 folds, or at least 4 folds, or at least 5 folds, or at least 6 folds, or at least 7 folds, or at least 8 folds, or at least 9 folds, or at least 10 folds, or at least 20 folds, or at least 50 folds, or at least 100 folds, or more of the biological activity of a reference anti-PilA antibody. In some embodiments, the biological activity comprises, or consists essentially of, or yet further consists of specifically recognizing and binding a PilA. Such biological activity can be measured by one of skill in the art via, for example, quantifying PilA affinity and/or specificity of the antibody and/or competitive binding a PilA compared to a reference antibody. Additionally or alternatively, the biological activity here comprises, or consists essentially of, or yet further consists of one or more of: preventing, inhibiting, disrupting, dispersing or treating a biofilm in a subject. In some embodiments, the biological activity comprises or consists essentially of, or yet further consists of the ability to kill H. influenzae bacteria. Other suitable activity may be selected from the following: reducing the virulence, inhibiting adherence, inhibiting twitching motility, inhibiting cell division, and/or inhibiting penetration into the epithelium of H. influenzae bacteria and/or enhance phagocytosis of the H. influenzae bacteria. In some embodiments, the anti-PilA antibody or a biologically active fragment thereof specifically binds and recognizes a PilA polypeptide or a biologically active fragment thereof. In vitro complement mediated bactericidal assay systems (Musher et al., Infect. Immun. 39: 297-304, 1983; Anderson et al., J. Clin. Invest. 51: 31-38, 1972) may be used to measure the bactericidal activity of anti-chimeric proteins antibodies.

### DNABII

Integration Host Factor (IHF), is a critical structural element of the bacterial biofilm matrix. IHF and HU (a histone-like protein) comprise the ubiquitous two-membered DNABII family of bacterial DNA-binding proteins. Genes that encode IHF and/or HU are present in the genome of every member of Eubacteria (Goodman et al., 2011). Hence, this target is not unique to NTHI but is instead species-independent due to its presence in all tested pathogen-formed biofilms to date, including each of the high priority ESKAPE pathogens (Enterococcus faecium, Staphylococcus aureus, Klebsiella pneumoniae, Acinetobacter baumannii, Pseudomonas aeruginosa and Enterobacter spp.) (Devaraj et al., 2018; Devaraj et al., 2015; Novotny et al., 2013a). Extracellular DNA (eDNA) and associated DNABII proteins are essential to the underlying architecture and structural integrity of these biofilms (Flemming and Wingender, 2010; Goodman et al., 2011; Jurcisek and Bakaletz, 2007; Whitchurch et al., 2002). Within the biofilm matrix, crossed strands of eDNA are stabilized by IHF and HU (Swinger and Rice, 2004). The result is a lattice-like eDNA scaffold that supports and maintains the biofilm architecture. Exposure of bacterial biofilms to antibody against DNABII proteins destabilizes the eDNA matrix and causes collapse of the biofilm structure (Goodman et al., 2011). The mechanism for this outcome is induction of an equilibrium shift wherein DNABII molecules in the milieu that surrounds the biofilm are sequestered due to formation of an antibody complex, thus DNABII proteins within the eDNA matrix release (Brockson et al., 2014; Novotny et al., 2016). The result is a sudden, complete collapse of the eDNA scaffold and release of the biofilm-resident bacteria that begins within 3 min of exposure to anti-DNABII antibodies in vitro (Brockson et al., 2014; Novotny et al., 2019). Vaccination-induced antibodies against DNABII proteins disrupt pre-existing biofilms in a chinchilla model of NTHI-induced OM which permits clearance by host immune effectors (Goodman et al., 2011; Novotny et al., 2019; Novotny et al., 2016). Moreover, therapeutic treatment with anti-DNABII antibodies resolves osteolytic peri-implantitis in a rat model of pre-existing Aggregatibacter actinomycetemcomitans biofilms (Freire et al., 2017), and also eradicates aggregate biofilms of P. aeruginosa from the murine lung (Novotny et al., 2016).

The term “nucleoid associated protein” or “NAP” as used herein refers to a class of proteins that affect the dynamic spatial organization of nucleic acids in the nucleoid of prokaryotic cells. These proteins organize the genome through effecting DNA bending, binding and aggregation. Certain NAPs are DNA binding proteins and may be associated with the biofilm including, DNABII proteins, DPS (Genbank Accession No.: CAA49169), H-NS (Genbank Accession No.: CAA47740), Hfq (Genbank Accession No.: ACE63256), CbpA (Genbank Accession No.: BAA03950) and CbpB (Genbank Accession No.: NP-418813). Of the NAPs, DNABII proteins are distinct and generally have strong sequence identity with alpha helical dimerization domains and may comprise anti-parallel beta ribbons, which often have NPXT (wherein X is any amino acid or may be selected from amino acids Q, R, K, S, or T) comprising tips that bind and intercalate into the minor groove of DNA and kink it. The functional protomer is a dimer of identical or homologous subunits.

The DNABII family is a member of a class of proteins referred to as nucleoid associated proteins (NAPs), bacterial proteins that, in part, shape the intracellular bacterial nucleoid (Browning et al. (2010) Curr. Opin. Microbiol. 13:773-780). In addition, this family is ubiquitous, expressed by virtually all eubacteria. All characterized family members to date function as either a homodimer or heterodimer of subunits. The family is divided into two types, HU (histone-like protein) and IHF (integration host factor) with B. cenocepacia capable of expressing both (strain J2315 genes: BCAL3530, hupA; BCAL1585, hupB; BCAL1487, ihfA and BCAL2949, ihfb). The primary distinction between these family members is that HU binds DNA in a sequence independent manner, while IHF binds a consensus sequence (WATCAANNNNTTR where W is A or T and R is a purine, SEQ ID NO.) conserved across genera (Swinger et al. (2004) Curr. Opin. Struct. Biol. 14:28-35)). All DNABII proteins bind to and bend DNA considerably e.g. E. coli IHF can bend DNA into a virtual U-turn (Rice et al. (1996) Cell 87: 1295-1306). In addition, all family members have a preference for pre-bent or curved DNA structures e.g. Holliday junctions, a cruciform-like structure central to DNA recombination. In fact, DNABII proteins function as accessory factors facilitating all intracellular DNA functions, including gene expression, recombination, repair and replication (Swinger et al. (2004) Curr. Opin. Struct. Biol. 14:28-35).

A “DNABII polypeptide or protein” intends a DNA-binding protein or polypeptide that is composed of DNA-binding domains and thus have a specific or general affinity for microbial DNA. In one aspect, they bind DNA in the minor grove. Non-limiting examples of DNABII proteins are an integration host factor (If) protein and a histone-like protein from E. coli strain U93 (HU). Other DNA binding proteins that may be associated with the biofilm include DPS (Genbank Accession No.: CAA49169), H-NS (Genbank Accession No.: CAA47740), Hfq (Genbank Accession No.: ACE63256), CbpA (Genbank Accession No.: BAA03950) and CbpB (Genbank Accession No.: NP-418813).

An “integration host factor” or “IHF” protein is a bacterial protein that is used by bacteriophages to incorporate their DNA into the host bacteria. They also bind extracellular microbial DNA. The genes that encode the IHF protein subunits in E. coli are himA (Genbank Accession No.: POA6X7.1) and himD (POA6Y1.1) genes. Homologs for these genes are found in other organisms. In certain embodiments, the term “IHF” refers to one or both of the two IHF subunits: integration host factor subunit alpha (IfA or IhfA) and integration host factor subunit beta (IHFB or IhfB).

“HU” or “histone-like protein from E. coli strain U93” refers to a class of heterodimeric proteins typically associate with E. coli. HU proteins are known to bind DNA junctions. Related proteins have been isolated from other microorganisms. The complete amino acid sequence of E. coli HU was reported by Laine et al. (1980) Eur. J. Biochem 103(3) 447-481. Antibodies to the HU protein are commercially available from Abeam. The genes that encode the HU protein subunits in E. coli are hupA and hupB corresponding to SEQ ID NOs: 29 and 30, respectively. Homologs for these genes are found in other organisms, and peptides corresponding to these genes from other organisms can be found in Table 10 of WO 2011/123396.

The term “surface antigens” or “surface proteins” refers to proteins or peptides on the surface of cells such as bacterial cells. Examples of outer membrane proteins such as OMP P5 (Genbank Accession No.: YP-004139079.1), OMP P2 (Genbank Accession No.: ZZX87199.1) and OMP P26 (Genbank Accession No.: YP-665091.1) whereas examples of surface antigens are rsPilA or recombinant soluble PilA (Genbank Accession No.: EFU96734.1) and Type IV Pilin (Genbank Accession No.: Yp-003864351.1).

In some embodiments of any aspect as disclosed herein, the DNABII is selected from an integration host factor (IHF) or a histone-like protein (HU) protein. In some embodiments of any aspect as disclosed herein, DNABII comprises, or consists essentially of, or yet further consists of a wild type DNABII or a biologically active fragment thereof. In some embodiments, a biologically active fragment of a DNABII comprises, or consists essentially of, or yet further consists of a chimer as disclosed herein. In some embodiments, a DNABII comprises, or consists essentially of, or yet further consists of SEQ ID NO. 1 through 348 of WO2014/201305 (each of the sequences is incorporated herein by reference in its entirety), or a fragment or an equivalent of each thereof. Other suitable DNABII or a biologically active fragment thereof produce by gram(+) and gram(−) bacteria can be found in Table 8 in U.S. Pat. No. 8,999,291, incorporated herein by reference and WO2014/201305, WO2017/023863, WO2017/066719, WO2018/129092, WO2018/129078, PCT Patent Application No. PCT/US2020/041082 and PCT/US2021/040576. Antibodies that bind these proteins and fragments thereof are referenced as anti-DNABII antibodies.

A sequence alignment of relevant portions of the DNA binding proteins of various embodiments is published as Table 9 in U.S. Pat. No. 8,999,291, incorporated herein by reference. Bold letters indicate an exact match to consensus, light gray lettering indicates a conservative amino acid change, and lightly or darkly shaded sequences are highly conserved across species. Gray shaded undefined sequences at the amino and/or carboxy-terminal are undefined amino acids that do not share consensus sequences. This table is based on information previously published in Obeto et al. (1994) Biochimie 76:901-908. The fragment “ARM” is denoted at the bottom of the table and polypeptide fragment comprising, or consisting essentially of, or yet further consisting of these fragments or their equivalents have biological activity as noted herein.

Non-limiting examples of such include isolated or recombinant polypeptides comprising the amino acid sequences listed in the table below or fragments or equivalents thereof, and equivalents of each thereof. Further non-limiting examples include an isolated or recombinant polypeptide comprising, or alternatively consisting essentially of, or yet further amino acids AARKGINPKTKKSISIPARKVVRF (SEQ ID NO: ). In yet a further aspect, this disclosure provides mutated or recombinant HU antigenic polypeptides having the corresponding amino acid(s) to the P. gingivalis amino acids at positions 61 and/or 64 modified to I (or alternatively another hydrophobic amino acid such as V or F) and/or K. Biological equivalents of these polypeptides are further included in this disclosure with the proviso that the sequences do not include whole, isolated wildtype sequences.

A listing of non-limiting exemplary HU protein sequences from a variety of species.

| SEQ ID | Species | Ref. No. | Exemplary Sequence |
|---|---|---|---|
| SEQ ID NO | Tannerella | WP_041591297.1 | MNKTEFINAVAEKAGLSKVDGKKAVEAM |
| | forsythia | | VKTIQGEMKKGEKVSILGFGSFSVVEKAS |
| | | | RKGVNPQTKKVINIPARKVIKFKPGTDL |
| SEQ ID NO | Porphyromonas | WP_004334454.1 | MNKTEFIAAVAEKAGLTKADAQRAVNAF |
| | endodontalis | | TEVVKETMEKGDRLPLVGFGTFSVSQRKA |
| | | | REGKNPRTGETIKIAARKVVHFKPGANLD |
| | | | LK |
| SEQ ID NO | Porphyromonas | WP_025003580.1 | MNKSEFIAEVAAKAGMTKVDAQKSVNAF |
| | macacae | | IEVIQEQMKKGEKVALLGFGTFSVTQKAA |
| | | | RTGINPKTKKAIKIPARKAVKFKAGSALDV |
| SEQ ID NO | Bergeyella | WP_002662711.1 | MNKTDFIAAVAEKANLTKADAQRAVNAF |
| | zoohelcum | | AEVVTEQMNAGEKIALIGFGTFSVSERAA |
| | | | RKGINPATKQPINIPAKMVAKFKPGT |
| SEQ ID NO | Parabacteroides | WP_008154292.1 | MNKTEFINAVAEKSGLSKVDAKKAVEAF |
| | johnsonii | | VETVSSELKEGGKVALLGFGSFSVAEKAA |
| | | | RKGVNPKTKQPIEIPARKSVKFKAGAEL |
| SEQ ID NO | Porphyromonas | WP_025837655.1 | MNKTEFISAVAEKAGATKVDTKAIVDAA |
| | cangingivalis | | VAVIAEEMKKGEKVAILGFGTFSVVERAK |
| | | | REGFNPRTKEKIKIPARKIVKFKPGSDLDI |
| SEQ ID NO | Bacteroides | WP_025073728.1 | MNKSELISAMATEAQMSKADAKRALEAFI |
| | faccichinchillae | | TSVTNAMKAGDKVSLVGFGTFAVSERAA |
| | | | RTGINPSTKASITIPAKKVAKFKPGAEL |
| SEQ ID NO | Odoribacter | WP_013611412.1 | MNKAQLIDAIAEKAGLTKADSKKALEAFV |
| | splanchnicus | | ETVGEALKGGDKVALIGFGSFSVSERSARS |
| | | | GRNPQTGKTITIPAKKVVKFKAGAEL |
| SEQ ID NO | Bacteroides | WP_027325691.1 | MNKSELVSAMAAEAQMSKADAKKALDA |
| | pyogenes | | FISSVTKAMKAGDKVSLVGFGTFSVSERS |
| | | | ARTGINPSTKATITIPAKKVAKFKAGAEL |
| SEQ ID NO | Saprospira | WP_015691662.1 | MNKGDLIDKIAEAAGLKKADAAAALNAT |
| | grandis | | LETIADTLKAGQKITLVGFGTEDVNYRAA |
| | | | RKGINPSTQKEIQISDKVTVKFKAG |
| SEQ ID NO | Porphyromonas | WP_004583766.1 | MNKTDFIAAVAEKANLTKADAQRAVNAF |
| | gingivalis | | AEVVTEQMNAGEKIALIGFGTFSVSERAA |
| | | | RKGINPKTKKSISIPARKVVRFKPGSTLELK |

Non-limiting examples of suitable polypeptide fragments, include but are not limited to:

(a) FLEEIRLSLESGQDVKLSGF;
(b) RPGRNPKTGDVVPVSARRVV;
(c) RTGRNPQTGAEIQIAASKVP;
(d) TLSAKEIENMVKDILEFISQ;
(e) RGFGSFSLHHRQPRLGRNPK;
(f) FSLHHRQPRLGRNPKTGDSV;
(g) KKQAKAALEATLDAITASLKEG;
(h) VNERAARTGRNPQTGAEIQIAA;

(i) a polypeptide comprising the amino acid sequence NPXT; or
j) an equivalent of (a) through (i), wherein an equivalent comprises an amino acid sequence having at least about 80% homology or amino acid identity thereto, or an amino acid encoded by polynucleotide that hybridizes under conditions of high stringency to a polynucleotide encoding the amino acid sequence or its complement, wherein conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water.

As used herein, the term “chimer” or “chimeric peptide” refers to a recombinant polypeptide comprising or alternatively consisting essentially of, or yet further consisting of, two or more fragments or domains of a DNABII polypeptide conjugated directly or indirectly (such as via a linker) with each other. In one embodiment, the domains are conformational tip domains and/or conformational tail domains. Additionally or alternatively, the two or more fragments or domains is derived from the same or different DNABII polypeptide(s). In one embodiment, the chimeric peptide comprises or alternatively consists essentially of, or yet further consists of, a tip domain of IhfA and a tip domain of IhfB conjugated directly or indirectly (such as via a linker) with each other. See, e.g., U.S. Pat. No. 11,104,723, incorporated herein by reference. In another embodiment, the chimeric peptide comprises or alternatively consists essentially of, or yet further consists of, a tail domain of IhfA and a tail domain of IhfB conjugated directly or indirectly (such as via a linker) with each other. See, e.g., U.S. Pat. No. 11,104,723, incorporated herein by reference. “A conformational tip domain” of a polypeptide refers to a polypeptide that comprises a primary amino acid sequence wherein the structure has an anti-parallel beta ribbon with a sharp turn that is typically mediated by a proline residue. The “tip” of an IHF polypeptide is shown in FIG. 1 of WO2018/129078.

“A conformational tip domain” of a polypeptide refers to a polypeptide that comprises a primary amino acid sequence wherein the structure has an anti-parallel beta ribbon with a sharp turn that is typically mediated by a proline residue.

Table below shows examples of conformational tip domain polypeptides.

| Abbreviation | | | |
|---|---|---|---|
| (Protein | | | SEQ |
| name) | Bacterial strain | Sequence | ID NO: |
| Ec_HimA | E. coli K12-MG1655 | FDLRDKNQRPGRNPKTGEDI | |
| (b1712) | | | |
| Salm_HimA | Salmonella enteric | FDLRDKNQRPGRNPKTGEDI | |
| (Sty1771) | serovar typhi CT18 | | |
| Vc_HimA | V. cholera El Toz | FDLRDKNERPGRNPKTGEDI | |
| (VC_0273) | N16961 | | |
| Pa_HimA | P. aeruginosa | FDLRDKRQRPGRNPKTGEEI | |
| (NMB_1302) | | | |
| Hi_HimA | H. influenzae KW20 Rd | FELRDKSSRPGRNPKTGDVV | |
| (HI1221) | | | |
| Aa_IHFalpha | Aggregatibacter | FELRDKASRPGRNPKTGESV | |
| (YP_003255965) | actinomycetemcomitans | | |
| | D11S-1 | | |
| Mc HimA | Moraxella catarrhalis | FELKDKKPRPGRNPKTGESV | |
| (YP_003626307) | RH4 | | |
| Ng_IHFalpha | N. gonorrhoeae FA1090 | FQLRDKPQRPGRNPKTGEEV | |
| (NGO603) | (Oklahoma) | | |
| Nm HimA | N. meningitides MC5B | FQLRDKPQRPGRNPKTGEEV | |
| (NMB_0729) | | | |
| Bc IHFA | Burkholderia | FQLRDKPQRPGRNPKTGEAI | |
| (Bcen2424_1481) | cenocepacia HI2424 | | |
| Bp IHFA | Burkholderia | FQLRDKPQRPGRNPNTGEAI | |
| (BURPS668_1718) | pseudomallei 668 | | |
| Bpert IhfA | Bordetella pertusis | FQVRDKPPRPGRNPKTGETI | |
| (BP2572) | Tohama 1 | | |
| Pm HimA | Prevotella | FEVKKRLERVMVNPSTGLRM | |
| | melaninogenica ATCC | | |
| | 25845 | | |
| Pi HimA | Prevotella intermedia | FEVKKRLERIMTNPATGLRM | |
| (PIN_0345) | 17 | | |
| Tp Dbp II | Treponema palladium | FESRVRKASVGKSINTGEVV | |
| (TP_0251) | Nichols | | |
| Pm Hup | Prevotella | FKVQAVKPRESVNVNTGERV | |
| | melaninogenica ATCC | | |
| | 25845 | | |
| Pi hypo | Prevotella intermedia | FKVQAVKPRESVNVNTGERV | |
| (PIN_0343) | 17 | | |
| Sa_HU | S. aureus MW2 | FEVRERAARKGRNPQTGKEI | |
| (MW1362) | | | |
| Ec hupA | E. coli K12-MG.1655 | FKVNHRAERTGRNPQTGKEI | |
| Se_Hup | S. epidermidis RP62A | FEVRERAARKGRNPQTGKEI | |
| (SERP1041) | | | |
| Ss Hu (1310) | S. sobrinus 6715 | FEVRERAARKGRNPQTGAEI | |
| Spyog_HU | S. pyogenes | FEVRERAARKGRNPQTGAEI | |
| (Spy1239) | MGAS10270 | | |
| Sgall_HlpA | S. gallolyticus UCN34 | FEVRERAARKGRNPQTGEEI | |
| (YP_003430069) | (S. bovis) | | |
| GBS_Hup | S. agalactiae (Group B | FEVRERAARKGRNPQTGAEI | |
| (SAG_0505) | Strep) 2603V/R | | |
| Spneu_HU | S. pneumoniae R6 | FEVRERAERKGRNPQTGKEM | |
| (spr1020) | | | |
| Sg_HlpA | S. gordonii Challis | FEVRERAARKGRNPQTGKEI | |
| (SGO_0701) | NCTC7868 | | |
| Sm_HU | S. mutans UA159 | FEVRERAARKGRNPQTGEEI | |
| (Smu_589) | | | |
| Ef Hup | Enterococcus faecalis | FEVRERAARKGRNPQTGQEI | |
| (Efl 550) | VS83 | | |
| Hi_HupA | H. influenzae KW20 Rd | FKVNERAARTGRNPQTGAEI | |
| (HI0430) | | | |
| Vc_HupA | V. cholera El Toz | FKVNHRSARTGRNPQTGEEI | |
| (VC_0273) | N16961 | | |
| Pa_HupB | P. aeruginosa | FAVKERAARTGRNPQTGKPI | |
| Aa HU | Aggregatibacter | FKVNARKARTGRNPQTGAEI | |
| | actinomycetemcomitans | | |
| | D11S-1 | | |
| Vc_HupB | V. cholera El Toz | FSVRTRAARTGRNPKTGEEI | |
| (VC_1919) | N16961 | | |
| Ec hupB | E. coli K12-MG.1655 | FAVKERAARTGRNPQTGKEI | |
| Mc HupB | Moraxella catarrhalis | FSVKERAARMGRNPKTGEAI | |
| (YP_003626775) | RH4 | | |
| Bpert HupB | Bordetella pertusis | FAVSARAARTGRNPRTGETI | |
| (BP3530) | Tohama 1 | | |
| Mc HimD | Moraxella catarrhalis | FCLHHRSARIARNPRTGESV | |
| (YP_003627027) | RH4 | | |
| Pm HupB | Prevotella | FATTERPAHEGINPRSKEKI | |
| (PREME0022_2103) | melaninogenica ATCC | | |
| | 25845 | | |
| Pi Hup | Prevotella intermedia | YSVTERPAHEGINPATKQKI | |
| (PIN_A0704) | 17 | | |
| Td HU | Treponema denticola | DFAVLHGRKNARNPKTGEAV | |
| (TDE_1709) | ATCC 35405 | | |
| Pg_Hup-1 | P. gingivalis W83 | FSVSERAARKGINPKTKKSI | |
| (PG_0121) | | | |
| Hp_Hup | H. pylori 26695 | FETAEQKGKEGKVPGSDKTY | |
| (Hp0835) | | | |
| Pm HupA | Prevotella | SFIVKHRAEKTARNISKNTTI | |
| (PREME0022_0268) | melaninogenica ATCC | | |
| | 25845 | | |
| Pi Hup-2 | Prevotella intermedia | SFIVKHRAEKTARNISKNTTI | |
| (PIN_A1504) | 17 | | |
| Pg_Hup-2 | P. gingivalis W83 | FIVKERAEKTARNISKQTTI | |
| (PG_1258) | | | |
| Mt HU | Mycobacterium | FEQRRRAARVARNPRTGETV | |
| (MT_3064) | tuberculosis CDC1551 | | |
| Ms Hup | Mycobacterium | FEQRRRAARVARNPRTGETV | |
| (MSMEG_2389) | smegmatis MC2 | | |
| Ec_HimD | E. coli K12-MG1655 | FSLHYRAPRTGRNPKTGDKV | |
| (b0912) | | | |
| Salm_HimD | Salmonella enteric | FSLHYRAPRTGRNPKTGDKV | |
| (Sty0982) | serovar typhi CT18 | | |
| Vc_HipB | V. cholera El Toz | FSLHYREPRVGRNPKTGDKV | |
| (VC_1914) | N16961 | | |
| Pa_HimD | P. aeruginosa | FSLHYRAPRVGRNPKTGESV | |
| (PA3161) | | | |
| Hi_HimD | H. influenzae KW20 Rd | FSLHHRQPRLGRNPKTGDSV | |
| (HI1313) | | | |
| Aa_IHFB | Aggregatibacter | FSLHCRQPRIGRNPKTGEQV | |
| (YP_003256209) | actinomycetemcomitans | | |
| | D11S-1 | | |
| Ng_IHFβ | N. gonorrhoeae FA1090 | FDLNHRPARIGRNPKTGERV | |
| (NGO603) | (Oklahoma) | | |
| Nm HimD | N. meningitides MC5B | FDLNHRPARIGRNPKTGERV | |
| Bc IHFB | Burkholderia | FGLNRRPARVGRNPKSGEKV | |
| (Bcen2424_1048) | cenocepacia HI2424 | | |
| Bp IHFB | Burkholderia | FGLNRRPARVGRNPKSGEKV | |
| (BURPS668_2881) | pseudomallei 668 | | |
| Bpert IhfB | Bordetella pertusis | FSLSQRSPRIGRNPKSGEQV | |
| (BP0951) | Tohama 1 | | |
| Bb_Hbb | B. burgdorferi B31 | FEVRKRKGRLNARNPQTGEYV | |
| (BB_0232) | | | |

A “tip fragment” of a DNABII polypeptide intends a DNABII polypeptide that, using IHFalpha and IHFbeta as examples, forms the two arms of the proteins. Non-limiting examples of such include the tip fragment of IhfA (also referred to herein as A tip fragment): NFELRDKSSRPGRNPKTGDVV, SEQ ID NO: , and the tip fragment of IhfB (also referred to herein as B tip fragment): SLHHRQPRLGRNPKTGDSVNL (SEQ ID NO: ) or FSLHHRQPRLGRNPKTGDSV (SEQ ID NO: ).

A “tail fragment” of a DNABII polypeptide intends a region of the protein that is both exposed to the bulk medium and not occluded by DNA or other polypeptides.

In certain embodiments, the tip-chimeric peptide IhfA5-mIhfB4_{NTHI} comprises, or consists essentially of, or yet further consists of: a polypeptide sequence of

(SEQ ID NO: )
RPGRNPX₁TGDVVPVSARRVV-X-FSLHHRQPRLGRNPX₁TGDSV,

wherein “X” is an optional amino acid linker sequence, optionally comprising, or consisting essentially of, or yet further consisting of between 1 to 20 amino acids; and wherein “X₁” is any amino acid or alternatively “X₁” is selected from the amino acids Q, R, K, S, or T. In a further aspect, “X₁” is a K or Q. In a further embodiment, the tip-chimeric peptide IhfA5-mIhfB4_{NTHI} comprises, or consists essentially of, or yet further consists of: a polypeptide sequence of RPGRNPKTGDVVPVSARRVV-X-FSLHHRQPRLGRNPKTGDSV (SEQ ID NO: ), wherein “X” is an optional amino acid linker sequence optionally comprising, or consisting essentially of, or yet further consisting of between 1 to 20 amino acids. In yet a further embodiment, the tip-chimeric peptide IhfA5-mIhfB4_{NTHI} comprises or consists essentially of, or yet further consists of: a polypeptide sequence of RPGRNPKTGDVVPVSARRVVGPSLFSLHHRQPRLGRNPKTGDSV (SEQ ID NO: , in some embodiments, which is referred to herein as the tip chimer).

In certain embodiments, the tail-chimeric peptide IhfA3-IhfB2NTHI comprises, or consists essentially of, or yet further consists of: a polypeptide sequence of FLEEIRLSLESGQDVKLSGF-X-TLSAKEIENMVKDILEFISQ (SEQ ID NO: ), wherein “X” is an optional amino acid linker sequence optionally comprising, or consisting essentially of, or yet further consisting of between 1 to 20 amino acids. In certain embodiments, the linker is selected from any one or more those as disclosed herein. In one embodiment, the tail-chimeric peptide IhfA3-IhfB2_{NTHI} comprises, or consists essentially of, or yet further consists of FLEEIRLSLESGQDVKLSGFGPSLTLSAKEIENMVKDILEFISQ (SEQ ID NO:

Further provided as polypeptides are those disclosed as A1 to A4 and A6 and B1 to B6, disclosed below, which do not contain the conformation tip domain, and equivalents of these polypeptides from different organisms identified herein that produce a DNABII polypeptide. The sequences comprise:

(SEQ ID NO. );
MATITKLDIIEYLSDKYHLS
(also referred to herein as A1;
(SEQ ID NO. );
KYHLSKQDTKNVVENFLEEI
(also referred to herein as A2;
(SEQ ID NO. );
FLEEIRLSLESGQDVKLSGF
(also referred to herein as A3;
(SEQ ID NO. );
KLSGFGNFELRDKSSRPGRN
(also referred to herein as A4;
(SEQ ID NO. );
ARRVVTFKPGQKLRARVEKTK
(also referred to herein as A6;
(SEQ ID NO. )
MTKSELMEKLSAKQPTLSAK
(also referred to herein as B1;
(SEQ ID NO. )
TLSAKEIENMVKDILEFISQ
(also referred to herein as B2;
(SEQ ID NO. )
EFISQSLENGDRVEVRGFGS
(also referred to herein as B3;
(SEQ ID NO. )
RGFGSFSLHHRQPRLGRNPK
(also referred to herein as B4;
(SEQ ID NO. );
GRNPKTGDSVNLSAKSVPYF
and(also referred to herein as B5;
and
(SEQ ID NO. );
SVPYFKAGKELKARVDVQA
(also referred to herein as B6;

Non-limiting examples of DNABII polypeptides include an IHF or HU alpha or beta polypeptide; an IHF alpha polypeptide; Moraxella catarrhalis HU; E. coli HupA, HupB, himA, himD; E. faecalis HU (such as V583).

Also provided are recombinant polypeptides that comprise, or alternatively consist essentially of, or yet further consist of, between 3 and 5 conformational tip domains that can be produced by the same or different bacterial species, the amino acids sequences of which can be the same (e.g., all A5 amino acid sequences) or at least 2 or at least 3 or at least 4 or all 5 having different amino acid sequences (e.g., various combinations of A5 and mB4 and equivalents and NPX₁T containing fragments of each thereof), wherein X₁ is any amino acid, or in one aspect, an amino selected from the amino acids Q, R, K, S, or T. The conformational tip domains in the recombinant polypeptides can be in a linear or branched conformation. They can further comprise a detectable and/or a purification label linked thereto. The structural orientation of the tip domains can be “head” to tail; tail to head wherein the polypeptide comprises 3 or more tip domains, any combination of head to tails, e.g., head-head-head; tail-head-heard; tail-head-tail, wherein the amine terminus of the wild-type sequence is the “head” and the carboxy terminus of the wild-type sequence is the “tail” of the polypeptide. In one aspect, the polypeptides in sum can be between 41 and 120 amino acids in length.

Non-limiting examples of equivalent polypeptides, include a polypeptide having at least 60%, or alternatively at least 65%, or alternatively at least 70%, or alternatively at least 75%, or alternatively 80%, or alternatively at least 85%, or alternatively at least 90%, or alternatively at least 95% identity thereto or for polypeptide sequences, or a polypeptide which is encoded by a polynucleotide or its complement that hybridizes under conditions of high stringency to a polynucleotide encoding such polypeptide sequences. Conditions of high stringency are described herein and incorporated herein by reference. Applicants have determined that the bolded and underlined amino acids are heavily conserved and therefore in one aspect, are not modified or altered in designing an equivalent polypeptide. Additional examples of equivalent polypeptides include, for example DKSSRPGRNPX₁TGDVVAASARR (SEQ ID NO.:), wherein “X₁” is any amino acid or alternatively “X₁” is selected from the amino acids Q, R, K, S, or T.

Equivalent polypeptides, one kind of biologically active fragment, also include a polypeptide consisting of, or comprising the above noted polypeptides with the addition of up to 25, or alternatively 20, or alternatively 15, or alternatively up to 10, or alternatively up to 5 random amino acids on either the amine or carboxy termini (or on both). In another aspect, they equivalent polypeptide includes a polypeptide consisting of, or comprising the above noted polypeptides with the addition of up to 25, or alternatively 20, or alternatively 15, or alternatively up to 10, or alternatively up to 5 amino acids on either the amine or carboxy termini (or on both) selected from the adjacent amino acids of the corresponding wild-type sequence and equivalents of the wild-type adjacent amino acids.

### Anti-DNABII Antibodies

In some embodiments, the anti-DNABII antibody or biologically active fragment thereof recognizes and binds to a DNABII or a biologically active fragment thereof, optionally selected from one or more of: a DNABII A5 peptide, a DNABII mB4 peptide, or a recombinant polypeptide comprising the A5 and the mB4 peptides (i.e., the tip chimer).

In some embodiments, a biologically active fragment of the DNABII has at least about 50%, or at least about 60%, or at least about 70%, or at least about 80%, or at least about 85%, or at least about 90%, or at least about 95%, or at least about 96%, or at least about 97%, or at least about 98%, or at least about 99%, or at least about 100%, or at least 1.5 folds, or at least 2 folds, or at least 3 folds, or at least 4 folds, or at least 5 folds, or at least 6 folds, or at least 7 folds, or at least 8 folds, or at least 9 folds, or at least 10 folds, or at least 20 folds, or at least 50 folds, or at least 100 folds, or more of the biological activity of a reference anti-DNABII antibody. In some embodiments, the biological activity comprises, or consists essentially of, or yet further consists of specifically recognizing and binding a DNABII or a biologically active fragment thereof. Such biological activity can be measured by one of skill in the art via, for example, quantifying DNABII affinity and/or specificity of the antibody and/or competitive binding a DNABII or a biologically active fragment compared to a reference antibody. Additionally or alternatively, the biological activity here comprises, or consists essentially of, or yet further consists of one or more of: preventing, inhibiting, disrupting, dispersing or treating a biofilm in a subject.

Suitable anti-DNABII antibodies or biologically active fragments thereof can be found in U.S. Pat. No. 11,104,723, WO2014/201305, WO2017/023863, WO2017/066719, WO2018/129092, WO2018/129078, and PCT Publication No. WO 2021/007260, each of which is enclosed herein by reference in its entirety.

Non-limiting examples of anti-IHF antibodies are described herein, and in one aspect, is one or more of an antibody that specifically recognizes and binds a polypeptide identified herein, or the Arm fragment identified therein (identified above), or an equivalent of such polypeptide or a polynucleotide or polypeptide comprising one or more of the sequences:. TCTCAACGATTTA (SEQ ID NO.); WATCAANNNNTTR (where W is A or T, N is any nucleotide and R is a A or G; (SEQ ID NO.); MATITKLDIIEYLSDKYHLS (also referred to herein as hIFA1; (SEQ ID NO.); KYHLSKQDTKNVVENFLEEI (also referred to herein as hIFA2; (SEQ ID NO.); FLEEIRLSLESGQDVKLSGF (also referred to herein as hIFA3; (SEQ ID NO.); KLSGFGNFELRDKSSRPGRN (also referred to herein as hIFA4; (SEQ ID NO.); RPGRNPKTGDVVPVSARRVV (also referred to herein as hIFA5; (SEQ ID NO.); ARRVVTFKPGQKLRARVEKTK (also referred to herein as hIFA6; (SEQ ID NO.), or an equivalent thereof or a polynucleotide or peptide having at least 60%, or alternatively at least 65%, or alternatively at least 70%, or alternatively at least 75%, or alternatively 80%, or alternatively at least 85%, or alternatively at least 90%, or alternatively at least 95% identity thereto or for polypeptide sequences, which is encoded by a polynucleotide or its complement that hybridizes under conditions of high stringency to a polynucleotide encoding such polypeptide sequences. Conditions of high stringency are described above and incorporated herein by reference. Applicants have determined that the bolded and underlined amino acids are heavily conserved and therefore in one aspect, are not modified or altered in designing an equivalent polypeptide. Additional examples of equivalent polypeptides include, for example a polypeptide consisting of or comprising the above noted polypeptides with the addition of up to 25, or alternatively 20, or alternatively 15, or alternatively up to 10, or alternatively up to 5 random amino acids on either the amine or carboxy termini (or on both).

In certain aspects, the disclosure relates to an antibody or antigen binding fragment that specifically recognizes or binds an isolated or recombinant polypeptide consisting essentially of an amino acid sequence selected from: Phe Leu Glu Glu Ile Arg Leu Ser Leu Glu Ser Gly Gln Asp Val Lys Leu Ser Gly Phe (SEQ ID NO: 64), FSLHHRQPRLGR [symbol] GDSV (SEQ ID NO: 65), VNERAARTGR [symbol] GAEIQIAA (SEQ ID NO: 66), Lys Lys Gln Ala Lys Ala Ala Leu Glu Ala Thr Leu Asp Ala Ile Thr Ala Ser Leu Lys Glu Gly (SEQ ID NO. 67), a polypeptide comprising the amino acid sequence NPXT, or an equivalent of each thereof. In a further aspect, the isolated or recombinant polypeptide comprises at least 15, or alternatively at least 18, or alternatively at least 20 amino acids in total. In a further aspect, the NPXT sequence is not the terminal amino acids of the polypeptide. In some embodiments, the antibody or antigen binding fragment is not a polyclonal antibody.

In addition, antibodies can be generated against the “tip” region of a DNABII protein, that in one aspect contains or is altered to contain turn of the antiparallel beta ribbon and/or the sequence NPXT, wherein “X” refers to any amino acid. In some embodiments, X is selected from the amino acids Q, R, K, S, or T. Such antibodies may be generated using a fragment of the DNABII protein comprising the NPXT sequence, optionally flanked by between about 0, 1, 2, 3, 4, 5, 6, 7, 8, 9, 10, 11, 12, 13, 14, 15, or 16 amino acids on one or both sides of said sequence. Non-limiting examples of such are disclosed herein with the consensus amino acid sequence NPXT, in underlined and bolded text. In another aspect, antibodies may be generated against the tip region of a DNABII protein providing the consensus sequence NPXT, wherein “X” is any amino acid or alternatively X is selected from the amino acids Q, R, K, S, or T. A skilled artisan will appreciate that in any one of these sequences, the residue in the X position (marked with a [symbol] in the below [symbol] examples) may be substituted with any amino acid—e.g., Q, R, K, S, or T. Examples include:

SEQ ID NO. 68:
Haemophilus influenzae IhfA, A5 fragment:
RPGR [symbol] GDVVPVSARRVV.
SEQ ID NO. 69:
Haemophilus influenzae HU, A5 fragment:
RTGR [symbol] GAEIQIAASKVP.
SEQ ID NO. 65:
Haemophilus influenzae IhfB, modified B4 (mB4)
fragment:
FSLHHRQPRLGR [symbol] GDSV.
SEQ ID NO. 70:
Haemophilus influenzae IhfA, A tip fragment:
NFELRDKSSRPGR [symbol] GDVV.
SEQ ID NO. 71:
Haemophilus influenzae IhfB, B tip fragment:
SLHHRQPRLGR [symbol] GDSVNL.
SEQ ID NO. 66
Haemophilus influenzae HU, fragment:
VNERAARTGR [symbol] GAEIQIAA.

In some embodiments, the polypeptide comprising NPXT is at least about 20 amino acids long and the NPXT is centrally in the sequence. Non-limiting examples of such sequences include SEQ ID NOs. 68, 69, and 66. Alternatively, the polypeptides having the NPXT motif (e.g., as noted above) can be modified to remove the NPXT motif either by substitution or deletion of one or more amino acids and monoclonal antibodies can be raised against these polypeptides. Applicants have determined that antibodies raised against DNABII polypeptides lacking the NPXT motif are useful in diagnostic methods to image and monitor biofilm formation and/or disruption. In one aspect, a kit is provided comprising an antibody raised against or that binds a DNABII having the NPXT motif and an antibody raised against or binds a DNABII (e.g. a modified or naturally occurring polypeptide) that lacks the NPXT motif. For example a kit can comprise an antibody that recognizes and bind the polypeptide A5, B4, or mB4 (therapeutic) can be combined in a kit with an antibody that recognizes and bind the polypeptide A3 or B2 (diagnostic). The kit is useful for diagnosis, treatment and monitoring biofilm treatment.

In certain aspects, the disclosure relates to an antibody or antigen binding fragment that specifically recognizes or binds an isolated or recombinant polypeptide that comprises or consisting essentially of an amino acid sequence selected from: SEQ ID NOs. 64 to 65, SEQ ID NO. 66, SEQ ID NO. 67, a polypeptide comprising the amino acid sequence NPXT, or an equivalent each thereof. In some embodiments, the antibody or antigen binding fragment is not a polyclonal antibody. Non-limiting exemplary antibodies produced by the disclosed hybridomas are disclosed in Table below. The hybridoma cell lines that produce monoclonal antibodies that specifically recognize and bind Haemophilus influenzae IhfA fragment A5 (SEQ ID NO. 68), IhfB fragment B4 (Arg Gly Phe Gly Ser Phe Ser Leu His His Arg Gln Pro Arg Leu Gly Arg Asn Pro Lys, SEQ ID NO. 72), and IhfB fragment mB4 (SEQ ID NO. 65) were deposited with American Type Culture Collection (ATCC) under the listed Accession Numbers in Table below and pursuant to the provisions of the Budapest Treaty on Jul. 30, 2015; the respective hybridoma cell lines are listed in Table below. Further non-limiting exemplary antibodies include those that specifically recognize and bind Haemophilus influenzae IhfA fragment A3 (SEQ ID NO. 64) or IhfB fragment B2 (Thr Leu Ser Ala Lys Glu Ile Glu Asn Met Val Lys Asp Ile Leu Glu Phe Ile Ser Gln SEQ ID NO. 73) produced by hybridoma cell lines IhfA3 NTHI 9B10.F2.H3, IhfB2 NTHI 7A4.E4.G4, and IhfB2 NTHI 7A4.E4.G11 (these hybridomas were deposited with American Type Culture Collection (ATCC) under the Accession Numbers listed in Table below and pursuant to the provisions of the Budapest Treaty on Aug. 1, 2016); and an antibody that specifically recognizes or binds a polypeptide comprising SEQ ID NO. 66, SEQ ID NO. 67, a polypeptide comprising the amino acid sequence NPXT, or an equivalent of each thereof.

| Specificity | SEQ ID NO. | ATCC Accession No. | Hybridoma Cell Line |
|---|---|---|---|
| IhfA frag. A5 | SEQ ID NO. 68 | PTA-122334 | IhfA5 NTHI 14G8.F5.G6 |
| IhfB frag. B4 | SEQ ID NO. 72 | PTA-122336 | IhfB4 NTHI 4E11.E5.G2 |
| IhfB frag. mB4 | SEQ ID NO. 65 | PTA-122335 | mIhfB4 NTHI |
| | | | 12E6.F8.D12.D5 |
| IhfA frag. A3 | SEQ ID NO. 64 | | IhfA3 NTHI 9B10.F2.H3 |
| IhfB frag. B2 | SEQ ID NO. 73 | | IhfB2 NTHI 7A4.E4.G4 |
| | | | IhfB2 NTHI 7A4.E4.G11 |

In one aspect, the present disclosure provides an isolated antibody that is at least 85% identical to an antibody selected from the group consisting of (i) the antibody produced by hybridoma cell line IhfA5 NTHI 14G8.F5.G6, (ii) the antibody produced by hybridoma cell line IhfB4 NTHI 4E11.E5.G2, and (iii) the antibody produced by hybridoma cell line mIhfB4 NTHI 12E6.F8.D12.D5.

In one aspect, the present disclosure provides an isolated antibody comprising the CDRs of (i) the antibody produced by hybridoma cell line IhfA5 NTHI 14G8.F5.G6, (ii) the antibody produced by hybridoma cell line IhfB4 NTHI 4E11.E5.G2, or (iii) the antibody produced by hybridoma cell line mIhfB4 NTHI 12E6.F8.D12.D5. In one aspect, the present disclosure provides an isolated antibody that has CDR that are at least 85% identical to (i) the antibody produced by hybridoma cell line IhfA5 NTHI 14G8.F5.G6, (ii) the antibody produced by hybridoma cell line IhfB4 NTHI 4E11.E5.G2, or (iii) the antibody produced by hybridoma cell line mIhfB4 NTHI 12E6.F8.D12.D5.

In some aspects of the antibodies provided herein, the HC variable domain sequence comprises the HC variable domain sequence of (i) the antibody produced by hybridoma cell line IhfA5 NTHI 14G8.F5.G6, (ii) the antibody produced by hybridoma cell line IhfB4 NTHI 4E11.E5.G2, or (iii) the antibody produced by hybridoma cell line MIhfB4 NTHI 12E6.F8.D12.D5; and/or the LC variable domain sequence comprises the LC variable domain sequence of (i) the antibody produced by hybridoma cell line IhfA5 NTHI 14G8.F5.G6, (ii) the antibody produced by hybridoma cell line IhfB4 NTHI 4E11.E5.G2, or (iii) the antibody produced by hybridoma cell line mIhfB4 NTHI 12E6.F8.D12.D5.

In some aspects of the antibodies provided herein, the HC variable domain sequence comprises a HC variable domain sequence at least 85% identical to a HC variable domain sequence of (i) the antibody produced by hybridoma cell line IhfA5 NTHI 14G8.F5.G6, (ii) the antibody produced by hybridoma cell line IhfB4 NTHI 4E11.E5.G2, and (iii) the antibody produced by hybridoma cell line mIhfB4 NTHI 12E6.F8.D12.D5; and/or the LC variable domain sequence comprises a LC variable domain sequence at least 85% identical to the of LC variable domain sequence (i) the antibody produced by hybridoma cell line IhfA5 NTHI 14G8.F5.G6, (ii) the antibody produced by hybridoma cell line IhfB4 NTHI 4E11.E5.G2, and (iii) the antibody produced by hybridoma cell line mIhfB4 NTHI 12E6.F8.D12.D5.

In one aspect, the present disclosure provides an isolated antibody comprising a heavy chain (HC) variable domain sequence and a light chain (LC) variable domain sequence, wherein the heavy chain and light chain immunoglobulin variable domain sequences form an antigen binding site that binds to an epitope of a DNABII protein.

In some embodiments, the heavy chain variable region comprises a CDRH1 sequence comprising, or alternatively consisting essentially of, or yet further consisting of, an amino acid sequence comprising the CDRH1 of any one of the following antibodies: (i) the antibody produced by hybridoma cell line IhfA5 NTHI 14G8.F5.G6, (ii) the antibody produced by hybridoma cell line IhfB4 NTHI 4E11.E5.G2, and (iii) the antibody produced by hybridoma cell line mIhfB4 NTHI 12E6.F8.D12.D5.

In some embodiments, the heavy chain variable region comprises a CDRH2 sequence comprising, or alternatively consisting essentially of, or yet further consisting of, an amino acid sequence comprising the CDRH2 of any one of the following antibodies: (i) the antibody produced by hybridoma cell line IhfA5 NTHI 14G8.F5.G6, (ii) the antibody produced by hybridoma cell line IhfB4 NTHI 4E11.E5.G2, and (iii) the antibody produced by hybridoma cell line mIhfB4 NTHI 12E6.F8.D12.D5.

In some embodiments, the heavy chain variable region comprises a CDRH3 sequence comprising, or alternatively consisting essentially of, or yet further consisting of, an amino acid sequence comprising the CDRH3 of any one of the following antibodies: (i) the antibody produced by hybridoma cell line IhfA5 NTHI 14G8.F5.G6, (ii) the antibody produced by hybridoma cell line IhfB4 NTHI 4E11.E5.G2, and (iii) the antibody produced by hybridoma cell line mIhfB4 NTHI 12E6.F8.D12.D5.

In some embodiments, the heavy chain variable region comprises, or alternatively consists essentially of, or yet further consists of, the amino acid sequence comprising the heavy chain variable region sequence of any one of the following antibodies: (i) the antibody produced by hybridoma cell line IhfA5 NTHI 14G8.F5.G6, (ii) the antibody produced by hybridoma cell line IhfB4 NTHI 4E11.E5.G2, and (iii) the antibody produced by hybridoma cell line mIhfB4 NTHI 12E6.F8.D12.D5.

In some embodiments, the light chain variable region comprises a CDRL1 sequence comprising, or alternatively consisting essentially of, or yet further consisting of, an amino acid sequence comprising the CDRL1 of any one of the following antibodies: (i) the antibody produced by hybridoma cell line IhfA5 NTHI 14G8.F5.G6, (ii) the antibody produced by hybridoma cell line IhfB4 NTHI 4E11.E5.G2, and (iii) the antibody produced by hybridoma cell line mIhfB4 NTHI 12E6.F8.D12.D5.

In some embodiments, the light chain variable region comprises a CDRL2 sequence comprising, or alternatively consisting essentially of, or yet further consisting of, an amino acid sequence comprising the CDRL2 of any one of the following antibodies: (i) the antibody produced by hybridoma cell line IhfA5 NTHI 14G8.F5.G6, (ii) the antibody produced by hybridoma cell line IhfB4 NTHI 4E11.E5.G2, and (iii) the antibody produced by hybridoma cell line mIhfB4 NTHI 12E6.F8.D12.D5.

In some embodiments, the light chain variable region comprises a CDRL3 sequence comprising, or alternatively consisting essentially of, or yet further consisting of, an amino acid sequence comprising the CDRL3 of any one of the following antibodies: (i) the antibody produced by hybridoma cell line IhfA5 NTHI 14G8.F5.G6, (ii) the antibody produced by hybridoma cell line IhfB4 NTHI 4E11.E5.G2, and (iii) the antibody produced by hybridoma cell line mIhfB4 NTHI 12E6.F8.D12.D5

In some embodiments, the light chain variable region comprises, or alternatively consists essentially of, or yet further consists of, the polypeptide encoded by the polynucleotide sequence comprising the light chain variable region sequence of any one of the following antibodies: (i) the antibody produced by hybridoma cell line IhfA5 NTHI 14G8.F5.G6, (ii) the antibody produced by hybridoma cell line IhfB4 NTHI 4E11.E5.G2, and (iii) the antibody produced by hybridoma cell line mIhfB4 NTHI 12E6.F8.D12.D5.

In some embodiments, the heavy chain variable region of the antibody or fragment thereof of comprises a CDRH1 sequence comprising, or alternatively consisting essentially of, or yet further consisting of, an amino acid sequence comprising FSLTSYS (SEQ ID NO.), such as but not limited to an amino acid sequence beginning with, ending with, or consisting essentially of FSLTSYSV (SEQ ID NO.), FSLTSYSVH (SEQ ID NO.), GFSLTSYS (SEQ ID NO.), or a biological equivalent each thereof.

In some embodiments, the heavy chain variable region of the antibody or fragment thereof comprises a CDRH1 sequence comprising, or alternatively consisting essentially of, or yet further consisting of, an amino acid sequence comprising FNIKDYY (SEQ ID NO. 110), such as but not limited to an amino acid sequence beginning with, ending with, or consisting essentially of FNIKDYYM (SEQ ID NO.), FNIKDYYMH (SEQ ID NO.), GFNIKDYY (SEQ ID NO.), or a biological equivalent each thereof.

In some embodiments, the heavy chain variable region of the antibody or a fragment thereof comprises a CDRH2 sequence comprising, or alternatively consisting essentially of, or yet further consisting of, an amino acid sequence comprising IWAGGST (SEQ ID NO.), such as but not limited to an amino acid sequence beginning with, ending with, or consisting essentially of VIWAGGST (SEQ ID NO.), GVIWAGGST (SEQ ID NO.), LGVIWAGGST (SEQ ID NO.), WLGVIWAGGST (SEQ ID NO.), IWAGGSTN (SEQ ID NO.), VIWAGGSTN (SEQ ID NO.), GVIWAGGSTN (SEQ ID NO.), LGVIWAGGSTN (SEQ ID NO.), WLGVIWAGGSTN (SEQ ID NO.), IWAGGSTNY (SEQ ID NO.), VIWAGGSTNY (SEQ ID NO.), GVIWAGGSTNY (SEQ ID NO.), LGVIWAGGSTNY (SEQ ID NO.),WLGVIWAGGSTNY (SEQ ID NO.), or a biological equivalent each thereof.

In some embodiments, the heavy chain variable region of the antibody or a fragment thereof comprises a CDRH2 sequence comprising, or alternatively consisting essentially of, or yet further consisting of, an amino acid sequence comprising IDPENDDT (SEQ ID NO.), such as but not limited to an amino acid sequence beginning with, ending with, or consisting essentially of WIDPENDDT (SEQ ID NO.), GWIDPENDDT (SEQ ID NO.), IGWIDPENDDT (SEQ ID NO.), WIGWIDPENDDT (SEQ ID NO.), IDPENDDTE (SEQ ID NO.), WIDPENDDTE (SEQ ID NO.), GWIDPENDDTE (SEQ ID NO.), IGWIDPENDDTE (SEQ ID NO.), WIGWIDPENDDTE (SEQ ID NO.), IDPENDDTEY (SEQ ID NO.), WIDPENDDTEY (SEQ ID NO.) GWIDPENDDTEY (SEQ ID NO.), IGWIDPENDDTEY (SEQ ID NO.), WIGWIDPENDDTEY (SEQ ID NO.), or a biological equivalent each thereof.

In some embodiments, the heavy chain variable region of the antibody or a fragment thereof comprises a CDRH3 sequence comprising, or alternatively consisting essentially of, or yet further consisting of, an amino acid sequence comprising REDS (SEQ ID NO.), such as but not limited to an amino acid sequence beginning with, ending with, or consisting essentially of AREDS (SEQ ID NO.) or a biological equivalent thereof.

In some embodiments, the heavy chain variable region of the antibody or a fragment thereof comprises a CDRH3 sequence comprising, or alternatively consisting essentially of, or yet further consisting of, an amino acid sequence comprising TELGAY (SEQ ID NO.) or a biological equivalent thereof.

In some embodiments, the heavy chain variable region of the antibody or a fragment thereof comprises, or alternatively consists essentially of, or yet further consists of, the polypeptide encoded by the below noted polynucleotide sequences: Gaggtgcagctgcaggagtctggacctggcctggtgacgccctcacagagcctgtccatgacttgcactgtctctgggttttcattaac cagctatagtgtacactgggttcgccagcctccaggaaagagtctggagtggctgggagtaatatgggctggtggaagcacaaattat aattcggctctcatgtccagactgagcatcagcaaagacaactccaagagccaagttttcttaaaaatggacagtctgcaaactgatgac acagccatatactactgtgccagagaggactcctggggtcaaggaacctcagtcaccgtctcctca (SEQ ID NO.) or a biological equivalent thereof.

In some embodiments, the heavy chain variable region of the antibody or a fragment thereof comprises, or alternatively consists essentially of, or yet further consists of, the amino acid sequence: EVQLQESGPGLVTPSQSLSMTCTVSGFSLTSYSVHWVRQPPGKSLEWLGVIWAGGST NYNSALMSRLSISKDNSKSQVFLKMDSLQTDDTAIYYCAREDSWGQGTSVTVSS (SEQ ID NO.) or a biological equivalent thereof.

In some embodiments, the heavy chain variable region of the antibody or a fragment thereof comprises, or alternatively consists essentially of, or yet further consists of, the polypeptide encoded by the below noted polynucleotide sequences: Gaggtgcagctgcaggagtctggggcagagcttgtgaggtcaggggcctcagtcaagttgtcctgcacagcttctggcttcaacatta aagactactatatgcactgggtgaagcagaggcctgaacagggcctggagtggattggatggattgatcctgaaaatgatgatactga atatgtcccgaagttccagggcaaggccagtatgactgcagacacatcctccaacacagcctacctgcagctcagcagcctgacatct gaggacactgccgtctattactgtacagagctcggagcttactggggccaggggactctggtc (SEQ ID NO.) or a biological equivalent thereof.

In some embodiments, the heavy chain variable region of the antibody or a fragment thereof comprises, or alternatively consists essentially of, or yet further consists of, the amino acid sequence: EVQLQESGAELVRSGASVKLSCTASGFNIKDYYMHWVKQRPEQGLEWIGWIDPEND DTEYVPKFQGKASMTADTSSNTAYLQLSSLTSEDTAVYYCTELGAYWGQGTLV (SEQ ID NO.) or a biological equivalent thereof.

In some embodiments, the light chain variable region of the antibody or a fragment thereof comprises a CDRL1 sequence comprising, or alternatively consisting essentially of, or yet further consisting of, an amino acid sequence comprising QNVGTN (SEQ ID NO.), such as but not limited to an amino acid sequence beginning with, ending with, or consisting essentially of QNVGTNV (SEQ ID NO.), QNVGTNVA (SEQ ID NO.), or a biological equivalent each thereof.

In some embodiments, the light chain variable region of the antibody or a fragment thereof comprises a CDRL1 sequence comprising, or alternatively consisting essentially of, or yet further consisting of, an amino acid sequence comprising QSLLDSNGKTY (SEQ ID NO.), such as but not limited to an amino acid sequence beginning with, ending with, or consisting essentially of QSLLDSNGKTYL (SEQ ID NO.), QSLLDSNGKTYLN (SEQ ID NO.), or a biological equivalent each thereof.

In some embodiments, the light chain variable region of the antibody or a fragment thereof comprises a CDRL2 sequence comprising, or alternatively consisting essentially of, or yet further consisting of, an amino acid sequence comprising SAS (SEQ ID NO.), such as but not limited to an amino acid sequence beginning with, ending with, or consisting essentially of YSAS (SEQ ID NO.), IYSAS (SEQ ID NO.), LIYSAS (SEQ ID NO.), ALIYSAS (SEQ ID NO.), SASY (SEQ ID NO.), YSASY (SEQ ID NO.), IYSASY (SEQ ID NO.), LIYSASY (SEQ ID NO.), ALIYSASY (SEQ ID NO.), SASYR (SEQ ID NO.), YSASYR (SEQ ID NO.), IYSASYR (SEQ ID NO.), LIYSASYR (SEQ ID NO.), ALIYSASYR (SEQ ID NO.), SASYRY (SEQ ID NO.), YSASYRY (SEQ ID NO.), IYSASYRY (SEQ ID NO.), LIYSASYRY (SEQ ID NO.), ALIYSASYRY (SEQ ID NO.), SASYRYS (SEQ ID NO.), YSASYRYS (SEQ ID NO.), IYSASYRYS (SEQ ID NO.), LIYSASYRYS (SEQ ID NO.), ALIYSASYRYS (SEQ ID NO.), or a biological equivalent each thereof.

In some embodiments, the light chain variable region of the antibody or a fragment thereof comprises a CDRL2 sequence comprising, or alternatively consisting essentially of, or yet further consisting of, an amino acid sequence comprising LVS (SEQ ID NO.), such as but not limited to an amino acid sequence beginning with, ending with, or consisting essentially of YLVS (SEQ ID NO.), IYLVS (SEQ ID NO.), LIYLVS (SEQ ID NO.), RLIYLVS (SEQ ID NO.), LVSK (SEQ ID NO.), YLVSK (SEQ ID NO.), IYLVSK (SEQ ID NO.), LIYLVSK (SEQ ID NO.), RLIYLVSK (SEQ ID NO.), LVSKL (SEQ ID NO.), YLVSKL (SEQ ID NO.), IYLVSKL (SEQ ID NO.), LIYLVSKL (SEQ ID NO.), RLIYLVSKL (SEQ ID NO.), LVSKLD (SEQ ID NO.), YLVSKLD (SEQ ID NO.), IYLVSKLD (SEQ ID NO.), LIYLVSKLD (SEQ ID NO.), RLIYLVSKLD (SEQ ID NO.), LVSKLDS (SEQ ID NO.), YLVSKLDS (SEQ ID NO.), IYLVSKLDS (SEQ ID NO.), LIYLVSKLDS (SEQ ID NO.), RLIYLVSKLDS (SEQ ID NO.), or a biological equivalent each thereof.

In some embodiments, the light chain variable region of the antibody or a fragment thereof comprises a CDRL3 sequence comprising, or alternatively consisting essentially of, or yet further consisting of, an amino acid sequence comprising QQYNSYP (SEQ ID NO.), such as but not limited to an amino acid sequence beginning with, ending with, or consisting essentially of QQYNSYPT (SEQ ID NO.), or a biological equivalent thereof.

In some embodiments, the light chain variable region of the antibody or a fragment thereof comprises a CDRL3 sequence comprising, or alternatively consisting essentially of, or yet further consisting of, an amino acid sequence comprising WQSTHFPH (SEQ ID NO.), such as but not limited to an amino acid sequence beginning with, ending with, or consisting essentially of WQSTHFPHT (SEQ ID NO.) or a biological equivalent thereof.

In some embodiments, the light chain variable region of the antibody or fragment thereof comprises, or alternatively consists essentially of, or yet further consists of, the polypeptide encoded by the polynucleotide sequence: gacattgtgatgacccagtctcaaaaattcatgtccacatcagtaggagacagggtcagcgtcacctgcaaggccagtcagaatgtgg gtactaatgtagcctggtatcaacagaaaccagggcaatctcctaaagcactgatttactcggcatcctaccggtacagtggagtccctg atcgcttcacaggcagtggatctgggacagatttcactctcaccatcagcaatgtgcagtctgaagacttggcagagtatttctgtcagc aatataacagctatcccacgttcggaggggggaccaagttggaaataaaa (SEQ ID NO.) or a biological equivalent thereof.

In some embodiments, the light chain variable region of the antibody or fragment thereof comprises, or alternatively consists essentially of, or yet further consists of, the amino acid sequence: DIVMTQSQKFMSTSVGDRVSVTCKASQNVGTNVAWYQQKPGQSPKALIYSASYRYS GVPDRFTGSGSGTDFTLTISNVQSEDLAEYFCQQYNSYPTFGGGTKLEIK (SEQ ID NO.) or a biological equivalent thereof.

In some embodiments, the light chain variable region comprises, or alternatively consists essentially of, or yet further consists of, the polypeptide encoded by the polynucleotide sequence: Gatgttgtgatgacccagattccactcactttgtcggttaccattggacaaccagcctccatctcttgcaagtcaagtcagagcctcttag atagtaatggaaagacatatttgaattggttgtttcagaggccaggccagtctccaaagcgcctaatctatctggtgtctaaactggactct ggagtccctgacaggttcactggcagtggatcagggacagatttcacactgaaaatcagcagagttgaggctgaggatttgggaattta ttattgctggcaaagtacacattttcctcacacgttcggaggggggaccaagttggaaatcaaa (SEQ ID NO.) or a biological equivalent thereof.

In some embodiments, the light chain variable region comprises, or alternatively consists essentially of, or yet further consists of, the amino acid sequence: DVVMTQIPLTLSVTIGQPASISCKSSQSLLDSNGKTYLNWLFQRPGQSPKRLIYLVSKL DSGVPDRFTGSGSGTDFTLKISRVEAEDLGIYYCWQSTHFPHTFGGGTKLEIK (SEQ ID NO.) or a biological equivalent thereof.

Exemplary antibodies comprising the disclosed CDR sequences and heavy and light chain variable sequences are disclosed in Tables below, respectively. Alternate CDR predictions may be made based on the heavy and/or light chain sequences—e.g., based on the Kabat, Clothia, AbM, or contact definitions of CDR specificity; details of these CDR prediction methods are known in the art (see, e.g., bioinf.org.uk/abs/#cdrid) and/or commercially available. Those disclosed in Table are the result of utilizing the CDR prediction algorithms provided by the Ofran Lab (Paratome available at ofranservices.biu.ac.il/site/services/paratome/index.html) and Green Mountain Antibodies' CDR prediction program.

| ANTIBODY | CDRH1 | CDRH2 | CDRH3 | CDRL1 | CDRL2 | CDRL3 |
|---|---|---|---|---|---|---|
| Ihf A5 | Phe Ser | Ile Trp | Arg Glu | Gln Asn | Ser Ala | Gln Gln |
| | Leu Thr | Ala Gly | Asp Ser or | Val Gly | Ser or Ala | Tyr Asn |
| | Ser Tyr | Gly Ser | Ala Arg | Thr Asn | Leu Ile | Ser Tyr |
| | Ser Val | Thr or Trp | Glu Asp | or Gln | Tyr Ser | Pro or Gln |
| | His or | Leu Gly | Ser | Asn Val | Ala Ser | Gln Tyr |
| | Gly Phe | Val Ile | | Gly Thr | Tyr Arg | Asn Ser |
| | Ser Leu | Trp Ala | | Asn Val | Tyr Ser | Tyr Pro |
| | Thr Ser | Gly Gly | | Ala | | Thr |
| | Tyr Ser | Ser Thr | | | | |
| | | Asn Tyr | | | | |
| ANTIBODY | CDRH1 | CDRH2 | CDRH3 | CDRL1 | CDRL2 | CDRL3 |
| Ihf mB4 | Phe Asn | Ile Asp | Thr Glu | Gln Ser | Leu Val | Trp Gln |
| | Ile Lys | Pro Glu | Leu Gly | Leu Leu | Ser or Arg | Ser Thr |
| | Asp Tyr | Asn Asp | Ala Tyr | Asp Ser | Leu Ile | His Phe |
| | Tyr Met | Asp Thr | | Asn Gly | Tyr Leu | Pro His or |
| | His or | or Trp Ile | | Lys Thr | Val Ser | Trp Gln |
| | Gly Phe | Gly Trp | | Tyr Leu or | Lys Leu | Ser Thr |
| | Asn Ile | Ile Asp | | Gln Ser | Asp Ser | His Phe |
| | Lys Asp | Pro Glu | | Leu Leu | | Pro His |
| | Tyr Tyr | Asn Asp | | Asp Ser | | Thr |
| | | Asp Thr | | Asn Gly | | |
| | | Glu Tyr | | Lys Thr | | |
| | | | | Tyr Leu | | |
| | | | | Asn | | |

| ANTIBODY | Heavy Chain Variable Region | Light Chain Variable Region |
|---|---|---|
| Ihf A5 | Glu Val Gln Leu Gln Glu Ser | Asp Ile Val Met Thr Gln Ser |
| | Gly Pro Gly Leu Val Thr Pro | Gln Lys Phe Met Ser Thr Ser |
| | Ser Gln Ser Leu Ser Met Thr | Val Gly Asp Arg Val Ser Val |
| | Cys Thr Val Ser Gly Phe Ser | Thr Cys Lys Ala Ser Gln Asn |
| | Leu Thr Ser Tyr Ser Val His | Val Gly Thr Asn Val Ala Trp |
| | Trp Val Arg Gln Pro Pro Gly | Tyr Gln Gln Lys Pro Gly Gln |
| | Lys Ser Leu Glu Trp Leu Gly | Ser Pro Lys Ala Leu Ile Tyr |
| | Val Ile Trp Ala Gly Gly Ser | Ser Ala Ser Tyr Arg Tyr Ser |
| | Thr Asn Tyr Asn Ser Ala Leu | Gly Val Pro Asp Arg Phe Thr |
| | Met Ser Arg Leu Ser Ile Ser | Gly Ser Gly Ser Gly Thr Asp |
| | Lys Asp Asn Ser Lys Ser Gln | Phe Thr Leu Thr Ile Ser Asn |
| | Val Phe Leu Lys Met Asp Ser | Val Gln Ser Glu Asp Leu Ala |
| | Leu Gln Thr Asp Asp Thr Ala | Glu Tyr Phe Cys Gln Gln Tyr |
| | Ile Tyr Tyr Cys Ala Arg Glu | Asn Ser Tyr Pro Thr Phe Gly |
| | Asp Ser Trp Gly Gln Gly Thr | Gly Gly Thr Lys Leu Glu Ile |
| | Ser Val Thr Val Ser Ser | Lys |
| Ihf mB4 | Glu Val Gln Leu Gln Glu Ser | Asp Val Val Met Thr Gln Ile |
| | Gly Ala Glu Leu Val Arg Ser | Pro Leu Thr Leu Ser Val Thr |
| | Gly Ala Ser Val Lys Leu Ser | Ile Gly Gln Pro Ala Ser Ile |
| | Cys Thr Ala Ser Gly Phe Asn | Ser Cys Lys Ser Ser Gln Ser |
| | Ile Lys Asp Tyr Tyr Met His | Leu Leu Asp Ser Asn Gly Lys |
| | Trp Val Lys Gln Arg Pro Glu | Thr Tyr Leu Asn Trp Leu Phe |
| | Gln Gly Leu Glu Trp Ile Gly | Gln Arg Pro Gly Gln Ser Pro |
| | Trp Ile Asp Pro Glu Asn Asp | Lys Arg Leu Ile Tyr Leu Val |
| | Asp Thr Glu Tyr Val Pro Lys | Ser Lys Leu Asp Ser Gly Val |
| | Phe Gln Gly Lys Ala Ser Met | Pro Asp Arg Phe Thr Gly Ser |
| | Thr Ala Asp Thr Ser Ser Asn | Gly Ser Gly Thr Asp Phe Thr |
| | Thr Ala Tyr Leu Gln Leu Ser | Leu Lys Ile Ser Arg Val Glu |
| | Ser Leu Thr Ser Glu Asp Thr | Ala Glu Asp Leu Gly Ile Tyr |
| | Ala Val Tyr Tyr Cys Thr Glu | Tyr Cys Trp Gln Ser Thr His |
| | Leu Gly Ala Tyr Trp Gly Gln | Phe Pro His Thr Phe Gly Gly |
| | Gly Thr Leu Val | Gly Thr Lys Leu Glu Ile Lys |

In one aspect, the present disclosure provides an isolated antibody that is at least 85%, or alternatively at least 90%, or alternatively at least 95%, identical to an antibody selected from the group consisting of Ihf A5, Ihf mB4, or a biological equivalent each thereof.

In one aspect, the present disclosure provides an isolated antibody comprising the CDRs of Ihf A5. In one aspect, the present disclosure provides an isolated antibody that is at least 85%, or alternatively at least 90%, or alternatively at least 95% identical to Ihf A5 or a biological equivalent thereof.

In one aspect, the present disclosure provides an isolated antibody comprising the CDRs of Ihf mB4. In one aspect, the present disclosure provides an isolated antibody that is at least 85%, or alternatively at least 90%, or alternatively at least 95%, identical to Ihf mB4 or a biological equivalent thereof.

In some aspects of the antibodies provided herein, the HC variable domain sequence comprises a variable domain sequence of Ihf A5 and the LC variable domain sequence comprises a variable domain sequence of Ihf A5.

In some aspects of the antibodies provided herein, the HC variable domain sequence comprises a variable domain sequence of Ihf mB4 and the LC variable domain sequence comprises a variable domain sequence of Ihf mB4.

Further provided herein are isolated polynucleotides encoding the above noted polypeptides, vector and host cells containing same, as well as methods for recombinant production of the polypeptides using recombinant cell systems as known in the art and described herein.

In another aspect of the present technology, the isolated antibody includes one or more of the following characteristics:
(a) the light chain immunoglobulin variable domain sequence comprises one or more CDRs that are at least 85%, or alternatively at least 90%, or alternatively at least 95% identical to a CDR of a light chain variable domain of any of the disclosed light chain sequences;
(b) the heavy chain immunoglobulin variable domain sequence comprises one or more CDRs that are at least 85%, or alternatively at least 90%, or alternatively at least 95% identical to a CDR of a heavy chain variable domain of any of the disclosed heavy chain sequences;
(c) the light chain immunoglobulin variable domain sequence is at least 85%, or alternatively at least 90%, or alternatively at least 95% identical to a light chain variable domain of any of the disclosed light chain sequences;
(d) the HC immunoglobulin variable domain sequence is at least 85%, or alternatively at least 90%, or alternatively at least 95% identical to a heavy chain variable domain of any of the disclosed light chain sequences; and
(e) the antibody binds an epitope that overlaps with an epitope bound by any of the disclosed sequences.

In some aspects, the antibodies comprise a heavy chain constant region that is at least 80% identical to the heavy chain constant region sequence of any one of the following antibodies: (i) the antibody produced by hybridoma cell line IhfA5 NTHI 14G8.F5.G6, (ii) the antibody produced by hybridoma cell line IhfB4 NTHI 4E11.E5.G2, and (iii) the antibody produced by hybridoma cell line mIhfB4 NTHI 12E6.F8.D12.D5.

In some aspects, the antibodies comprise a light chain constant region that is at least 80% identical to the light chain constant region sequence of any one of the following antibodies: (i) the antibody produced by hybridoma cell line IhfA5 NTHI 14G8.F5.G6, (ii) the antibody produced by hybridoma cell line IhfB4 NTHI 4E11.E5.G2, and (iii) the antibody produced by hybridoma cell line mIhfB4 NTHI 12E6.F8.D12.D5.

Non-limiting exemplary antibodies include those disclosed herein, for example those generated against the disclosed polypeptide fragments, including but not limited to:

| | |
|---|---|
| (a) | FLEEIRLSLESGQDVKLSGF; |
| (b) | RPGRNPKTGDVVPVSARRVV; |
| (c) | RTGRNPQTGAEIQIAASKVP; |
| (d) | TLSAKEIENMVKDILEFISQ; |
| (e) | RGFGSFSLHHRQPRLGRNPK; |
| (f) | FSLHHRQPRLGRNPKTGDSV; |
| (g) | KKQAKAALEATLDAITASLKEG; |
| (h) | VNERAARTGRNPQTGAEIQIAA; |

(i) a polypeptide comprising the amino acid sequence NPXT; or
(j) an equivalent of (a) through (i), wherein an equivalent comprises an amino acid sequence having at least about 80% homology or amino acid identity thereto, or an amino acid encoded by polynucleotide that hybridizes under conditions of high stringency to a polynucleotide encoding the amino acid sequence or its complement, wherein conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water.

This disclosure provides an antibody fragment (e.g., a Fab fragment or an antigen binding fragment). This disclosure also provides an isolated polypeptide that comprises, or alternatively consists essentially of, or yet further consists of the amino acid sequence of the Fab fragment or antigen binding fragment (e.g., a Fab fragment) wherein the fragment or the polypeptide binds and/or specifically recognizes a DNABII polypeptide and/or biofilm component comprising a DNABII protein or polypeptide.

The present disclosure provides an isolated antibody comprising a heavy chain (HC) variable domain sequence and a light chain (LC) variable domain sequence, wherein the heavy chain and light chain immunoglobulin variable domain sequences form an antigen binding site that binds to an epitope of a DNABII protein. In certain embodiments, the antibody or fragment thereof binds to a DNABII peptide (such as the tip region of the DNABII peptide including but not limited to: a tip region of IHF or HU, a tip region of IHFA or IHFB, and/or the tip-chimeric peptide IhfA5-mIhfB4_{NTHI}; and/or the tail region of the DNABII peptide, including but not limited to: a tail region of IHF or HU, a tail region of IHFA or IHFB, and/or the tail-chimeric peptide IhfA3-IhfB2_{NTHI}). In one embodiment, the antibody or fragment thereof binds to the tip-chimeric peptide IhfA5-mIhfB4_{NTHI}. In another embodiment, the antibody or fragment thereof binds to the tail-chimeric peptide IhfA3-IhfB2_{NTHI}.

In one aspect, provided herein are antibodies and antigen binding fragments thereof that comprise, or consist essentially of or yet consist of, a heavy chain (HC) immunoglobulin variable domain sequence comprising, consisting essentially of, or consisting of, a sequence selected from the group of amino acid (aa) 25 to aa 144 of SEQ ID NOs: 1-6, 13, 24 or 26 or an equivalent of each thereof, and/or a light chain (LC) immunoglobulin variable domain sequence comprising, consisting essentially of, or consisting of, a sequence selected from the group of aa 21 to aa 132 of SEQ ID NOs: 7-9, 14 or 25, aa 21 to aa 126 of SEQ ID NOs: 10-12 or 27, or an equivalent of each thereof.

In a further aspect, provided are antibodies and antigen binding fragments thereof that comprise, or consist essentially of or yet consist of, a heavy chain (HC) comprising, consisting essentially of, or consisting of, a sequence selected from the group of aa 25 to aa 473 of SEQ ID NOs: 1-6, 13, 24 or 26 or an equivalent of each thereof, and/or a light chain (LC) comprising, consisting essentially of, or consisting of, a sequence selected from the group of aa 21 to aa 239 of SEQ ID NOs: 7-9, 14, or 25, aa 21 to aa 233 of SEQ ID NOs: 10-12 or 27, or an equivalent of each thereof.

In yet a further aspect, provided are antibodies and antigen binding fragments thereof that comprise, or consist essentially of or yet consist of, a heavy chain (HC) comprising, consisting essentially of, or consisting of, a sequence selected from the group of SEQ ID NOs: 1-6, 13, 24 or 26 or an equivalent of each thereof, and/or a light chain (LC) comprising, consisting essentially of, or consisting of, a sequence selected from the group of SEQ ID NOs: 7-12, 14, 25, or 27, or an equivalent of each thereof.

In another aspect, provided herein are antibodies and antigen binding fragments thereof that comprise, or consist essentially of or yet consist of, a heavy chain (HC) immunoglobulin variable domain sequence comprising, consisting essentially of, or consisting of, a sequence selected from the group of amino acid (aa) 25 to 144 of SEQ ID NO: 1-6, 13, 24 or 26, or an equivalent thereof, and/or a light chain (LC) immunoglobulin variable domain sequence comprising, consisting essentially of, or consisting of, a sequence selected from the group of aa 21 to aa 132 of SEQ ID NO: 6-9, 14 or 25, aa 21 to aa 126 of SEQ ID NOs: 10-12, or 27, or an equivalent of each thereof.

In yet another aspect, provided is an antibody or a fragment thereof that comprises or consists essentially of, or yet further consists of: any one or any two or all three CDRs of a sequence selected from the group of: SEQ ID NOs: 1-6, 13, 24 or 26, or an equivalent of each thereof; and/or any one or any two or all three CDRs of a sequence selected from the group of: SEQ ID NOs: 7-12, 14, 25 or 27, or an equivalent of each thereof.

In another aspect, provided herein are antibodies and antigen binding fragments thereof that comprise, or consist essentially of or yet consist of, a heavy chain (HC) immunoglobulin variable domain sequence comprising, consisting essentially of, or consisting of a sequence selected from the group of aa 25 to aa 144 of SEQ ID NO: 13, 24 or 26, or an equivalent thereof, and a light chain (LC) immunoglobulin variable domain sequence comprising, consisting essentially of, or consisting of, a sequence selected from the group of aa 21 to aa 132 of SEQ ID NOs: 14 or 25, aa 21 to aa 126 of SEQ ID NO: 27, or an equivalent thereof. In a further aspect, provided herein are antibodies and antigen binding fragments thereof that comprise, or consist essentially of or yet consist of, a heavy chain (HC) immunoglobulin variable domain sequence comprising, consisting essentially of, or consisting of a sequence selected from the group of aa 25 to aa 144 of SEQ ID NOs: 1-6, 13, 24 or 26, or an equivalent of each thereof, and/or a light chain (LC) immunoglobulin variable domain sequence comprising, consisting essentially of, or consisting of a sequence selected from the group of aa 21 to aa 132 of SEQ ID NOs: 7-9, 14 or 25, aa 21 to aa 126 of SEQ ID NOs: 10-12 or 27, or an equivalent of each thereof.

Also provided are antibodies and antigen binding fragments thereof that comprise, or consist essentially of, or yet consist of a heavy chain (HC) immunoglobulin variable domain sequence that comprises, consisting essentially of, or consisting of, an amino acid sequence of aa 25 to aa 144 of SEQ ID NO: 1 or an equivalent thereof and a light chain (LC) immunoglobulin variable domain sequence that comprises, consisting essentially of, or consisting of, an amino acid sequence of any one of aa 21 to aa 132 of SEQ ID NOs: 7-9, 14 or 25, aa 21 to aa 126 of SEQ ID NOs: 10-12 or 27, or an equivalent of each thereof. In another aspect, provided herein are antibodies or fragments thereof comprising, or alternatively consisting essentially, or consisting of, a heavy chain (HC) immunoglobulin variable domain sequence that comprises, consisting essentially of, or consisting of, an amino acid sequence of aa 25 to aa 144 of SEQ ID NO: 2 or an equivalent thereof and a light chain (LC) immunoglobulin variable domain sequence that comprises, consists essentially of, or alternatively consists of, an amino acid sequence of any one of aa 21 to aa 132 of SEQ ID NOs: 7-9, 14 or 25, aa 21 to aa 126 of SEQ ID NOs: 10-12 or 27, or an equivalent of each thereof. In a further aspect, provided herein are antibodies and antigen binding fragments thereof that comprise, or consist essentially of, or yet consist of, a heavy chain (HC) immunoglobulin variable domain sequence comprises, consisting essentially of, or consisting of, an amino acid sequence of aa 25 to aa 144 of SEQ ID NO: 3 or an equivalent thereof and a light chain (LC) immunoglobulin variable domain sequence comprises, consisting essentially of, or consisting of, an amino acid sequence of any one of aa 21 to aa 132 of SEQ ID NOs: 7-9, 14 or 25, aa 21 to aa 126 of SEQ ID NOs: 10-12 or 27, or an equivalent of each thereof.

In a yet further aspect, provided herein are antibodies and antigen binding fragments thereof that comprise, or consist essentially of, or yet consist of, a heavy chain (HC) immunoglobulin variable domain sequence that comprises, consisting essentially of, or consisting of, an amino acid sequence of aa 25 to aa 144 of SEQ ID NO: 4 or an equivalent thereof and a light chain (LC) immunoglobulin variable domain sequence that comprises, consisting essentially of, or consisting of, an amino acid sequence of any one of aa 21 to aa 132 of SEQ ID NOs: 7-9, 14 or 25, aa 21 to aa 126 of SEQ ID NOs: 10-12 or 27, or an equivalent of each thereof. Also provided are antibodies and antigen binding fragments thereof comprising, or alternatively consisting essentially of, or yet consisting of, a heavy chain (HC) immunoglobulin variable domain sequence that comprises, consisting essentially of, or consisting of, an amino acid sequence of aa 25 to aa 144 of SEQ ID NO: 5 or an equivalent thereof and a light chain (LC) immunoglobulin variable domain sequence that comprises, consisting essentially of, or consisting of, an amino acid sequence of any one of aa 21 to aa 132 of SEQ ID NOs: 7-9, 14 or 25, aa 21 to aa 126 of SEQ ID NOs: 10-12 or 27, or an equivalent of each thereof. Yet further provided are antibodies and antigen binding fragments thereof comprising, or alternatively consisting essentially or consisting of, a heavy chain (HC) immunoglobulin variable domain sequence that comprises, consisting essentially of, or consisting of, an amino acid sequence of aa 25 to aa 144 of SEQ ID NO: 6 or an equivalent thereof, and a light chain (LC) immunoglobulin variable domain sequence that comprises, consisting essentially of, or consisting of, an amino acid sequence of any one of aa 21 to aa 132 of SEQ ID NOs: 7-9, 14 or 25, aa 21 to aa 126 of SEQ ID NOs: 10-12 or 27, or an equivalent of each thereof.

In another aspect, provided herein are antibodies or antigen binding fragments thereof, that comprise, or consisting essentially of, or consisting of, a heavy chain (HC) immunoglobulin variable domain sequence that comprises, consisting essentially of, or consisting of an amino acid sequence of aa 25 to aa 144 of any one of SEQ ID NOs: 1-6, 13, 24 or 26, or an equivalent of each thereof, and a light chain (LC) immunoglobulin variable domain sequence comprises, consisting essentially of, or consisting of, an amino acid sequence of aa 21 to aa 132 of SEQ ID NO 7, or an equivalent thereof. Yet further provided are antibodies or antigen binding fragments thereof comprising, or consisting essentially of, or yet further consisting of, a heavy chain (HC) immunoglobulin variable domain sequence that comprises, consisting essentially of, or consisting of, an amino acid sequence of aa 25 to aa 144 of any one of SEQ ID NOs: 1-6, 13, 24 or 26 or an equivalent of each thereof, and a light chain (LC) immunoglobulin variable domain sequence that comprises, consisting essentially of, or consisting of, an amino acid sequence of aa 21 to aa 132 of SEQ ID NO: 8, or an equivalent thereof. In another aspect, also provided are antibodies or antigen binding fragments thereof that comprise, or consist essentially of, or yet further consist of, a heavy chain (HC) immunoglobulin variable domain sequence that comprises, consisting essentially of, or consisting of, an amino acid sequence of aa 25 to aa 144 of any one of SEQ ID NOs: 1-6, 13, 24 or 26, or an equivalent of each thereof, and a light chain (LC) immunoglobulin variable domain sequence that comprises, consisting essentially of, or consisting of, an amino acid sequence of aa 21 to aa 132 of SEQ ID NO: 9, or an equivalent thereof.

In a further aspect, also provided are antibodies or antigen binding fragments thereof, that comprise, or consist essentially of, or yet further consist of, a heavy chain (HC) immunoglobulin variable domain sequence that comprises, or alternatively consists essentially of, or yet further consists of, an amino acid sequence of aa 25 to aa 144 of any one of SEQ ID NOs: 1-6, 13, 24 or 26, or an equivalent of each thereof, and a light chain (LC) immunoglobulin variable domain sequence that comprises, or consists essentially of, or yet further consists of, an amino acid sequence of aa 21 to aa 126 of SEQ ID NO: 10, or an equivalent thereof. Also provided are antibodies and antigen binding fragments thereof that comprise, or consist essentially of, or yet further consist of, a heavy chain (HC) immunoglobulin variable domain sequence that comprises, or consist essentially of, or yet further consists of, an amino acid sequence of aa 25 to aa 144 of any one of f SEQ ID NOs: 1-6, 13, 24 or 26, or an equivalent of each thereof, and a light chain (LC) immunoglobulin variable domain that comprises, or consists essentially thereof, or consists of, an amino acid sequence of aa 21 to aa 126 of SEQ ID NO: 11, or an equivalent thereof. Also provided are antibodies and antigen binding fragments thereof, comprising, or consisting essentially of, or yet further consisting of, a heavy chain (HC) immunoglobulin variable domain sequence that comprises, or consists essentially thereof, or consisting of, an amino acid sequence of aa 25 to aa 144 of any one of SEQ ID NOs: 1-6, 13, 24 or 26, or an equivalent of each thereof, and the light chain (LC) immunoglobulin variable domain sequence comprises an amino acid sequence of aa 21 to aa 126 of SEQ ID NO: 12, or an equivalent thereof.

In one aspect, provided herein are antibodies and antigen binding fragments thereof are provided, that comprise or alternatively consist essentially of, or yet further consist of, a heavy chain (HC) immunoglobulin variable domain sequence that comprises, or alternatively consists essentially of, or yet further consists of, an amino acid sequence of aa 25 to aa 144 of SEQ ID NO: 1 or an equivalent thereof and a light chain (LC) immunoglobulin variable domain sequence that comprises, or alternatively consists essentially thereof, or consists of, an amino acid sequence of aa 21 to aa 132 of SEQ ID NO: 7, or an equivalent thereof. In one embodiment, antibodies and antigen binding fragments thereof are provided, that comprise or alternatively consist essentially of, or yet further consist of, a heavy chain (HC) immunoglobulin variable domain sequence that comprises, or alternatively consists essentially of, or yet further consists of, an amino acid sequence of aa 25 to aa 144 of SEQ ID NO: 1 or an equivalent thereof and a light chain (LC) immunoglobulin variable domain sequence that comprises, or alternatively consists essentially thereof, or consists of, an amino acid sequence of aa 21 to aa 132 of SEQ ID NO: 8, or an equivalent thereof. In an another embodiment, provided herein are antibodies and antigen binding fragments thereof that comprise, or alternatively consist essentially thereof, or consist of, a heavy chain (HC) immunoglobulin variable domain sequence that comprises, or consists essentially thereof, or consists of, an amino acid sequence of aa 25 to aa 144 of SEQ ID NO: 1 or an equivalent thereof and a light chain (LC) immunoglobulin variable domain sequence that comprises, or consists essentially thereof, or consists of, an amino acid sequence of aa 21 to aa 132 of SEQ ID NO: 9, or an equivalent thereof. In some embodiments, the antibodies or antigen binding fragments are anti tip chimer (i.e., specifically binding and recognizing the tip chimer).

In an another aspect, provided herein are antibodies and antigen binding fragments thereof that comprise, or alternatively consist essentially thereof, or consist of a heavy chain (HC) immunoglobulin variable domain sequence that comprises, or alternatively consists essentially thereof, or consists of, an amino acid sequence of aa 25 to aa 144 of SEQ ID NO: 2 or an equivalent thereof and a light chain (LC) immunoglobulin variable domain sequence that comprises, or consists essentially thereof, of consists of, an amino acid sequence of aa 21 to aa 132 of SEQ ID NO: 7, or an equivalent thereof. In an another aspect, provided herein are antibodies and antigen binding fragments thereof that comprise, or alternatively consist essentially thereof, or consist of a heavy chain (HC) immunoglobulin variable domain sequence comprises an amino acid sequence of aa 25 to aa 144 of SEQ ID NO: 2 or an equivalent thereof, and a light chain (LC) immunoglobulin variable domain sequence that comprises, or consists essentially thereof, or yet further consists of, an amino acid sequence of aa 21 to aa 132 of SEQ ID NO: 8, or an equivalent thereof. In a further another aspect, provided herein are antibodies and antigen binding fragments thereof that comprise, or alternatively consist essentially thereof, or consist of a heavy chain (HC) immunoglobulin variable domain sequence that comprises, or consisting essentially thereof, or consisting of, an amino acid sequence of aa 25 to aa 144 of SEQ ID NO: 2 or an equivalent thereof, and a light chain (LC) immunoglobulin variable domain sequence that comprises, consisting essentially thereof, or consists of, an amino acid sequence of aa 21 to aa 132 of SEQ ID NO: 9, or an equivalent thereof. In some embodiments, the antibodies or antigen binding fragments are anti tip chimer (i.e., specifically binding and recognizing the tip chimer).

In an another aspect, provided herein are antibodies and antigen binding fragments thereof that comprise, or alternatively consist essentially thereof, or consist of a heavy chain (HC) immunoglobulin variable domain sequence that comprises, or consists essentially thereof, or consists of, an amino acid sequence of aa 25 to aa 144 of SEQ ID NO: 3 or an equivalent thereof and a light chain (LC) immunoglobulin variable domain sequence comprises, or consists of an amino acid sequence of aa 21 to aa 132 of SEQ ID NO: 7, or an equivalent thereof. In a further aspect, provided herein are antibodies and antigen binding fragments thereof that comprise, or alternatively consist essentially thereof, or consist of a heavy chain (HC) immunoglobulin variable domain sequence that comprises, or consists essentially of, or yet further consists of, an amino acid sequence of aa 25 to aa 144 of SEQ ID NO: 3 or an equivalent thereof, and the light chain (LC) immunoglobulin variable domain sequence comprises, or consists essentially thereof, or consists of, an amino acid sequence of aa 21 to aa 132 of SEQ ID NO: 8, or an equivalent thereof. In one embodiment, provided herein are antibodies and antigen binding fragments thereof that comprise, or alternatively consist essentially thereof, or consist of a heavy chain (HC) immunoglobulin variable domain sequence that comprises, or consists essentially thereof, or consists of, an amino acid sequence of aa 25 to aa 144 of SEQ ID NO: 3 or an equivalent thereof and the light chain (LC) immunoglobulin variable domain sequence comprises an amino acid sequence of aa 21 to aa 132 of SEQ ID NO: 9, or an equivalent thereof. In some embodiments, the antibodies or antigen binding fragments are anti tip chimer (i.e., specifically binding and recognizing the tip chimer).

Also provided are antibodies and antigen binding fragments thereof that comprise, or alternatively consist essentially thereof, or consist of a heavy chain (HC) immunoglobulin variable domain sequence that comprises, or consists essentially thereof, or consists of, an amino acid sequence of aa 25 to aa 144 of SEQ ID NO: 4 or an equivalent thereof and a light chain (LC) immunoglobulin variable domain sequence comprises, or consists essentially thereof, or consists of, an amino acid sequence of aa 21 to aa 126 of SEQ ID NO: 10, or an equivalent thereof. In an another aspect, provided herein are antibodies and antigen binding fragments thereof that comprise, or alternatively consist essentially thereof, or consist of a heavy chain (HC) immunoglobulin variable domain sequence that comprises, or consists essentially thereof, or consists of, an amino acid sequence of aa 25 to aa 144 of SEQ ID NO: 4 or an equivalent thereof, and a light chain (LC) immunoglobulin variable domain sequence that comprises, or consists essentially thereof, or consists of, an amino acid sequence of aa 21 to aa 126 of SEQ ID NO: 11, or an equivalent thereof. In a further aspect, provided herein are antibodies and antigen binding fragments thereof that comprise, or alternatively consist essentially thereof, or consist of a heavy chain (HC) immunoglobulin variable domain sequence that comprises, or consists essentially thereof, or consists of, an amino acid sequence of aa 25 to aa 144 of SEQ ID NO: 4 or an equivalent thereof, and a light chain (LC) immunoglobulin variable domain sequence that comprises, or consists essentially thereof, or consists thereof, an amino acid sequence of aa 21 to aa 126 of SEQ ID NO: 12, or an equivalent thereof. In some embodiments, the antibodies or antigen binding fragments are anti tail chimer (i.e., specifically binding and recognizing the tail chimer).

In one embodiment, provided herein are antibodies and antigen binding fragments thereof that comprise, or alternatively consist essentially thereof, or consist of a heavy chain (HC) immunoglobulin variable domain sequence that comprises, or consists essentially thereof, or consists of, an amino acid sequence of aa 25 to aa 144 of SEQ ID NO: 5 or an equivalent thereof, and a light chain (LC) immunoglobulin variable domain sequence that comprises, or consists essentially thereof, or consists of, an amino acid sequence of aa 21 to aa 126 of SEQ ID NO: 10, or an equivalent thereof. In an another embodiment, provided herein are antibodies and antigen binding fragments thereof that comprise, or alternatively consist essentially thereof, or consist of a heavy chain (HC) immunoglobulin variable domain sequence that comprises, or consists essentially thereof, or consists of, an amino acid sequence of aa 25 to aa 144 of SEQ ID NO: 5 or an equivalent thereof, and a light chain (LC) immunoglobulin variable domain sequence that comprises, or consists essentially thereof, or consists of, an amino acid sequence of aa 21 to aa 126 of SEQ ID NO: 11, or an equivalent thereof. In a further aspect, provided herein are antibodies and antigen binding fragments thereof that comprise, or alternatively consist essentially thereof, or consist of a heavy chain (HC) immunoglobulin variable domain sequence that comprises, or consists essentially thereof, or consists of, an amino acid sequence of aa 25 to aa 144 of SEQ ID NO: 5 or an equivalent thereof, and a light chain (LC) immunoglobulin variable domain sequence that comprises, or consists essentially thereof, an amino acid sequence of aa 21 to aa 126 of SEQ ID NO: 12, or an equivalent thereof. In some embodiments, the antibodies or antigen binding fragments are anti tail chimer (i.e., specifically binding and recognizing the tail chimer).

In another embodiment, provided herein are antibodies and antigen binding fragments thereof that comprise, or alternatively consist essentially thereof, or consist of a heavy chain (HC) immunoglobulin variable domain sequence that comprises, or consists essentially thereof, or consists of, an amino acid sequence of aa 25 to aa 144 of SEQ ID NO: 6 or an equivalent thereof, and a light chain (LC) immunoglobulin variable domain sequence that comprises, or consists essentially thereof, or consists of, an amino acid sequence of aa 21 to aa 126 of SEQ ID NO: 10, or an equivalent thereof. In one aspect, provided herein are antibodies and antigen binding fragments thereof that comprise, or alternatively consist essentially thereof, or consist of a heavy chain (HC) immunoglobulin variable domain sequence that comprises, or consisting essentially thereof, or consists thereof, an amino acid sequence of aa 25 to aa 144 of SEQ ID NO: 6 or an equivalent thereof, and a light chain (LC) immunoglobulin variable domain sequence that comprises, or consists essentially thereof, or consists of, an amino acid sequence of aa 21 to aa 126 of SEQ ID NO: 11, or an equivalent thereof. In one embodiment, provided herein are antibodies and antigen binding fragments thereof that comprise, or alternatively consist essentially thereof, or consist of a heavy chain (HC) immunoglobulin variable domain sequence that comprises, consisting essentially thereof, or consisting of, an amino acid sequence of aa 25 to aa 144 of SEQ ID NO: 6 or an equivalent thereof, and a light chain (LC) immunoglobulin variable domain sequence that comprises, consists essentially thereof, or consists of, an amino acid sequence of aa 21 to aa 126 of SEQ ID NO: 12, or an equivalent thereof. In some embodiments, the antibodies or antigen binding fragments are anti tail chimer (i.e., specifically binding and recognizing the tail chimer).

In one aspect, provided is an antibody or a fragment thereof, that comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 24 or an equivalent thereof, and/or a light chain (LC) immunoglobulin variable domain sequence comprising, or consisting essentially of, or yet further consisting of an amino acid sequence of aa 21 to aa 132 of SEQ ID NO: 25 or an equivalent thereof. In a further embodiment, the antibody or fragment thereof binds to a tip region of a DNABII peptide (including but not limited to: a tip region of IHF or HU, a tip region of IHFA or IHFB, and/or the tip-chimeric peptide IhfA5-mIhfB4_{NTHI}). In one embodiment, the antibody or fragment thereof binds to the tip-chimeric peptide IhfA5-mIhfB4_{NTHI}. In yet a further embodiment, the fragment is an antigen binding fragment. In some embodiments, the antibodies or antigen binding fragments are anti tip chimer (i.e., specifically binding and recognizing the tip chimer). In one embodiment, the antibody or fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 1 or an equivalent thereof, and/or a light chain (LC) immunoglobulin variable domain sequence comprising, or consisting essentially of, or yet further consisting of an amino acid sequence of aa 21 to aa 132 of SEQ ID NO: 7 or an equivalent thereof. In another embodiment, the antibody or fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 1 or an equivalent thereof, and/or a light chain (LC) immunoglobulin variable domain sequence comprising, or consisting essentially of, or yet further consisting of an amino acid sequence of aa 21 to aa 132 of SEQ ID NO: 8 or an equivalent thereof. In yet another embodiment, the antibody or fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 1 or an equivalent thereof, and/or a light chain (LC) immunoglobulin variable domain sequence comprising, or consisting essentially of, or yet further consisting of an amino acid sequence of aa 21 to aa 132 of SEQ ID NO: 9 or an equivalent thereof. In one embodiment, the antibody or fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 2 or an equivalent thereof, and/or a light chain (LC) immunoglobulin variable domain sequence comprising, or consisting essentially of, or yet further consisting of an amino acid sequence of aa 21 to aa 132 of SEQ ID of NO: 7 or an equivalent thereof. In another embodiment, the antibody or fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 2 or an equivalent thereof, and/or a light chain (LC) immunoglobulin variable domain sequence comprising, or consisting essentially of, or yet further consisting of an amino acid sequence of aa 21 to aa 132 of SEQ ID NO: 8 or an equivalent thereof. In yet another embodiment, the antibody or fragment thereof comprises or consists essentially of, or yet further consists of: a the heavy chain (HC) immunoglobulin variable domain sequence comprising, or consisting essentially of, or yet further consisting of an amino acid sequence of aa 25 to aa 144 of SEQ ID NO: 2 or an equivalent thereof, and/or a light chain (LC) immunoglobulin variable domain sequence comprising, or consisting essentially of, or yet further consisting of an amino acid sequence of aa 21 to aa 132 of SEQ ID NO: 9 or an equivalent thereof. In one embodiment, the antibody or fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 3 or an equivalent thereof, and/or a light chain (LC) immunoglobulin variable domain sequence comprising, or consisting essentially of, or yet further consisting of an amino acid sequence of aa 21 to aa 132 of SEQ ID NO: 7 or an equivalent thereof. In another embodiment, the antibody or fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 3 or an equivalent thereof, and/or a light chain (LC) immunoglobulin variable domain sequence comprising, or consisting essentially of, or yet further consisting of an amino acid sequence of aa 21 to aa 132 of SEQ ID NO: 8 or an equivalent thereof. In yet another embodiment, the antibody or fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 3 or an equivalent thereof, and/or a light chain (LC) immunoglobulin variable domain sequence comprising, or consisting essentially of, or yet further consisting of an amino acid sequence of aa 21 to aa 132 of SEQ ID NO: 9 or an equivalent thereof.

In another aspect, provided is an antibody or a fragment thereof, that comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 26 or an equivalent thereof, and a light chain (LC) immunoglobulin variable domain sequence comprising, or consisting essentially of, or yet further consisting of an amino acid sequence of aa 21 to aa 126 of SEQ ID NO: 27 or an equivalent thereof. In a further embodiment, the antibody or fragment thereof binds to a tail region of a DNABII peptide (including but not limited to: a tail region of IHF or HU, a tail region of IHFA or IHFB, and/or the tail-chimeric peptide IhfA3-IhfB2_{NTHI}). In one embodiment, the antibody of fragment thereof binds to the tail-chimeric peptide IhfA3-IhfB2_{NTHI}. In yet a further embodiment, the fragment is an antigen binding fragment. In some embodiments, the antibodies or antigen binding fragments are anti tail chimer (i.e., specifically binding and recognizing the tail chimer). In one embodiment, the antibody or fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 4 or an equivalent thereof, and/or a light chain (LC) immunoglobulin variable domain sequence comprising, or consisting essentially of, or yet further consisting of an amino acid sequence of aa 21 to aa 126 of SEQ ID NO: 10 or an equivalent thereof. In another embodiment, the antibody or fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 4 or an equivalent thereof, and/or a light chain (LC) immunoglobulin variable domain sequence comprising, or consisting essentially of, or yet further consisting of an amino acid sequence of aa 21 to aa 126 of SEQ ID NO: 11 or an equivalent thereof. In yet another embodiment, the antibody or fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 4 or an equivalent thereof, and/or a light chain (LC) immunoglobulin variable domain sequence comprising, or consisting essentially of, or yet further consisting of an amino acid sequence of aa 21 to aa 126 of SEQ ID NO: 12 or an equivalent thereof. In one embodiment, the antibody or fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 5 or an equivalent thereof, and/or a light chain (LC) immunoglobulin variable domain sequence comprising, or consisting essentially of, or yet further consisting of an amino acid sequence of aa 21 to aa 126 of SEQ ID NO: 10 or an equivalent thereof. In another embodiment, the antibody or fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 5 or an equivalent thereof, and/or a light chain (LC) immunoglobulin variable domain sequence comprising, or consisting essentially of, or yet further consisting of an amino acid sequence of aa 21 to aa 126 of SEQ ID NO: 11 or an equivalent thereof. In yet another embodiment, the antibody or fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 5 or an equivalent thereof, and/or a light chain (LC) immunoglobulin variable domain sequence comprising, or consisting essentially of, or yet further consisting of an amino acid sequence of aa 21 to aa 126 of SEQ ID NO: 12 or an equivalent thereof. In one embodiment, the antibody or fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 6 or an equivalent thereof, and/or a light chain (LC) immunoglobulin variable domain sequence comprising, or consisting essentially of, or yet further consisting of an amino acid sequence of aa 21 to aa 126 of SEQ ID NO: 10 or an equivalent thereof. In another embodiment, the antibody or fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 6 or an equivalent thereof, and/or a light chain (LC) immunoglobulin variable domain sequence comprising, or consisting essentially of, or yet further consisting of an amino acid sequence of aa 21 to aa 126 of SEQ ID NO: 11 or an equivalent thereof. In yet another embodiment, the antibody or fragment thereof comprises or consists essentially of, or yet further consists of: a heavy chain (HC) immunoglobulin variable domain sequence comprising, or consisting essentially of, or yet further consisting of an amino acid sequence of aa 25 to aa 144 of SEQ ID NO: 6 or an equivalent thereof, and/or a light chain (LC) immunoglobulin variable domain sequence comprising, or consisting essentially of, or yet further consisting of an amino acid sequence of aa 21 to aa 126 of SEQ ID NO: 12 or an equivalent thereof.

In one aspect, provided is an antibody or a fragment thereof, that comprises or consists essentially of, or yet further consists of: a heavy chain (HC) comprising, or consisting essentially of, or yet further consisting of an amino acid sequence of SEQ ID NO: 24 or an equivalent thereof, and/or a light chain (LC) comprising, or consisting essentially of, or yet further consisting of an amino acid sequence of SEQ ID NO: 25 or an equivalent thereof. In a further embodiment, the antibody or fragment thereof binds to a tip region of a DNABII peptide (including but not limited to: a tip region of IHF or HU, a tip region of IHFA or IHFB, and/or the tip-chimeric peptide IhfA5-mIhfB4_{NTHI}). In one embodiment, the antibody or fragment thereof binds to the tip-chimeric peptide IhfA5-mIhfB4_{NTHI}. In some embodiments, the antibodies or antigen binding fragments are anti tip chimer (i.e., specifically binding and recognizing the tip chimer). In yet a further embodiment, the fragment is an antigen binding fragment. In one embodiment, the antibody or fragment thereof comprises or consists essentially of, or yet further consists of: a heavy chain (HC) comprising, or consisting essentially of, or yet further consisting of an amino acid sequence of SEQ ID NO: 1 or an equivalent thereof, and/or a light chain (LC) comprising, or consisting essentially of, or yet further consisting of an amino acid sequence of SEQ ID NO: 7 or an equivalent thereof. In another embodiment, the antibody or fragment thereof comprises or consists essentially of, or yet further consists of: a heavy chain (HC) comprising, or consisting essentially of, or yet further consisting of an amino acid sequence of SEQ ID NO: 1 or an equivalent thereof, and/or a light chain (LC) comprising, or consisting essentially of, or yet further consisting of an amino acid sequence of SEQ ID NO: 8 or an equivalent thereof. In yet another embodiment, the antibody or fragment thereof comprises or consists essentially of, or yet further consists of: a heavy chain (HC) comprising, or consisting essentially of, or yet further consisting of an amino acid sequence of SEQ ID NO: 1 or an equivalent thereof, and/or a light chain (LC) comprising, or consisting essentially of, or yet further consisting of an amino acid sequence of SEQ ID NO: 9 or an equivalent thereof. In one embodiment, the antibody or fragment thereof comprises or consists essentially of, or yet further consists of: a heavy chain (HC) comprising, or consisting essentially of, or yet further consisting of an amino acid sequence of SEQ ID NO: 2 or an equivalent thereof, and/or a light chain (LC) comprising, or consisting essentially of, or yet further consisting of an amino acid sequence of SEQ ID of NO: 7 or an equivalent thereof. In another embodiment, the antibody or fragment thereof comprises or consists essentially of, or yet further consists of: a heavy chain (HC) comprising, or consisting essentially of, or yet further consisting of an amino acid sequence of SEQ ID NO: 2 or an equivalent thereof, and/or a light chain (LC) comprising, or consisting essentially of, or yet further consisting of an amino acid sequence of SEQ ID NO: 8 or an equivalent thereof. In yet another embodiment, the antibody or fragment thereof comprises or consists essentially of, or yet further consists of: a the heavy chain (HC) comprising, or consisting essentially of, or yet further consisting of an amino acid sequence of SEQ ID NO: 2 or an equivalent thereof, and/or a light chain (LC) comprising, or consisting essentially of, or yet further consisting of an amino acid sequence of SEQ ID NO: 9 or an equivalent thereof. In one embodiment, the antibody or fragment thereof comprises or consists essentially of, or yet further consists of: a heavy chain (HC) comprising, or consisting essentially of, or yet further consisting of an amino acid sequence of SEQ ID NO: 3 or an equivalent thereof, and/or a light chain (LC) comprising, or consisting essentially of, or yet further consisting of an amino acid sequence of SEQ ID NO: 7 or an equivalent thereof. In another embodiment, the antibody or fragment thereof comprises or consists essentially of, or yet further consists of: a heavy chain (HC) comprising, or consisting essentially of, or yet further consisting of an amino acid sequence of SEQ ID NO: 3 or an equivalent thereof, and/or a light chain (LC) comprising, or consisting essentially of, or yet further consisting of an amino acid sequence of SEQ ID NO: 8 or an equivalent thereof. In yet another embodiment, the antibody or fragment thereof comprises or consists essentially of, or yet further consists of: a heavy chain (HC) comprising, or consisting essentially of, or yet further consisting of an amino acid sequence of SEQ ID NO: 3 or an equivalent thereof, and/or a light chain (LC) comprising, or consisting essentially of, or yet further consisting of an amino acid sequence of SEQ ID NO: 9 or an equivalent thereof.

In another aspect, provided is an antibody or a fragment thereof, that comprises or consists essentially of, or yet further consists of: a heavy chain (HC) comprising, or consisting essentially of, or yet further consisting of an amino acid sequence of SEQ ID NO: 26 or an equivalent thereof, and a light chain (LC) comprising, or consisting essentially of, or yet further consisting of an amino acid sequence of SEQ ID NO: 27 or an equivalent thereof. In a further embodiment, the antibody or fragment thereof binds to a tail region of a DNABII peptide (including but not limited to: a tail region of IHF or HU, a tail region of IHFA or IHFB, and/or the tail-chimeric peptide IhfA3-IhfB2_{NTHI}). In one embodiment, the antibody of fragment thereof binds to the tail-chimeric peptide IhfA3-IhfB2_{NTHI}. In some embodiments, the antibodies or antigen binding fragments are anti tail chimer (i.e., specifically binding and recognizing the tail chimer). In yet a further embodiment, the fragment is an antigen binding fragment. In one embodiment, the antibody or fragment thereof comprises or consists essentially of, or yet further consists of: a heavy chain (HC) comprising, or consisting essentially of, or yet further consisting of an amino acid sequence of SEQ ID NO: 4 or an equivalent thereof, and/or a light chain (LC) comprising, or consisting essentially of, or yet further consisting of an amino acid sequence of SEQ ID NO: 10 or an equivalent thereof. In another embodiment, the antibody or fragment thereof comprises or consists essentially of, or yet further consists of: a heavy chain (HC) comprising, or consisting essentially of, or yet further consisting of an amino acid sequence of SEQ ID NO: 4 or an equivalent thereof, and/or a light chain (LC) comprising, or consisting essentially of, or yet further consisting of an amino acid sequence of SEQ ID NO: 11 or an equivalent thereof. In yet another embodiment, the antibody or fragment thereof comprises or consists essentially of, or yet further consists of: a heavy chain (HC) comprising, or consisting essentially of, or yet further consisting of an amino acid sequence of SEQ ID NO: 4 or an equivalent thereof, and/or a light chain (LC) comprising, or consisting essentially of, or yet further consisting of an amino acid sequence of SEQ ID NO: 12 or an equivalent thereof. In one embodiment, the antibody or fragment thereof comprises or consists essentially of, or yet further consists of: a heavy chain (HC) comprising, or consisting essentially of, or yet further consisting of an amino acid sequence of SEQ ID NO: 5 or an equivalent thereof, and/or a light chain (LC) comprising, or consisting essentially of, or yet further consisting of an amino acid sequence of SEQ ID NO: 10 or an equivalent thereof. In another embodiment, the antibody or fragment thereof comprises or consists essentially of, or yet further consists of: a heavy chain (HC) comprising, or consisting essentially of, or yet further consisting of an amino acid sequence of SEQ ID NO: 5 or an equivalent thereof, and/or a light chain (LC) comprising, or consisting essentially of, or yet further consisting of an amino acid sequence of SEQ ID NO: 11 or an equivalent thereof. In yet another embodiment, the antibody or fragment thereof comprises or consists essentially of, or yet further consists of: a heavy chain (HC) comprising, or consisting essentially of, or yet further consisting of an amino acid sequence of SEQ ID NO: 5 or an equivalent thereof, and/or a light chain (LC) comprising, or consisting essentially of, or yet further consisting of an amino acid sequence of SEQ ID NO: 12 or an equivalent thereof. In one embodiment, the antibody or fragment thereof comprises or consists essentially of, or yet further consists of: a heavy chain (HC) comprising, or consisting essentially of, or yet further consisting of an amino acid sequence of SEQ ID NO: 6 or an equivalent thereof, and/or a light chain (LC) comprising, or consisting essentially of, or yet further consisting of an amino acid sequence of SEQ ID NO: 10 or an equivalent thereof. In another embodiment, the antibody or fragment thereof comprises or consists essentially of, or yet further consists of: a heavy chain (HC) comprising, or consisting essentially of, or yet further consisting of an amino acid sequence of SEQ ID NO: 6 or an equivalent thereof, and/or a light chain (LC) comprising, or consisting essentially of, or yet further consisting of an amino acid sequence of SEQ ID NO: 11 or an equivalent thereof. In yet another embodiment, the antibody or fragment thereof comprises or consists essentially of, or yet further consists of: a heavy chain (HC) comprising, or consisting essentially of, or yet further consisting of an amino acid sequence of SEQ ID NO: 6 or an equivalent thereof, and/or a light chain (LC) comprising, or consisting essentially of, or yet further consisting of an amino acid sequence of SEQ ID NO: 12 or an equivalent thereof.

In one aspect, provided is an antibody or a fragment thereof, that comprises or consists essentially of, or yet further consists of: a heavy chain (HC) comprising, or consisting essentially of, or yet further consisting of an amino acid sequence of aa 25 to aa 473 of SEQ ID NO: 24 or an equivalent thereof, and/or a light chain (LC) comprising, or consisting essentially of, or yet further consisting of an amino acid sequence of aa 21 to aa 239 of SEQ ID NO: 25 or an equivalent thereof. In a further embodiment, the antibody or fragment thereof binds to a tip region of a DNABII peptide (including but not limited to: a tip region of IHF or HU, a tip region of IHFA or IHFB, and/or the tip-chimeric peptide IhfA5-mIhfB4_{NTHI}). In one embodiment, the antibody or fragment thereof binds to the tip-chimeric peptide IhfA5-mIhfB4_{NTHI}. In some embodiments, the antibodies or antigen binding fragments are anti tip chimer (i.e., specifically binding and recognizing the tip chimer). In yet a further embodiment, the fragment is an antigen binding fragment. In one embodiment, the antibody or fragment thereof comprises or consists essentially of, or yet further consists of: a heavy chain (HC) comprising, or consisting essentially of, or yet further consisting of an amino acid sequence of aa 25 to aa 473 of SEQ ID NO: 1 or an equivalent thereof, and/or a light chain (LC) comprising, or consisting essentially of, or yet further consisting of an amino acid sequence of aa 21 to aa 239 of SEQ ID NO: 7 or an equivalent thereof. In another embodiment, the antibody or fragment thereof comprises or consists essentially of, or yet further consists of: a heavy chain (HC) comprising, or consisting essentially of, or yet further consisting of an amino acid sequence of aa 25 to aa 473 of SEQ ID NO: 1 or an equivalent thereof, and/or a light chain (LC) comprising, or consisting essentially of, or yet further consisting of an amino acid sequence of aa 21 to aa 239 of SEQ ID NO: 8 or an equivalent thereof. In yet another embodiment, the antibody or fragment thereof comprises or consists essentially of, or yet further consists of: a heavy chain (HC) comprising, or consisting essentially of, or yet further consisting of an amino acid sequence of aa 25 to aa 473 of SEQ ID NO: 1 or an equivalent thereof, and/or a light chain (LC) comprising, or consisting essentially of, or yet further consisting of an amino acid sequence of aa 21 to aa 239 of SEQ ID NO: 9 or an equivalent thereof. In one embodiment, the antibody or fragment thereof comprises or consists essentially of, or yet further consists of: a heavy chain (HC) comprising, or consisting essentially of, or yet further consisting of an amino acid sequence of aa 25 to aa 473 of SEQ ID NO: 2 or an equivalent thereof, and/or a light chain (LC) comprising, or consisting essentially of, or yet further consisting of an amino acid sequence of aa 21 to aa 239 of SEQ ID of NO: 7 or an equivalent thereof. In another embodiment, the antibody or fragment thereof comprises or consists essentially of, or yet further consists of: a heavy chain (HC) comprising, or consisting essentially of, or yet further consisting of an amino acid sequence of aa 25 to aa 473 of SEQ ID NO: 2 or an equivalent thereof, and/or a light chain (LC) comprising, or consisting essentially of, or yet further consisting of an amino acid sequence of aa 21 to aa 239 of SEQ ID NO: 8 or an equivalent thereof. In yet another embodiment, the antibody or fragment thereof comprises or consists essentially of, or yet further consists of: a the heavy chain (HC) comprising, or consisting essentially of, or yet further consisting of an amino acid sequence of aa 25 to aa 473 of SEQ ID NO: 2 or an equivalent thereof, and/or a light chain (LC) comprising, or consisting essentially of, or yet further consisting of an amino acid sequence of aa 21 to aa 239 of SEQ ID NO: 9 or an equivalent thereof. In one embodiment, the antibody or fragment thereof comprises or consists essentially of, or yet further consists of: a heavy chain (HC) comprising, or consisting essentially of, or yet further consisting of an amino acid sequence of aa 25 to aa 473 of SEQ ID NO: 3 or an equivalent thereof, and/or a light chain (LC) comprising, or consisting essentially of, or yet further consisting of an amino acid sequence of aa 21 to aa 239 of SEQ ID NO: 7 or an equivalent thereof. In another embodiment, the antibody or fragment thereof comprises or consists essentially of, or yet further consists of: a heavy chain (HC) comprising, or consisting essentially of, or yet further consisting of an amino acid sequence of aa 25 to aa 473 of SEQ ID NO: 3 or an equivalent thereof, and/or a light chain (LC) comprising, or consisting essentially of, or yet further consisting of an amino acid sequence of aa 21 to aa 239 of SEQ ID NO: 8 or an equivalent thereof. In yet another embodiment, the antibody or fragment thereof comprises or consists essentially of, or yet further consists of: a heavy chain (HC) comprising, or consisting essentially of, or yet further consisting of an amino acid sequence of aa 25 to aa 473 of SEQ ID NO: 3 or an equivalent thereof, and/or a light chain (LC) comprising, or consisting essentially of, or yet further consisting of an amino acid sequence of aa 21 to aa 239 of SEQ ID NO: 9 or an equivalent thereof.

In another aspect, provided is an antibody or a fragment thereof, that comprises or consists essentially of, or yet further consists of: a heavy chain (HC) comprising, or consisting essentially of, or yet further consisting of an amino acid sequence of aa 25 to aa 473 of SEQ ID NO: 26 or an equivalent thereof, and a light chain (LC) comprising, or consisting essentially of, or yet further consisting of an amino acid sequence of aa 21 to aa 233 of SEQ ID NO: 27 or an equivalent thereof. In a further embodiment, the antibody or fragment thereof binds to a tail region of a DNABII peptide (including but not limited to: a tail region of IHF or HU, a tail region of IHFA or IHFB, and/or the tail-chimeric peptide IhfA3-IhfB2_{NTHI}). In one embodiment, the antibody of fragment thereof binds to the tail-chimeric peptide IhfA3-IhfB2_{NTHI}. In some embodiments, the antibodies or antigen binding fragments are anti tail chimer (i.e., specifically binding and recognizing the tail chimer). In yet a further embodiment, the fragment is an antigen binding fragment. In one embodiment, the antibody or fragment thereof comprises or consists essentially of, or yet further consists of: a heavy chain (HC) comprising, or consisting essentially of, or yet further consisting of an amino acid sequence of aa 25 to aa 473 of SEQ ID NO: 4 or an equivalent thereof, and/or a light chain (LC) comprising, or consisting essentially of, or yet further consisting of an amino acid sequence of aa 21 to aa 233 of SEQ ID NO: 10 or an equivalent thereof. In another embodiment, the antibody or fragment thereof comprises or consists essentially of, or yet further consists of: a heavy chain (HC) comprising, or consisting essentially of, or yet further consisting of an amino acid sequence of aa 25 to aa 473 of SEQ ID NO: 4 or an equivalent thereof, and/or a light chain (LC) comprising, or consisting essentially of, or yet further consisting of an amino acid sequence of aa 21 to aa 233 of SEQ ID NO: 11 or an equivalent thereof. In yet another embodiment, the antibody or fragment thereof comprises or consists essentially of, or yet further consists of: a heavy chain (HC) comprising, or consisting essentially of, or yet further consisting of an amino acid sequence of aa 25 to aa 473 of SEQ ID NO: 4 or an equivalent thereof, and/or a light chain (LC) comprising, or consisting essentially of, or yet further consisting of an amino acid sequence of aa 21 to aa 233 of SEQ ID NO: 12 or an equivalent thereof. In one embodiment, the antibody or fragment thereof comprises or consists essentially of, or yet further consists of: a heavy chain (HC) comprising, or consisting essentially of, or yet further consisting of an amino acid sequence of aa 25 to aa 473 of SEQ ID NO: 5 or an equivalent thereof, and/or a light chain (LC) comprising, or consisting essentially of, or yet further consisting of an amino acid sequence of aa 21 to aa 233 of SEQ ID NO: 10 or an equivalent thereof. In another embodiment, the antibody or fragment thereof comprises or consists essentially of, or yet further consists of: a heavy chain (HC) comprising, or consisting essentially of, or yet further consisting of an amino acid sequence of aa 25 to aa 473 of SEQ ID NO: 5 or an equivalent thereof, and/or a light chain (LC) comprising, or consisting essentially of, or yet further consisting of an amino acid sequence of aa 21 to aa 233 of SEQ ID NO: 11 or an equivalent thereof. In yet another embodiment, the antibody or fragment thereof comprises or consists essentially of, or yet further consists of: a heavy chain (HC) comprising, or consisting essentially of, or yet further consisting of an amino acid sequence of aa 25 to aa 473 of SEQ ID NO: 5 or an equivalent thereof, and/or a light chain (LC) comprising, or consisting essentially of, or yet further consisting of an amino acid sequence of aa 21 to aa 233 of SEQ ID NO: 12 or an equivalent thereof. In one embodiment, the antibody or fragment thereof comprises or consists essentially of, or yet further consists of: a heavy chain (HC) comprising, or consisting essentially of, or yet further consisting of an amino acid sequence of aa 25 to aa 473 of SEQ ID NO: 6 or an equivalent thereof, and/or a light chain (LC) comprising, or consisting essentially of, or yet further consisting of an amino acid sequence of aa 21 to aa 233 of SEQ ID NO: 10 or an equivalent thereof. In another embodiment, the antibody or fragment thereof comprises or consists essentially of, or yet further consists of: a heavy chain (HC) comprising, or consisting essentially of, or yet further consisting of an amino acid sequence of aa 25 to aa 473 of SEQ ID NO: 6 or an equivalent thereof, and/or a light chain (LC) comprising, or consisting essentially of, or yet further consisting of an amino acid sequence of aa 21 to aa 233 of SEQ ID NO: 11 or an equivalent thereof. In yet another embodiment, the antibody or fragment thereof comprises or consists essentially of, or yet further consists of: a heavy chain (HC) comprising, or consisting essentially of, or yet further consisting of an amino acid sequence of aa 25 to aa 473 of SEQ ID NO: 6 or an equivalent thereof, and/or a light chain (LC) comprising, or consisting essentially of, or yet further consisting of an amino acid sequence of aa 21 to aa 233 of SEQ ID NO: 12 or an equivalent thereof.

In one aspect, provided is an antibody or a fragment thereof that comprises or consists essentially of, or yet further consists of: any one or any two or all three CDRs of a sequence selected from the group of: SEQ ID NOs: 1-3 or 24, or an equivalent of each thereof; and/or any one or any two or all three CDRs of a sequence selected from the group of: SEQ ID NOs: 7-9 or 25, or an equivalent of each thereof. In a further embodiment, the antibody or fragment thereof binds to a tip region of a DNABII peptide (including but not limited to: a tip region of IHF or HU, a tip region of IHFA or IHFB, and/or the tip-chimeric peptide IhfA5-mIhfB4_{NTHI}). In one embodiment, the antibody or fragment thereof binds to the tip-chimeric peptide IhfA5-mIhfB4_{NTHI}. In some embodiments, the antibodies or antigen binding fragments are anti tip chimer (i.e., specifically binding and recognizing the tip chimer). In yet a further embodiment, the fragment is an antigen binding fragment. In one embodiment, provided is an antibody or a fragment thereof that comprises or consists essentially of, or yet further consists of: all three CDRs of a sequence selected from the group of: SEQ ID NOs: 1-3 or 24, or an equivalent of each thereof, and/or all three CDRs of a sequence selected from the group of: SEQ ID NOs: 7-9 or 25, or an equivalent of each thereof.

In another embodiment, provided is an antibody or a fragment thereof that comprises or consists essentially of, or yet further consists of: any one or any two or all three CDRs of a sequence selected from the group of: SEQ ID NOs: 4-6 or 26, or an equivalent of each thereof; and/or any one or any two or all three CDRs of a sequence selected from the group of: SEQ ID NOs: 10-12 or 27, or an equivalent of each thereof. In a further embodiment, the antibody or fragment thereof binds to a tail region of a DNABII peptide (including but not limited to: a tail region of IHF or HU, a tail region of IHFA or IHFB, and/or the tail-chimeric peptide IhfA3-IhfB2_{NTHI}). In one embodiment, the antibody of fragment thereof binds to the tail-chimeric peptide IhfA3-IhfB2_{NTHI}. In some embodiments, the antibodies or antigen binding fragments are anti tail chimer (i.e., specifically binding and recognizing the tail chimer). In yet a further embodiment, the fragment is an antigen binding fragment. In one embodiment, provided is an antibody or a fragment thereof that comprises or consists essentially of, or yet further consists of: all three CDRs of a sequence selected from the group of: SEQ ID NOs: 4-6 or 26, or an equivalent of each thereof, and/or all three CDRs of a sequence selected from the group of: SEQ ID NOs: 10-12 or 27, or an equivalent of each thereof.

In certain embodiments, the antibody or fragment thereof as provided herein further comprises one or more signal peptide(s). In one embodiment, the signal peptide comprises or consists essentially of, or yet further consists of amino acid (aa) 1 to aa 24 of any one of SEQ ID NOs: 1-6, 13, 24 or 26. In another embodiment, the signal peptide comprises or consists essentially of, or yet further consists of aa 1 to aa 20 of any one of SEQ ID NOs: 7-12, 14, 25 and 27. In a further embodiment, the signal peptide is located at the amino terminus of the light chain variable region. Additionally or alternatively, the same signal peptide or a different signal peptide is located at the amino terminus of the heavy chain variable region.

The antibody or fragment thereof as provided herein may be monospecific or bispecific. In one embodiment, the antibody or fragment thereof is trispecific, or tetraspecific, or pentaspecific. Additionally or alternatively, the antibody is selected from the group of an IgA (such as an IgA1 or an IgA2), an IgD, an IgE, an IgG (such as an IgG1, an IgG2, an IgG3, or an IgG4), or an IgM antibody. In one embodiment, the antibody further comprises a constant region selected from the group of: an IgA constant region (such as an IgA1 constant region or an IgA2 constant region), an IgD constant region, an IgE constant region, an IgG constant region (such as an IgG1 constant region, an IgG2 constant region, an IgG3 constant region, or an IgG4 constant region) or an IgM constant region.

In certain embodiments, an equivalent to an amino acid sequence comprises or consists essentially of, or yet further consists of a polypeptide having at least about 80% (including about 80% to 100%, at least about 85%, at least about 90%, at least about 91%, at least about 92%, at least about 93%, at least about 94%, at least about 95%, at least about 96%, at least about 97%, at least about 98%, or at least about 99%) amino acid identity to the amino acid sequence. Additionally or alternatively, an equivalent to the amino acid sequence comprises or consists essentially of, or yet further consists of a polypeptide that is encoded by a polynucleotide that hybridizes under conditions of high stringency to the complement of the polynucleotide encoding the amino acid sequence. In a further embodiment, an equivalent to an amino acid sequence comprises or consists essentially of, or yet further consists of a polypeptide at least 80% (including about 80% to 100%, at least about 85%, at least about 90%, at least about 91%, at least about 92%, at least about 93%, at least about 94%, at least about 95%, at least about 96%, at least about 97%, at least about 98%, or at least about 99%) identity to the amino acid sequence. In certain embodiments, an equivalent to an amino acid sequence (such as an antibody or a fragment thereof, or any one or more of SEQ ID NOs: 1-14 and 24-26 or a fragment thereof as disclosed herein, including but not limited to: 25 to aa 144 of SEQ ID NOs: 13, 24 or 26, aa 21 to aa 132 of SEQ ID NOs: 14 or 25, aa 21 to aa 126 of SEQ ID NO: 27, aa 25 to aa 473 of SEQ ID NOs: 13, 24 or 26, aa 21 to aa 239 of SEQ ID NOs: 14 or 25, aa 21 to aa 233 of SEQ ID NO: 27) comprises, or consists essentially of, or yet further consists of a polypeptide comprises one or more or all CDRs of the amino acid sequence. Additionally or alternatively, the polypeptide is at least about 80% (including about 80% to 100%, at least about 85%, at least about 90%, at least about 91%, at least about 92%, at least about 93%, at least about 94%, at least about 95%, at least about 96%, at least about 97%, at least about 98%, or at least about 99%) amino acid identity to the amino acid sequence.

In certain embodiments, the equivalent to an amino acid sequence, such as an antibody, a fragment thereof, a complementarity-determining region (CDR) thereof, or a CDR-containing polypeptide, lacks an amino acid difference to the amino acid sequence in the CDR(s). However, the equivalent to an amino acid sequence, such as an antibody, a fragment thereof, a CDR thereof, or a CDR-containing polypeptide, may comprises one or more of (for example but not limited to 1, 2, 3, 4, 5, 6, 7, 8, 9, 10, 11, 12, 13, 14, 15, 16, 17, 18, 19, 20, 21, 22, 23, 24 or 25) amino acid differences compared to the amino acid sequence in the non-CDR region(s) with the proviso that the three-dimensional arrangement of the CDR(s) and/or the CDRs is/are retained. In certain embodiments, the equivalent polypeptide to an amino acid sequence, such as an antibody, a fragment thereof, a CDR thereof, or a CDR-containing polypeptide, is at least about 80% (including about 80% to 100%, at least about 85%, at least about 90%, at least about 91%, at least about 92%, at least about 93%, at least about 94%, at least about 95%, at least about 96%, at least about 97%, at least about 98%, or at least about 99%) amino acid identity to the amino acid sequence with the proviso that the three-dimensional arrangement of the CDR(s) and/or the CDR(s) is/are retained.

Non-limiting examples of such non-CDR regions includes a framework region (FR), a constant region, an Fc region, a pFc′ region, a constant heavy chain (CH) domain (such as CH1, CH2, CH3 or CH4), a constant light chain (CL) domain, or a hinge region. In one embodiment, such amino acid differences may be a conservative amino acid substitution and/or does not change the three-dimensional arrangement of the antibody, fragment thereof, CDR thereof, or the CDR-containing polypeptide. In another embodiment, the equivalent may comprises a conservative amino acid substitution in the boundaries of a CDR, such as one or two amino acid(s) at the amino termini, the carboxyl termini or both of the CDR.

In one aspect, provided is one or more of CDRs (such as any 1, or 2, or 3, or 4, or 5, or 6 CDR(s)) of an antibody or fragment thereof as disclosed herein. In one embodiment, provided is a set of CDRs comprising or alternatively consisting essentially of, or yet further consisting of one or more of CDRs (such as any 1, or 2, or 3, or 4, or 5, or 6 CDR(s)) of an antibody or fragment thereof as disclosed herein. In one embodiment, provided is a set of CDRs comprising, or alternatively consisting essentially of, or yet further consisting of CDRL1, CDRL2, and CDRL3 of a variable region as disclosed herein. In a further embodiment, provided is a set of CDRs comprising, or alternatively consisting essentially of, or yet further consisting of CDRH1, CDRH2, and CDRH3 of a variable region as disclosed herein. In yet a further embodiment, provided is a set of CDRs comprising, or alternatively consisting essentially of, or yet further consisting of CDRL1, CDRL2, and CDRL3 of a variable region as disclosed herein and CDRH1, CDRH2, CDRH3 of another variable region as disclosed herein. In certain embodiments, the CDR set constitutes a paratope. Additionally or alternatively, the CDR set specifically binds to a DNABII peptide (such as the tip region and/or the tail region, including but not limited to: a tip region of IHF or HU, a tip region of IHFA or IHFB, the tip-chimeric peptide IhfA5-mIhfB4_{NTHI}, a tail region of IHF or HU, a tail region of IHFA or IHFB, and/or the tail-chimeric peptide IhfA3-IhfB2_{NTHI}). In a further embodiment, provided is an antibody, a fragment thereof, or an equivalent of each thereof, comprising, or alternatively consisting essentially of, or yet further consisting of any one or more CDRs as disclosed herein. In yet a further embodiment, provided is an antibody, a fragment thereof, or an equivalent of each thereof, comprising, or alternatively consisting essentially of, or yet further consisting of a CDR set as disclosed herein.

In certain embodiments, CDRs of an anti-tip chimer antibody are shown in SEQ ID NOs: 1-3, 7-9, 13 and 14 are illustrated in the following table. The CDRs of an anti tail chimer are shown in SEQ ID NOs: 4-6 and 10-12. In certain embodiments, CDRH1 of any one of SEQ ID NOs: 1-6, 13, 24 or 26 comprises or consists essentially of, or yet further consists of amino acid (aa) 50 to aa 57 of SEQ ID NO: 1-6, 13, 24 or 26, respectively. In certain embodiments, CDRH2 of any one of SEQ ID NOs: 1-6, 13, 24 or 26 comprises or consists essentially of, or yet further consists of amino acid (aa) 75 to aa 82 of SEQ ID NO: 1-6, 13, 24 or 26, respectively. In certain embodiments, CDRH3 of any one of SEQ ID NOs: 1-6, 13, 24 or 26 comprises or consists essentially of, or yet further consists of amino acid (aa) 121 to aa 133 of SEQ ID NO: 1-6, 13, 24 or 26, respectively. In certain embodiments, CDRL1 of any one of SEQ ID NOs: 7-9, 14 or 25 comprises or consists essentially of, or yet further consists of amino acid (aa) 47 to aa 57 of SEQ ID NO: 7-9, 14 or 25, respectively. In certain embodiments, CDRL2 of any one of SEQ ID NOs: 7-9, 14 or 25 comprises or consists essentially of, or yet further consists of amino acid (aa) 75 to aa 77 of SEQ ID NO: 7-9, 14 or 25, respectively. In certain embodiments, CDRL3 ofany one of SEQ ID NOs: 7-9, 14 or 25 comprises or consists essentially of, or yet further consists of amino acid (aa) 114 to aa 122 of SEQ ID NO: 7-9, 14 or 25, respectively. In certain embodiments, CDRL1 of any one of SEQ ID NOs: 10-12 or 27 comprises or consists essentially of, or yet further consists of amino acid (aa) 47 to aa 52 of SEQ TD NO: 10-12 or 27, respectively. In certain embodiments, CDRL2 of any one of SEQ TD NOs: 10-12 or 27 comprises or consists essentially of, or yet further consists of amino acid (aa) 70 to aa 72 of SEQ TD NO: 10-12 or 27, respectively. In certain embodiments, CDRL3 of any one of SEQ ID NOs: 10-12 or 27 comprises or consists essentially of, or yet further consists of amino acid (aa) 109 to aa 116 of SEQ ID NO: 10-12 or 27, respectively.

| SEQ ID NO: | CDR1 | CDR2 | CDR3 | Variable Region |
|---|---|---|---|---|
| 4 | GFTFSRYG | ISSGGSYT | ERHGGDGYWYFDV | EVQLVESGGGLVKPGGSLRLSCAASGFTFSRYGMSW |
| | | | | VRQAPGKGLEWVATISSGGSTYTYYTDSVKGRFTIS |
| | | | | RDNAKNSLYLAMNSLRAEDTAVYYCERHGGDGYWYF |
| | | | | DVWGQGTMVTVSS |
| 5 | GFTFSRYG | ISSGGSYT | ERHGGDGYWYFDV | EVQLVESGGGLVKPGGSLRLSCAASGFTFSRYGMSW |
| | | | | VRQAPGKGLEWVSTISSGGSTYTYYTDSVKGRFTIS |
| | | | | RDNAKNSLYLQMNSLRAEDTAVYYCERHGGDGYWYF |
| | | | | DVWGQGTMVTVSS |
| 6 | GFTFSRYG | ISSGGSYT | ERHGGDGYWYDFV | EVQLVESGGGLVQPGRSLRLSCTASGFTFSRYGMSW |
| | | | | VRQAPGKGLEWVATISSGGSTYTYYTDSVKGRFTIS |
| | | | | RDNAKNILYLQMNSLKTEDTAVYYCERHGGDGYWYF |
| | | | | DVWGQGTMVTVSS |
| 10 | QDISNY | YTS | QQGNPLRT | DIQMTQSPSSLSASVGDRVTITCRASQDISNYLNWY |
| | | | | QQKPGKAVKLLIYYTSRLHSGVPSRFSGSGSGTDYT |
| | | | | LTISSLQPEDFATYFCQQGNPLRTFGGGTKVEIK |
| 11 | QDISNY | YTS | QQGNPLRT | DIQMTQSPSSLSASVGDRVTITCRASQDISNYLNWY |
| | | | | QQKPGKAVKLLIYYTSRLHSGVPSRFSGSGSGTDYT |
| | | | | LTISSLQPEDFATYYCQQGNPLRTFGGGTKVEIK |
| 12 | QDISNY | YTS | QQGNPLRT | DIVMTQSPATLSLSPGERATLSCRASQDISNYLNWY |
| | | | | QQKPGQAVRLLIYYTSRLHSGIPARFSGSGSGTDYT |
| | | | | LTISSLEPEDFAVYFCQQGNPLRTFGGGTKVRIK |
| 1 | GFTFRTYA | IGSDRRHT | VGPYDGYYGEFDY | EVKLVESGGGLVQPGGSLRLSCAASGFTFRTYAMSW |
| | | | | VRQAPGKGLEWVATIGSDRRHTYYPDSVKGRFTISR |
| | | | | DNAKNTLYLQMNSLRAEDTAVYYCVGPYDGYYGEFD |
| | | | | YWGQGTLVTVSS |
| 2 | GFTFRTYA | IGSDRRHT | VGPYDGYYGEFDY | EVQLVESGGGLVQPGGSLRLSCAASGFTFRTYAMSW |
| | | | | VRQAPGKGLEWVATIGSDRRHTYYPDSVKGRFTISR |
| | | | | DNSKNTLYLQMNSLRAEDTAVYYCVGPYDGYYGEFD |
| | | | | YWGQGLTVTVSS |
| 3 | GFTFRTYA | IGSDRRHT | VGPYDGYYGEFDY | EVKLVQSGAEVKKPGASVKVSCKASGFTFRTYAMSW |
| | | | | VRQAPGQRLEWVATIGSDRRHTYYPDKFQGRVTITR |
| | | | | DNAKNTLYMELSSLRSEDTAVYYCVGPYDGYYGEFD |
| | | | | YWGQGTLVTVSS |
| 7 | QSLLDSDG | LVS | WQGTHFPYT | DVVMTQSPLSLPVTLGQPASISCRSSQSLLDSDGKT |
| | KTF | | | FLNWLQQRPGQSPRRLIYLVSKLDSGVPDRFSGSGS |
| | | | | GTDFTLKISRVEAEDVGVYYCWQGTHFPYTFGQGTK |
| | | | | LEIK |
| 8 | QSLLDSDG | LVS | WQGTHFPYT | DVVMTQSPLSLPVTLGQPASISCRSSQSLLDSDGKT |
| | KTF | | | FLNWLQQRPGQSPRRLIYLVSKRDSGVPDRFSGSGS |
| | | | | GTDFTLKISRVEAEDVGVYYCWQGTHFPYTFGQGTK |
| | | | | LEIK |
| 9 | QSLLDSDG | LVS | WQGTHFPYT | DVVMTQSPDSLAVSLGERATINCKSSQSLLDSDGKT |
| | KTF | | | FLNWLQQKPGQPPKRLIYLVSKLDSGVPDRFSGSGS |
| | | | | GTDFTLTISSLQAEDVAVYYCWQGTHFPYTFGQGTK |
| | | | | LEIK |

In certain embodiments, provided are CDRs as identified in the following two Tables below.

| SEQ | | | 1 | 2 | 3 | 4 | 5 | 6 | 7 | 8 |
|---|---|---|---|---|---|---|---|---|---|---|
| 1 | VH1 | CDR1 | AASGFTFRTY | GFTFRTYA | GFTFRTY | AASGFTFRTY | GFTFRTYA | GFTFRTY | GFTFRTYA | GFTFRTY |
| | | | AMS | | | AMS | | | | |
| | | CDR2 | TIGSDRRHTY | IGSDRRHT | GSDRRH | TIGSDRRHTY | IGSDRRHT | GSDRRH | IGSDRRHT | GSDRRH |
| | | CDR3 | Not | Not | Not | Not | Not | Not | VGPYDGYY | VGPYDGYY |
| | | | determined | determined | determined | determined | determined | determined | GEFDY | GEFDY |
| 2 | VH2 | CDR1 | AASGFTFRTY | GFTFRTYA | GFTFRTY | AASGFTFRTY | GFTFRTYA | GFTFRTY | GFTFRTYA | GFTFRTY |
| | | | AMS | | | AMS | | | | |
| | | CDR2 | TIGSDRRHTY | IGSDRRHT | GSDRRH | TIGSDRRHTY | IGSDRRHT | GSDRRH | IGSDRRHT | GSDRRH |
| | | CDR3 | Not | Not | Not | Not | Not | Not | VGPYDGYY | VGPYDGYY |
| | | | determined | determined | determined | determined | determined | determined | GEFDY | GEFDY |
| 3 | VH3 | CDR1 | KASGFTFRTY | GFTFRTYA | GFTFRTY | KASGFTFRTY | GFTFRTYA | GFTFRTY | GFTFRTYA | GFTFRTY |
| | | | AMS | | | AMS | | | | |
| | | CDR2 | TIGSDRRHTY | IGSDRRHT | GSDRRH | TIGSDRRHTY | IGSDRRHT | GSDRRH | IGSDRRHT | GSDRRH |
| | | | | | | | | | Y | |
| | | CDR3 | Not | Not | Not | Not | Not | Not | VGPYDGYY | VGPYDGYY |
| | | | determined | determined | determined | determined | determined | determined | GEFDY | GEFDY |
| 7 | VL1 | CDR1 | RSSQSLLDSD | QSLLDSDGKT | RSSQSLLDSD | RSSQSLLDSD | QSLLDSDGKT | RSSQSLLDSD | QSLLDSDG | QSLLDSDG |
| | | | GKTFLN | F | GFTFLN | GFTKLN | F | GKTFLN | KTF | KTF |
| | | CDR2 | YLVSKLDS | LVS | LVSKLDS | YLVSKLDS | LVS | LVSKLDS | LVS | LVS |
| | | CDR3 | WQGTHFPYT | WQGTHFPYT | WQGTHFPYT | WQGTHFPYT | WQGTHFPYT | WQGTHFPYT | WQGTHFPY | WQGTHFPY |
| | | | | | | | | | T | T |
| 8 | VL2 | CDR1 | RSSQSLLDSD | QSLLDSDGKT | RSSQSLLDSD | RSSQSLLDSD | QSLLDSDGKT | RSSQSLLDSD | QSLLDSDG | QSLLDSDG |
| | | | GKTFLN | F | GKTFLN | GKTFLN | F | GKTFLN | KTF | KTF |
| | | CDR2 | YLVSKRDS | LVS | LVSKRDS | YLVSKRDS | LVS | LVSKRDS | LVS | LVS |
| | | CDR3 | WQGTHFPYT | WQGTHFPYT | WQGTHFPYT | WQGTHFPYT | WQGTHFPYT | WQGTHFPYT | WQGTHFPY | WQGTHFPY |
| | | | | | | | | | T | T |
| 9 | VL3 | CDR1 | KSSQSLLDSD | QSLLDSDGKT | KSSQSLLDSD | KSSQSLLDSD | QSLLDSDKGT | KSSQSLLDSD | QSLLDSDG | QSLLDSDG |
| | | | GFKTLN | F | GKTFLN | GKTFLN | F | GKTFLN | KTF | KTF |
| | | CDR2 | YLVSKLDS | LVS | LVSKLDS | YLVSKLDS | LVS | LVSKLDS | LVS | LVS |
| | | CDR3 | WQGTHFPYT | WQGTHFPYT | WQGTHFPYT | WQGTHFPYT | WQGTHFPYT | WQGTHFPYT | WQGTFHPY | WQGTHFPY |
| | | | | | | | | | T | T |
| 4 | VH1 | CDR1 | AASGFTFSRY | GFTFSRYG | GFTFSRY | AASGFTFSRY | GFTFSRYG | GFTFSRY | GFTFSRYG | GFTFSRY |
| | | | GMS | | | GMS | | | | |
| | | CDR2 | TISSGGSYTY | ISSGGSYT | SSGGSY | TISSGGSYTY | ISGGGSYT | SSGGSY | ISSGGSYT | SSGGSY |
| | | CDR3 | Not | Not | Not | Not | Not | Not | ERHGGDGY | ERHGGDGY |
| | | | determined | determined | determined | determined | determined | determined | WYFDV | WYFDV |
| 5 | VH2 | CDR1 | AASGFTFSRY | GFTFSRYG | GFTFSRY | AASGFTFSRY | GFTFSRYG | GFTFSRY | GFTFSRYG | GFTFSRY |
| | | | GMS | | | GMS | | | | |
| | | CDR2 | TISSGGSYTY | ISSGGSYT | SSGGSY | TISSGGSYTY | ISSGGSYT | SSGGSY | ISSGGSYT | SSGGSY |
| | | CDR3 | Not | Not | Not | Not | Not | Not | ERHGGDGY | ERHGGDGY |
| | | | determined | determined | determined | determined | determined | determined | WYFDV | WYFDV |
| 6 | VH3 | CDR1 | TASGFTFSRY | GFTFSRYG | GFTFSRY | TASGFTFSRY | GFTFSRYG | GFTFSRY | GFTFSRYG | GFTFSRY |
| | | | GMS | | | GMS | | | | |
| | | CDR2 | TISSGGSYTY | ISSGGSYT | SSGGSY | TISSGGSYTY | ISSGGSYT | SSGGSY | ISSGGSYT | SSGGSY |
| | | CDR3 | Not | Not | Not | Not | Not | Not | ERHGGDGY | ERHGGDGY |
| | | | determined | determined | determined | determined | determined | determined | WYFDV | WYFDV |
| 10 | VL1 | CDR1 | RASQDISNYL | QDISNY | RASQDISNYL | RASQDISNYL | QDISNY | RASQDISNYL | QDISNY | QDISNY |
| | | | N | | N | N | | N | | |
| | | CDR2 | YYTSRLHS | YTS | YTSRLHS | YYTSRLHS | YTS | YTSRLHS | YTS | YTS |
| | | CDR3 | QQGNPLRT | QQGNPLRT | QQGNPLRT | QQGNPLRT | QQGNPLRT | QQGNPLRT | QQGNPLRT | QQGNPLRT |
| 11 | VL2 | CDR1 | RASQDISNYL | QDISNY | RASQDISNYL | RASQDISNY | QDISNY | RASQDISNYL | QDISNY | QDISNY |
| | | | N | | N | LN | | N | | |
| | | CDR2 | YYTSRLHS | YTS | YTSRLHS | YYTSRLHS | YTS | YTSRLHS | YTS | YTS |
| | | CDR3 | QQGNPLRT | QQGNPLRT | QQGNPLRT | QQGNPLRT | QQGNPLRT | QQGNPLRT | QQGNPLRT | QQGNPLRT |
| 12 | VL3 | CDR1 | RASQDISNYL | QDISNY | RASQDISNYL | RASQDISNYL | QDISNY | RASQDISNYL | QDISNY | QDISNY |
| | | | N | | N | N | | N | | |
| | | CDR2 | YYTSRLHS | YTS | YTSRLHS | YYTSRLHS | YTS | YTSRLHS | YTS | YTS |
| | | CDR3 | QQGNPLRT | QQGNPLRT | QQGNPLRT | QQGNPLRT | QQGNPLRT | QQGNPLRT | QQGNPLRT | QQGNPLRT |

SEQ: SEQ ID NO

In certain embodiments, provided is an alternative CDR that is a CDR as identified herein further comprising an additional 1 amino acid, or alternatively 2 amino acids, or alternatively 3 amino acids, or alternatively 4 amino acids, or alternatively 5 amino acids, or alternatively 6 amino acids, or alternatively 7 amino acids, or alternatively 8 amino acids at its amino terminus, or carboxyl terminus or both in the corresponding variable region sequence. Additionally or alternatively, provided is an alternative CDR that is a CDR as identified herein having 1 amino acid, or alternatively 2 amino acids, or alternatively 3 amino acids, or alternatively 4 amino acids, or alternatively 5 amino acids, or alternatively 6 amino acids, or alternatively 7 amino acids, or alternatively 8 amino acids truncated at its amino terminus, or carboxyl terminus or both in the corresponding variable region sequence. For example, CDR1 of SEQ ID NO: 1 may be amino acid 50 to amino acid 57 of SEQ ID NO: 1. However, the CDR1 of SEQ ID NO: 1 can also start from amino acid 42, or 43, or 44, or 45, or 46, or 47, or 48, or 49, or 50, or 51, or 52, or 53, or 54, or 55, or 56, or 57, or 58 of SEQ ID NO: 1. Further, CDR1 of SEQ ID NO: 1 can end at amino acid 49, or 50, or 51, or 52, or 53, or 54, or 55, or 56, or 57, or 58, or 59, or 60, or 61, or 62, or 63, or 64, or 65 of SEQ ID NO: 1 with proviso that the CDR1 ends after its start. Additionally or alternatively, the CDR is about 1, or alternatively about 2, or alternatively about 3, or alternatively about 4, or alternatively about 5, or alternatively about 6, or alternatively about 7, or alternatively about 8, or alternatively about 9, or alternatively about 10, or alternatively about 11, or alternatively about 12, or alternatively about 13, or alternatively about 14, or alternatively about 15 amino acids long.

In certain embodiments, CDR2 of any one of SEQ ID NOs: 1-6 comprises or consists essentially of, or yet further consists of amino acid 71 to amino acid 85 of each of SEQ ID NOs: 1-6, respectively. In certain embodiments, CDR3 of any one of SEQ ID NOs: 1-6 comprises or consists essentially of, or yet further consists of amino acid 121 to amino acid 133 of each of SEQ ID NOs: 1-6, respectively. In certain embodiments, CDR2 of any one of SEQ ID NOs: 7-9 comprises or consists essentially of, or yet further consists of amino acid 71 to amino acid 81 of each of SEQ ID NOs: 7-9, respectively. In certain embodiments, CDR3 of any one of SEQ ID NOs: 7-9 comprises or consists essentially of, or yet further consists of amino acid 114 to amino acid 121 of each of SEQ ID NOs: 7-9, respectively. In certain embodiments, CDR2 of any one of SEQ ID NOs: 10-12 comprises or consists essentially of, or yet further consists of amino acid 66 to amino acid 76 of each of SEQ ID NOs: 10-12, respectively. In certain embodiments, CDR3 of any one of SEQ ID NOs: 10-12 comprises or consists essentially of, or yet further consists of amino acid 109 to amino acid 115 of each of SEQ ID NOs: 10-12, respectively.

In certain embodiments, CDR1 of any one of SEQ ID NOs: 1-6 comprises or consists essentially of, or yet further consists of amino acid 50 to amino acid 57 of each of SEQ ID NOs: 1-6, respectively. In certain embodiments, CDR2 of any one of SEQ ID NOs: 1-6 comprises or consists essentially of, or yet further consists of amino acid 75 to amino acid 82 of each of SEQ ID NOs: 1-6, respectively. In certain embodiments, CDR3 of any one of SEQ ID NOs: 1-6 comprises or consists essentially of, or yet further consists of amino acids 121 and 122 of each of SEQ ID NOs: 1-6, respectively. In certain embodiments, CDR1 of any one of SEQ ID NOs: 7-9 comprises or consists essentially of, or yet further consists of amino acid 47 to amino acid 57 of each of SEQ ID NOs: 7-9, respectively. In certain embodiments, CDR2 of any one of SEQ ID NOs: 7-9 comprises or consists essentially of, or yet further consists of amino acid 75 to amino acid 77 of each of SEQ ID NOs: 7-9, respectively. In certain embodiments, CDR3 of any one of SEQ ID NOs: 7-9 comprises or consists essentially of, or yet further consists of amino acids 114 and 120 of each of SEQ ID NOs: 7-9, respectively. In certain embodiments, CDR1 of any one of SEQ ID NOs: 10-12 comprises or consists essentially of, or yet further consists of amino acid 47 to amino acid 52 of each of SEQ ID NOs: 10-12, respectively. In certain embodiments, CDR2 of any one of SEQ ID NOs: 10-12 comprises or consists essentially of, or yet further consists of amino acid 70 to amino acid 72 of each of SEQ ID NOs: 10-12, respectively. In certain embodiments, CDR3 of any one of SEQ ID NOs: 10-12 comprises or consists essentially of, or yet further consists of amino acids 109 and 110 of each of SEQ ID NOs: 10-12, respectively.

In certain embodiments, CDR1 of any one of SEQ ID NOs: 1-6 comprises or consists essentially of, or yet further consists of amino acid 47 to amino acid 59 of each of SEQ ID NOs: 1-6, respectively. In certain embodiments, CDR2 of any one of SEQ ID NOs: 1-6 comprises or consists essentially of, or yet further consists of amino acid 74 to amino acid 83 of each of SEQ ID NOs: 1-6, respectively. In certain embodiments, CDR1 of any one of SEQ ID NOs: 7-9 comprises or consists essentially of, or yet further consists of amino acid 44 to amino acid 59 of each of SEQ ID NOs: 7-9, respectively. In certain embodiments, CDR2 of any one of SEQ ID NOs: 7-9 comprises or consists essentially of, or yet further consists of amino acid 74 to amino acid 81 of each of SEQ ID NOs: 7-9, respectively. In certain embodiments, CDR3 of any one of SEQ ID NOs: 7-9 comprises or consists essentially of, or yet further consists of amino acids 114 and 122 of each of SEQ ID NOs: 7-9, respectively. In certain embodiments, CDR1 of any one of SEQ ID NOs: 10-12 comprises or consists essentially of, or yet further consists of amino acid 44 to amino acid 54 of each of SEQ ID NOs: 10-12, respectively. In certain embodiments, CDR2 of any one of SEQ ID NOs: 10-12 comprises or consists essentially of, or yet further consists of amino acid 79 to amino acid 76 of each of SEQ ID NOs: 10-12, respectively. In certain embodiments, CDR3 of any one of SEQ ID NOs: 10-12 comprises or consists essentially of, or yet further consists of amino acids 109 and 116 of each of SEQ ID NOs: 10-12, respectively.

In one aspect, provided is one or more of variable region(s) of an antibody or fragment thereof as disclosed herein, and/or one or more of equivalent(s) of the variable regions(s). In a further embodiment, provided is an antibody, a fragment thereof, or an equivalent of each thereof, comprising, or alternatively consisting essentially of, or yet further consisting of any one or any two or more of: the variable regions as disclosed herein and/or one or more of equivalent(s) of the variable regions(s). Additionally or alternatively, the one or more of variable region(s) specifically binds to a DNABII peptide (such as the tip region and/or the tail region, including but not limited to: a tip region of IHF or HU, a tip region of IHFA or IHFB, the tip-chimeric peptide IhfA5-mIhfB4_{NTHI}, a tail region of IHF or HU, a tail region of IHFA or IHFB, and/or the tail-chimeric peptide IhfA3-IhfB2_{NTHI}). In certain embodiments, the variable region is selected from the following: amino acid (aa) 25 to aa 144 of SEQ ID NOs: 1-6, 13, 24 and 26; aa 21 to aa 132 of SEQ ID NOs: 7-9, 14, and 25; aa 21 to aa 126 of SEQ ID NOs: 10-12 or 27; amino acid 24 to amino acid 144 of SEQ ID NOs: 1-6, 13, 24 and 26; amino acid 20 to amino acid 132 of SEQ ID NOs: 7-12, 14, 25 and 27; amino acid 20 to amino acid 126 of SEQ ID NOs: 7-12, 14, 25 and 27.

In a further embodiment, the variable region or an equivalent thereof is a variable region as disclosed herein further comprising an additional 1 amino acid, or alternatively 2 amino acids, or alternatively 3 amino acids, or alternatively 4 amino acids, or alternatively 5 amino acids, or alternatively 6 amino acids, or alternatively 7 amino acids, or alternatively 8 amino acids at its amino terminus, or carboxyl terminus or both in the corresponding sequence provided herein with a SEQ ID NO. Additionally or alternatively, the variable region or an equivalent thereof is a variable region as disclosed herein having 1 amino acid, or alternatively 2 amino acids, or alternatively 3 amino acids, or alternatively 4 amino acids, or alternatively 5 amino acids, or alternatively 6 amino acids, or alternatively 7 amino acids, or alternatively 8 amino acids truncated at its amino terminus, or carboxyl terminus or both in the corresponding sequence provided herein with a SEQ ID NO. For example, of SEQ ID NO: 1 may be amino acid 50 to amino acid 57 of SEQ ID NO: 1. However, the variable region or an equivalent thereof relating to the variable region consisting of amino acid 24 to amino acid 144 of SEQ ID NO: 1 can also start from amino acid 16, or 17, or 18, or 19, or 20, or 21, or 22, or 23, or 24, or 25, or 26, or 27, or 28, or 29, or 30, or 31, or 32 of SEQ ID NO: 1. Further, the variable region or an equivalent thereof relating to the variable region consisting of amino acid 24 to amino acid 144 of SEQ ID NO: 1 can end at amino acid 136, or 137, or 138, or 139, or 140, or 141, or 142, or 143, or 144, or 145, or 146, or 147, or 148, or 149, or 150, or 151, or 152 of SEQ ID NO: 1 with proviso that the variable region ends after its start. Additionally or alternatively, the variable region is about 90 amino acids long to about 200 amino acids long, for example, about 100 amino acid long, or alternatively about 110 amino acid long, or alternatively about 120 amino acid long, or alternatively about 130 amino acid long, or alternatively about 140 amino acid long, or alternatively about 150 amino acid long, or alternatively about 160 amino acid long, or alternatively about 170 amino acid long, or alternatively about 180 amino acid long, or alternatively about 190 amino acid long, or alternatively about 200 amino acid long.

Additionally or alternatively, the equivalent to an antibody or a fragment thereof comprises one or more of (for example but not limited to 1, 2, 3, 4, 5, 6, 7, 8, 9, 10, 11, 12, 13, 14, 15, 16, 17, 18, 19, 20, 21, 22, 23, 24 or 25) amino acid differences compared to the antibody or a fragment thereof in the regions other than the variable domain(s) (referred to herein as non-VH regions). Such non-VH regions include, but are not limited to, a constant region, an Fc region, a pFc′ region, a constant heavy chain (CH) domain (such as CH1, CH2, CH3 or CH4), a constant light chain (CL) domain, or a hinge region. It would be understood by one of skill in the art that an antibody, a fragment thereof, or an equivalent of each thereof as disclosed herein, may be further modified in the non-VH regions (such as for increasing the assembly of a heavy chain with a light chain, conjugating directly or indirectly to a detectable or purification marker or a drug, increasing or decreasing activation of complement, enhancing or reducing antibody-dependent cellular cytotoxicity (ADCC), or increasing or decreasing activation and recruitment of an immune cell), providing a further equivalent.

In certain embodiments, the equivalent further comprises up to 50, or alternatively up to 30, or alternatively up to 25, or alternatively up to 20, or alternatively up to 15, or alternatively up to 10, or alternatively up to 5 random amino acids on either the amino or carboxyl termini or on both. In certain embodiments, the equivalent to an amino acid sequence comprises or consists essentially of, or yet further consists of the amino acid sequence truncated at the amino or carboxyl termini or both, for example, by 1, 2, 3, 4, 5, 6, 7, 8, 9, 10, 11, 12, 13, 14, 15, 20 or 25 amino acids. Such addition or truncation may not change the three-dimensional arrangement of the CDR(s) and/or the three-dimensional arrangement of the antibody, fragment thereof, CDR thereof, or the CDR-containing polypeptide.

In certain embodiments, an antibody or a fragment thereof comprises a signal peptide at the amino terminus of VH and/or the amino terminus of VL. In one embodiment, the VH signal peptide is different to the VL signal peptide. In another embodiment, the VH signal peptide is the same compared to the VL signal peptide. In a further embodiment, the signal peptide comprises or consists essentially of, or yet further consists of an amino acid sequence of amino acids 1-24 of SEQ ID NO: 1. In yet a further embodiment, the signal peptide comprises or consists essentially of, or yet further consists of an amino acid sequence of amino acids 1-20 of SEQ ID NO: 7. In certain embodiments, the equivalent to an antibody or a fragment thereof comprises a signal peptide which is different from the signal peptide(s) of the antibody with the proviso that the signal peptide of the equivalent directs VH and/or VL to the same cellular location as the signal peptide(s) of the antibody.

In certain embodiments, the equivalent to an antibody or a fragment thereof retains at least 50% (such as at least 60%, at least 70%, at least 80%, at least 90%, at least 95%, or at least 99%) of or improves one or more of functional activities of the antibody or fragment. Such functional activities include but are not limited to binding specificity, binding avidity and/or affinity to a DNABII peptide (such as the tip region and/or the tail region, including but not limited to: a tip region of IHF or HU, a tip region of IHFA or IHFB, the tip-chimeric peptide IhfA5-mIhfB4_{NTHI}, a tail region of IHF or HU, a tail region of IHFA or IHFB, and/or the tail-chimeric peptide IhfA3-IhfB2_{NTHI}), preventing formation of a biofilm in vivo or in vitro, or disrupting a biofilm in vivo or in vitro. Methods of quantifying such functional activities are illustrated in the examples.

In a further aspect, provided is an antibody or a fragment thereof that competes for binding to an epitope with an antibody or a fragment thereof as disclosed herein. The antibody or fragment thereof may be a polyclonal, a monoclonal and/or a humanized antibody.

In one aspect, the antibody is a bispecific antibody or a trispecific, tetraspecific or pentaspecific antibody. In a further aspect, the antibody is an IgA, an IgD, an IgE, an IgG or an IgM antibody. In another aspect, the antibodies further comprise a constant region selected from an IgA, an IgD, an IgE, an IgG or an IgM constant region. In a specific aspect, the constant region is an IgG1 constant region. In another aspect, provided herein are antibodies that compete for binding to an epitope with an antibody as disclosed herein. These can be identified using conventional techniques, e.g. a competitive ELISA.

The antibodies as disclosed herein can be polyclonal, monoclonal or humanized. In one aspect, the antibodies bind the “tip” region of a DNABII polypeptide, e.g., HU or IHF (such as IhfA and IhfB). In a further aspect, the antibodies bind the “tail” region of a DNABII polypeptide, e.g., HU or IHF (such as IhfA and IhfB). As noted above, this disclosure provides antigen binding fragments. The antigen binding fragments are any one of Fab, F(ab′)₂, Fab′, scFv, or Fv, that can be prepared using conventional techniques known to those of skill in the art. In some of the aspects of the antibodies provided herein, the antibody is soluble Fab. In another aspect, this disclosure provides a Fab fragment of the antibody as disclosed herein, wherein the antibody specifically binds the tip region of a DNABII peptide (including but not limited to: a tip region of IHF or HU, a tip region of IHFA or IHFB, and/or the tip-chimeric peptide IhfA5-mIhfB4_{NTHI}). In one aspect of the disclosure the DNABII is an IHF or an HU peptide. In a specific aspect, the DNABII is an IHF peptide.

As noted above, this disclosure provides equivalents to antibodies and antigen binding fragments. An equivalent can comprise a polypeptide having at least 80% amino acid identity to polypeptide, or a polypeptide that is encoded by a polynucleotide that hybridizes under conditions of high stringency to the complement of a polynucleotide encoding the polypeptide.

In some of the aspects of the antibodies provided herein, the antibody, fragment thereof, polypeptide or CDR binds a DNABII protein (including but not limited to: a tip region of IHF or HU, a tip region of IHFA or IHFB, the tip-chimeric peptide IhfA5-mIhfB4_{NTHI}, a tail region of IHF or HU, a tail region of IHFA or IHFB, and/or the tail-chimeric peptide IhfA3-IhfB2_{NTHI}) at a half maximal effective concentration (EC₅₀) of less than 500 ng/mL, or alternatively less than 250 ng/mL, or alternatively less than 200 ng/mL, or alternatively less than 150 ng/mL, or alternatively less than 100 ng/mL, or alternatively less than 90 ng/mL, or alternatively less than 80 ng/mL, or alternatively less than 70 ng/mL, or alternatively less than 65 ng/mL, or alternatively less than 60 ng/mL, or alternatively less than 55 ng/mL, or alternatively less than 50 ng/mL, or alternatively less than 45 ng/mL, or alternatively less than 40 ng/mL, or alternatively less than 35 ng/mL, or alternatively less than 30 ng/mL. In a further embodiment, such EC₅₀ is determined using the ELISA methods shown in the Examples.

In some of the aspects of the antibodies provided herein, the antibody, fragment thereof, polypeptide or CDR binds a DNABII protein (including but not limited to: a tip region of IHF or HU, a tip region of IHFA or IHFB, the tip-chimeric peptide IhfA5-mIhfB4_{NTHI}, a tail region of IHF or HU, a tail region of IHFA or IHFB, and/or the tail-chimeric peptide IhfA3-IhfB2_{NTHI}) with an equilibrium constant K_{D} of less than 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. In one embodiment, the antibody, fragment thereof, polypeptide or CDR binds a DNABII protein with a K_{D} of less than 1000 nM, or alternatively less than 900 nM, or alternatively less than 800 nM, or alternatively less than 700 nM, or alternatively less than 600 nM, or alternatively less than 500 nM, or alternatively less than 400 nM, or alternatively less than 300 nM, or alternatively less than 200 nM, or alternatively less than 100 nM, or alternatively less than 90 nM, or alternatively less than 80 nM, or alternatively less than 70 nM, or alternatively less than 60 nM, or alternatively less than 50 nM, or alternatively less than 40 nM, or alternatively less than 30 nM, or alternatively less than 20 nM, or alternatively less than 15 nM, or alternatively less than 10 nM, or alternatively less than 9 nM, or alternatively less than 8 nM, or alternatively less than 7 nM, or alternatively less than 6 nM, or alternatively less than 5 nM, or alternatively less than 4 nM, or alternatively less than 3 nM, or alternatively less than 2 nM, or alternatively less than 1 nM. In one embodiment, such K_{D} is determined using the surface plasmon resonance (SPR) methods shown in the Examples. In some of the aspects of the antibodies provided herein, the antigen binding site specifically binds to a DNABII protein.

In some of the aspects of the antibodies provided herein, the antibody, fragment thereof, polypeptide or CDR binds a DNABII protein (including but not limited to: a tip region of IHF or HU, a tip region of IHFA or IHFB, the tip-chimeric peptide IhfA5-mIhfB4_{NTHI}, a tail region of IHF or HU, a tail region of IHFA or IHFB, and/or the tail-chimeric peptide IhfA3-IhfB2_{NTHI}) with a K_{off} of less than 1.0E-02 s⁻¹, or alternatively less than 9.0E-03 s⁻¹, or alternatively less than 8.0E-03 s⁻¹, or alternatively less than 7.0E-03 s⁻¹, or alternatively less than 6.0E-03 s⁻¹, or alternatively less than 5.0E-03 s⁻¹, or alternatively less than 4.0E-03 s⁻¹, or alternatively less than 3.0E-03 s⁻¹, or alternatively less than 2.0E-03 s⁻¹ or alternatively less than 1.0E-03 s⁻¹, or alternatively less than 9.0E-04 s⁻¹, or alternatively less than 8.0E-04 s⁻¹, or alternatively less than 7.0E-04 s⁻¹, or alternatively less than 6.0E-04 s⁻¹, or alternatively less than 5.0E-04 s⁻¹, or alternatively less than 4.0E-04 s⁻¹, or alternatively less than 3.0E-04 s⁻¹, or alternatively less than 2.0E-04 s⁻¹ or alternatively less than 1.0E-04 s⁻¹, or alternatively less than 9.0E-05 s⁻¹, or alternatively less than 8.0E-05 s⁻¹, or alternatively less than 7.0E-05 s⁻¹, or alternatively less than 6.0E-05 s⁻¹, or alternatively less than 5.0E-05 s⁻¹, or alternatively less than 4.0E-05 s⁻¹, or alternatively less than 3.0E-05 s⁻¹, or alternatively less than 2.0E-05 s⁻¹ or alternatively less than 1.0E-05 s⁻¹. In one embodiment, such K_{off} is determined using the surface plasmon resonance (SPR) methods shown in the Examples.

In some of the aspects of the antibodies provided herein, the antibody, fragment thereof, polypeptide or CDR binds a DNABII protein (including but not limited to: a tip region of IHF or HU, a tip region of IHFA or IHFB, the tip-chimeric peptide IhfA5-mIhfB4_{NTHI}, a tail region of IHF or HU, a tail region of IHFA or IHFB, and/or the tail-chimeric peptide IhfA3-IhfB2_{NTHI}) with a Kₒₙ of less than 9.0E-02 M⁻¹ s⁻¹, or alternatively less than 8.0E-02 M⁻¹ s⁻¹, or alternatively less than 7.0E-02 M⁻¹ s⁻¹, or alternatively less than 6.0E-02 M⁻¹ s⁻¹, or alternatively less than 5.0E-02 M⁻¹ s⁻¹, or alternatively less than 4.0E-02 M⁻¹ s⁻¹, or alternatively less than 3.0E-02 M⁻¹ s⁻¹, or alternatively less than 2.0E-02 M⁻¹ s⁻¹ or alternatively less than 1.0E-02 M⁻¹ s⁻¹, or alternatively less than 9.0E-03 M⁻¹ s⁻¹, or alternatively less than 8.0E-03 M⁻¹ s⁻¹, or alternatively less than 7.0E-03 M⁻¹ s⁻¹, or alternatively less than 6.0E-03 M⁻¹ s⁻¹, or alternatively less than 5.0E-03 M⁻¹ s⁻¹, or alternatively less than 4.0E-03 M⁻¹ s⁻¹, or alternatively less than 3.0E-03 M⁻¹ s⁻¹, or alternatively less than 2.0E-03 M⁻¹ s⁻¹ or alternatively less than 1.0E-03 M⁻¹ s⁻¹, or alternatively less than 9.0E-04 M⁻¹ s⁻¹, or alternatively less than 8.0E-04 M⁻¹ s⁻¹, or alternatively less than 7.0E-04 M⁻¹ s⁻¹, or alternatively less than 6.0E-04 M⁻¹ s⁻¹, or alternatively less than 5.0E-04 M⁻¹ s⁻¹, or alternatively less than 4.0E-04 M⁻¹ s⁻¹, or alternatively less than 3.0E-04 M⁻¹ s⁻¹, or alternatively less than 2.0E-04 M⁻¹ s⁻¹ or alternatively less than 1.0E-04 M⁻¹ s⁻¹, or alternatively less than 9.0E-05 M⁻¹ s⁻¹, or alternatively less than 8.0E-05 M⁻¹ s⁻¹, or alternatively less than 7.0E-05 M⁻¹ s⁻¹, or alternatively less than 6.0E-05 M⁻¹ s⁻¹, or alternatively less than 5.0E-05 M⁻¹ s⁻¹, or alternatively less than 4.0E-05 M⁻¹ s⁻¹, or alternatively less than 3.0E-05 M⁻¹ s⁻¹, or alternatively less than 2.0E-05 M⁻¹ s⁻¹ or alternatively less than 1.0E-05 M⁻¹ s⁻¹. In one embodiment, such Kₒₙ is determined using the surface plasmon resonance (SPR) methods shown in the Examples.

In some aspects of this invention, the association constant K_{A} for the IhfA5-mIhfB4_{NTHI} Tip chimeric peptide (in 1/M) is about 3E+05 to about 2E+08. In another aspect, the K_{A} is about 3E+05 to about 1E+08, or alternatively about 2E+05 to about 1E+08, or alternatively about 1E+05 to about 1E+08, or alternatively about 1E+06 to about 1E+08, or alternatively about 1E+07 to about 1E+08, or alternatively about 1E+04 to about 1E+09, alternatively about 1E+05 to about 1E+09, alternatively about 1E+06 to about 1E+09, alternatively about 1E+07 to about 1E+09, alternatively about 1E+08 to about 1E+09, alternatively about 1E+04 to about 1E+09, or alternatively about 1E+03 to about 1E+10.

In another aspect, the dissociation constant K_{D} for the IhfA5-mIhfB4_{NTHI} Tip chimeric peptide (in M) is about 5E-09 to about 3E-06, or alternatively about 1E-09 to about 1E-06, or alternatively about 1E-08 to about 1E-05, or alternatively about 1E-07 to about 1E-05, or alternatively about 1E-06 to about 1E-05, or alternatively about 1E-09 to about 1E-08, or alternatively about 1E-08 to about 1E-07, or alternatively about 1E-9 to about 1E-08, or alternatively about 1E-10 to about 1E-09, or alternatively about 1E-11 to about 1E-10.

In one aspect, the K_{A} for the IhfA3-IhfB2_{NTHI} Tail chimeric peptide (in 1/M) is from about 7E+06 to about 2E+09, or alternatively about 1E+05 to about 1E+08, or alternatively about 1E+06 to about 1E+08, or alternatively about 1E+07 to about 1E+08, or alternatively about 1E+04 to about 1E+09, alternatively about 1E+05 to about 1E+09, alternatively about 1E+06 to about 1E+09, alternatively about 1E+07 to about 1E+09, alternatively about 1E+08 to about 1E+09, alternatively about 1E+04 to about 1E+09, or alternatively about 1E+03 to about 1E+10, or alternatively about 1E+03 to about 1E+11, or alternatively about 1E+03 to about 1E+12, or alternatively about 1E+09 to about 1E+10, or alternatively about 1E+10 to about 1E+11, or alternatively about 1E+11 to about 1E+12.

In another aspect, the K_{D} for the IhfA3-IhfB2_{NTHI} Tail chimeric peptide (in M) is about 6E-10 to about 2E-07, or alternatively about 1E-09 to about 1E-06, or alternatively about 1E-08 to about 1E-05, or alternatively about 1E-07 to about 1E-05, or alternatively about 1E-06 to about 1E-05, or alternatively about 1E-09 to about 1E-08, or alternatively about 1E-08 to about 1E-07, or alternatively about 1E-9 to about 1E-08, or alternatively about 1E-10 to about 1E-09, or alternatively about 1E-11 to about 1E-10, or alternatively about 1E-11 to about 1E-12.

In some of the aspects of the antibodies provided herein, the antibody, fragment thereof, polypeptide or CDR that binds the tip region of a DNABII protein (including but not limited to: a tip region of IHF or HU, a tip region of IHFA or IHFB, and/or the tip-chimeric peptide IhfA5-mIhfB4_{NTHI}) reduces biomass of a biofilm in vitro by at least about 10%, or alternatively at least about 15%, or alternatively at least about 20%, or alternatively at least about 25%, or alternatively at least about 30%, or alternatively at least about 35%, or alternatively at least about 40%, or alternatively at least about 45%, or alternatively at least about 50%, or alternatively at least about 55%, or alternatively at least about 60%, or alternatively at least about 65%, or alternatively at least about 70%, or alternatively at least about 75%, or alternatively at least about 80%, or alternatively at least about 85%, or alternatively at least about 90%, or alternatively at least about 95%. In some of the aspects of the antibodies provided herein, the antibody, fragment thereof, polypeptide or CDR that binds the tail region of a DNABII protein (including but not limited to: a tail region of IHF or HU, a tail region of IHFA or IHFB, and/or the tail-chimeric peptide IhfA3-IhfB2_{NTHI}) reduces biomass of a biofilm in vitro by less than about 1%, or alternatively less than about 2%, or alternatively less than about 3%, or alternatively less than about 4%, or alternatively less than about 5%, or alternatively less than about 6%, or alternatively less than about 7%, or alternatively less than about 8%, or alternatively less than about 9%, or alternatively less than about 10%, or alternatively less than about 12%, or alternatively less than about 15%, or alternatively less than about 20%, or alternatively less than about 25%, or alternatively less than about 30%, or alternatively less than about 35%, or alternatively less than about 40%, or alternatively less than about 45%, or alternatively less than about 50%. In one embodiment, such biomass change is determined using the methods shown in Example 3 or 4.

In some of the aspects of the antibodies provided herein, the antibody, fragment thereof, polypeptide or CDR that binds the tip region of a DNABII protein (including but not limited to: a tip region of IHF or HU, a tip region of IHFA or IHFB, and/or the tip-chimeric peptide IhfA5-mIhfB4_{NTHI}) reduces bacterial load in a subject by at least about 10%, or alternatively at least about 15%, or alternatively at least about 20%, or alternatively at least about 25%, or alternatively at least about 30%, or alternatively at least about 35%, or alternatively at least about 40%, or alternatively at least about 45%, or alternatively at least about 50%, or alternatively at least about 55%, or alternatively at least about 60%, or alternatively at least about 65%, or alternatively at least about 70%, or alternatively at least about 75%, or alternatively at least about 80%, or alternatively at least about 85%, or alternatively at least about 90%, or alternatively at least about 91%, or alternatively at least about 92%, or alternatively at least about 93%, or alternatively at least about 94%, or alternatively at least about 95%, or alternatively at least about 96%, or alternatively at least about 97%, or alternatively at least about 98%, or alternatively at least about 99%. In some of the aspects of the antibodies provided herein, the antibody, fragment thereof, polypeptide or CDR that binds the tail region of a DNABII protein (including but not limited to: a tail region of IHF or HU, a tail region of IHFA or IHFB, and/or the tail-chimeric peptide IhfA3-IhfB2_{NTHI}) reduces bacterial load in a subject by less than about 1%, or alternatively less than about 2%, or alternatively less than about 3%, or alternatively less than about 4%, or alternatively less than about 5%, or alternatively less than about 6%, or alternatively less than about 7%, or alternatively less than about 8%, or alternatively less than about 9%, or alternatively less than about 10%, or alternatively less than about 12%, or alternatively less than about 15%, or alternatively less than about 20%, or alternatively less than about 25%, or alternatively less than about 30%, or alternatively less than about 35%, or alternatively less than about 40%, or alternatively less than about 45%, or alternatively less than about 50%. In one embodiment, such change in the bacterial load is determined using the methods shown in the Examples.

In some of the aspects of the antibodies provided herein, the antibody, fragment thereof, polypeptide or CDR that binds the tip region of a DNABII protein (including but not limited to: a tip region of IHF or HU, a tip region of IHFA or IHFB, and/or the tip-chimeric peptide IhfA5-mIhfB4_{NTHI}) reduces middle ear occlusion in a subject having otitis media (OM) by at least about 10%, or alternatively at least about 15%, or alternatively at least about 20%, or alternatively at least about 25%, or alternatively at least about 30%, or alternatively at least about 35%, or alternatively at least about 40%, or alternatively at least about 45%, or alternatively at least about 50%, or alternatively at least about 55%, or alternatively at least about 60%, or alternatively at least about 65%, or alternatively at least about 70%, or alternatively at least about 75%, or alternatively at least about 80%, or alternatively at least about 85%, or alternatively at least about 90%, or alternatively at least about 91%, or alternatively at least about 92%, or alternatively at least about 93%, or alternatively at least about 94%, or alternatively at least about 95%, or alternatively at least about 96%, or alternatively at least about 97%, or alternatively at least about 98%, or alternatively at least about 99%. In some of the aspects of the antibodies provided herein, the antibody, fragment thereof, polypeptide or CDR that binds the tail region of a DNABII protein (including but not limited to: a tail region of IHF or HU, a tail region of IHFA or IHFB, and/or the tail-chimeric peptide IhfA3-IhfB2_{NTHI}) reduces middle ear occlusion in a subject having otitis media (OM) by less than about 1%, or alternatively less than about 2%, or alternatively less than about 3%, or alternatively less than about 4%, or alternatively less than about 5%, or alternatively less than about 6%, or alternatively less than about 7%, or alternatively less than about 8%, or alternatively less than about 9%, or alternatively less than about 10%, or alternatively less than about 12%, or alternatively less than about 15%, or alternatively less than about 20%, or alternatively less than about 25%, or alternatively less than about 30%, or alternatively less than about 35%, or alternatively less than about 40%, or alternatively less than about 45%, or alternatively less than about 50%. In one embodiment, such change in middle ear occlusion is determined using the methods shown in the Examples in an experimental OM model.

In some of the aspects of the antibodies provided herein, the antibody, fragment thereof, polypeptide or CDR that binds the tip region of a DNABII protein (including but not limited to: a tip region of IHF or HU, a tip region of IHFA or IHFB, and/or the tip-chimeric peptide IhfA5-mIhfB4_{NTHI}) reduces the relative mucosal biofilm score and/or biomass score in a subject having a mucosal biofilm (such as having OM) by at least about 10%, or alternatively at least about 15%, or alternatively at least about 20%, or alternatively at least about 25%, or alternatively at least about 30%, or alternatively at least about 35%, or alternatively at least about 40%, or alternatively at least about 45%, or alternatively at least about 50%, or alternatively at least about 55%, or alternatively at least about 60%, or alternatively at least about 65%, or alternatively at least about 70%, or alternatively at least about 75%, or alternatively at least about 80%, or alternatively at least about 85%, or alternatively at least about 90%, or alternatively at least about 91%, or alternatively at least about 92%, or alternatively at least about 93%, or alternatively at least about 94%, or alternatively at least about 95%, or alternatively at least about 96%, or alternatively at least about 97%, or alternatively at least about 98%, or alternatively at least about 99%. In one embodiment, the antibody, fragment thereof, polypeptide or CDR that binds the tip region of a DNABII protein (including but not limited to: a tip region of IHF or HU, a tip region of IHFA or IHFB, and/or the tip-chimeric peptide IhfA5-mIhfB4_{NTHI}) reduces the relative mucosal biofilm score and/or biomass score in a subject having a mucosal biofilm (such as having OM) by at least about 0.5, or alternatively at least about 1, or alternatively at least about 1.5, or alternatively at least about 2, or alternatively at least about 2.5, or alternatively at least about 3, or alternatively at least about 3.5, or alternatively at least about 4. In some of the aspects of the antibodies provided herein, the antibody, fragment thereof, polypeptide or CDR that binds the tail region of a DNABII protein (including but not limited to: a tail region of IHF or HU, a tail region of IHFA or IHFB, and/or the tail-chimeric peptide IhfA3-IhfB2_{NTHI}) reduces the relative mucosal biofilm score and/or biomss score in a subject having a mucosal biofilm (such as having OM) by less than about 1%, or alternatively less than about 2%, or alternatively less than about 3%, or alternatively less than about 4%, or alternatively less than about 5%, or alternatively less than about 6%, or alternatively less than about 7%, or alternatively less than about 8%, or alternatively less than about 9%, or alternatively less than about 10%, or alternatively less than about 12%, or alternatively less than about 15%, or alternatively less than about 20%, or alternatively less than about 25%, or alternatively less than about 30%, or alternatively less than about 35%, or alternatively less than about 40%, or alternatively less than about 45%, or alternatively less than about 50%. In some of the aspects of the antibodies provided herein, the antibody, fragment thereof, polypeptide or CDR that binds the tail region of a DNABII protein (including but not limited to: a tail region of IHF or HU, a tail region of IHFA or IHFB, and/or the tail-chimeric peptide IhfA3-IhfB2_{NTHI}) reduces the relative mucosal biofilm score and/or biomss score in a subject having a mucosal biofilm (such as having OM) by less than about 0.1, or alternatively less than about 0.2, or alternatively less than about 0.3, or alternatively less than about 0.4, or alternatively less than about 0.5, or alternatively less than about 0.6, or alternatively less than about 0.7, or alternatively less than about 0.8, or alternatively less than about 0.9, or alternatively less than about 1, or alternatively less than about 1.5, or alternatively less than about 2, or alternatively less than about 2.5. In one embodiment, such score is determined using the methods shown in the Examples.

In certain embodiments, the DNABII protein is an HU or an IHF. In a further embodiment, the DNABII protein is an IhfA, an IhfB or both. In yet a further embodiment, the antibody, fragment thereof, polypeptide or CDR binds the tip region or the tail region of the DNABII protein (including but not limited to: a tip region of IHF or HU, a tip region of IHFA or IHFB, the tip-chimeric peptide IhfA5-mIhfB4_{NTHI}, a tail region of IHF or HU, a tail region of IHFA or IHFB, and/or the tail-chimeric peptide IhfA3-IhfB2_{NTHI}). In one embodiment, the antibody, fragment thereof, polypeptide or CDR binds the IhfA5-mIhfB4_{NTHI} Tip chimeric peptide. In another embodiment, the antibody, fragment thereof, polypeptide or CDR binds the IhfA3-IhfB2_{NTHI} Tail chimeric peptide.

In some of the aspects of the antibodies provided herein, the antibody is soluble Fab.

In some of the aspects of the antibodies provided herein, the HC and LC variable domain sequences are components of the same polypeptide chain. In some of the aspects of the antibodies provided herein, the HC and LC variable domain sequences are components of different polypeptide chains.

In some of the aspects of the antibodies provided herein, the antibody is a full-length antibody.

In some of the aspects of the antibodies provided herein, the antibody is chimeric or humanized.

In some of the aspects of the antibodies provided herein, the antibody comprises an Fc domain. In some of the aspects of the antibodies provided herein, the antibody is a non-human animal such as a rat, sheep, bovine, canine, feline or rabbit antibody. In some of the aspects of the antibodies provided herein, the antibody is a human or humanized antibody or is non-immunogenic in a human.

In some of the aspects of the antibodies provided herein, the antibody comprises a human antibody framework region. Examples of framework regions that can be fused to the LC and HC sequences are known in the art, examples of such are provided in SEQ ID NOs: 15-23, or equivalents of each thereof.

In other aspects, one or more amino acid residues in a CDR of the antibodies provided herein are substituted with another amino acid. The substitution may be “conservative” in the sense of being a substitution within the same family of amino acids. The naturally occurring amino acids may be divided into the following four families and conservative substitutions will take place within those families.
1) Amino acids with basic side chains: lysine, arginine, histidine.
2) Amino acids with acidic side chains: aspartic acid, glutamic acid
3) Amino acids with uncharged polar side chains: asparagine, glutamine, serine, threonine, tyrosine.
4) Amino acids with nonpolar side chains: glycine, alanine, valine, leucine, isoleucine, proline, phenylalanine, methionine, tryptophan, cysteine.

In another aspect, one or more amino acid residues are added to or deleted from one or more CDRs of an antibody. Such additions or deletions occur at the N or C termini of the CDR or at a position within the CDR.

By varying the amino acid sequence of the CDRs of an antibody by addition, deletion or substitution of amino acids, various effects such as increased binding affinity for the target antigen may be obtained.

It is to be appreciated that antibodies of the present disclosure comprising such varied CDR sequences still bind a DNABII protein with similar specificity and sensitivity profiles as the disclosed antibodies. This may be tested by way of the binding assays, such as ELISA or SPR.

In a further aspect, the antibodies are characterized by being both immunodominant and immunoprotective, as determined using appropriate assays and screens.

In another aspect, the antibodies can be modified by conventional techniques, that may in one aspect increase the half-life of the antibody, e.g., PEGylation, a PEG mimetic, polysialylation, HESylation or glycosylation.

The antibodies and antigen binding fragments can further comprise a detectable marker or a purification marker.

### Antibodies and Derivatives Thereof

This disclosure also provides an antibody that binds and/or specifically recognizes and binds an isolated polypeptide for use in the methods disclosed herein. The antibody can be any of the various antibodies described herein, non-limiting, examples of such include a polyclonal antibody, a monoclonal antibody, a chimeric antibody, a human antibody, a veneered antibody, a diabody, a humanized antibody, an antibody derivative, a recombinant humanized antibody, or an equivalent (such as a derivative) or fragment of each thereof. In one aspect, the fragment comprises, or alternatively consists essentially of, or yet further consists of the CDR of the antibody. In one aspect, the antibody is detectably labeled or further comprises a detectable label conjugated to it.

Also provided is a hybridoma cell line that produces a monoclonal antibody disclosed herein. Compositions comprising or alternatively consisting essentially of or yet further, consisting of one or more of the above embodiments are further provided herein. Further provided are polynucleotides that encode the amino acid sequence of the antibodies and fragments as well as methods to produce recombinantly or chemically synthesize the antibody polypeptides and fragments thereof. The antibody polypeptides can be produced in a eukaryotic or prokaryotic cell, or by other methods known in the art and described herein.

Examples of CDR sequences include without limitation comprise, consist essentially of, or yet further consist of, the following: the heavy chain variable region of the antibody or a fragment thereof comprises, or alternatively consists essentially of, or yet further consists of, the polypeptide encoded by the below polynucleotide sequence:

Antibodies can be generated using conventional techniques known in the art and are well-described in the literature. Several methodologies exist for production of polyclonal antibodies. For example, polyclonal antibodies are typically produced by immunization of a suitable mammal such as, but not limited to, chickens, goats, guinea pigs, hamsters, horses, mice, rats, and rabbits. An antigen is injected into the mammal, induces the B-lymphocytes to produce immunoglobulins specific for the antigen. Immunoglobulins may be purified from the mammal's serum.

Variations of this methodology include modification of adjuvants, routes and site of administration, injection volumes per site and the number of sites per animal for optimal production and humane treatment of the animal. For example, adjuvants typically are used to improve or enhance an immune response to antigens. Most adjuvants provide for an injection site antigen depot, which allows for a stow release of antigen into draining lymph nodes. Other adjuvants include surfactants which promote concentration of protein antigen molecules over a large surface area and immunostimulatory molecules. Non-limiting examples of adjuvants for polyclonal antibody generation include Freund's adjuvants, Ribi adjuvant system, and Titermax. Polyclonal antibodies can be generated using methods known in the art some of which are described in U.S. Pat. Nos. 7,279,559; 7,119,179; 7,060,800; 6,709,659; 6,656,746; 6,322,788; 5,686,073; and 5,670,153.

Antibody derivatives of the present disclosure can also be prepared by delivering a polynucleotide encoding an antibody disclosed herein to a suitable host such as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; and 5,304,489.

The term “antibody derivative” includes post-translational modification to linear polypeptide sequence of the antibody or fragment. For example, U.S. Pat. No. 6,602,684 B1 describes a method for the generation of modified glycol-forms of antibodies, including whole antibody molecules, antibody fragments, or fusion proteins that include a region equivalent to the Fc region of an immunoglobulin, having enhanced Fe-mediated cellular toxicity, and glycoproteins so generated.

The antibodies disclosed herein also include derivatives that are modified by the covalent attachment of any type of molecule to the antibody such that covalent attachment does not prevent the antibody from generating an anti-idiotypic response. Antibody derivatives include, but are not limited to, antibodies that have been modified by glycosylation, acetylation, pegylation, phosphorylation, amidation, derivatization by known protecting/blocking groups, proteolytic cleavage, linkage to a cellular ligand or other protein, etc. Additionally, the derivatives may contain one or more non-classical amino acids.

Antibody derivatives also can be prepared by delivering a polynucleotide disclosed herein to provide transgenic plants and cultured plant cells (e.g., but not limited to tobacco, maize, and duckweed) that produce such antibodies, specified portions or variants in the plant parts or in cells cultured therefrom. For example, Cramer et al. (1999) Curr. Top. Microbol. Immunol. 240:95-118 and references cited therein, describe the production of transgenic tobacco leaves expressing large amounts of recombinant proteins, e.g., using an inducible promoter. Transgenic maize has been used to express mammalian proteins at commercial production levels, with biological activities equivalent to those produced in other recombinant systems or purified from natural sources. See, e.g., Hood et al. (1999) Adv. Exp. Med. Biol. 464:127-147 and references cited therein. Antibody derivatives have also been produced in large amounts from transgenic plant seeds including antibody fragments, such as single chain antibodies (scFv's), including tobacco seeds and potato tubers. See, e.g., Conrad et al. (1998) Plant Mol. Biol. 38:101-109 and references cited therein. Thus, antibodies can also be produced using transgenic plants, according to know methods.

Antibody derivatives also can be produced, for example, by adding exogenous sequences to modify immunogenicity or reduce, enhance or modify binding, affinity, on-rate, off-rate, avidity, specificity, half-life, or any other suitable characteristic. Generally, part or all of the non-human or human CDR sequences are maintained while the non-human sequences of the variable and constant regions are replaced with human or other amino acids or variable or constant regions from other isotypes.

In general, the CDR residues are directly and most substantially involved in influencing antigen binding. Humanization or engineering of antibodies can be performed using any known method such as, but not limited to, those described in U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

Chimeric, humanized or primatized antibodies of the present disclosure can be prepared based on the sequence of a reference monoclonal antibody prepared using standard molecular biology techniques. DNA encoding the heavy and light chain immunoglobulins can be obtained from the hybridoma of interest and engineered to contain non-reference (e.g., human) immunoglobulin sequences using standard molecular biology techniques. For example, to create a chimeric antibody, the murine variable regions can be linked to human constant regions using methods known in the art (U.S. Pat. No. 4,816,567). To create a humanized antibody, the murine CDR regions can be inserted into a human framework using methods known in the art (U.S. Pat. Nos. 5,225,539 and 5,530,101; 5,585,089; 5,693,762; and 6,180,370). Similarly, to create a primatized antibody the murine CDR regions can be inserted into a primate framework using methods known in the art (WO 93/02108 and WO 99/55369).

Techniques for making partially to fully human antibodies are known in the art and any such techniques can be used. According to one embodiment, fully human antibody sequences are made in a transgenic mouse which has been engineered to express human heavy and light chain antibody genes. Multiple strains of such transgenic mice have been made which can produce different classes of antibodies. B cells from transgenic mice which are producing a desirable antibody can be fused to make hybridoma cell lines for continuous production of the desired antibody. (See for example, Russel et al. (2000) Infection and Immunity April 2000:1820-1826; Gallo et al. (2000) European J. of Immun. 30:534-540; Green (1999) J. of Immun. Methods 231:11-23; Yang et al. (1999A) J. of Leukocyte Biology 66:401-410; Yang (1999B) Cancer Research 59(6):1236-1243; Jakobovits (1998) Advanced Drug Reviews 31:33-42; Green and Jakobovits (1998) J. Exp. Med. 188(3):483-495; Jakobovits (1998) Exp. Opin. Invest. Drugs 7(4):607-614; Tsuda et al. (1997) Genomics 42:413-421; Sherman-Gold (1997) Genetic Engineering News 17(14); Mendez et al. (1997) Nature Genetics 15:146-156; Jakobovits (1996) Weir's Handbook of Experimental Immunology, The Integrated Immune System Vol. IV, 194.1-194.7; Jakobovits (1995) Current Opinion in Biotechnology 6:561-566; Mendez et al. (1995) Genomics 26:294-307; Jakobovits (1994) Current Biology 4(8):761-763; Arbones et al. (1994) Immunity 1(4):247-260; Jakobovits (1993) Nature 362(6417):255-258; Jakobovits et al. (1993) Proc. Natl. Acad. Sci. USA 90(6):2551-2555; and U.S. Pat. No. 6,075,181.)

The antibodies disclosed herein also can be modified to create chimeric antibodies. Chimeric antibodies are those in which the various domains of the antibodies' heavy and light chains are coded for by DNA from more than one species. See, e.g., U.S. Pat. No. 4,816,567.

Alternatively, the antibodies disclosed herein can also be modified to create veneered antibodies. Veneered antibodies are those in which the exterior amino acid residues of the antibody of one species are judiciously replaced or “veneered” with those of a second species so that the antibodies of the first species will not be immunogenic in the second species thereby reducing the immunogenicity of the antibody. Since the antigenicity of a protein is primarily dependent on the nature of its surface, the immunogenicity of an antibody could be reduced by replacing the exposed residues which differ from those usually found in another mammalian species. This judicious replacement of exterior residues should have little, or no, effect on the interior domains, or on the interdomain contacts. Thus, ligand binding properties should be unaffected as a consequence of alterations which are limited to the variable region framework residues. The process is referred to as “veneering” since only the outer surface or skin of the antibody is altered, the supporting residues remain undisturbed.

The procedure for “veneering” makes use of the available sequence data for human antibody variable domains compiled by Kabat et al. (1987) Sequences of Proteins of Immunological interest, 4th ed., Bethesda, Md., National Institutes of Health, updates to this database, and other accessible U.S. and foreign databases (both nucleic acid and protein). Non-limiting examples of the methods used to generate veneered antibodies include EP 519596; U.S. Pat. No. 6,797,492; and described in Padlan et al. (1991) Mol. Immunol. 28(4-5):489-498.

The term “antibody derivative” also includes “diabodies” which are small antibody fragments with two antigen-binding sites, wherein fragments comprise a heavy chain variable domain (V_{H}) connected to a light chain variable domain (V_{L}) in the same polypeptide chain. (See for example, EP 404,097; WO 93/11161; and Hollinger et al. (1993) Proc. Natl. Acad. Sci. USA 90:6444-6448.) By using a linker that is too short to allow pairing between the two domains on the same chain, the domains are forced to pair with the complementary domains of another chain and create two antigen-binding sites. (See also, U.S. Pat. No. 6,632,926 to Chen et al., which discloses antibody variants that have one or more amino acids inserted into a hypervariable region of the parent antibody and a binding affinity for a target antigen which is at least about two fold stronger than the binding affinity of the parent antibody for the antigen).

The term “antibody derivative” further includes engineered antibody molecules, fragments and single domains such as scFv, dAbs, nanobodies, minibodies, Unibodies, and Affibodies & Hudson (2005) Nature Biotech 23(9):1126-36; U.S. Pat. Application Publication No. 2006/0211088; PCT International Application Publication No. WO 2007/059782; U.S. Pat. No. 5,831,012).

The term “antibody derivative” further includes “linear antibodies”. The procedure for making linear antibodies is known in the art and described in Zapata et al. (1995) Protein Eng. 8(10):1057-1062. Briefly, these antibodies comprise a pair of tandem Ed segments (V_{H}—C_{H}1—V_{H}—C_{H}1) which form a pair of antigen binding regions. Linear antibodies can be bispecific or monospecific.

The antibodies disclosed herein can be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

Antibodies of the present disclosure include naturally purified products, products of chemical synthetic procedures, and products produced by recombinant techniques from a eukaryotic host, including, for example, yeast, higher plant, insect and mammalian cells, or alternatively from a prokaryotic host as described above. A number of antibody production systems are described in Birch & Radner (2006) Adv. Drug Delivery Rev. 58: 671-685.

If an antibody being tested binds with protein or polypeptide, then the antibody being tested and the antibodies provided by this disclosure are equivalent. It also is possible to determine without undue experimentation, whether an antibody has the same specificity as the antibody disclosed herein by determining whether the antibody being tested prevents an antibody disclosed herein from binding the protein or polypeptide with which the antibody is normally reactive. If the antibody being tested competes with the antibody disclosed herein as shown by a decrease in binding by the monoclonal antibody disclosed herein, then it is likely that the two antibodies bind to the same or a closely related epitope. Alternatively, one can pre-incubate the antibody disclosed herein with a protein with which it is normally reactive, and determine if the antibody being tested is inhibited in its ability to bind the antigen. If the antibody being tested is inhibited then, in all likelihood, it has the same, or a closely related, epitopic specificity as the antibody disclosed herein.

The term “antibody” also is intended to include antibodies of all immunoglobulin isotypes and subclasses. Particular isotypes of a monoclonal antibody can be prepared either directly by selecting from an initial fusion, or prepared secondarily, from a parental hybridoma secreting a monoclonal antibody of different isotype by using the sib selection technique to isolate class switch variants using the procedure described in Steplewski et al. (1985) Proc. Natl. Acad. Sci. USA 82:8653 or Spira et al. (1984) J. Immunol. Methods 74:307. Alternatively, recombinant DNA techniques may be used.

The isolation of other monoclonal antibodies with the specificity of the monoclonal antibodies described herein can also be accomplished by one of ordinary skill in the art by producing anti-idiotypic antibodies. Herlyn et al. (1986) Science 232:100. An anti-idiotypic antibody is an antibody which recognizes unique determinants present on the monoclonal antibody of interest.

In some aspects disclosed herein, it will be useful to detectably or therapeutically label the antibody. Suitable labels are described supra. Methods for conjugating antibodies to these agents are known in the art. For the purpose of illustration only, antibodies can be labeled with a detectable moiety such as a radioactive atom, a chromophore, a fluorophore, or the like. Such labeled antibodies can be used for diagnostic techniques, either in vivo, or in an isolated test sample.

The coupling of antibodies to low molecular weight haptens can increase the sensitivity of the antibody in an assay. The haptens can then be specifically detected by means of a second reaction. For example, it is common to use haptens such as biotin, which reacts avidin, or dinitrophenol, pyridoxal, and fluorescein, which can react with specific anti-hapten antibodies. See, Harlow and Lane (1988) supra.

The variable region of the antibodies of the present disclosure can be modified by mutating amino acid residues within the VH and/or VL CDR 1, CDR 2 and/or CDR 3 regions to improve one or more binding properties (e.g., affinity) of the antibody. Mutations may be introduced by site-directed mutagenesis or PCR-mediated mutagenesis and the effect on antibody binding, or other functional property of interest, can be evaluated in appropriate in vitro or in vivo assays. In certain embodiments, conservative modifications are introduced and typically no more than one, two, three, four or five residues within a CDR region are altered. The mutations may be amino acid substitutions, additions or deletions.

Framework modifications can be made to the antibodies to decrease immunogenicity, for example, by “backmutating” one or more framework residues to the corresponding germline sequence.

In addition, the antibodies disclosed herein may be engineered to include modifications within the Fc region to alter one or more functional properties of the antibody, such as serum half-fife, complement fixation, Fc receptor binding, and/or antigen-dependent cellular cytotoxicity. Such modifications include, but are not limited to, alterations of the number of cysteine residues in the hinge region to facilitate assembly of the light and heavy chains or to increase or decrease the stability of the antibody (U.S. Pat. No. 5,677,425) and amino acid mutations in the Fc hinge region to decrease the biological half-life of the antibody (U.S. Pat. No. 6,165,745).

Additionally, the antibodies disclosed herein may be chemically modified. Glycosylation of an antibody can be altered, for example, by modifying one or more sites of glycosylation within the antibody sequence to increase the affinity of the antibody for antigen (U.S. Pat. Nos. 5,714,350 and 6,350,861). Alternatively, to increase antibody-dependent cell-mediated cytotoxicity, a hypofucosylated antibody having reduced amounts of fucosyl residues or an antibody having increased bisecting GlcNac structures can be obtained by expressing the antibody in a host cell with altered glycosylation mechanism (Shields, R. L. et al. (2002) J. Biol. Chem. 277:26733-26740; Umana et al. (1999) Nat. Biotech. 17:176-180).

The antibodies disclosed herein can be pegylated to increase biological half-life by reacting the antibody or fragment thereof with polyethylene glycol (PEG) or a reactive ester or aldehyde derivative of PEG, under conditions in which one or more PEG groups become attached to the antibody or antibody fragment. Antibody pegylation may be carried out by an acylation reaction or an alkylation reaction with a reactive PEG molecule (or an analogous reactive water soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. The antibody to be pegylated can be an aglycosylated antibody. Methods for pegylating proteins are known in the art and can be applied to the antibodies disclosed herein (EP 0154316 and EP 0401384).

Additionally, antibodies may be chemically modified by conjugating or fusing the antigen-binding region of the antibody to serum protein, such as human serum albumin, to increase half-life of the resulting molecule. Such approach is for example described in EP 0322094 and EP 0486525.

The antibodies or fragments thereof of the present disclosure may be conjugated to a diagnostic agent and used diagnostically, for example, to monitor the development or progression of a disease and determine the efficacy of a given treatment regimen. Examples of diagnostic agents include enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody or fragment thereof, or indirectly, through a linker using techniques known in the art. Examples of suitable enzymes include horseradish peroxidase, alkaline phosphatase, beta-galactosidase, or acetylcholinesterase. Examples of suitable prosthetic group complexes include streptavidin/biotin and avidin/biotin. Examples of suitable fluorescent materials include umbelliferone, fluorescein, fluorescein isothiocyanate, rhodamine, dichlorotriazinylamine fluorescein, dansyl chloride or phycoerythrin. An example of a luminescent material includes luminol. Examples of bioluminescent materials include luciferase, luciferin, and aequorin. Examples of suitable radioactive material include ¹²⁵I, ³¹I, Indium-111, Lutetium-171, Bismuth-212, Bismuth-213, Astatine-211, Copper-62, Copper-64, Copper-67, Yttrium-90, Iodine-125, Iodine-131, Phosphorus-32, Phosphorus-33, Scandium-47, Silver-111, Gallium-67, Praseodymium-142, Samarium-153, Terbium-161, Dysprosium-166, Holmium-166, Rhenium-186, Rhenium-188, Rhenium-189, Lead-212, Radium-223, Actinium-225, Iron-59, Selenium-75, Arsenic-77, Strontium-89, Molybdenum-99, Rhodium-1105, Palladium-109, Praseodymium-143, Promethium-149, Erbium-169, Iridium-194, Gold-198, Gold-199, and Lead-211. Monoclonal antibodies may be indirectly conjugated with radiometal ions through the use of bifunctional chelating agents that are covalently linked to the antibodies. Chelating agents may be attached through amities (Meares et al. (1984) Anal. Biochem. 142:68-78); sulfhydral groups (Koyama (1994) Chem. Abstr. 120:217-262) of amino acid residues and carbohydrate groups (Rodwell et al. (1986) PNAS USA 83:2632-2636; Quadri et al. (1993) Nucl. Med. Biol. 20:559-570).

Further, the antibodies or fragments thereof of the present disclosure may be conjugated to a therapeutic agent. Suitable therapeutic agents include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin, antimetabolites (such as methotrexate, 6-mercaptopurine, 6-thioguanine, cytarabine, fludarabin, 5-fluorouracil, decarbazine, hydroxyurea, asparaginase, gemcitabinc, cladribine), alkylating agents (such as mechlorethamine, thioepa, chloramhucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin), antibiotics (such as dactinomycin (formerly actinomycin), bleomycin, daunorubicin (formerly daunomycin), doxorubicin, idarubicin, mithramycin, mitomycin, mitoxantrone, plicamycin, anthramycin (AMC)), diphtheria toxin and related molecules (such as diphtheria A chain and active fragments thereof and hybrid molecules), ricin toxin (such as ricin A or a deglycosylated ricin A chain toxin), cholera toxin, a Shiga-like toxin (SLT-I, SLT-II, SLT-IIV), LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins (PAPI, PAPII, and PAP-S), Momordica charantia inhibitor, curcin, crotin, Sapaonaria officinalis inhibitor, gelonin, mitogellin, restrietocin, phenomycin, enomycin toxins and mixed toxins.

Additional suitable conjugated molecules include ribonuclease (RNase), DNase I, an antisense nucleic acid, an inhibitory RNA molecule such as a siRNA molecule, an immunostimulatory nucleic acid, aptamers, ribozymes, triplex forming molecules, and external guide sequences. Aptamers are small nucleic acids ranging from 15-50 bases in length that fold into defined secondary and tertiary structures, such as stem-loops or G-quartets, and can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Ribozymes are nucleic acid molecules that are capable of catalyzing a chemical reaction, either intramolecularly or intermolecularly. Ribozymes typically cleave nucleic acid substrates through recognition and binding of the target substrate with subsequent cleavage. Triplex forming function nucleic acid molecules can interact with double-stranded or single-stranded nucleic acid by forming a triplex, in which three strands of DNA form a complex dependent on both Watson-Crick and Hoogsteen base-pairing. Triplex molecules can bind target regions with high affinity and specificity.

The functional nucleic acid molecules may act as effectors, inhibitors, modulators, and stimulators of a specific activity possessed by a target molecule, or the functional nucleic acid molecules may possess a de novo activity independent of any other molecules.

The therapeutic agents can be linked to the antibody directly or indirectly, using any of a large number of available methods. For example, an agent can be attached at the hinge region of the reduced antibody component via disulfide bond formation, using cross-linkers such as N-succinyl 3-(2-pyridyldithio)proprionate (SPDP), or via a carbohydrate moiety in the Fc region of the antibody (Yu et al. 1994 Int. J. Cancer 56: 244; Upeslacis et al., “Modification of Antibodies by Chemical Methods,” in Monoclonal antibodies: principles and applications, Birch et al. (eds.), pages 187-230 (Wiley-Liss, Inc. 1995); Price, “Production and Characterization of Synthetic Peptide-Derived Antibodies,” in Monoclonal antibodies: Production, engineering and clinical application, Ritter et al. (eds.), pages 60-84 (Cambridge University Press 1995)).

Techniques for conjugating therapeutic agents to antibodies are well known (Amon et al. “Monoclonal Antibodies For Immunotargeting Of Drugs In Cancer Therapy,” in Monoclonal Antibodies And Cancer Therapy; Reisfeld et al. (eds.), pp. 243-56 (Alan R. Liss, Inc. 1985); Hellstrom et al. “Antibodies For Drug Delivery,” in Controlled Drug Delivery (2nd Ed.); Robinson et al. (eds.), pp. 623-53 (Marcel Dekker, Inc. 1987); Thorpe “Antibody Carriers Of Cytotoxic Agents In Cancer Therapy: A Review,” in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody in Cancer Therapy,” in Monoclonal Antibodies For Cancer Detection And Therapy, Baldwin et al. (eds.), pp. 303-16 (Academic Press 1985), and Thorpe et al. “The Preparation And Cytotoxic Properties Of Antibody-Toxin Conjugates,” (1982) Immunol. Rev. 62:119-58).

The antibodies disclosed herein or antigen-binding regions thereof can be linked to another functional molecule such as another antibody or ligand for a receptor to generate a bi-specific or multi-specific molecule that binds to at least two or more different binding sites or target molecules. Linking of the antibody to one or more other binding molecules, such as another antibody, antibody fragment, peptide or binding mimetic, can be done, for example, by chemical coupling, genetic fusion, or noncovalent association. Multi-specific molecules can further include a third binding specificity, in addition to the first and second target epitope.

Bi-specific and multi-specific molecules can be prepared using methods known in the art. For example, each binding unit of the hi-specific molecule can be generated separately and then conjugated to one another. When the binding molecules are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitroberizoic acid) (DTNB), o-phenylenedimaleimide (oPDM), N-succinimidyl-3-(2-pyridyldithio)propionate (SPDP), and sulfosuccinimidyl 4-(N-maleimidomethyl)cyclohaxane-I-carboxylate (sulfo-SMCC) (Karpovsky et al. (1984) J. Exp. Med. 160:1686; Liu et al. (1985) Proc. Natl. Acad. Sci. USA 82:8648). When the binding molecules are antibodies, they can be conjugated by sulfhydryl bonding of the C-terminus hinge regions of the two heavy chains.

The antibodies or fragments thereof of the present disclosure may be linked to a moiety that is toxic to a cell to which the antibody is bound to form “depleting” antibodies. These antibodies are particularly useful in applications where it is desired to deplete an NK cell.

The antibodies disclosed herein may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

The antibodies also can be bound to many different carriers. Thus, this disclosure also provides compositions containing the antibodies and another substance, active or inert. Examples of well-known carriers include glass, polystyrene, polypropylene, polyethylene, dextran, nylon, amylase, natural and modified cellulose, polyacrylamide, agarose, and magnetite. The nature of the carrier can be either soluble or insoluble for purposes disclosed herein. Those skilled in the art will know of other suitable carriers for binding monoclonal antibodies, or will be able to ascertain such, using routine experimentation.

In certain aspects, the disclosure relates to an antibody or antigen binding fragment that specifically recognizes or binds a tip or tail domain of a DNABII protein or fragment thereof, the tail fragment or tip fragment. The DNABII protein or fragment thereof can be an IHF or an HU polypeptide.

Functional Analysis with Antibodies

Antibodies disclosed herein can be used to purify the polypeptides disclosed herein and to identify biological equivalent polypeptide and/or polynucleotides. They also can be used to identify agents that modify the function of the polypeptides disclosed herein. These antibodies include polyclonal antisera, monoclonal antibodies, and various reagents derived from these preparations that are familiar to those practiced in the art and described above.

Antibodies that neutralize the activities of proteins encoded by identified genes can also be used in vivo and in vitro to demonstrate function by adding such neutralizing antibodies into in vivo and in vitro test systems. They also are useful as pharmaceutical agents to modulate the activity of polypeptides disclosed herein.

Various antibody preparations can also be used in analytical methods such as ELISA assays or Western blots to demonstrate the expression of proteins encoded by the identified genes by test cells in vitro or in vivo. Fragments of such proteins generated by protease degradation during metabolism can also be identified by using appropriate polyclonal antisera with samples derived from experimental samples.

The antibodies disclosed herein may be used for vaccination or to boost vaccination, alone or in combination with peptides or protein-based vaccines or dendritic-cell based vaccines.

The general structure of antibodies is known in the art and will only be briefly summarized here. An immunoglobulin monomer comprises two heavy chains and two light chains connected by disulfide bonds. Each heavy chain is paired with one of the light chains to which it is directly bound via a disulfide bond. Each heavy chain comprises a constant region (which varies depending on the isotype of the antibody) and a variable region. The variable region comprises three hypervariable regions (or complementarity determining regions) which are designated CDRH1, CDRH2 and CDRH3 and which are supported within framework regions. Each light chain comprises a constant region and a variable region, with the variable region comprising three hypervariable regions (designated CDRL1, CDRL2 and CDRL3) supported by framework regions in an analogous manner to the variable region of the heavy chain.

The hypervariable regions of each pair of heavy and light chains mutually cooperate to provide an antigen binding site that is capable of binding a target antigen. The binding specificity of a pair of heavy and light chains is defined by the sequence of CDR1, CDR2 and CDR3 of the heavy and light chains. Thus once a set of CDR sequences (i.e., the sequence of CDR1, CDR2 and CDR3 for the heavy and light chains) is determined which gives rise to a particular binding specificity, the set of CDR sequences can, in principle, be inserted into the appropriate positions within any other antibody framework regions linked with any antibody constant regions in order to provide a different antibody with the same antigen binding specificity.

Antibodies for the production of the Fab fragments can be generated using conventional techniques known in the art and are well-described in the literature. Several methodologies exist for production of polyclonal antibodies. For example, polyclonal antibodies are typically produced by immunization of a suitable mammal such as, but not limited to, chickens, goats, sheep, guinea pigs, hamsters, horses, dogs, mice, rats, and rabbits. An antigen is injected into the mammal, and induces the B-lymphocytes to produce immunoglobulins specific for the antigen. Immunoglobulins may be purified from the mammal's serum. Antibodies specific to an IHFα and/or an IHFβ subunit can be generated by injection of polypeptides corresponding to different epitopes of IHFα and IHFβ. For example, antibodies can be generated using the 20 amino acids of each subunit such as fragments A3 and A5 of IHF, respectively, fragment A5 of HU, fragments B2, B4, and mB4 of IHF.

Variations of this methodology include modification of adjuvants, routes and site of administration, injection volumes per site and the number of sites per animal for optimal production and humane treatment of the animal. For example, adjuvants typically are used to improve or enhance an immune response to antigens. Most adjuvants provide for an injection site antigen depot, which allows for a stow release of antigen into draining lymph nodes. Other adjuvants include surfactants which promote concentration of protein antigen molecules over a large surface area and immunostimulatory molecules. Non-limiting examples of adjuvants for polyclonal antibody generation include Freund's adjuvants, Ribi adjuvant system, MPL derived adjuvants, those derived from the heat labile enterotoxin of E. coli (e.g., dmLT=double mutant labile toxin), and Titermax. Polyclonal antibodies can be generated using methods known in the art some of which are described in U.S. Pat. Nos. 7,279,559; 7,119,179; 7,060,800; 6,709,659; 6,656,746; 6,322,788; 5,686,073; and 5,670,153.

Monoclonal antibodies can be generated using conventional hybridoma techniques known in the art and well-described in the literature. For example, a hybridoma is produced by fusing a suitable immortal cell line (e.g., a myeloma cell line such as, but not limited to, Sp2/0, Sp2/0-AG14, NSO, NS1, NS2, AE-1, L.5, P3X63Ag8,653, Sp2 SA3, Sp2 MAI, Sp2 SS1, Sp2 SA5, U397, MIA 144, ACT IV, MOLT4, DA-1, JURKAT, WEHI, K-562, COS, RAJI, NIH 313, HL-60, MLA 144, NAMAIWA, NEURO 2A, CHO, PerC.6, YB2/O) or the like, or heteromyelomas, fusion products thereof, or any cell or fusion cell derived there from, or any other suitable cell line as known in the art (see, those at the following web addresses, e.g., atcc.org, lifetech.com, last accessed on Nov. 26, 2007), with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. Antibody producing cells can also be obtained from the peripheral blood or, in particular embodiments, the spleen or lymph nodes, of humans or other suitable animals that have been immunized with the antigen of interest. Any other suitable host cell can also be used for expressing-heterologous or endogenous nucleic acid encoding an antibody, specified fragment or variant thereof, of the present disclosure. The fused cells (hybridomas) or recombinant cells can be isolated using selective culture conditions or other suitable known methods, and cloned by limiting dilution or cell sorting, or other known methods. Some embodiments disclosed herein relate to specific hybridomas that produce monoclonal antibodies to IHF fragments; non-limiting examples include IhfA5 NTHI 14G8.F5.G6 (ATCC No. PTA-122334), IhfB4 NTHI 4E11.E5.G2 (ATCC No. PTA-122336), mIhfB4 NTHI 12E6.F8.D12.D5 (ATCC No. PTA-122335).

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, cDNA, or the like, display library; e.g., as available from various commercial vendors such as MorphoSys (Martinsreid/Planegg, Del.), BioInvent (Lund, Sweden), Affitech (Oslo, Norway) using methods known in the art. Art known methods are described in the patent literature some of which include U.S. Pat. Nos. 4,704,692; 5,723,323; 5,763,192; 5,814,476; 5,817,483; 5,824,514; 5,976,862. Alternative methods rely upon immunization of transgenic animals (e.g., SCID mice, Nguyen et al. (1977) Microbiol. Immunol. 41:901-907 (1997); Sandhu et al. (1996) Crit. Rev. Biotechnol. 16:95-118; Eren et al. (1998) Mumma 93:154-161) that are capable of producing a repertoire of human antibodies, as known in the art and/or as described herein. Such techniques, include, but are not limited to, ribosome display, e.g., Wanes et al. (1997) Proc. Natl. Acad. Sci. USA, 94:4937-4942; Hanes et al. (1998) Proc. Natl. Acad. Sci. USA 95:14130-14135); single cell antibody producing technologies (e.g., selected lymphocyte antibody method (“SLAM”) (U.S. Pat. No. 5,627,052, Wen et al. (1987) J. Immunol 17:887-892; Babcook et al. (1996) Proc. Natl. Acad. Sci. USA 93:7843-7848); gel microdroplet and flow cytometry (Powell et al. (1990) Biotechnol. 8:333-337; One Cell Systems, (Cambridge, Mass.); Gray et al. (1995) J. Imm. Meth. 182:155-163; and Kenny et al. (1995) Bio. Technol. 13:787-790); B-cell selection (Steenbakkers et al. (1994) Molec. Biol. Reports 19:125-134). An additional method relies on the isolation of human antibodies from human serum, using techniques known in the art, see U.S. Pat. No. 7,939,344.

Antibodies may also be produced by inducing in vivo production in the lymphocyte population or by screening recombinant immunoglobulin libraries or panels of highly specific binding reagents (Orlandi et al. (1989) PNAS 86: 3833-3837; Winter et al. (1991) Nature 349: 293-299).

Alternatively, techniques for the production of single chain antibodies may be used. Single chain antibodies (scFᵥs) comprise a heavy chain variable region and a light chain variable region connected with a linker peptide (typically around 5 to 25 amino acids in length). In the scFᵥ, the variable regions of the heavy chain and the light chain may be derived from the same antibody or different antibodies. scFᵥs may be synthesized using recombinant techniques, for example by expression of a vector encoding the scFᵥ in a host organism such as E. coli. DNA encoding scFᵥ can be obtained by performing amplification using a partial DNA encoding the entire or a desired amino acid sequence of a DNA selected from a DNA encoding the heavy chain or the variable region of the heavy chain of the above-mentioned antibody and a DNA encoding the light chain or the variable region of the light chain thereof as a template, by PCR using a primer pair that defines both ends thereof, and further performing amplification combining a DNA encoding a polypeptide linker portion and a primer pair that defines both ends thereof, so as to ligate both ends of the linker to the heavy chain and the light chain, respectively. An expression vector containing the DNA encoding scFᵥ and a host transformed by the expression vector can be obtained according to conventional methods known in the art.

Antigen binding fragments, for example the F(ab′)₂ fragments, can be produced by pepsin digestion of the antibody molecule and the Fab fragments which can be generated by reducing the disulfide bridges of the F(ab′)₂ fragments. Fab fragments, in particular murine Fab fragments produced from murine monoclonal antibodies, can also be produced by digestion of the antibody with the thiol protease ficin in the presence of cysteine. Fab fragments, in particular rabbit Fab fragments produced from rabbit polyclonal antibodies, may also be produced by digestion of the antibody with the protease papain in the presence of cysteine-HCl. Alternatively, Fab expression libraries may be constructed to allow rapid and easy identification of monoclonal Fab fragments with the desired specificity (Huse et al. (1989) Science 256:1275-1281). Upon generation of the fragment, the fragment can be isolated and sequenced using conventional techniques and the amino acid sequence is determined.

Antibody derivatives of the present disclosure can also be prepared by delivering a polynucleotide encoding an antibody fragment, antigen binding fragment or polypeptide as disclosed herein to a suitable host such as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; and 5,304,489.

The parental antibodies from which the antibody fragments, antigen binding fragments and polypeptides as disclosed herein, also include derivatives that are modified by the covalent attachment of any type of molecule to the antibody such that covalent attachment does not prevent the antibody from generating an anti-idiotypic response. Antibody derivatives include, but are not limited to, antibodies that have been modified by glycosylation, acetylation, pegylation, phosphorylation, amidation, derivatization by known protecting/blocking groups, proteolytic cleavage, linkage to a cellular ligand or other protein, etc. Non-limiting examples of the modified antibodies contemplated herein are aglycosylated whole antibody molecules, antibody fragments, or fusion proteins that include a region equivalent to the Fc region of an immunoglobulin. Such aglycosylate forms can be generated, for example, in host cells that lack the ability to modify proteins with N-linked glycans or by mutating N-linked consensus sites on the antibody of interest. Additionally, the derivatives may contain one or more non-classical amino acids.

Antibody derivatives of the parental antibodies also can be prepared by delivering a polynucleotide disclosed herein to provide transgenic plants and cultured plant cells (e.g., but not limited to tobacco, maize, and duckweed) that produce such antibodies, specified portions or variants in the plant parts or in cells cultured therefrom. For example, Cramer et al. (1999) Curr. Top. Microbol. Immunol. 240:95-118 and references cited therein, describe the production of transgenic tobacco leaves expressing large amounts of recombinant proteins, e.g., using an inducible promoter. Transgenic maize have been used to express mammalian proteins at commercial production levels, with biological activities equivalent to those produced in other recombinant systems or purified from natural sources. See, e.g., Hood et al. (1999) Adv. Exp. Med. Biol. 464:127-147 and references cited therein. Antibody derivatives have also been produced in large amounts from transgenic plant seeds including antibody fragments, such as single chain antibodies (scFᵥ's), including tobacco seeds and potato tubers. See, e.g., Conrad et al. (1998) Plant Mol. Biol. 38:101-109 and references cited therein. Thus, antibodies can also be produced using transgenic plants, according to known methods.

Antibody derivatives of the parental antibodies also can be produced, for example, by adding exogenous sequences to modify immunogenicity or to reduce, enhance or modify binding, affinity, on-rate, off-rate, avidity, specificity, half-life, or any other suitable characteristic. Generally part or all of the non-human or human CDR sequences are maintained while the non-human sequences of the variable and constant regions are replaced with human or other amino acids.

Humanization or engineering of antibodies can be performed using any known method such as, but not limited to, those described in U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

Further provided are the isolated polypeptides comprising, or alternatively consisting essentially of, or yet further consisting of, the Fab fragment or antigen binding fragment, or a polypeptide as disclosed herein that further comprises a detectable or a purification label, as described herein.

Chimeric, humanized or primatized parental antibodies of the antibody fragments, antigen binding fragments or polypeptides of the present disclosure can be prepared based on the sequence of a reference monoclonal antibody prepared using standard molecular biology techniques. Such antibodies may be used to generate the antibody fragments (e.g., Fab fragment or antigen binding fragment) of the present disclosure.

DNA encoding the heavy and light chain immunoglobulins can be obtained from the hybridoma of interest and engineered to contain non-reference (e.g., human) immunoglobulin sequences using standard molecular biology techniques. For example, to create a chimeric antibody, the murine variable regions can be linked to human constant regions using methods known in the art (U.S. Pat. No. 4,816,567). To create a humanized antibody, the murine CDR regions can be inserted into a human framework using methods known in the art (U.S. Pat. Nos. 5,225,539 and 5,530,101; 5,585,089; 5,693,762; and 6,180,370). Similarly, to create a primatized antibody the murine CDR regions can be inserted into a primate framework using methods known in the art (PCT International Pat. Application Publication Nos. WO 93/02108 and WO 99/55369).

Techniques for making partially to fully human antibodies are known in the art and any such techniques can be used. According to one embodiment, fully human antibody sequences are made in a transgenic mouse which has been engineered to express human heavy and light chain antibody genes. Multiple strains of such transgenic mice have been made which can produce different classes of antibodies. B cells from transgenic mice which are producing a desirable antibody can be fused to make hybridoma cell lines for continuous production of the desired antibody. (See, for example, Russel et al. (2000) Infection and Immunity April 2000:1820-1826; Gallo et al. (2000) European J. of Immun. 30:534-540; Green (1999) J. of Immun. Methods 231:11-23; Yang et al. (1999A) J. of Leukocyte Biology 66:401-410; Yang (1999B) Cancer Research 59(6):1236-1243; Jakobovits (1998) Advanced Drug Reviews 31:33-42; Green and Jakobovits (1998) J. Exp. Med. 188(3):483-495; Jakobovits (1998) Exp. Opin. Invest. Drugs 7(4):607-614; Tsuda et al. (1997) Genomics 42:413-421; Sherman-Gold (1997) Genetic Engineering News 17(14); Mendez et al. (1997) Nature Genetics 15:146-156; Jakobovits (1996) Weir's Handbook of Experimental Immunology, The Integrated Immune System Vol. IV, 194.1-194.7; Jakobovits (1995) Current Opinion in Biotechnology 6:561-566; Mendez et al. (1995) Genomics 26:294-307; Jakobovits (1994) Current Biology 4(8):761-763; Arbones et al. (1994) Immunity 1(4):247-260; Jakobovits (1993) Nature 362(6417):255-258; Jakobovits et al. (1993) Proc. Natl. Acad. Sci. USA 90(6):2551-2555; and U.S. Pat. No. 6,075,181.)

The antibodies disclosed herein also can be modified to create chimeric antibodies. Chimeric antibodies are those in which the various domains of the antibodies' heavy and light chains are coded for by DNA from more than one species. See, e.g., U.S. Pat. No. 4,816,567.

Alternatively, the antibodies disclosed herein can also be modified to create veneered antibodies. Veneered antibodies are those in which the exterior amino acid residues of the antibody of one species are judiciously replaced or “veneered” with those of a second species so that the antibodies of the first species will not be immunogenic in the second species thereby reducing the immunogenicity of the antibody. Since the antigenicity of a protein is primarily dependent on the nature of its surface, the immunogenicity of an antibody could be reduced by replacing the exposed residues which differ from those usually found in another mammalian species antibodies. This judicious replacement of exterior residues should have little, or no, effect on the interior domains, or on the interdomain contacts. Thus, ligand binding properties should be unaffected as a consequence of alterations which are limited to the variable region framework residues. The process is referred to as “veneering” since only the outer surface or skin of the antibody is altered, the supporting residues remain undisturbed.

The procedure for “veneering” makes use of the available sequence data for human antibody variable domains compiled by Kabat et al. (1987) Sequences of Proteins of Immunological interest, 4^{th} ed., Bethesda, Md., National Institutes of Health, updates to this database, and other accessible U.S. and foreign databases (both nucleic acid and protein). Non-limiting examples of the methods used to generate veneered antibodies include EP 519596; U.S. Pat. No. 6,797,492; and described in Padlan et al. (1991) Mol. Immunol. 28(4-5):489-498.

The antibody fragments, antigen binding fragments and polypeptides as disclosed herein can be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

Antibodies for the preparation the antibody fragments, antigen binding fragments or of the present disclosure include naturally purified products, products of chemical synthetic procedures, and products produced by recombinant techniques from a eukaryotic host, including, for example, yeast, higher plant, insect and mammalian cells, or alternatively from a prokaryotic host as described above. A number of antibody production systems are described in Birch & Radner (2006) Adv. Drug Delivery Rev. 58:671-685.

If an antibody fragment being tested binds with protein or polypeptide, then the antibody fragment being tested and the antibody fragments provided by this disclosure are equivalent. It also is possible to determine without undue experimentation, whether an antibody fragment has the same specificity as the antibody fragment disclosed herein by determining whether the antibody being tested prevents an antibody fragment disclosed herein from binding the protein or polypeptide with which the antibody fragment is normally reactive. If the antibody fragment being tested competes with the antibody fragment disclosed herein as shown by a decrease in binding by the antibody fragment disclosed herein, then it is likely that the two antibody fragments bind to the same or a closely related epitope. Alternatively, one can pre-incubate the antibody fragment disclosed herein with a protein with which it is normally reactive, and determine if the antibody fragment being tested is inhibited in its ability to bind the antigen. If the antibody fragment being tested is inhibited then, in all likelihood, it has the same, or a closely related, epitopic specificity as the antibody fragment disclosed herein.

The term “antibody” and “antibody fragment” also is intended to include antibodies of all immunoglobulin isotypes and subclasses. Particular isotypes of a monoclonal antibody can be prepared either directly by selecting from an initial fusion, or prepared secondarily, from a parental hybridoma secreting a monoclonal antibody of different isotype by using the sib selection technique to isolate class switch variants using the procedure described in Steplewski et al. (1985) Proc. Natl. Acad. Sci. USA 82:8653 or Spira et al. (1984) J. Immunol. Methods 74:307. Alternatively, recombinant DNA techniques may be used.

The isolation of other monoclonal antibodies for use in generating antibody fragments, antigen binding fragments and polypeptides, with the specificity of the monoclonal antibodies described herein can also be accomplished by one of ordinary skill in the art by producing anti-idiotypic antibodies. Herlyn et al. (1986) Science 232:100. An anti-idiotypic antibody is an antibody that recognizes unique determinants present on the monoclonal antibody of interest.

In some aspects disclosed herein, it will be useful to detectably or therapeutically label the antibody fragment, antigen binding fragment or polypeptide. Suitable labels are described herein. Methods for conjugating antibodies and polypeptides to these agents are known in the art. For the purpose of illustration only, the antibody fragment, antigen binding fragment or polypeptide can be labeled with a detectable moiety such as a radioactive atom, a chromophore, a fluorophore, or the like. Such labeled antibodies can be used for diagnostic techniques, either in vivo, or in an isolated test sample.

The coupling of antibodies, fragments and polypeptides to low molecular weight haptens can increase the sensitivity of the antibody fragment or polypeptide in an assay. The haptens can then be specifically detected by means of a second reaction. For example, it is common to use haptens such as biotin, which reacts avidin, or dinitrophenol, pyridoxal, and fluorescein, which can react with specific anti-hapten antibodies. See, Harlow and Lane (1988) supra.

The variable region of the parental antibodies of the present disclosure can be modified by mutating amino acid residues within the V_{H} and/or V_{L} CDR 1, CDR 2 and/or CDR 3 regions to improve one or more binding properties (e.g., affinity) of the antibody. Mutations may be introduced by site-directed mutagenesis or PCR-mediated mutagenesis and the effect on antibody binding, or other functional property of interest, can be evaluated in appropriate in vitro or in vivo assays. In certain embodiments, conservative modifications are introduced and typically no more than one, two, three, four or five residues within a CDR region are altered. The mutations may be amino acid substitutions, additions or deletions.

Framework modifications can be made to the parental antibodies to decrease immunogenicity, for example, by “backmutating” one or more framework residues to the corresponding germline sequence.

In addition, the antibody fragments, antigen binding fragments or polypeptides disclosed herein may be engineered to include modifications within the Fc region to alter one or more functional properties of the antibody, such as serum half-fife, complement fixation, Fc receptor binding, and/or antigen-dependent cellular cytotoxicity. Such modifications include, but are not limited to, alterations of the number of cysteine residues in the hinge region to facilitate assembly of the light and heavy chains or to increase or decrease the stability of the antibody (U.S. Pat. No. 5,677,425) and amino acid mutations in the Fc hinge region to decrease die biological half-life of the antibody (U.S. Pat. No. 6,165,745).

Additionally, the antibody fragments disclosed herein may be chemically modified. Glycosylation of an antibody can be altered, for example, by modifying one or more sites of glycosylation within the antibody sequence to increase the affinity of the antibody for antigen (U.S. Pat. Nos. 5,714,350 and 6,350,861). Alternatively, to increase antibody-dependent cell-mediated cytotoxicity, a hypofucosylated antibody having reduced amounts of fucosyl residues or an antibody having increased bisecting GlcNac structures can be obtained by expressing the antibody in a host cell with altered glycosylation mechanism (Shields et al. (2002) J. Biol. Chem. 277:26733-26740; Umana et al. (1999) Nat. Biotech. 17:176-180).

The antibodies fragment, antigen binding fragment or polypeptide as disclosed herein can be pegylated to increase biological half-life by reacting the antibody or fragment thereof with polyethylene glycol (PEG) or a reactive ester or aldehyde derivative of PEG, under conditions in which one or more PEG groups become attached to the antibody or antibody fragment. Pegylation may be carried out by an acylation reaction or an alkylation reaction with a reactive PEG molecule (or an analogous reactive water soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. The antibody to be pegylated can be an aglycosylated antibody. Methods for pegylating proteins are known in the art and can be applied to the antibodies disclosed herein (EP 0154316 and EP 0401384).

Additionally, the antibody binding fragment, antigen binding fragment or polypeptides may be chemically modified by conjugating or fusing the antigen-binding region to serum protein, such as human serum albumin, to increase half-life of the resulting molecule. Such approach is for example described in EP 0322094 and EP 0486525.

The antibody fragment, antigen binding fragment or polypeptide as disclosed herein may be conjugated to a diagnostic agent and used diagnostically, for example, to monitor the development or progression of a disease and determine the efficacy of a given treatment regimen. Examples of diagnostic agents include enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody or fragment thereof, or indirectly, through a linker using techniques known in the art. Examples of suitable enzymes include horseradish peroxidase, alkaline phosphatase, beta-galactosidase, or acetylcholinesterase. Examples of suitable prosthetic group complexes include streptavidin/biotin and avidin/biotin. Examples of suitable fluorescent materials include umbelliferone, fluorescein, fluorescein isothiocyanate, rhodamine, dichlorotriazinylamine fluorescein, dansyl chloride or phycoerythrin. An example of a luminescent material includes luminol. Examples of bioluminescent materials include luciferase, luciferin, and aequorin. Examples of suitable radioactive material include ¹²I, ¹³¹I, Indium-111, Lutetium-171, Bismuth-212, Bismuth-213, Astatine-211, Copper-62, Copper-64, Copper-67, Yttrium-90, Iodine-125, Iodine-131, Phosphorus-32, Phosphorus-33, Scandium-47, Silver-111, Gallium-67, Praseodymium-142, Samarium-153, Terbium-161, Dysprosium-166, Holmium-166, Rhenium-186, Rhenium-188, Rhenium-189, Lead-212, Radium-223, Actinium-225, Iron-59, Selenium-75, Arsenic-77, Strontium-89, Molybdenum-99, Rhodium-1105, Palladium-109, Praseodymium-143, Promethium-149, Erbium-169, Iridium-194, Gold-198, Gold-199, and Lead-211. Monoclonal antibodies may be indirectly conjugated with radiometal ions through the use of bifunctional chelating agents that are covalently linked to the antibodies. Chelating agents may be attached through amities (Meares et al. (1984) Anal. Biochem. 142:68-78); sulfhydral groups (Koyama 1994 Chem. Abstr. 120: 217262t) of amino acid residues and carbohydrate groups (Rodwell et al. (1986) PNAS USA 83:2632-2636; Quadri et al. (1993) Nucl. Med. Biol. 20:559-570).

Further, the antibody fragment, antigen binding fragment or polypeptide of the present disclosure may be conjugated to a therapeutic agent. Suitable therapeutic agents include antibiotics or antimicrobials for example, or host defense peptides (e.g., specifically targeted antimicrobial peptides (STAMPs)).

Additional suitable conjugated molecules include ribonuclease (RNase), DNase, an antisense nucleic acid, an inhibitory RNA molecule such as a siRNA molecule, an immunostimulatory nucleic acid, aptamers, ribozymes, triplex forming molecules, and external guide sequences. Aptamers are small nucleic acids ranging from 15-50 bases in length that fold into defined secondary and tertiary structures, such as stem-loops or G-quartets, and can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Ribozymes are nucleic acid molecules that are capable of catalyzing a chemical reaction, either intra-molecularly or inter-molecularly. Ribozymes typically cleave nucleic acid substrates through recognition and binding of the target substrate with subsequent cleavage. Triplex forming function nucleic acid molecules can interact with double-stranded or single-stranded nucleic acid by forming a triplex, in which three strands of DNA form a complex dependent on both Watson-Crick and Hoogsteen base-pairing. Triplex molecules can bind target regions with high affinity and specificity. Suitable conjugated molecules may further include any protein that binds to DNA provided that it does not create or stabilize biofilm architecture; it is envisioned that at least a subset of such proteins may facilitate the kinetics of binding for the agents disclosed herein.

The functional nucleic acid molecules may act as effectors, inhibitors, modulators, and stimulators of a specific activity possessed by a target molecule, or the functional nucleic acid molecules may possess a de novo activity independent of any other molecules.

The therapeutic agents can be linked to the fragment or polypeptide directly or indirectly, using any of a large number of available methods. For example, an agent can be attached at the hinge region of the reduced antibody component via disulfide bond formation, using cross-linkers such as N-succinyl 3-(2-pyridyldithio) proprionate (SPDP), or via a carbohydrate moiety in the Fc region of the antibody (Yu et al. (1994) Int. J. Cancer 56: 244; Upeslacis et al., “Modification of Antibodies by Chemical Methods,” in Monoclonal antibodies: principles and applications, Birch et al. (eds.), pages 187-230 (Wiley-Liss, Inc. 1995); Price, “Production and Characterization of Synthetic Peptide-Derived Antibodies,” in Monoclonal antibodies: Production, Engineering and Clinical Application, Ritter et al. (eds.), pages 60-84 (Cambridge University Press 1995)).

Techniques for conjugating therapeutic agents to antibodies and antibody fragments are well known (Amon et al., “Monoclonal Antibodies For Immunotargeting of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al, (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody in Cancer Therapy”, in Monoclonal Antibodies For Cancer Detection And Therapy, Baldwin et al. (eds.), pp. 303-16 (Academic Press 1985), and Thorpe et al. (1982) Immunol. Rev. 62:119-58).

The fragments or polypeptides can be linked to another functional molecule such as another antibody or ligand for a receptor to generate a bi-specific or multi-specific molecule that binds to at least two or more different binding sites or target molecules. Linking of the antibody to one or more other binding molecules, such as another antibody, antibody fragment, peptide or binding mimetic, can be done, for example, by chemical coupling, genetic fusion, or non-covalent association. Multi-specific molecules can further include a third binding specificity, in addition to the first and second target epitope.

Bi-specific and multi-specific molecules can be prepared using methods known in the art. For example, each binding unit of the hi-specific molecule can be generated separately and then conjugated to one another. When the binding molecules are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitroberizoic acid) (DTNB), o-phenylenedimaleimide (oPDM), N-succinimidyl-3-(2-pyridyldithio)propionate (SPDP), and sulfosuccinimidyl 4-(N-maleimidomethyl)cyclohaxane-I-carboxylate (sulfo-SMCC) (Karpovsky et al. (1984) J. Exp. Med. 160:1686; Liu et al. (1985) Proc. Natl. Acad. Sci. USA 82:8648). When the binding molecules are antibodies, they can be conjugated by sulfhydryl bonding of the C-terminus hinge regions of the two heavy chains.

The antibody fragment, antigen binding fragment or polypeptide as disclosed herein may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

The antibody fragment, antigen binding fragment or polypeptide also can be bound to many different carriers. Thus, this disclosure also provides compositions containing the fragments or polypeptides and another substance, active or inert. Examples of well-known carriers include glass, polystyrene, polypropylene, polyethylene, dextran, nylon, amylase, natural and modified cellulose, polyacrylamide, agarose, and magnetite. The nature of the carrier can be either soluble or insoluble for purposes disclosed herein. Those skilled in the art will know of other suitable carriers for binding monoclonal antibodies, or will be able to ascertain such, using routine experimentation.

Compositions comprising, or alternatively consisting essentially of, or yet further consisting of, one or more of the above embodiments are further provided herein. Further provided are polynucleotides that encode the amino acid sequence of the antibody fragment, the antigen binding fragment or polypeptides, as well as methods to produce recombinantly or chemically synthesize the fragments and polypeptides. The antibody polypeptides and fragments can be produced in a eukaryotic or a prokaryotic cell, or by other methods known in the art and briefly described herein.

The fragments and isolated polypeptides comprising the fragments disclosed herein may be selected such that they have a high level of epitope binding specificity and high binding affinity to the biofilm. In general, the higher the binding affinity of an antibody or antibody fragment or isolated polypeptides comprising the antibody fragments, the more stringent wash conditions can be performed in an immunoassay to remove nonspecifically bound material without removing the target. Accordingly, the fragments and isolated polypeptides comprising the antibody fragments of the present technology useful in the disclosed methods usually have binding affinities of at least 10⁻⁶, 10⁻⁷, 10⁻¹, 10⁻¹, 10⁻¹⁰, 10⁻¹¹, or 10⁻¹² M. In certain aspects, the fragments and isolated polypeptides comprising the antibody fragments have a sufficient kinetic on-rate to reach equilibrium under standard conditions in at least 12 hours, at least 5 hours, at least 1 hour, or at least 30 minutes. In another aspect, the affinity of the antibody or antigen binding fragment is less than or about 1000 picoMole (pM), less than about 900 pM, less than about 800 pM, less than about 700 pM, less than about 600 pM, less than about 500 pM, less than about 400 pM, less than about 300 pM, less than about 200 pM, less than about 100 pM, less than about 50 pM, less than about 40 pM, less than about 30 pM, less than about 20 pM, or about 10 pM, or about 9 pM, or about 8 pM, or alternatively less than about 4 pM, or alternatively less than about 2 pM.

In any of the above embodiments, a peptide linker can be added to the N-terminus or C-terminus of the antibody fragment, antigen binding fragment or polypeptide as disclosed herein. A “linker” or “peptide linker” refers to a peptide sequence linked to either the N-terminus or the C-terminus of a polypeptide sequence. In one aspect, the linker is from about 1 to about 20 amino acid residues long or alternatively 2 to about 10, about 3 to about 5 amino acid residues long. An example of a peptide linker is Gly-Pro-Ser-Leu-Lys-Leu (SEQ ID NO. 43). Other examples include Gly-Gly-Gly (SEQ ID NO. 44); Gly-Pro-Ser-Leu (SEQ ID NO. 45); Gly-Pro-Ser (SEQ ID NO. 46); Pro-Ser-Leu-Lys (SEQ ID NO. 47); Gly-Pro-Ser-Leu-Lys (SEQ ID NO. 48), and Ser-Leu-Lys-Leu (SEQ ID NO. 49).

### Antibiotics

In some embodiments of any aspect as disclosed herein, the β-lactam antibiotic comprises, or consists essentially of, or yet further consists of amoxicillin plus clavulanic acid (AMC). In some embodiments of any aspect as disclosed herein, the β-lactam antibiotic comprises, or consists essentially of, or yet further consists of one or more of: Benzathine, benzylpenicillin (Penicillin G), Benzathine penicillin G, Benzathine penicillin V, Phenoxymethylpenicillin (penicillin V), Procaine penicillin, Pheneticillin, Cloxacillin, Dicloxacillin, Flucloxacillin, Methicillin, Nafcillin, Oxacillin, Temocillin, Amoxicillin, Ampicillin, Mecillinam, Piperacillin, Carbenicillin, Ticarcillin, Carboxypenicillin, Ureidopenicillin, Azlocillin, Mezlocillin; Cefazolin, Cephalexin, Cephalosporin C, Cephalothin, Cefapirin, Cefaclor, Cefamandole, Cefuroxime, Cefotetan, Cefoxitin, Cefixime, Cefotaxime, Cefpodoxime, Ceftazidime, Ceftriaxone, Cefdinir, Cefepime, Cefpirome, Ceftaroline; Biapenem, Doripenem, Ertapenem, Faropenem, Imipenem, Meropenem, Panipenem, Razupenem, Tebipenem, Thienamycin; Aztreonam, Tigemonam, Nocardicin A, or Tabtoxinine β-lactam.

In some embodiments of any aspect as disclosed herein, the sulfonamide antibiotic comprises, or consists essentially of, or yet further consists of trimethoprim plus sulfamethoxazole (TMP-SMX). In some embodiments of any aspect as disclosed herein, the sulfonamide antibiotic comprises, or consists essentially of, or yet further consists of one or more of the following: Sulfafurazole; Sulfacetamide, Sulfadiazine, Sulfadimidine, Sulfafurazole (sulfisoxazole), Sulfisomidine (sulfaisodimidine), Sulfamethoxazole, Sulfamoxole, Sulfanitran, Sulfadimethoxine, Sulfamethoxypyridazine, Sulfametoxydiazine, Sulfadoxine, Sulfametopyrazine, or Terephtyl.

In some embodiments, the antibiotic comprises, or consists essentially of, or yet further consists of one or more of the following: ampicillin, amoxicillin-clavulanate, and Cefdinir.

### Administration Protocols

Routes of administration applicable to the methods disclosed herein include intranasal, intramuscular, urethrally, intratracheal, subcutaneous, intradermal, transdermal, topical application, intravenous, rectal, nasal, oral, inhalation, and other enteral and parenteral routes of administration. Routes of administration may be combined, if desired, or adjusted depending upon the agent and/or the desired effect. An active agent can be administered in a single dose or in multiple doses. Embodiments of these methods and routes suitable for delivery include systemic or localized routes. In general, routes of administration suitable for the methods disclosed herein include, but are not limited to, direct injection, enteral, parenteral, or inhalational routes.

Parenteral routes of administration other than inhalation administration include, but are not limited to, topical, transdermal, subcutaneous, intramuscular, intraorbital, intracapsular, intraspinal, intrasternal, and intravenous routes, i.e., any route of administration other than through the alimentary canal. Parenteral administration can be conducted to effect systemic or local delivery of the inhibiting agent. Where systemic delivery is desired, administration typically involves invasive or systemically absorbed topical or mucosal administration of pharmaceutical preparations.

The agents disclosed herein can also be delivered to the subject by enteral administration. Enteral routes of administration include, but are not limited to, oral and rectal (e.g., using a suppository) delivery.

Methods of administration of the active through the skin or mucosa include, but are not limited to, topical application of a suitable pharmaceutical preparation, transcutaneous transmission, transdermal transmission, injection and epidermal administration. For transdermal transmission, absorption promoters or iontophoresis are suitable methods. Iontophoretic transmission may be accomplished using commercially available “patches” that deliver their product continuously via electric pulses through unbroken skin for periods of several days or more.

In various embodiments of the methods disclosed herein, the interfering agent will be administered by inhalation, injection or orally on a continuous, daily basis, at least once per day (QD), and in various embodiments two (BID), three (TID), or even four times a day. Typically, the therapeutically effective daily dose will be at least about 1 mg, or at least about 10 mg, or at least about 100 mg, or about 200 to about 500 mg, and sometimes, depending on the compound, up to as much as about 1 g to about 2.5 g.

Dosing of can be accomplished in accordance with the methods disclosed herein using capsules, tablets, oral suspension, suspension for intra-muscular injection, suspension for intravenous infusion, get or cream for topical application, or suspension for intra-articular injection.

Dosage, toxicity and therapeutic efficacy of compositions described herein can be determined by standard pharmaceutical procedures in cell cultures or experimental animals, for example, to determine the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. In certain embodiments, compositions exhibit high therapeutic indices. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies (in certain embodiments, within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In some embodiments, an effective amount of a composition sufficient for achieving a therapeutic or prophylactic effect, ranges from about 0.000001 mg per kilogram body weight per administration to about 10,000 mg per kilogram body weight per administration. Suitably, the dosage ranges are from about 0.0001 mg per kilogram body weight per administration to about 100 mg per kilogram body weight per administration. Administration can be provided as an initial dose, followed by one or more “booster” doses. Booster doses can be provided a day, two days, three days, a week, two weeks, three weeks, one, two, three, six or twelve months after an initial dose. In some embodiments, a booster dose is administered after an evaluation of the subject's response to prior administrations.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

When practiced in vitro, the methods are useful to screen for or confirm agents having the same, similar or opposite ability as the polypeptides, polynucleotides, antibodies or fragments thereof, host cells, small molecules and compositions disclosed herein. Alternatively, they can be used to identify which agent is best suited to treat a microbial infection. For example, one can screen for new agents or combination therapies by having two samples containing for example, the DNABII polypeptide and microbial DNA and the agent to be tested. The second sample contains the DNABII polypeptide and microbial DNA and an agent known to active, e.g., an anti-IHF antibody or a small molecule to serve as a positive control. In a further aspect, several samples are provided and the agents are added to the system in increasing dilutions to determine the optimal dose that would likely be effective in treating a subject in the clinical setting. As is apparent to those of skill in the art, a negative control containing the DNABII polypeptide and the microbial DNA can be provided. In a further aspect, the DNABII polypeptide and the microbial DNA are detectably labeled, for example with luminescent molecules that will emit a signal when brought into close contact with each other. The samples are contained under similar conditions for an effective amount of time for the agent to inhibit, compete or titrate the interaction between the DNABII polypeptide and microbial DNA and then the sample is assayed for emission of signal from the luminescent molecules. If the sample emits a signal, then the agent is not effective to inhibit binding.

In another aspect, the in vitro method is practiced in a miniaturized chamber slide system wherein the microbial (such as a bacterial) isolate causing an infection could be isolated from the human/animal then cultured to allow it to grow as a biofilm in vitro, see for example experiments below. The agent (such as anti-IHF antibody) or potential agent is added alone or in combination with another agent to the culture with or without increasing dilutions of the potential agent or agent such as an anti-IHF (or other antibody, small molecule, agent, etc.) to find the optimal dose that would likely be effective at treating that patient when delivered to the subject where the infection existed. As apparent to those of skill in the art, a positive and negative control can be performed simultaneously.

In a further aspect, the method is practiced in a high throughput platform with the agent (such as anti-IHF antibody) and/or potential agent (alone or in combination with another agent) in a flow cell. The agent (such as anti-IHF antibody) or potential agent is added alone or in combination with another agent to the culture with or without increasing dilutions of the potential agent or agent such as an anti-IHF (or other antibody, small molecule, agent, etc.) to find the optimal dose that would likely be effective at treating that patient when delivered to the subject where the infection existed. Biofilm isolates are sonicated to separate biofilm bacteria from DNABII polypeptide such as IHF bound to microbial DNA. The DNABII polypeptide-DNA complexes are isolated by virtue of the anti-IHF antibody on the platform. The microbial DNA is then be released with e.g., a salt wash, and used to identify the biofilm bacteria added. The freed DNA is then identified, e.g., by PCR sequenced. If DNA is not freed, then the agent(s) successfully performed or bound the microbial DNA. If DNA is found in the sample, then the agent did not interfere with DNABII polypeptide-microbial DNA binding. As is apparent to those of skill in the art, a positive and/or negative control can be simultaneously performed.

In another aspect one or more of the agents or antibodies disclosed herein are used in a method of detecting a biofilm in vivo. In further embodiments, the agents or antibodies are detectably labeled, for example with a luminescent or fluorescent molecule. Further applications of the methods disclosed herein include methods of use of such agents or antibodies to image a biofilm using, for example, a detectably labeled primary agent or antibody which provides a detectable signal upon binding to the biofilm or a detectably labeled secondary antibody which binds to the primary agent or antibody when it is bound to the biofilm.

The above methods also can be used as a diagnostic test since it is possible that a given bacterial species will respond better to reversal of its biofilm by one agent more than another, this rapid high throughput assay system could allow one skilled the art to assay a panel of possible agents to identify the most efficacious of the group.

The advantage of these methods is that most clinical microbiology labs in hospitals are already equipped to perform these sorts of assays (i.e., determination of MIC, MBC values) using bacteria that are growing in liquid culture (or planktonically). As is apparent to those of skill in die art, bacteria generally do not grow planktonically when they are causing diseases. Instead they are growing as a stable biofilm and these biofilms are significantly more resistant to treatment by antibiotics, antibodies or other therapeutics. This resistance is why most MIC/MBC values fail to accurately predict efficacy in vivo. Thus, by determining what “dose” of agent could reverse a bacterial biofilm in vitro (as described above) Applicants' pre-clinical assay would be a more reliable predictor of clinical efficacy, even as an application of personalized medicine.

In addition to the clinical setting, the methods can be used to identify the microbe causing the infection and/or confirm effective agents in an industrial setting.

In a further aspect of the above methods, an antibiotic or antimicrobial known to inhibit growth of the underlying infection is added sequentially or concurrently, to determine if the infection can be inhibited. It is also possible to add the agent to the microbial DNA or DNABII polypeptide before adding the complex to assay for biofilm inhibition.

When practiced in vivo in non-human animal such as a chinchilla, the method provides a pre-clinical screen to identify agents that can be used alone or in combination with other agents to break down biofilms.

In another aspect, provided herein is a method of inhibiting, dissolving preventing or breaking down a biofilm in a subject by administering to the subject an effective amount of a polypeptide, polynucleotide, vector, host cell, antibody or antigen binding fragment thereof, thereby inhibiting, preventing, dissolving or breaking down the microbial biofilm.

Alternatively or additionally, methods of inhibiting, dissolving preventing or breaking down a biofilm may be practiced in vitro and/or ex vivo and involve providing a sample of the biofilm—taken from a subject or generated in vitro—and administering an effective amount of a polypeptide, polynucleotide, vector, host cell, antibody or antigen binding fragment thereof, thereby inhibiting, preventing or breaking down the microbial biofilm. Similarly, the compositions disclosed herein may be used in method embodiments for inhibiting, preventing, or breaking down microbial biofilms on surfaces colonized by biofilms such as, but not limited to, hospital instruments, industrial equipment, and other materials not comprised of living tissue.

In some embodiments the methods disclosed herein comprise, or alternatively consist essentially of, or yet further comprise, administering one or more of a polypeptide, polynucleotide, vector, host cell, antibody or antigen binding fragment thereof, alone or in combination. In further embodiments of the disclosed methods, the agents may be administered simultaneously. In alternative embodiments, the antibodies are administered sequentially.

Also provided herein is a method for inducing an immune response in or conferring passive immunity on subject in need thereof, comprising, or alternatively consisting essentially of, or yet further consisting of, administering to the subject an effective amount of one or more of a polypeptide, polynucleotide, vector, host cell, antibody or antigen binding fragment thereof as disclosed herein.

In some embodiments, the antibody or antigen binding fragment is not a polyclonal antibody.

In a further aspect, the methods further comprise, or alternatively consist essentially of, or yet further consist of administering to the subject an effective amount of one or more of an antimicrobial, an antigenic peptide or an adjuvant.

A non-limiting example of an antimicrobial agent are antibodies directed against vaccine component such as a surface antigen, e.g., an OMP P5, rsPilA, OMP 26, OMP P2, or Type IV Pilin protein (see Jurcisek and Bakaletz (2007) J. Bacteriology 189(10):3868-3875; Murphy et al. (2009) The Pediatric Infectious Disease Journal 28:S121-S126; Novotny et al. (2015) Mol Microbiol. 96(2):276-92).

The agents and compositions disclosed herein can be concurrently or sequentially administered with other antimicrobial agents and/or surface antigens. In one particular aspect, administration is locally to the site of the infection by direct injection or by inhalation for example. Other non-limiting examples of administration include by one or more method comprising transdermally, urethrally, sublingually, rectally, vaginally, ocularly, subcutaneous, intramuscularly, intraperitoneally, intranasally, by inhalation or orally.

These microbial infections may be present in the upper, mid and lower airway (otitis, sinusitis, bronchitis but also exacerbations of chronic obstructive pulmonary disease (COPD), chronic cough, complications of and/or primary cause of cystic fibrosis (CF) and community acquired pneumonia (CAP). Thus, by practicing the in vivo methods disclosed herein, these diseases and complications from these infections can also be prevented or treated.

Infections might also occur in the oral cavity (caries, periodontitis) and caused by Streptococcus mutans, Porphyromonas gingivalis, Aggregatibacter actinomvctemcomitans. Infections might also be localized to the skin (abscesses, ‘staph’ infections, impetigo, secondary infection of burns, Lyme disease) and caused by Staphylococcus aureus, Staphylococcus epidermidis, Pseudomonas aeruginosa and Borrelia burdorferi. Infections of the urinary tract (UTI) can also be treated and are typically caused by Escherichia coli. Infections of the gastrointestinal tract (GI) (diarrhea, cholera, gall stones, gastric ulcers) are typically caused by Salmonella enterica serovar, Vibrio cholerae and Helicobacter pylori. Infections of the genital tract include and are typically caused by Neisseria gonorrhoeae. Infections can be of the bladder or of an indwelling device caused by Enterococcus faecalis. Infections associated with implanted prosthetic devices, such as artificial hip or knee replacements, or dental implants, or medical devices such as pumps, catheters, stents, or monitoring systems, typically caused by a variety of bacteria, can be treated by the methods disclosed herein. These devices can be coated or conjugated to an agent as described herein. Thus, by practicing the in vivo methods disclosed herein, these diseases and complications from these infections can also be prevented or treated.

Infections caused by Streptococcus agalactiae can also be treated by the methods disclosed herein and it is the major cause of bacterial septicemia in newborns. Infections caused by Neisseria meningitidis which can cause meningitis can also be treated.

In some embodiments, the biologically active fragment of an antibody comprises, or consists essentially of, or yet further consists of an isolated polypeptide comprising, or alternatively consisting essentially of, or yet further consisting of heavy and light chains of the antibody, an antigen binding fragment thereof, CDRs and equivalents of each thereof with the addition of up to 25, or alternatively 20, or alternatively 15, or alternatively up to 10, or alternatively up to 5 random amino acids on either the amino or carboxyl termini (or on both). The biologically active fragment can further comprise a detectable or purification marker.

In some embodiments, the biologically active fragment of a peptide and/or an antibody comprises, or consists essentially of, or yet further consists of an isolated or recombinant polypeptide comprising, or alternatively consisting essentially of, or yet further consisting of, one, two or more, or three or more, four or more, five or more, six or more, seven or more, eight or more, nine or more, ten or more, eleven or more, twelve or more, thirteen or more of, fourteen or more of, or all of the polypeptides as disclosed herein, or a fragment or an equivalent of each thereof.

In any of the above embodiments, a peptide linker can be added to the N-terminus or C-terminus of the polypeptide. A “linker” or “peptide linker” refers to a peptide sequence linked to either the N-terminus or the C-terminus of a polypeptide sequence. In one aspect, the linker is from about 1 to about 20 amino acid residues long or alternatively 2 to about 10, about 3 to about 5 amino acid residues long. An example of a peptide linker is Gly-Pro-Ser-Leu-Lys-Leu (SEQ ID NO:). Other examples include Gly-Gly-Gly; Gly-Pro-Ser-Leu (SEQ ID NO:); Gly-Pro-Ser; Pro-Ser-Leu-Lys (SEQ ID NO:); Gly-Pro-Ser-Leu-Lys (SEQ ID NO:) and Ser-Leu-Lys-Leu (SEQ ID NO:).

The isolated polypeptides disclosed herein are intended to include recombinantly produced polypeptides and proteins from prokaryotic and eukaryotic host cells, as well as muteins, analogs and fragments thereof, examples of such cells are described above. In some embodiments, the term also includes antibodies and anti-idiotypic antibodies as described herein. Such polypeptides can be isolated or produced using the methods known in the art and briefly described herein.

It is understood that functional equivalents or variants of the wild type polypeptide or protein also are within the scope of this disclosure, for example, those having conservative amino acid substitutions of the amino acids.

In a further aspect, the polypeptides are conjugated or linked to a detectable label or an agent to increase the half-life of the polypeptide, e.g., PEGylation a PEG mimetic, polysialyation, HESylation or glycosylation. Suitable labels are known in the art and described herein.

In a yet further aspect, the polypeptides with or without a detectable label can be contained or expressed on the surface of a host prokaryotic or eukaryotic host cell, such as a dendritic cell.

The proteins and polypeptides are obtainable by a number of processes known to those of skill in the art, which include purification, chemical synthesis and recombinant methods. Polypeptides can be isolated from preparations such as host cell systems by methods such as immunoprecipitation with antibody, and standard techniques such as gel filtration, ion-exchange, reversed-phase, and affinity chromatography. For such methodology, see for example Deutscher et al. (1999) Guide To Protein Purification: Methods In Enzymology (Vol. 182, Academic Press). Accordingly, this disclosure also provides the processes for obtaining these polypeptides as well as the products obtainable and obtained by these processes.

The polypeptides also can be obtained by chemical synthesis using a commercially available automated peptide synthesizer such as those manufactured by Perkin/Elmer/Applied Biosystems, Inc., Model 430A or 431A, Foster City, Calif, USA. The synthesized polypeptide can be precipitated and further purified, for example by high performance liquid chromatography (HPLC). Accordingly, this disclosure also provides a process for chemically synthesizing the proteins disclosed herein by providing the sequence of the protein and reagents, such as amino acids and enzymes and linking together the amino acids in the proper orientation and linear sequence.

Alternatively, the proteins and polypeptides can be obtained by well-known recombinant methods as described, for example, in Sambrook et al. (1989) supra, using a host cell and vector systems described herein.

Also provided by this application are the polypeptides described herein conjugated to a detectable agent for use in the diagnostic methods. In some embodiments, antibodies that are specific for the tail regions of the DNABII polypeptides (including but not limited to: a tail region of IHF or HU, a tail region of IHFA or IHFB, and/or the tail-chimeric peptide IhfA3-IhfB2_{NTHI}) are particularly useful in diagnostic assays for the detection of biofilms and can be used alone or in combination of one or more antibodies as described herein. In one aspect, antibodies specific for the tail regions are used as a companion diagnostic for an antibody that is specific for a tip region of the DNABII polypeptide (including but not limited to: a tip region of IHF or HU, a tip region of IHFA or IHFB, and/or the tip-chimeric peptide IhfA5-mIhfB4_{NTHI}).

It is well known to those skilled in the art that modifications can be made to the peptides disclosed herein to provide them with altered properties. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

Peptides disclosed herein can be modified to include unnatural amino acids. Thus, the peptides may comprise D-amino acids, a combination of and L-amino acids, and various “designer” amino acids (e.g., beta-methyl amino acids, C-alpha-methyl amino acids, and N-alpha-methyl amino acids, etc.) to convey special properties to peptides. Additionally, by assigning specific amino acids at specific coupling steps, peptides with alpha-helices, beta turns, beta sheets, gamma-turns, and cyclic peptides can be generated. Generally, it is believed that alpha-helical secondary structure or random secondary structure may be of particular use.

The polypeptides disclosed herein also can be combined with various solid phase carriers, such as an implant, a stent, a paste, a gel, a dental implant, or a medical implant or liquid phase carriers, such as beads, sterile or aqueous solutions, pharmaceutically acceptable carriers, pharmaceutically acceptable polymers, liposomes, micelles, suspensions and emulsions. Examples of non-aqueous solvents include propyl ethylene glycol, polyethylene glycol and vegetable oils. When used to prepare antibodies or induce an immune response in vivo, the carriers also can include an adjuvant that is useful to non-specifically augment a specific immune response. A skilled artisan can easily determine whether an adjuvant is required and select one. However, for the purpose of illustration only, suitable adjuvants include, but are not limited to Freund's Complete and Incomplete, mineral salts and polynucleotides. Other suitable adjuvants include monophosphoryl lipid A (MPL), mutant derivatives of the heat labile enterotoxin of E. coli, mutant derivatives of cholera toxin, CPG oligonucleotides, and adjuvants derived from squalene.

This disclosure also provides a pharmaceutical composition comprising or alternatively consisting essentially of, or yet further consisting of, any of a polypeptide, analog, mutein, or fragment disclosed herein, alone or in combination with each other or other agents, such an antibiotic and an acceptable carrier or solid support. These compositions are useful for various diagnostic and therapeutic methods as described herein.

### Polynucleotides

Also provided are isolated or recombinant polynucleotides encoding one or more of the above-identified antibodies, fragments thereof, CDRs, isolated or recombinant polypeptides and their respective complementary strands. Vectors comprising the isolated or recombinant polynucleotides are further provided examples of which are known in the art and briefly described herein. In one aspect where more than one isolated or recombinant polynucleotide is to be expressed as a single unit, the isolated or recombinant polynucleotides can be contained within a polycistronic vector. The polynucleotides can be DNA, RNA, mRNA or interfering RNA, such as siRNA, miRNA or dsRNA. Such polynucleotide and vectors can be used to deliver the antibody, polypeptide, or a biologically active fragment of each thereof to a subject in needed via administering to the subject and expressing the antibody, polypeptide or biologically active fragment.

In another aspect, this disclosure provides an interfering agent that is a polynucleotide that interferes with the binding of the DNA to a polypeptide or protein in a microbial biofilm, or a four-way junction polynucleotide resembling a Holliday junction, a 3 way junction polynucleotide resembling a replication fork, a polynucleotide that has inherent flexibility or bent polynucleotide which can treat or inhibit DNABII polynucleotide (HU or IHF) from binding to microbial DNA as well treat, prevent or inhibit biofilm formation and associated infections and disorders. One of skill in the art can make such polynucleotides using the information provided herein and knowledge of those of skill in the art. See Goodman and Kay (1999) J. Biological Chem. 274(52):37004-37011 and Kamashev and Rouviere-Yaniv (2000) EMBO J. 19(23):6527-6535.

The disclosure further provides the isolated or recombinant polynucleotide operatively linked to a promoter of RNA transcription, as well as other regulatory sequences for replication and/or transient or stable expression of the DNA or RNA. As used herein, the term “operatively linked” means positioned in such a manner that the promoter will direct transcription of RNA off the DNA molecule. Examples of such promoters are SP6, T4 and T7. In certain embodiments, cell-specific promoters are used for cell-specific expression of the inserted polynucleotide. Vectors which contain a promoter or a promoter/enhancer, with termination codons and selectable marker sequences, as well as a cloning site into which an inserted piece of DNA can be operatively linked to that promoter are known in the art and commercially available. For general methodology and cloning strategies, see Gene Expression Technology (Goeddel ed., Academic Press, Inc. (1991)) and references cited therein and Vectors: Essential Data Series (Gacesa and Ramji, eds., John Wiley & Sons, N.Y. (1994)) which contains maps, functional properties, commercial suppliers and a reference to GenEMBL accession numbers for various suitable vectors.

In one embodiment, polynucleotides derived from the polynucleotides disclosed herein encode polypeptides or proteins having diagnostic and therapeutic utilities as described herein as well as probes to identify transcripts of the protein that may or may not be present. These nucleic acid fragments can by prepared, for example, by restriction enzyme digestion of larger polynucleotides and then labeled with a detectable marker. Alternatively, random fragments can be generated using nick translation of the molecule. For methodology for the preparation and labeling of such fragments, see, Sambrook et al. (1989) supra.

Expression vectors containing these nucleic acids are useful to obtain host vector systems to produce proteins and polypeptides. It is implied that these expression vectors must be replicable in the host organisms either as episomes or as an integral part of the chromosomal DNA. Non-limiting examples of suitable expression vectors include plasmids, yeast vectors, viral vectors and liposomes. Adenoviral vectors are particularly useful for introducing genes into tissues in vivo because of their high levels of expression and efficient transformation of cells both in vitro and in vivo. When a nucleic acid is inserted into a suitable host cell, e.g., a prokaryotic or a eukaryotic cell and the host cell replicates, the protein can be recombinantly produced. Suitable host cells will depend on the vector and can include mammalian cells, animal cells, human cells, simian cells, insect cells, yeast cells, and bacterial cells constructed using known methods. See, Sambrook et al. (1989) supra. In addition to the use of viral vector for insertion of exogenous nucleic acid into cells, the nucleic acid can be inserted into the host cell by methods known in the art such as transformation for bacterial cells; transfection using calcium phosphate precipitation for mammalian cells; or DEAE-dextran; electroporation; or microinjection. See, Sambrook et al. (1989) supra, for methodology. Thus, this disclosure also provides a host cell, e.g., a mammalian cell, an animal cell (rat or mouse), a human cell, or a prokaryotic cell such as a bacterial cell, containing a polynucleotide encoding a protein or polypeptide or antibody.

A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment disclosed herein that is a polynucleotide encompasses both the double-stranded form and each of two complementary single-stranded forms known or predicted to make up the double-stranded form.

When the vectors are used in an method as disclosed herein as gene therapy in vivo or ex vivo, a pharmaceutically acceptable vector, such as a replication-incompetent retroviral or adenoviral vector, are exemplary (but non-limiting) and may be of particular use. Pharmaceutically acceptable vectors containing the nucleic acids disclosed herein can be further modified for transient or stable expression of the inserted polynucleotide. As used herein, the term “pharmaceutically acceptable vector” includes, but is not limited to, a vector or delivery vehicle having the ability to selectively target and introduce the nucleic acid into dividing cells. An example of such a vector is a “replication-incompetent” vector defined by its inability to produce viral proteins, precluding spread of the vector in the infected host cell. An example of a replication-incompetent retroviral vector is LNL6 (Miller et al. (1989) BioTechniques 7:980-990). The methodology of using replication-incompetent retroviruses for retroviral-mediated gene transfer of gene markers has been established. (Bordignon (1989) PNAS USA 86:8912-8952; Culver (1991) PNAS USA 88:3155; and Rill (1991) Blood 79(10):2694-2700).

This disclosure also provides genetically modified cells that contain and/or express the polynucleotides disclosed herein. The genetically modified cells can be produced by insertion of upstream regulatory sequences such as promoters or gene activators (see, U.S. Pat. No. 5,733,761). In one embodiment, the modified cells are eukaryotic cells or prokaryotic cells.

The polynucleotides can be conjugated to a detectable marker, e.g., an enzymatic label or a radioisotope for detection of nucleic acid and/or expression of the gene in a cell. A wide variety of appropriate detectable markers are known in the art, including fluorescent, radioactive, enzymatic or other ligands, such as avidin/biotin, which are capable of giving a detectable signal. In one aspect, one will likely desire to employ a fluorescent label or an enzyme tag, such as urease, alkaline phosphatase or peroxidase, instead of radioactive or other environmentally undesirable reagents. In the case of enzyme tags, calorimetric indicator substrates can be employed to provide a means visible to the human eye or spectrophotometrically, to identify specific hybridization with complementary nucleic acid-containing samples. Thus, this disclosure further provides a method for detecting a single-stranded polynucleotide or its complement, by contacting target single-stranded polynucleotide with a labeled, single-stranded polynucleotide (a probe) which is a portion of the polynucleotide disclosed herein under conditions permitting hybridization (optionally moderately stringent hybridization conditions) of complementary single-stranded polynucleotides, or optionally, under highly stringent hybridization conditions. Hybridized polynucleotide pairs are separated from un-hybridized, single-stranded polynucleotides. The hybridized polynucleotide pairs are detected using methods known to those of skill in the art and set forth, for example, in Sambrook et al. (1989) supra.

The polynucleotide embodied in this disclosure can be obtained using chemical synthesis, recombinant cloning methods, PCR, or any combination thereof. Methods of chemical polynucleotide synthesis are known in the art and need not be described in detail herein. One of skill in the art can use the sequence data provided herein to obtain a desired polynucleotide by employing a DNA synthesizer or ordering from a commercial service.

The polynucleotides disclosed herein can be isolated or replicated using PCR. The PCR technology is the subject matter of U.S. Pat. Nos. 4,683,195; 4,800,159; 4,754,065; and 4,683,202 and described in PCR: The Polymerase Chain Reaction (Mullis et al. eds., Birkhauser Press, Boston (199.4)) or MacPherson et al. (1991) and (1995) supra, and references cited therein. Alternatively, one of skill in the art can use the sequences provided herein and a commercial DNA synthesizer to replicate the DNA. Accordingly, this disclosure also provides a process for obtaining the polynucleotides disclosed herein by providing the linear sequence of the polynucleotide, nucleotides, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, one of skill in the art can insert the poly-nucleotide into a suitable replication vector and insert the vector into a suitable host cell (prokaryotic or eukaryotic) for replication and amplification. The DNA so amplified can be isolated from the cell by methods known to those of skill in the art. A process for obtaining polynucleotides by this method is further provided herein as well as the polynucleotides so obtained.

RNA can be obtained by first inserting a DNA polynucleotide into a suitable host cell. The DNA can be delivered by any appropriate method, e.g., by the use of an appropriate gene delivery vehicle (e.g., liposome, plasmid or vector) or by electroporation. When the cell replicates and the DNA is transcribed into RNA; the RNA can then be isolated using methods known to those of skill in the art, for example, as set forth in Sambrook et al. (1989) supra. For instance, mRNA can be isolated using various lytic enzymes or chemical solutions according to the procedures set forth in Sambrook et al. (1989) supra, or extracted by nucleic-acid-binding resins following the accompanying instructions provided by manufactures.

Polynucleotides exhibiting sequence complementarity or homology to a polynucleotide disclosed herein are useful as hybridization probes or as an equivalent of the specific polynucleotides identified herein. Since the full coding sequence of the transcript is known, any portion of this sequence or homologous sequences can be used in the methods disclosed herein.

It is known in the art that a “perfectly matched” probe is not needed for a specific hybridization. Minor changes in probe sequence achieved by substitution, deletion or insertion of a small number of bases do not affect the hybridization specificity. In general, as much as 20% base-pair mismatch (when optimally aligned) can be tolerated. In some embodiments, a probe useful for detecting the aforementioned mRNA is at least about 80% identical to the homologous region. In some embodiments, the probe is 85% identical to the corresponding gene sequence after alignment of the homologous region; in some embodiments, it exhibits 90% identity.

These probes can be used in radioassays (e.g., Southern and Northern blot analysis) to detect, prognose, diagnose or monitor various cells or tissues containing these cells. The probes also can be attached to a solid support or an array such as a chip for use in high throughput screening assays for the detection of expression of the gene corresponding a polynucleotide disclosed herein. Accordingly, this disclosure also provides a probe comprising or corresponding to a polynucleotide disclosed herein, or its equivalent, or its complement, or a fragment thereof, attached to a solid support for use in high throughput screens.

The total size of fragment, as well as the size of the complementary stretches, will depend on the intended use or application of the particular nucleic acid segment. Smaller fragments will generally find use in hybridization embodiments, wherein the length of the complementary region may be varied, such as between at least 5 to 10 to about 100 nucleotides, or even full length according to the complementary sequences one wishes to detect.

Nucleotide probes having complementary sequences over stretches greater than 5 to 10 nucleotides in length are generally well suited, so as to increase stability and selectivity of the hybrid, and thereby improving the specificity of particular hybrid molecules obtained. In certain embodiments, one can design polynucleotides having gene-complementary stretches of 10 or more or more than 50 nucleotides in length, or even longer where desired. Such fragments may be readily prepared by, for example, directly synthesizing the fragment by chemical means, by application of nucleic acid reproduction technology, such as the PCR technology with two priming oligonucleotides as described in U.S. Pat. No. 4,603,102 or by introducing selected sequences into recombinant vectors for recombinant production. In one aspect, a probe is about 50-75 or more alternatively, 50-100, nucleotides in length.

The polynucleotides of the present disclosure can serve as primers for the detection of genes or gene transcripts that are expressed in cells described herein. In this context, amplification means any method employing a primer-dependent polymerase capable of replicating a target sequence with reasonable fidelity. Amplification may be carried out by natural or recombinant DNA-polymerases such as T7 DNA polymerase, Klenow fragment of E. coli DNA polymerase, and reverse transcriptase. For illustration purposes only, a primer is the same length as that identified for probes.

One method to amplify polynucleotides is PCR and kits for PCR amplification are commercially available. After amplification, the resulting DNA fragments can be detected by any appropriate method known in the art, e.g., by agarose gel electrophoresis followed by visualization with ethidium bromide staining and ultraviolet illumination.

Methods for administering an effective amount of a gene delivery vector or vehicle to a cell have been developed and are known to those skilled in the art and described herein. Methods for detecting gene expression in a cell are known in the art and include techniques such as in hybridization to DNA microarrays, in situ hybridization, PCR, RNase protection assays and Northern blot analysis. Such methods are useful to detect and quantify expression of the gene in a cell. Alternatively, expression of the encoded polypeptide can be detected by various methods. In particular, it is useful to prepare polyclonal or monoclonal antibodies that are specifically reactive with the target polypeptide. Such antibodies are useful for visualizing cells that express the polypeptide using techniques such as immunohistology, ELISA, and Western blotting. These techniques can be used to determine expression level of the expressed polynucleotide.

### Production Methods

Also provided are methods to produce the antibodies, fragments, CDRs, or polypeptides comprising, or alternatively consisting of, or yet further consisting of, culturing a host cell comprising a polynucleotide encoding the antibody, antigen binding fragment, polypeptide, or CDR under conditions for expression of the polynucleotide, and optionally isolating the antibody, fragment, CDR and/or polypeptide from the cell and/or culture. Additionally provided is a host cell comprising a polynucleotide encoding the antibody, antigen binding fragment, polypeptide, or CDR under conditions for expression of the polynucleotide. In one embodiment, the host cell is a eukaryotic cell or a prokaryotic cell. In a further embodiment, the host cell is a mammalian cell.

### Compositions

Compositions are further provided. The compositions comprise a carrier and one or more of an isolated polypeptide disclosed herein, an isolated polynucleotide disclosed herein, a vector disclosed herein, an isolated host cell disclosed herein, a small molecule or an antibody, and/or an antigen binding fragment disclosed herein. The carriers can be one or more of a solid support or a pharmaceutically acceptable carrier. The compositions can further comprise an adjuvant or other components suitable for administrations as vaccines. In one aspect, the compositions are formulated with one or more pharmaceutically acceptable excipients, diluents, carriers and/or adjuvants. In addition, embodiments of the compositions of the present disclosure include one or more of an isolated polypeptide disclosed herein, an isolated polynucleotide disclosed herein, a vector disclosed herein, a small molecule, an isolated host cell disclosed herein, or an antibody of the disclosure, formulated with one or more pharmaceutically acceptable substances.

For oral preparations, any one or more of an isolated or recombinant polypeptide as described herein, an isolated or recombinant polynucleotide as described herein, a vector as described herein, an isolated host cell as described herein, a small molecule or an antibody or fragment thereof as described herein can be used alone or in pharmaceutical formulations disclosed herein comprising, or consisting essentially of, the compound in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical formulations and unit dose forms suitable for oral administration are particularly useful in the treatment of chronic conditions, infections, and therapies in which the patient self-administers the drug. In one aspect, the formulation is specific for pediatric administration.

The disclosure provides pharmaceutical formulations in which the one or more of an isolated polypeptide disclosed herein, an isolated polynucleotide disclosed herein, a vector disclosed herein, an isolated host cell disclosed herein, or an antibody disclosed herein can be formulated into preparations for injection in accordance with the disclosure by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives or other antimicrobial agents. A non-limiting example of such is a antimicrobial agent such as other vaccine components such as surface antigens, e.g., an OMP P5, OMP 26, OMP P2, or Type IV Pilin protein (see Jurcisek and Bakaletz (2007) J. of Bacteriology 189(10):3868-3875 and Murphy, T F, Bakaletz, L O and Smeesters, P R (2009) The Pediatric Infectious Disease Journal, 28:S121-S126) and antibacterial agents. For intravenous administration, suitable carriers include physiological bacteriostatic water, Cremophor EL™ (BASF, Parsippany, N.J.), or phosphate buffered saline (PBS). In all cases, a composition for parenteral administration must be sterile and should be fluid to the extent that easy syringability exists.

Aerosol formulations provided by the disclosure can be administered via inhalation and can be propellant or non-propellant based. For example, embodiments of the pharmaceutical formulations disclosed herein comprise a compound disclosed herein formulated into pressurized acceptable propellants such as dichlorodifluoromethane, propane, nitrogen and the like. For administration by inhalation, the compounds can be delivered in the form of an aerosol spray from a pressurized container or dispenser which contains a suitable propellant, e.g., a gas such as carbon dioxide, or a nebulizer. A non-limiting example of a non-propellant is a pump spray that is ejected from a closed container by means of mechanical force (i.e., pushing down a piston with one's finger or by compression of the container, such as by a compressive force applied to the container wall or an elastic force exerted by the wall itself, e.g., by an elastic bladder).

Suppositories disclosed herein can be prepared by mixing a compound disclosed herein with any of a variety of bases such as emulsifying bases or water-soluble bases. Embodiments of this pharmaceutical formulation of a compound disclosed herein can be administered rectally via a suppository. The suppository can include vehicles such as cocoa butter, carbowaxes and polyethylene glycols, which melt at body temperature, yet are solidified at room temperature.

Unit dosage forms for oral or rectal administration, such as syrups, elixirs, and suspensions, may be provided wherein each dosage unit, for example, teaspoonful, tablespoonful, tablet or suppository, contains a predetermined amount of the composition containing one or more compounds disclosed herein. Similarly, unit dosage forms for injection or intravenous administration may comprise a compound disclosed herein in a composition as a solution in sterile water, normal saline or another pharmaceutically acceptable carrier.

Embodiments of the pharmaceutical formulations disclosed herein include those in which one or more of an isolated polypeptide disclosed herein, an isolated polynucleotide disclosed herein, a vector disclosed herein, a small molecule for use in the disclosure, an isolated host cell disclosed herein, or an antibody or fragment thereof as disclosed herein is formulated in an injectable composition. Injectable pharmaceutical formulations disclosed herein are prepared as liquid solutions or suspensions; or as solid forms suitable for solution in, or suspension in, liquid vehicles prior to injection. The preparation may also be emulsified or the active ingredient encapsulated in liposome vehicles in accordance with other embodiments of the pharmaceutical formulations disclosed herein.

In an embodiment, one or more of an isolated polypeptide disclosed herein, an isolated polynucleotide disclosed herein, a vector disclosed herein, an isolated host cell disclosed herein, or an antibody disclosed herein is formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present disclosure. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, delivery of a compound disclosed herein can be accomplished using any of a variety of refillable, pump systems. Pumps provide consistent, controlled release over time. In some embodiments, a compound disclosed herein is in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to, a subdermal, subcutaneous, intramuscular, or other suitable site within a subject's body. Subcutaneous implantation sites are used in some embodiments because of convenience in implantation and removal of the drug delivery device.

Drug release devices suitable for use in the disclosure may be based on any of a variety of modes of operation, polymers such as for example poly(glycolide-co-lactide) (PGLA) that is commercially available from a number of vendors, e.g., BioDegmer and Sigma-Aldrich. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer (e.g., PGLA) and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present disclosure. Examples of such devices include those described in, for example, U.S. Pat. Nos. 4,692,147; 4,360,019; 4,487,603; 4,360,019; 4,725,852; and the like. In general, a subject treatment method can be accomplished using any of a variety of refillable, non-exchangeable pump systems. Pumps and other convective systems may be utilized due to their generally more consistent, controlled release over time. Osmotic pumps are used in some embodiments due to their combined advantages of more consistent controlled release and relatively small size (see, e.g., PCT International Application Publication No. WO 97/27840 and U.S. Pat. Nos. 5,985,305 and 5,728,396). Exemplary osmotically-driven devices suitable for use in the disclosure include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like. A further exemplary device that can be adapted for the present disclosure is the Synchromed infusion pump (Medtronic).

In some embodiments, the drug delivery device is an implantable device. The drug delivery device can be implanted at any suitable implantation site using methods and devices well known in the art. As noted herein, an implantation site is a site within the body of a subject at which a drug delivery device is introduced and positioned. Implantation sites include, but are not necessarily limited to a subdermal, subcutaneous, intramuscular, or other suitable site within a subject's body.

Suitable excipient vehicles for a compound disclosed herein are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pa., 17th edition, 1985. The composition or formulation to be administered will, in any event, contain a quantity of the compound adequate to achieve the desired state in the subject being treated.

Compositions of the present disclosure include those that comprise a sustained-release or controlled release matrix. In addition, embodiments of the present disclosure can be used in conjunction with other treatments that use sustained-release formulations. As used herein, a sustained-release matrix is a matrix made of materials, usually polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids such as phenylatanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix.

In another embodiment, the polypeptide, antibody or fragment thereof (as well as combination compositions) is delivered in a controlled release system. For example, a compound disclosed herein may be administered using intravenous infusion, an implantable osmotic pump, a transdermal patch, liposomes, or other modes of administration. In one embodiment, a pump may be used (Sefton (1987) CRC Crit. Ref Biomed. Eng. 14:201; Buchwald et al. (1980) Surgery 88:507; Saudek et al. (1989) N. Engl. J. Med. 321:574). In another embodiment, polymeric materials are used. In yet another embodiment a controlled release system is placed in proximity of the therapeutic target, i.e., the liver, thus requiring only a fraction of the systemic dose. In yet another embodiment, a controlled release system is placed in proximity of the therapeutic target, thus requiring only a fraction of the systemic. Other controlled release systems are discussed in the review by Langer (1990) Science 249:1527-1533.

In another embodiment, the compositions of the present disclosure (as well as combination compositions separately or together) include those formed by impregnation of an inhibiting agent described herein into absorptive materials, such as sutures, bandages, and gauze, or coated onto the surface of solid phase materials, such as surgical staples, zippers and catheters to deliver the compositions. Other delivery systems of this type will be readily apparent to those skilled in the art in view of the instant disclosure.

The present disclosure provides methods and compositions for the administration of a one or more of an interfering agent to a host (e.g., a human) for the treatment of a microbial infection. In various embodiments, these methods disclosed herein span almost any available method and route suitable for drug delivery, including in vivo and ex vivo methods, as well as systemic and localized routes of administration.

### Formulations and Co-Formulations

The disclosure provided herein contemplates specific formulations and co-formulations of the agents disclosed herein along with a pharmaceutically acceptable excipient, such as those disclosed herein above.

In specific aspects, the disclosure provides for formulations or co-formulations comprising antibodies or antigen binding fragments thereof that specifically recognize or bind an isolated or recombinant polypeptides. Antibodies disclosed herein may be selected such that they have a high level of epitope binding specificity and high binding affinity to the biofilm. In general, the higher the binding affinity of an antibody, the more stringent wash conditions can be performed in an immunoassay to remove nonspecifically bound material without removing the target. Accordingly, the antibodies of the present technology useful in the disclosed methods usually have binding affinities of at least 10⁻⁶, 10⁻⁷, 10⁻¹, 10⁻¹, 10⁻¹⁰, 10⁻¹¹, or 10⁻¹² M. In certain aspects, the antibodies have a sufficient kinetic on-rate to reach equilibrium under standard conditions in at least 12 hours, at least 5 hours, at least 1 hour, or at least 30 minutes. In another aspect, the affinity of the antibody or antigen binding fragment is less than or about 1000 picoMole (pM), 900 pM, 800 pM, 700 pM, 600 pM, 500 pM, 400 pM, 300 pM, 200 pM, about 100 pM, 50 pM, 40 pM, 30 pM, 20 pM, 10 pM, 9 pM, 8 pM, 7 pM, 6 pM, 5 pM, or 4 pM.

In some embodiments, the antibodies or antigen binding fragments thereof are present in the formulation at a concentration from about 0.1 mg/mL to about 200 mg/mL, or alternatively from about 1 to about 150 mg/mL, or alternatively about 2 mg/mL to about 100 mg/mL, or alternatively about 3 mg/mL to about 80 mg/mL, or alternatively about 4 mg/mL to about 50 mg/mL, or alternatively about 5 mg/mL to about 20 mg/mL. In some embodiments, the antibodies are present at a concentration of at least about 1 mg/mL, or alternatively at least about 2 mg/mL, at least about 3 mg/mL, or alternatively at least about 4 mg/mL, or alternatively at least about 5 mg/mL, or alternatively at least about 6 mg/mL, or alternatively at least about 7 mg/mL, or alternatively at least about 8 mg/mL, or alternatively at least about 9 mg/mL, or alternatively at least about 10 mg/mL, or alternatively at least about 15 mg/mL, or alternatively at least about 20 mg/mL, or alternatively at least about 30 mg/mL, or alternatively at least about 40 mg/mL, or alternatively at least about 50 mg/mL, or alternatively at least about 60 mg/mL, or alternatively at least about 70 mg/mL, or alternatively at least about 80 mg/mL, or alternatively at least about 90 mg/mL, or alternatively at least about 100 mg/mL, or alternatively at least about 120 mg/mL, or alternatively at least about 150 mg/mL or alternatively at least about 200 mg/mL. In some embodiments, at least one of the plurality of antibodies is present at a concentration of at least about 1 mg/mL, or alternatively at least about 2 mg/mL, or alternatively at least about 3 mg/mL, or alternatively at least about 4 mg/mL, or alternatively at least about 5 mg/mL, or alternatively at least about 6 mg/mL, or alternatively at least about 7 mg/mL, or alternatively at least about 8 mg/mL, or alternatively at least about 9 mg/mL, or alternatively at least about 10 mg/mL, or alternatively at least about 15 mg/mL, or alternatively at least about 20 mg/mL, or alternatively at least about 30 mg/mL, or alternatively at least about 40 mg/mL, or alternatively at least about 50 mg/mL, or alternatively at least about 60 mg/mL, or alternatively at least about 70 mg/mL, or alternatively at least about 80 mg/mL, or alternatively at least about 90 mg/mL, or alternatively at least about 100 mg/mL, or alternatively at least about 120 mg/mL, or alternatively at least about 150 mg/mL, or alternatively at least about 200 mg/mL.

In some embodiments, wherein multiple different antibodies are included an antibody co-formulation, the different antibodies may be present in substantially equal concentrations. In another aspect of such embodiments, the different antibodies one or more of the antibodies may be present in a substantially higher concentration than the other antibodies, e.g., ratios of about 1.5:1, or alternatively about 1.5:1:1, or alternatively about 1.5:1:1:1, or alternatively about 2:1, or alternatively about 2:1:1, or alternatively about 2:1:1:1, or alternatively at least about 2.5:1, or alternatively at least about 2.5:1:1, or alternatively at least about 2.5:1:1:1.

In some embodiments the co-formulation comprises, or alternatively consists essentially of, or yet further comprises an antibody that specifically recognizes or binds an isolated or recombinant polypeptide consisting essentially of a two or more of A5 a fragment or equivalent thereof (e.g., in duplicate or in combination with another such as mB4) or two or more of mB4 polypeptide, a fragment or equivalent thereof or mB4 in combination with A5, a fragment or an equivalent of each thereof. In some embodiments, one or more antibodies in the formulation is not a polyclonal antibody. In some embodiments, this formulation is used as a therapeutic.

In some embodiments the co-formulation comprises, or alternatively consists essentially of, or yet further comprises an antibody that specifically recognizes or binds an isolated or recombinant polypeptide consisting essentially of two or more of A1 to A4 or A6 or B1 to B6, or a fragment or equivalent thereof. In some embodiments, one or more antibodies in the formulation is not a polyclonal antibody. In some embodiments, this formulation is used as a diagnostic.

Methods of stably formulating antibody formulations and co-formulations can be made according to techniques disclosed in the art—see, e.g., U.S. Pat. Publication No. US 2011/0059079.

It is also possible to confer short-term protection to a host by passive immunotherapy via the administration of preformed antibody against a chimeric protein of the invention. Thus, antibodies of the invention may be used in passive immunotherapy. Human immunoglobulin is preferred in human medicine because a heterologous immunoglobulin may provoke an immune response to its foreign immunogenic components. Such passive immunization could be used on an emergency basis for immediate protection of unimmunized individuals subject to special risks.

### Screening Assays

The present disclosure provides methods for screening for equivalent agents, such as equivalent monoclonal antibodies to a polyclonal antibody as described herein and various agents that modulate the activity of the active agents and pharmaceutical compositions disclosed herein or the function of a polypeptide or peptide product encoded by the polynucleotide disclosed herein. For the purposes of this disclosure, an “agent” is intended to include, but not be limited to a biological or chemical compound such as a simple or complex organic or inorganic molecule (referred to herein as a small molecule, such as a nucleic acid), a peptide, a protein (e.g., antibody), a polynucleotide anti-sense) or a ribozyme. A vast array of compounds can be synthesized, for example polymers, such as polypeptides and polynucleotides, and synthetic organic compounds based on various core structures, and these are also included in the term “agent.” In addition, various natural sources can provide compounds for screening, such as plant or animal extracts, and the like. It should be understood, although not always explicitly stated that the agent is used alone or in combination with another agent, having the same or different biological activity as the agents identified by the inventive screen.

The potential inhibitory or binding effect (i.e., interaction or association) of an agent such as a small molecule compound may be analyzed prior to its actual synthesis and testing by the use of computer modeling techniques. If the theoretical structure of the given compound suggests insufficient interaction and association between it and microbial DNA in the biofilm and/or DNABII protein, synthesis and testing of the agent can be obviated. However, if computer modeling indicates a strong interaction, the agent can then be synthesized and tested for its ability to bind to or inhibit the interaction using various methods such as in vitro or in vivo experiments. Methods of testing an agent's ability to inhibit or titrate a biofilm, alone or in connection with another agent, are disclosed herein. In this manner, synthesis of inoperative agents and compounds can be avoided.

One skilled in the art may use any of several methods to screen chemical or biological entities or fragments for their ability to associate with DNABII or microbial DNA and more particularly with the specific binding sites. Selected fragments or chemical entities may then be positioned in a variety of orientations, or docked, within an individual binding site of DNA or DNABII polypeptide. Docking may be accomplished using software such as QUANTA, SYBYL, followed by energy minimization and molecular dynamics with standard molecular mechanical force fields, such as CHARMM and AMBER.

Commercial computer programs are also available for in silico design. Examples include, without limitation, GRID (Oxford University, Oxford, UK), MCSS (Molecular Simulations, Burlington, Mass.), AUTODOCK (Scripps Research Institute, La Jolla, Calif.), DOCK (University of California, San Francisco, Calif), GLIDE (Schrodinger Inc.), FlexX (Tripos Inc.) and GOLD (Cambridge Crystallographic Data Centre).

Once an agent or compound has been designed or selected by the above methods, the efficiency with which that agent or compound may bind to each other can be tested and optimized by computational evaluation. For example, an effective DNABII fragment or may demonstrate a relatively small difference in energy between its bound and free states (i.e., a small deformation energy of binding).

A compound designed or selected can be further computationally optimized so that in its bound state it may optionally lack repulsive electrostatic interaction with the target protein. Such non-complementary (e.g., electrostatic) interactions include repulsive charge-charge, dipole-dipole, and charge-dipole interactions. Specifically, the sum of all electrostatic interactions between the agent and DNABII and/or microbial DNA in the biofilm when the agent or compound is bound to either agent, optionally making a neutral or favorable contribution to the enthalpy of binding.

Computer softwares are also available in the art to evaluate compound deformation energy and electrostatic interaction. Examples include, without limitation, Gaussian 92 [Gaussian, Inc., Pittsburgh, Pa.]; AMBER [University of California at San Francisco]; QUANTA/CHARMM [Molecular Simulations, Inc., Burlington, Mass.]; and Insight II/Discover [Biosym Technologies Inc., San Diego, Calif.].

Once a binding agent has been optimally selected or designed, as described above, substitutions may then be made in some of its atoms or side groups in order to improve or modify its binding properties. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds may then be analyzed for efficiency of fit to the DNABII protein and/or microbial DNA in the biofilm by the same computer methods described in detail, above.

Certain embodiments relate to a method for screening small molecules capable of interacting with the protein or polynucleotide disclosed herein. For the purpose of this disclosure, “small molecules” are molecules having low molecular weights (MW) that are, in one embodiment, capable of binding to a protein of interest thereby altering the function of the protein. In some embodiments, the MW of a small molecule is no more than 1,000. Methods for screening small molecules capable of altering protein function are known in the art. For example, a miniaturized arrayed assay for detecting small molecule-protein interactions in cells is discussed by You et al. (1997) Chem. Biol. 4:961-968.

To practice the screening method in vitro, suitable cell culture or tissue infected with the microbial to be treated are first provided. The cells are cultured under conditions (temperature, growth or culture medium and gas (CO₂)) and for an appropriate amount of time to attain exponential proliferation without density dependent constraints. It also is desirable to maintain an additional separate cell culture that is not infected as a control.

As is apparent to one of skill in the art, suitable cells can be cultured in micro-titer plates and several agents can be assayed at the same time by noting genotypic changes, phenotypic changes or a reduction in microbial titer.

When the agent is a composition other than a DNA or RNA, such as a small molecule as described above, the agent can be directly added to the cell culture or added to culture medium for addition. As is apparent to those skilled in the art, an “effective” a mount must be added which can be empirically determined,

When the agent is an antibody or antigen binding fragment, the agent can be contacted or incubated with the target antigen and polyclonal antibody as described herein under conditions to perform a competitive ELISA. Such methods are known to the skilled artisan.

The assays also can be performed in a subject. When the subject is an animal such as a rat, chinchilla, mouse or simian, the method provides a convenient animal model system that can be used prior to clinical testing of an agent in a human patient. In this system, a candidate agent is a potential drug if symptoms of the disease or microbial infection is reduced or eliminated, each as compared to untreated, animal having the same infection. It also can be useful to have a separate negative control group of cells or animals that are healthy and not treated, which provides a basis for comparison.

The agents and compositions can be used in the manufacture of medicaments and for the treatment of humans and other animals by administration in accordance with conventional procedures, such as an active ingredient in pharmaceutical compositions.

### Combination Therapies

The compositions and related methods of the present disclosure may be used in combination with the administration of other therapies. These include, but are not limited to, the administration of DNase enzymes, antibiotics, antimicrobials, anti-infectives, anti-fungals, anti-parasitics, anti-virals, or other antibodies.

In some embodiments, the methods and compositions include a deoxyribonuclease (DNase) enzyme that acts synergistically with the anti-DNABII antibody. A DNase is any enzyme that catalyzes the cleavage of phosphodiester linkages in the DNA backbone. Three non-limiting examples of DNase enzymes that are known to target not only cruciform structures, but also a variety of secondary structure of DNA include DNAse I, T4 EndoVII, T7 Endo I, RuvABC, and RusA. In certain embodiments, the effective amount of anti-DNABII antibody needed to destabilize the biofilm is reduced when combined with a DNase. When administered in vitro, the DNase can be added directly to the assay or in a suitable buffer known to stabilize the enzyme. The effective Unit dose of DNase and the assay conditions may vary, and can be optimized according to procedures known in the art.

In other embodiments, the methods and compositions can be combined with antibiotics and/or antimicrobials. Antimicrobials are substances that kill or inhibit the growth of microorganisms such as bacteria, fungi, or protozoans. Although biofilms are generally resistant to the actions of antibiotics, compositions and methods described herein can be used to sensitize the infection involving a biofilm to traditional therapeutic methods for treating infections. In other embodiments, the use of antibiotics or antimicrobials in combination with methods and compositions described herein allow for the reduction of the effective amount of the antimicrobial and/or biofilm reducing agent. Some non-limiting examples of antimicrobials and antibiotics useful in combination with methods of the current disclosure include amoxicillin, amoxicillin-clavulanate, cefdinir, azithromycin, and sulfamethoxazole-trimethoprim. The therapeutically effective dose of the antimicrobial and/or antibiotic in combination with the biofilm reducing agent can be readily determined by traditional methods. In some embodiments the dose of the antimicrobial agent in combination with the biofilm reducing agent is the average effective dose which has been shown to be effective in other bacterial infections, for example, bacterial infections wherein the etiology of the infection does not include a biofilm. In other embodiments, the dose is 0.1, 0.15, 0.2, 0.25, 0.30, 0.35, 0.40, 0.45, 0.50, 0.55, 0.60, 0.65, 0.70, 0.75, 0.8, 0.85, 0.9, 0.95, 1.1, 1.2, 1.3, 1.4, 1.5, 1.6, 1.7, 1.8, 1.9, 2.0, 2.5, 3.0 or 5 times the average effective dose. The antibiotic or antimicrobial can be added prior to, concurrent with, or subsequent to the addition of the anti-DNABII antibody.

In other embodiments, the methods and compositions can be combined with antibodies that treat the bacterial infection. One example of an antibody useful in combination with the methods and compositions described herein is an antibody directed against an unrelated outer membrane protein (i.e., OMP P5). Treatment with this antibody alone does not debulk a biofilm in vitro. Combined therapy with this antibody and a biofilm reducing agent results in a greater effect than that which could be achieved by either reagent used alone at the same concentration. Other antibodies that may produce a synergistic effect when combined with a biofilm reducing agent or methods to reduce a biofilm include anti-rsPilA anti-OMP26, anti-OMP P2, and anti-whole OMP preparations.

The compositions and methods described herein can be used to sensitize the bacterial infection involving a biofilm to common therapeutic modalities effective in treating bacterial infections without a biofilm but are otherwise ineffective in treating bacterial infections involving a biofilm. In other embodiments, the compositions and methods described herein can be used in combination with therapeutic modalities that are effective in treating bacterial infections involving a biofilm, but the combination of such additional therapy and biofilm reducing agent or method produces a synergistic effect such that the effective dose of either the biofilm reducing agent or the additional therapeutic agent can be reduced. In other instances, the combination of such additional therapy and biofilm reducing agent or method produces a synergistic effect such that the treatment is enhanced. An enhancement of treatment can be evidenced by a shorter amount of time required to treat the infection.

The additional therapeutic treatment can be added prior to, concurrent with, or subsequent to methods or compositions used to reduce the biofilm, and can be contained within the same formation/composition or as a separate formulation/composition.

### Kits

Kits containing the agents and instructions necessary to perform the in vitro and in vivo methods as described herein also are claimed. Accordingly, the disclosure provides kits for performing these methods which may include an antibody, antibody fragment, polypeptide, polynucleotide, vector or host cell, as well as instructions for carrying out the methods disclosed herein such as collecting a sample and/or performing the screen, and/or analyzing the results, and/or administration of an effective amount of an antibody, antibody fragment, polypeptide, polynucleotide, vector or host cell, as defined herein. These can be used alone or in combination with other suitable antimicrobial agents.

For example, a kit can comprise, or alternatively consist essentially of, or yet further consist of any one or more of agents identified above, e.g., antibody, antibody fragment, polypeptide, polynucleotide, vector or host cell, and instructions for use. The kit can further comprise one or more of an adjuvant, an antigenic peptide or an antimicrobial. Examples of carriers include a liquid carrier, a pharmaceutically acceptable carrier, a solid phase carrier, a pharmaceutically acceptable carrier, a pharmaceutically acceptable polymer, a liposome, a micelle, an implant, a stent, a paste, a gel, a dental implant, or a medical implant.

Kits containing the agents and instructions necessary to perform the in vitro and in vivo methods as described herein also are claimed. Accordingly, the disclosure provides kits for performing these methods which may include an agent disclosed herein as well as instructions for carrying out the methods disclosed herein such as collecting a sample and/or performing the screen, and/or analyzing the results, and/or administration of an effective amount of an agent as defined herein. These can be used alone or in combination with other suitable antimicrobial agents.

For example, a kit can comprise, or alternatively consist essentially of, or yet further consist of any one or more agent identified above, e.g., an agent of the group of an isolated or recombinant polypeptide or a fragment or an equivalent of each thereof; an isolated or recombinant polynucleotide encoding any one of the above noted polypeptides; an antibody or fragment thereof; and instructions for use. The kit can further comprising one or more of an adjuvant, an antigenic peptide or an antimicrobial. Examples of carriers include a liquid carrier, a pharmaceutically acceptable carrier, a solid phase carrier, a pharmaceutically acceptable carrier, a pharmaceutically acceptable polymer, a liposome, a micelle, an implant, a stent, a paste, a gel, a dental implant, or a medical implant.

The following examples are intended to illustrate, and not limit the embodiments disclosed herein.

### Experimental Methods

### Experiment No. 1—Nontypeable Haemophilus influenzae Newly Released (NRel) from Biofilms by Antibody-Mediated Dispersal Versus Antibody-Mediated Disruption are Phenotypically Distinct

Despite the fact that the mechanisms and kinetics of the two targeted antisera used here are very different, the outcome of exposure of NTHI biofilms to anti-rsPilA antibodies or anti-DNABII antibodies is release of NTHI from biofilm residence into the surrounding milieu. Pioneering work from several laboratories reveals that bacteria released from a biofilm demonstrate a distinct phenotype from their biofilm-resident or planktonic counterparts (Chua et al., 2014; Marks et al., 2013; Sauer et al., 2004). Of note, a common characteristic of these released bacteria is sensitivity to antibiotic killing greater than that shown by even planktonically grown bacteria (Brockson et al., 2014; Chambers et al., 2017; Goodwine et al., 2019; Mokrzan et al., 2018). Intriguingly, whereas P. aeruginosa released from a biofilm by exposure to either glutamate or nitric oxide showed variable sensitivity to tobramycin and/or colistin compared to each other, both populations were more sensitive to antibiotic killing than their planktonic counterparts (Chambers et al., 2017; Petrova and Sauer, 2016). Applicant showed that NTHI and M. catarrhalis released from a dual species biofilm by anti-rsPilA antibodies are significantly more sensitive to killing by either trimethoprim plus sulfamethoxazole or clarithromycin, respectively, than their agar-grown counterparts (Mokrzan et al., 2018). Moreover, NTHI biofilms exposured to anti-DNABII antibodies in combination with antibiotics significantly augments killing of the newly released NTHI by all three first-line antibiotics used to treat OM (e.g. ampicillin, amoxicillin-clavulanate, cefdinir) in vitro (Brockson et al., 2014). Further, Applicant's in vivo studies demonstrate that treatment with anti-DNABII antibodies in combination with the aminoglycoside antibiotic tobramycin confers an added benefit to the eradication of P. aeruginosa from the murine lung compared to treatment with antibodies or antibiotic alone (Novotny et al., 2016).

Herein Applicant used NTHI as a model organism to further characterize the phenotype of newly released bacteria, hereafter referred to as ‘NRel’. Given the unique ways in which NTHI are released from biofilms by anti-rsPilA compared to anti-DNABII antibodies, Applicant used comparative analysis of abundances of all expressed proteins (as determined by quantitative mass spectrometry), targeted transcriptomics, flow cytometry and susceptibility to killing by a sulfonamide or β-lactam antibiotic to investigate whether anti-rsPilA induced NRel NTHI (Novotny et al., 2015) were phenotypically different than anti-DNABII induced NRel NTHI (Brockson et al., 2014), despite the genetic identity of these two populations.

Incubation of NTHI Biofilms with Anti-rsPilA or Anti-IHF Antibodies Released NTHI from Biofilm Residence

To date, Applicant has shown extensive differences between the two mechanisms and outcomes of anti-rsPilA or anti-DNABII antibody-mediated release of biofilm-resident bacteria to the NRel state and here added two new discriminators, presented in Table 2 (Devaraj et al., 2015; Goodman et al., 2011; Novotny et al., 2013a; Novotny et al., 2016). To now expand further on this understanding, Applicant first quantified the number of NTHI released from biofilm residence after incubation with rabbit polyclonal IgG isolated from either anti-rsPilA or anti-IHF serum. Sterile culture medium or an equivalent concentration of polyclonal IgG recovered from either naive serum or from antiserum against the NTHI adhesin outer membrane protein P5 (OMP P5), none of which disperse established NTHI biofilms (Mokrzan et al., 2018; Novotny et al., 2015), served as negative controls. Applicant's prior work reveals that NTHI biofilms established for 16 h and incubated with anti-rsPilA are maximally dispersed after 6 h (Mokrzan et al., 2018; Novotny et al., 2015), whereas complete collapse of a similarly aged biofilm is achieved after 15 min with anti-IHF (Novotny et al., 2019). Thus, for this assay, NRel induced by anti-rsPilA or anti-IHF were collected for enumeration at either 6 h or 15 min, respectively (Mokrzan et al., 2018; Novotny et al., 2019; Novotny et al., 2015).

TABLE 2
Differences between mechanisms of NTHI release from biofilm-residence to
the NRel state as mediated by anti-rsPilA or anti-IHF.

| | anti-rsPilA | anti-IHF |
|---|---|---|
| Antigen location | NTHI surface (Bakaletz | Biofilm structural linchpin |
| | et al., 2005) | (Goodman et al., 2011) |
| NTHI contribution | Active - | Passive - artificially induced |
| | native/programmed | |
| | (Novotny et al., 2015) | |
| Release speed | Slow (Novotny et al., | Fast (Brockson et al., 2014) |
| | 2015) | |
| Release mode | Top-down (Novotny et | All at once (Novotny et al., |
| | al., 2015) | 2019) |
| NTHI-specific antigen? | Yes | No, species-agnostic |
| | | (Goodman et al., 2011) |
| NRel in lag phase? | No (this study) | Yes (this study) |
| Released as aggregates | No (this study) | Yes (this study) |
| Term used to define release | Dispersal | Disruption |
| from biofilm | | |

The concentration of NTHI recovered from supermatants above biofilms that had been incubated with polyclonal IgG from either naive or anti-OMP P5 serum was similar to that when incubated with sterile sBHI (FIGS. 1a & 1b). This was anticipated as none of these three treatments were expected to significantly alter the normal equilibrium wherein bacteria go on/come off a biofilm as a natural part of biofilm growth and remodeling within the 6 h or 15 min incubation periods. Conversely, incubation with anti-rsPilA IgG for 6 h induced a significant >2-fold increase in the concentration of released NTHI (FIG. 1a, P<0.0001). Similarly, incubation with anti-IHF IgG for 15 min resulted in a significant >3-fold increase in concentration of released NTHI (P<0.0001) (FIG. 1b). The lower concentration of the 3 control populations, compared to those similarly depicted in FIG. 1a, reflects the shorter 15 min incubation period. Applicant refers herein to these populations of NTHI newly released from biofilm-residence as ‘anti-rsPilA NRel’ or ‘anti-IHF NRel’ to reflect their generation due to the action of two unique and specifically-targeted antibodies wherein the mechanisms and kinetics of release of biofilm resident NTHI are different (Table 2).

FACS Analysis of Anti-rsPilA and Anti-IHF NRel Populations

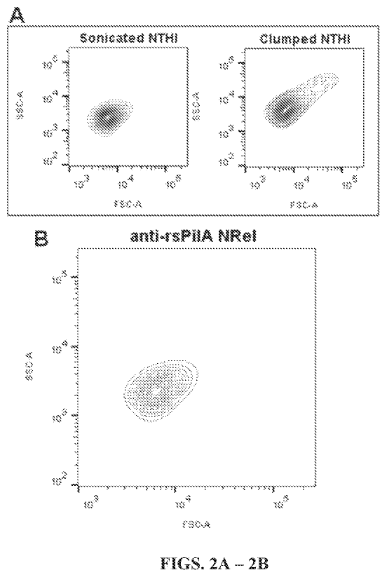

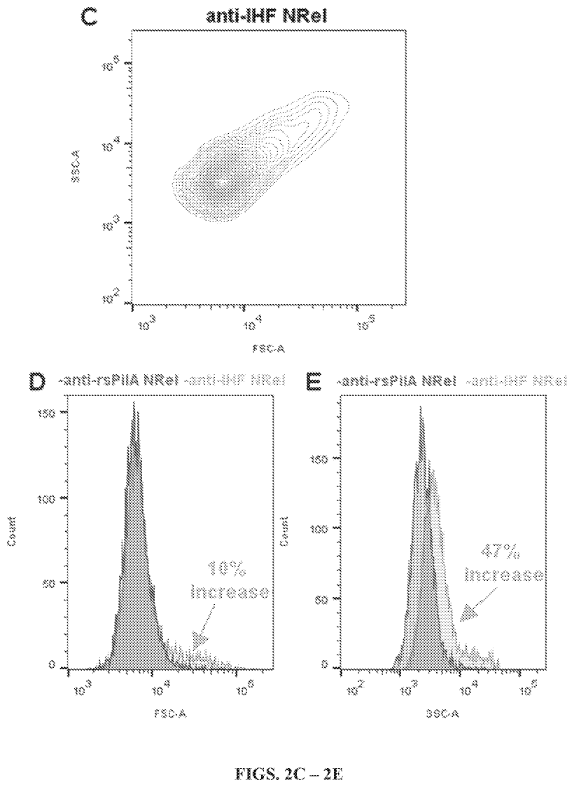

As hypothesized and based on gross observations of the NRel populations as they were recovered, the scatter profiles for anti-rsPilA NRel and anti-IHF NRel are also distinct. Applicant performed flow cytometry and examined the forward scatter and side scatter profiles for each NRel population to reveal potential differences in size and complexity. The scatter profile for a suspension of NTHI that was briefly sonicated prior to assessment revealed a population of cells of similar size (i.e. single cells), whereas a larger and more complex population (i.e. aggregates) was additionally observed in the sample with bacterial aggregates, indicated by greater side scatter and forward scatter (FIG. 2a). Anti-rsPilA NRel appear to be released as individual cells (FIG. 2b), as a single, uniform population was revealed. Conversely, the scatter profile for anti-IHF NRel included a larger population, indicative of bacterial aggregates and/or biofilm remnants (FIG. 2c). Moreover, the anti-IHF NRel population was 10% larger in size (FIG. 2d) and 47% more complex (FIG. 2e) compared to the anti-rsPilA NRel, further evidence of distinct character of each population. Moreover, these data fit well with the described differences for how each antiserum mediates release of NTHI from biofilm residence (Table 2) as the programmed release mediated via dispersal would indeed favor release as individual cells, whereas the rapid physical collapse of the biofilm to release NTHI via disruption would favor release as aggregates.

Proteomic Expression Profiles of Anti-rsPilA and Anti-IHF NRel were Distinct from Both their Planktonically Grown Counterparts and Importantly, from Each Other

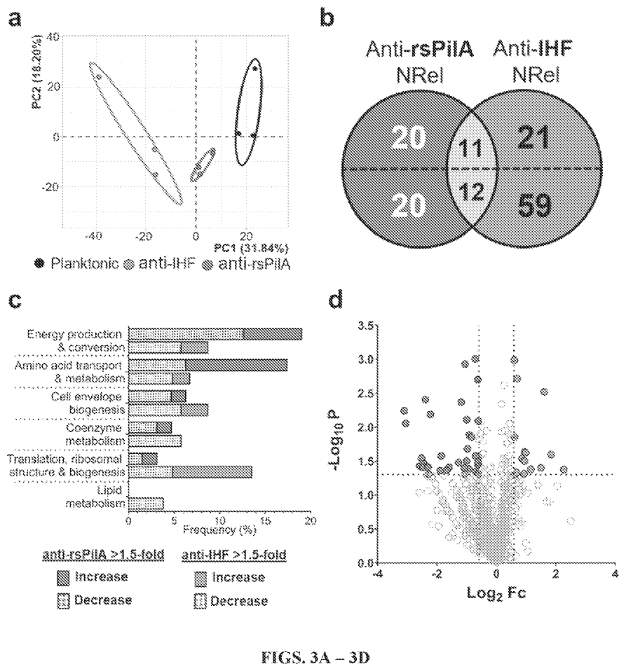

Given Applicant's ultimate interest in the relative sensitivity of NRel NTHI to killing by antibiotics, next Applicant questioned how the anti-rsPilA and anti-IHF NRel populations compared phenotypically to not only planktonically grown NTHI (the population used clinically to determine antibiotic sensitivities, or MIC values), but also to each other. To begin to address this central question, Applicant examined the proteomic expression profiles of anti-rsPilA NRel, anti-IHF NRel and planktonic NTHI (grown statically in broth to mid-log phase) by mass spectrometric analysis. The total proteomic expression profiles for anti-rsPilA and anti-IHF NRel were different from planktonic NTHI, as shown by the discrete locations of each population on the principal component analysis (PCA) plot (FIG. 3a). Moreover, although Applicant had anticipated that NRel population proteomic profiles would be different from planktonic cells (Brockson et al., 2014; Mokrzan et al., 2018), the two NRel populations were also very different from each other, as highlighted by the 95% confidence ellipses, despite genetic identity.

Due to the overall global changes in protein expression between the anti-rsPilA or anti-IHF NRel and planktonically grown NTHI, Applicant next examined proteins with differences in abundance between the two NRel populations, after each was first compared to planktonic NTHI. There was a total of 63 and 103 differentially expressed proteins (DEPs) with a significant >1.5-fold increase or decrease in abundance compared to planktonic NTHI (P<0.05) in anti-rsPilA NRel and anti-IHF NRel populations, respectively (FIG. 3b & Table 1). Moreover, anti-rsPilA NRel expressed 40 proteins and anti-IHF NRel expressed 80 proteins with uniquely significant differences in abundance compared to planktonic NTHI, which provided further evidence of the difference between the two NRel populations (FIG. 3b).

TABLE 1
Summary of proteins with significant (P < 0.05) 1.5-fold increase or decrease
in abundance of NRel versus planktonically grown NTHI or anti-IHF versus anti-rsPilA
comparisons.

| | | | | |
|---|---|---|---|---|
| COG category* | Protein^{†} | Functiont^{†} | Acc. No.^{δ} | Fc^{‡} |

anti-rsPilA NRel/Planktonic

| | | | | |
|---|---|---|---|---|
| Energy | ^{φ}TrxA | thioredoxin-like protein | AAX88118.1 | 5.1 |
| production & | | | | |
| conversion | | | | |
| | LctP | putative L-lactate permease | AAX88216.1 | 3.4 |
| | NdhA | NADH dehydrogenase | AAX87796.1 | 1.9 |
| | LldD | L-lactate dehydrogenase | AAX88789.1 | 1.8 |
| | FumC | fumarate hydratase class II | AAX88535.1 | −1.6 |
| | GpsA | glycerol-3-phosphate dehydrogenase | AAX87756.1 | −1.7 |
| | ^{φ}DsbE | thiol:disulfide interchange protein | AAX88104.1 | −2.3 |
| | NrfC | Fe-S-cluster-containing hydrogenase | AAX88077.1 | −2.3 |
| | | component 1 | | |
| | PpC | phosphoenolpyruvate carboxylase | AAX88226.1 | −2.5 |
| | NapA | periplasmic nitrate reductase | AAX87401.1 | −3.8 |
| | GlpC | anaerobic glycerol-3-phosphate | AAX87714.1 | −4.0 |
| | | dehydrogenase subunit C | | |
| | GlpA | anaerobic glycerol-3-phosphate | AAX87716.1 | −4.1 |
| | | dehydrogenase subunit A | | |
| Amino acid | TrpC | tryptophan biosynthesis protein | AAX88545.1 | 12.5 |
| transport & | | TrpCF | | |
| metabolism | | | | |
| | ^{δ}HisJ | probable amino-acid ABC | AAX88089.1 | 2.3 |
| | | transporter binding protein cystine | | |
| | | import | | |
| | TrpD | anthranilate | AAX88546.1 | 2.2 |
| | | phosphoribosyltransferase | | |
| | TrpA | tryptophan synthase alpha chain | AAX88491.1 | 1.9 |
| | TrpB | tryptophan synthase beta chain | AAX88492.1 | 1.8 |
| | NifS2 | cysteine desulfurase | AAX87433.1 | 1.6 |
| | NTHI1208 | Transglutaminase-like enzyme, | AAX88062.1 | 1.6 |
| | | putative cysteine protease | | |
| | AroK | shikimate kinase | AAX87264.1 | −1.5 |
| | ThrB | homoserine kinase | AAX87151.1 | −1.9 |
| | PepT | peptidase T | AAX88589.1 | −2.0 |
| | GlpB | anaerobic glycerol-3-phosphate | AAX87715.1 | −3.5 |
| | | dehydrogenase subunit B | | |
| Inorganic ion | Tbp1 | transferrin-binding protein 1 | AAX88031.1 | 9.4 |
| transport & | | | | |
| metabolism | | | | |
| | HfeB | putative ABC-type chelated iron | AAX87416.1 | 2.4 |
| | | transport system, ATPase | | |
| | | component | | |
| | TehB | tellurite resistance protein | AAX88653.1 | 2.3 |
| | HxuC | heme/hemopexin-binding protein C | AAX87321.1 | 2.1 |
| | HfeA | putative periplasmic chelated iron | AAX87417.1 | 2.1 |
| | | binding protein | | |
| | HitA | iron-utilization periplasmic protein | AAX87160.1 | 1.9 |
| | HktE | catalase | AAX87967.1 | 1.8 |
| | SodA | [Mn] superoxide dismutase | AAX88097.1 | 1.7 |
| | FtnB | ferritin like protein 2 | AAX88550.1 | −1.7 |
| | NrfA | cytochrome c552 | AAX88079.1 | −8.1 |
| | NrfB | cytochrome c-type protein | AAX88078.1 | −9.9 |
| Posttranslational | ^{φ}TrxA | thioredoxin-like protein | AAX88118.1 | 5.1 |
| modification, | | | | |
| protein turnover, | | | | |
| chaperones | | | | |
| | PdgX | peroxiredoxin/glutaredoxin | AAX87624.1 | 2.0 |
| | ClpB | ATP-dependent chaperone | AAX87905.1 | 1.7 |
| | OrfG | conserved hypothetical 21.9 kD | AAX87485.1 | 1.6 |
| | | protein in locus involved in | | |
| | | transformation | | |
| | RadA | DNA repair protein RadA homolog | AAX88271.1 | −1.8 |
| | ^{φ}DsbE | thiol:disulfide interchange protein | AAX88104.1 | −2.3 |
| | CcmA | heme exporter protein A | AAX88098.1 | −2.7 |
| Cell envelope | LpxC | UDP-3-O-[3-hydroxymyristoy1] N- | AAX88145.1 | 2.3 |
| biogenesis | | acetylglucosamine deacetylase | | |
| | LpsA | lipooligosaccharide glycosyl | AAX87813.1 | −1.5 |
| | | transferase | | |
| | FtsI | peptidoglycan synthetase | AAX88133.1 | −3.3 |
| | TolA | cell envelope integrity inner | AAX87438.1 | −4.0 |
| | | membrane protein | | |
| Coenzyme | NTHI0175 | conserved hypothetical protein | AAX87158.1 | 3.6 |
| metabolism | | SAM-dependent methyltransferase | | |
| | PdxH | pyridoxamine 5′-phosphate oxidase | AAX87909.1 | −1.6 |
| | MenC | O-succinylbenzoate synthase | AAX88008.1 | −2.7 |
| DNA replication, | RecN | DNA repair protein | AAX87073.1 | 1.7 |
| recombination, & | | | | |
| repair | | | | |
| | UvrB | UvrABC system protein B | AAX88673.1 | −1.6 |
| Translation, | RbfA | ribosome-binding factor A | AAX88605.1 | 1.5 |
| ribosomal | | | | |
| structure & | | | | |
| biogenesis | | | | |
| | PrfB | peptide chain release factor 2 | AAX88210.1 | −1.8 |
| Transcription | AsnC | regulatory protein AsnC | AAX87611.1 | 1.6 |
| | Sxy | DNA transformation protein TfoX | AAX87644.1 | −1.8 |
| Nucleotide | CyaA | Adenylate cyclase | AAX87755.1 | 2.3 |
| transport & | | | | |
| metabolism | | | | |
| Signal | ^{φ}HisJ | probable amino-acid ABC | AAX88089.1 | 2.3 |
| transduction | | transporter binding protein cystine | | |
| | | import | | |
| Intracellular | ExbD | transport protein | AAX87311.1 | 2.1 |
| trafficking | | | | |
| General function | NTHI1703 | conserved hypothetical | AAX88493.1 | 1.9 |
| prediction only | | oxidoreductase | | |
| | Hfq | host factor-I protein Hfq | AAX87465.1 | −1.7 |
| | NTHI0732 | predicted hydrolase of the HAD | AAX87648.1 | −1.9 |
| | | superfamily | | |
| | NTHI0053 | conserved hypothetical protein | AAX87048.1 | −1.9 |
| | NTHI0249 | conserved hypothetical protein | AAX87219.1 | −1.9 |
| Function | NTHI1214 | conserved putative gamma- | AAX88067.1 | 1.7 |
| unknown | | carboxymuconolactone | | |
| | | decarboxylase subunit | | |
| | NTHI1503 | hypothetical protein | AAX88314.1 | −1.6 |
| | NTHI0349 | conserved hypothetical protein | AAX87303.1 | −4.8 |

anti-IHF NRel/Planktonic

| | | | | |
|---|---|---|---|---|
| Translation, | RpsQ | 30S ribosomal protein S17 | AAX87834.1 | 2.8 |
| ribosomal | | | | |
| structure & | | | | |
| biogenesis | | | | |
| | NTHI0364 | conserved hypothetical protein | AAX87316.1 | 2.0 |
| | | ribosome-associated inhibitor A | | |
| | RpmE | 50S ribosomal protein L31 | AAX87806.1 | 1.9 |
| | RpsT | 30S ribosomal protein S20 | AAX88005.1 | 1.8 |
| | RpsJ | 30S ribosomal protein S10 | AAX87824.1 | 1.7 |
| | RplO | 50S ribosomal protein L15 | AAX87845.1 | 1.6 |
| | RplU | 50S ribosomal protein L21 | AAX87919.1 | 1.6 |
| | RpsE | 30S ribosomal protein S5 | AAX87843.1 | 1.6 |
| | RpsL | 30S ribosomal protein S12 | AAX87659.1 | 1.6 |
| | InfB | translation initiation factor IF-2 | AAX88610.1 | −1.6 |
| | RplW | 50S ribosomal protein L23 | AAX87827.1 | −2.0 |
| | InfA | translation initiation factor IF-1 | AAX87597.1 | −4.9 |
| | TruA | tRNA pseudouridine synthase A | AAX88218.1 | −5.7 |
| | PhoB | phosphate regulon transcriptional | AAX88556.1 | −5.8 |
| | | regulatory protein | | |
| Inorganic ion | HktE | catalase | AAX87967.1 | 2.0 |
| transport & | | | | |
| metabolism | | | | |
| | HgpB | hemoglobin-haptoglobin binding | AAX87692.1 | 2.0 |
| | | protein B | | |
| | HfeB | putative ABC-type chelated iron | AAX87416.1 | 2.0 |
| | | transport system, ATPase | | |
| | | component | | |
| | HfeA | putative periplasmic chelated iron | AAX87417.1 | 1.9 |
| | | binding protein | | |
| | ZnuA | high-affinity zinc uptake system | AAX87184.1 | 1.6 |
| | | protein | | |
| | TehB | tellurite resistance protein | AAX88653.1 | 1.5 |
| | CitT | citrate/succinate antiporter | AAX87023.1 | −1.7 |
| | Fur | ferric uptake regulation protein | AAX87247.1 | −1.7 |
| | NTHI1439 | conserved hypothetical phosphate | AAX88259.1 | −1.8 |
| | | transport regulator | | |
| | NrfB | cytochrome c-type protein | AAX88078.1 | −2.4 |
| | NrfA | cytochrome c552 | AAX88079.1 | −2.7 |
| Posttranslational | ^{φ}CydD | hypothetical ABC transporter, ATP- | AAX87693.1 | 2.4 |
| modification, | | binding protein | | |
| protein turnover, | | | | |
| chaperones | | | | |
| | PdgX | peroxiredoxin/glutaredoxin | AAX87624.1 | 1.8 |
| | GroES | 10 kDa chaperonin | AAX87591.1 | 1.8 |
| | TrxA | Thioredoxin | AAX87086.1 | 1.7 |
| | ClpB | ATP-dependent chaperone | AAX87905.1 | 1.6 |
| | Dj1A | DnaJ-like protein | AAX87329.1 | −1.8 |
| | PpiB | peptidyl-prolyl cis-trans isomerase B | AAX87081.1 | −1.9 |
| | ^{φ}PUreG | urease accessory protein UreG | AAX87585.1 | −2.0 |
| | ^{φ}DsbE | thiol:disulfide interchange protein | AAX88104.1 | −7.8 |
| Energy | LctP | putative L-lactate permease | AAX88216.1 | 2.8 |
| production & | | | | |
| conversion | | | | |
| | ^{φ}CydD | hypothetical ABC transporter, ATP- | AAX87693.1 | 2.4 |
| | | binding protein | | |
| | DlD | D-lactate dehydrogenase | AAX88704.1 | 2.1 |
| | PpC | phosphoenolpyruvate carboxylase | AAX88226.1 | −1.9 |
| | GlpA | anaerobic glycerol-3-phosphate | AAX87716.1 | −2.0 |
| | | dehydrogenase subunit A | | |
| | FdhX | formate dehydrogenase major | AAX87009.1 | −2.1 |
| | | subunit | | |
| | AtpH | ATP synthase delta chain | AAX87536.1 | −2.2 |
| | CitD | citrate lyase acyl carrier protein | AAX87027.1 | −3.3 |
| | ^{φ}DsbE | thiol:disulfide interchange protein | AAX88104.1 | −7.8 |
| Cell envelope | TonB | Periplasmic protein TonB, links | AAX87310.1 | 2.0 |
| biogenesis | | inner and outer membrane | | |
| | Pal | outer membrane protein P6 | AAX87436.1 | 1.6 |
| | | precursor | | |
| | OmpP2 | outer membrane protein P2 | AAX87199.1 | 1.5 |
| | | precursor | | |
| | LicC | phosphocholine cytidylyltransferase | AAX88398.1 | −1.8 |
| | LolA | outer-membrane lipoproteins carrier | AAX88274.1 | −2.0 |
| | | protein precursor | | |
| | FtsI | peptidoglycan synthetase | AAX88133.1 | −3.0 |
| | Lic2A | UDP-Gal--lipooligosaccharide | AAX87599.1 | −3.0 |
| | | galactosyltransferase | | |
| | LicD | phosphorylcholine transferase | AAX88397.1 | −5.7 |
| | NTHI1085 | predicted membrane bound zinc | AAX87956.1 | −6.0 |
| | | metalloprotease with PDZ domain | | |
| Amino acid | ^{φ}HisJ | probable amino-acid ABC | AAX88089.1 | 1.7 |
| transport & | | transporter binding protein cystine | | |
| metabolism | | import | | |
| | NTHI1208 | conserved hypothetical protein | AAX88062.1 | 1.5 |
| | | Transglutaminase-like enzyme, | | |
| | | putative cysteine protease | | |
| | OppF | oligopeptide transport ATP-binding | AAX88123.1 | −1.8 |
| | | protein | | |
| | GlpB | anaerobic glycerol-3-phosphate | AAX87715.1 | −2.0 |
| | | dehydrogenase subunit B | | |
| | AroK | shikimate kinase | AAX87264.1 | −2.2 |
| | ArtP | arginine transport ATP-binding | AAX88179.1 | −3.5 |
| | | protein | | |
| | AroE | shikimate 5-dehydrogenase | AAX87686.1 | −8.7 |
| Coenzyme | BioB | Biotin synthase | AAX88047.1 | −1.6 |
| metabolism | | | | |
| | RibA | GTP cyclohydrolase II | AAX87269.1 | −2.0 |
| | PdxH | pyridoxamine 5′-phosphate oxidase | AAX87909.1 | −2.6 |
| | CoaD | phosphopantetheine | AAX87681.1 | −3.7 |
| | | adenylyltransferase | | |
| | menC | O-succinylbenzoate synthase | AAX88008.1 | −4.3 |
| | BioF | 8-amino-7-oxononanoate synthase | AAX88384.1 | −5.3 |
| DNA replication, | RecN | DNA repair protein | AAX87073.1 | 1.9 |
| recombination, & | | | | |
| repair | | | | |
| | PolA | DNA polymerase I | AAX87902.1 | −1.7 |
| | DnaE | DNA polymerase III alpha subunit | AAX87791.1 | −2.0 |
| | RelB | DNA-damage-inducible protein J, | AAX87739.1 | −5.3 |
| | | negative regulator of translation | | |
| | DnaQ | DNA polymerase III, epsilon chain | AAX87197.1 | −5.8 |
| Transcription | ^{φ}UreG | urease accessory protein UreG | AAX87585.1 | −2.0 |
| | RpoZ | DNA-directed RNA polymerase | AAX88793.1 | −2.1 |
| | | omega chain | | |
| | GreA | transcription elongation factor GreA | AAX88453.1 | −2.2 |
| | RpoE | RNA polymerase sigma-E factor | AAX87635.1 | −3.3 |
| Lipid metabolism | PlsC | 1-acyl-sn-glycerol-3-phosphate | AAX87785.1 | −1.8 |
| | | acyltransferase | | |
| | TesB | Acyl-CoA thioesterase II | AAX87079.1 | −4.5 |
| | IspD | 2-C-methyl-D-erythritol 4- | AAX87703.1 | −5.0 |
| | | phosphate cytidylyltransferase | | |
| | IspF | 2-C-methyl-D-erythritol 2,4- | AAX87702.1 | −8.7 |
| | | cyclodiphosphate synthase | | |
| Signal | ^{φ}HisJ | probable amino-acid ABC | AAX88089.1 | 1.7 |
| transduction | | transporter binding protein cystine | | |
| | | import | | |
| | NTHI1437 | conserved hypothetical protein, SH3 | AAX88257.1 | −2.3 |
| | | domain | | |
| | DksA | DnaK suppressor protein | AAX87065.1 | −3.3 |
| | RseA | sigma-E factor negative regulatory | AAX87634.1 | −6.5 |
| | | protein homolog | | |
| Intracellular | ExbD | transport protein | AAX87311.1 | 2.3 |
| trafficking & | | | | |
| secretion | | | | |
| | TolB | Tol-Pal system protein TolB | AAX87437.1 | 1.7 |
| Cell division & | FtsE | cell division ATP-binding protein | AAX87817.1 | −3.6 |
| chromosome | | | | |
| partitioning | | | | |
| General function | NTHI0052 | conserved hypothetical FtsH- | AAX87047.1 | 5.2 |
| prediction only | | interacting integral membrane | | |
| | | protein | | |
| | NTHI0043 | conserved hypothetical membrane | AAX87038.1 | 1.6 |
| | | protein | | |
| | NTHI1199 | predicted ATPase | AAX88055.1 | −1.7 |
| | UuaP | ABC transporter ATP-binding | AAX88594.1 | −1.8 |
| | | protein | | |
| | Hfq | host factor-I protein Hfq | AAX87465.1 | −1.8 |
| | AbgA | aminobenzoyl-glutamate utilization | AAX87654.1 | −1.9 |
| | | protein A | | |
| | NTHI1590 | putative NAD(P)H oxidoreductase | AAX88393.1 | −2.0 |
| | NTHI0779 | probable ABC transporter, ATP- | AAX87689.1 | −2.5 |
| | | binding protein | | |
| | NTHI0053 | conserved hypothetical protein | AAX87048.1 | −7.0 |
| Function | NTHI0490 | conserved hypothetical protein | AAX87425.1 | −1.6 |
| unknown | | Putative negative regulator of RcsB- | | |
| | | dependent stress response | | |
| | NTHI0820 | conserved hypothetical protein | AAX87724.1 | −1.7 |
| | | Autotransporter translocation and | | |
| | | assembly factor TamB | | |
| | NTHI0487 | conserved hypothetical | AAX87422.1 | −2.1 |
| | | transcriptional regulator with an N- | | |
| | | terminal xre-type HTH domain | | |
| | NTHI0555 | conserved hypothetical protein | AAX87483.1 | −3.4 |
| | NTHI1025 | conserved hypothetical protein Cell | AAX87903.1 | −3.5 |
| | | division protein ZapA | | |
| | NTHI0436 | conserved hypothetical protein | AAX87377.1 | −5.1 |
| | | isoprenylcysteine carboxyl | | |
| | | methyltransferase | | |
| | NTHI1748 | conserved hypothetical protein | AAX88534.1 | −5.2 |
| | | Chalcone isomerase-like | | |
| | NTHI0319 | conserved hypothetical protein | AAX87278.1 | −10.2 |
| | | membrane protein YkgB | | |

anti-IHF NRel/anti-rsPilA NRel

| | | | | |
|---|---|---|---|---|
| Translation, | ^{φ}DeaD | Cold-shock DEAD-box protein A | AAX87292.1 | 2.2 |
| ribosomal | | homolog | | |
| structure & | | | | |
| biogenesis | | | | |
| | RpsT | 30S ribosomal protein S20 | AAX88005.1 | 1.9 |
| | NTHI1369 | putative translation factor, SUA5 | AAX88196.1 | 1.9 |
| | RpsQ | 30S ribosomal protein S17 | AAX87834.1 | 1.8 |
| | RplU | 50S ribosomal protein L21 | AAX87919.1 | 1.6 |
| | RpsE | 30S ribosomal protein S5 | AAX87843.1 | 1.5 |
| | RpsL | 30S ribosomal protein S12 | AAX87659.1 | 1.5 |
| | InfA | translation initiation factor IF-1 | AAX87597.1 | −5.5 |
| | PhoB | phosphate regulon transcriptional | AAX88556.1 | −5.5 |
| | | regulatory protein | | |
| Cell envelope | MurB | UDP-N- | AAX87327.1 | 1.9 |
| biogenesis | | acetylenolpyruvoylglucosamine | | |
| | | reductase | | |
| | Pal | outer membrane protein P6 | AAX87436.1 | 1.6 |
| | | precursor | | |
| | NTHI0291 | putative small-conductance | AAX87252.1 | −1.5 |
| | | mechanosensitive channel: | | |
| | | potassium efflux protein KefA | | |
| | LicD | phosphorylcholine transferase | AAX88397.1 | −4.8 |
| Transcription | ^{φ}DeaD | Cold-shock DEAD-box protein A | AAX87292.1 | 2.2 |
| | | homolog | | |
| | GreA | transcription elongation factor GreA | AAX88453.1 | −2.2 |
| | AsnC | regulatory protein AsnC | AAX87611.1 | −1.6 |
| DNA replication, | ^{φ}DeaD | Cold-shock DEAD-box protein A | AAX87292.1 | 2.2 |
| recombination, & | | homolog | | |
| repair | | | | |
| | RelB | DNA-damage-inducible protein J, | AAX87739.1 | −4.9 |
| | | negative regulator of translation | | |
| | UnG | uracil-DNA glycosylase | AAX87021.1 | −4.9 |
| Inorganic ion | NrfA | cytochrome c552 | AAX88079.1 | 3.0 |
| transport & | | | | |
| metabolism | | | | |
| | TehB | tellurite resistance protein | AAX88653.1 | −1.5 |
| | HugZ | putative heme iron utilization | AAX87900.1 | −1.9 |
| | | protein | | |
| Energy | ^{φ}CydD | hypothetical ABC transporter, ATP- | AAX87693.1 | 3.6 |
| production & | | binding protein | | |
| conversion | | | | |
| | GlpC | anaerobic glycerol-3-phosphate | AAX87714.1 | 2.8 |
| | | dehydrogenase subunit C | | |
| | DmsB | anaerobic dimethyl sulfoxide | AAX88060.1 | 1.5 |
| | | reductase chain B | | |
| Posttranslational | ^{φ}CydD | hypothetical ABC transporter, ATP- | AAX87693.1 | 3.6 |
| modification, | | binding protein | | |
| protein turnover, | | | | |
| chaperones | | | | |
| | Dj1A | DnaJ-like protein | AAX87329.1 | −2.0 |
| Lipid metabolism | PlsC | 1-acyl-sn-glycerol-3-phosphate | AAX87785.1 | −1.9 |
| | | acyltransferase | | |
| | IspF | 2-C-methyl-D-erythritol 2,4- | AAX87702.1 | −8.6 |
| | | cyclodiphosphate synthase | | |
| Coenzyme | PanF | sodium/pantothenate symporter | AAX88013.1 | −1.5 |
| metabolism | | | | |
| | RibA | GTP cyclohydrolase II | AAX87269.1 | −2.1 |
| | CoaD | phosphopantetheine | AAX87681.1 | −3.2 |
| | | adenylyltransferase | | |
| Nucleotide | GmK | guanylate kinase | AAX88794.1 | −1.5 |
| transport & | | | | |
| metabolism | | | | |
| | CmK | cytidylate kinase | AAX88701.1 | −2.0 |
| | PurU | formyltetrahydrofolate deformylase | AAX88279.1 | −2.0 |
| | DcD | deoxycytidine triphosphate | AAX87193.1 | −2.3 |
| | | deaminase | | |
| | CyaA | adenylate cyclase | AAX87755.1 | −4.7 |
| | OppF | oligopeptide transport ATP-binding | AAX88123.1 | −1.5 |
| | | protein | | |
| | TrpB | tryptophan synthase beta chain | AAX88492.1 | −1.8 |
| | HisI | histidine biosynthesis bifunctional | AAX87529.1 | −3.0 |
| | | protein HisIE | | |
| | TrpC | tryptophan biosynthesis protein | AAX88545.1 | −3.6 |
| | | TrpCF | | |
| | TrpD | anthranilate | AAX88546.1 | −5.2 |
| | | phosphoribosyltransferase | | |
| | AroE | shikimate 5-dehydrogenase | AAX87686.1 | −8.3 |
| Intracellular | SecF | protein-export membrane protein | AAX87299.1 | −2.0 |
| trafficking & | | SecF | | |
| secretion | | | | |
| Secondary | FabG | putative oxidoreductase | AAX87052.1 | −1.5 |
| metabolite | | | | |
| biosynthesis, | | | | |
| transport, & | | | | |
| catabolism | | | | |
| Cell division & | FtsE | cell division ATP-binding protein | AAX87817.1 | −3.7 |
| chromosome | | | | |
| partitioning | | | | |
| General function | NTHI0052 | conserved hypothetical FtsH- | AAX87047.1 | 4.8 |
| prediction only | | interacting integral membrane | | |
| | | protein | | |
| | NTHI0043 | conserved hypothetical membrane | AAX87038.1 | 2.0 |
| | | protein | | |
| | NTHI1199 | predicted ATPase | AAX88055.1 | −1.6 |
| | NTHI0779 | probable ABC transporter, ATP- | AAX87689.1 | −2.4 |
| | | binding protein | | |
| Function | NTHI0487 | conserved hypothetical | AAX87422.1 | −2.0 |
| unknown | | transcriptional regulator with an N- | | |
| | | terminal xre-type HTH domain | | |
| | NTHI0319 | conserved hypothetical protein | AAX87278.1 | −6.0 |
| | | membrane protein YkgB | | |

^{*}Clusters of Orthologous Groups of proteins
^{δ}NCBI GenBank Protein Accession Number (Clark, K. 2016 D67)
^{†}Annotation from NCBI GenBank protein accession number (Clark, K. 2016 D67) or annotation from Aziz, A. 2019 p1622
^{‡}Fc = Significant (P < 0.05) fold change, (−) fold decrease
^{φ}Within more than one COG category

Applicant next annotated the DEPs by Clusters of Orthologous Groups of proteins (COG) (Aziz et al., 2019; Tatusov et al., 2000) to assess the relative functions of the two NRel DEPs as a result of release from biofilm residence via distinct antibody-mediated dispersal versus disruption. For the anti-rsPilA NRels, annotation of the 63 DEPs revealed that the most frequently represented COG categories (36.5%) were involved in either energy production & conversion (19.0%) or amino acid transport & metabolism (17.5%) (FIG. 3c, anti-rsPilA bars & Table 1). Specific to energy production & conversion category, enzymes involved in glycolysis, tricarboxylic acid cycle, nitrogen metabolism, and anaerobic metabolism of glycerol were >1.5-fold decreased in abundance compared to planktonic NTHI. Whereas proteins involved in lactate uptake and utilization were >1.5 fold increased in abundance compared to planktonic NTHI (Table 1). In the same category, DEPs specific to anti-rsPilA NRel with >1.5-fold increase included tryptophan biosynthesis and cysteine metabolism enzymes. The collective differences in protein functional categories indicated that anti-rsPilA NRel were primarily in an active-adaptive energy utilization and amino acid metabolic state.

In contrast, within the anti-IHF NRel population, ‘translation, ribosomal structure & biogenesis’ was the most frequently represented COG category (13.6%) among the 103 DEPs, compared to planktonic NTHI (FIG. 3c, anti-IHF bars & Table 1). Specific to anti-IHF NRel DEPs, eight were 30S and 50S ribosomal proteins with >1.5-fold increase, and although ribosomal structural proteins were also increased, two translation initiation factors were decreased >1.5-fold (Table 1). Further, expression of each of the cell envelope biogenesis, coenzyme metabolism, and lipid metabolism COG category proteins were >1.5-fold decreased compared to planktonic NTHI, for example, Lic2A, LicC, and LicD, proteins responsible for modification of lipooligosaccharide (LOS) and decoration with a phosphorylcholine moiety (Poole et al., 2013; Swords et al., 2003), and the lipoprotein carrier protein LolA, which shuttles lipoproteins from the inner membrane to the outer membrane (Kaplan et al., 2018). Moreover, there were four anti-IHF NRel DEPs within the lipid metabolism category which were significantly >1.5-fold decreased in expression compared to planktonic NTHI (FIG. 3c). Additionally, coenzyme metabolism proteins required for biosynthesis of biotin, a cofactor in fatty acid biosynthesis, were also significantly >1.5-fold decreased in expression (Parsons and Rock, 2013). In contrast, the outer membrane lipoprotein OMP P6, involved in maintenance of outer membrane integrity and attachment to peptidoglycan, and the major outer membrane protein OMP P2 were significantly increased in expression compared to planktonic NTHI (Table 1) (Murphy et al., 2006; Sikkema and Murphy, 1992). Collectively, these data suggested that anti-IHF NRel contained abundant ribosomes for translation of proteins, however translation was limited due to the reduced translation initiation factor proteins. Further, anti-IHF NRel demonstrated decreased expression of LOS-modifying enzymes and lipid metabolism genes, with a concurrent increase in expression of outer membrane integrity maintenance proteins, which suggested differences in membrane composition of anti-IHF NRel compared to planktonically grown NTHI.

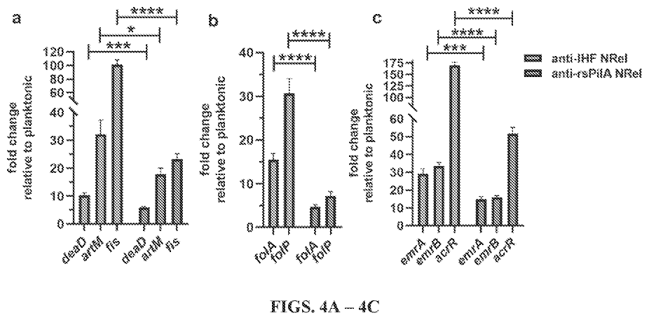

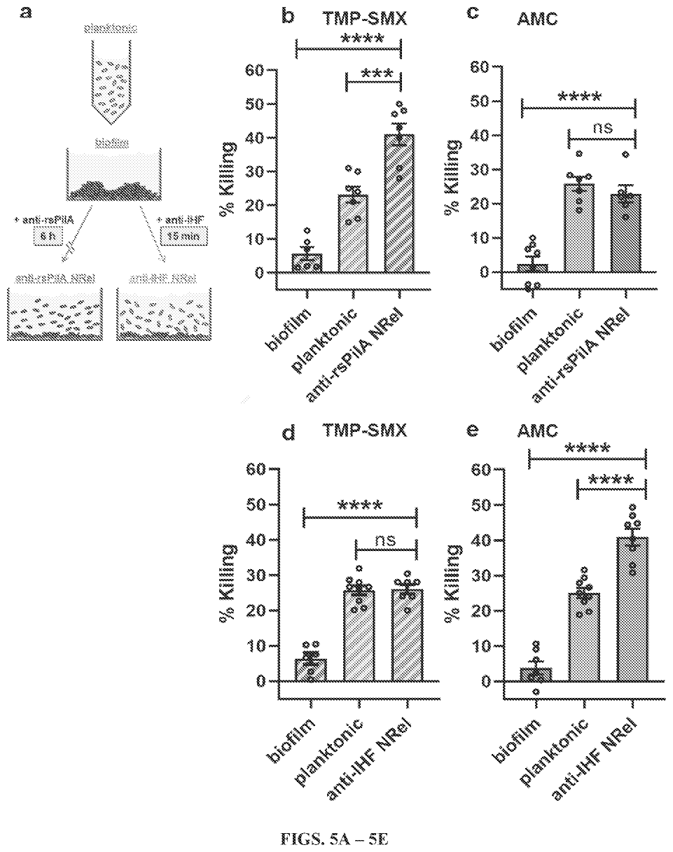

The increased abundance of ribosomal proteins observed in the anti-IHF NRel proteomic profile is also characteristic of bacteria in lag phase of growth (Rolfe et al., 2012). To determine whether anti-IHF NRel showed other similarities with lag phase bacteria, Applicant used qRT-PCR to examine the expression of three genes canonically expressed by bacteria in lag phase (Martinez-Antonio and Collado-Vides, 2003). Expression of deaD, artM, and fis was significantly (>2-fold) upregulated in anti-IHF NRel vs. planktonic NTHI (FIG. 4a). Interestingly, for all three of these genes, the fold increase in transcript abundance over planktonic NTHI was significantly greater for anti-IHF vs. anti-rsPilA NRel (P<0.05). Thus, that anti-IHF NRel appeared to be released from biofilm residence in a state which mimicked lag phase, presented another significant difference between the anti-IHF and anti-rsPilA phenotypes (Table 2), and again suggested that physical collapse of the biofilm structure resulted in rapid release while NTHI was metabolically more quiescent than anti-rsPilA NRel.

Because the proteomic expression profiles of the two NRel were different from each other (FIG. 3a), Applicant next conducted a direct comparison of DEPs between the two NRel populations as depicted by volcano plot (FIG. 3d). Fifty-one DEPs with >1.5-fold increase or decrease were identified amongst anti-IHF NRel versus anti-rsPilA NRel (FIG. 3d & Table 1). Of these, 15 proteins (29.4%) demonstrated a >1.5-fold increase in expression by anti-IHF NRel compared to anti-rsPilA NRel, and these proteins included five of the 30S and 50S ribosomal proteins described prior. Also, OMP P6 was more abundant in anti-IHF NRel compared to anti-rsPilA NRel. DEPs with a >1.5-fold decrease in the anti-IHF NRel compared to anti-rsPilA NRel (e.g. greater abundance in anti-rsPilA NRel) were characteristic of the abundantly expressed tryptophan biosynthesis proteins. These distinctions were in addition to relative differences in lipid metabolism proteins that had already been identified as decreased in expression in the anti-IHF NRel population compared to planktonic NTHI. Notably, this direct comparison of the two NRel proteomic expression profiles also revealed a significant increase in the peptidoglycan synthesis protein, MurB (Nikolaidis et al., 2014), within the anti-IHF NRel DEPs compared to anti-rsPilA NRel, which further suggested the altered cell envelope composition of the anti-IHF NRels (Table 1).

Anti-rsPilA or Anti-IHF NRel were Significantly More Sensitive to Killing by a Specific Antibiotic than Planktonic NTHI

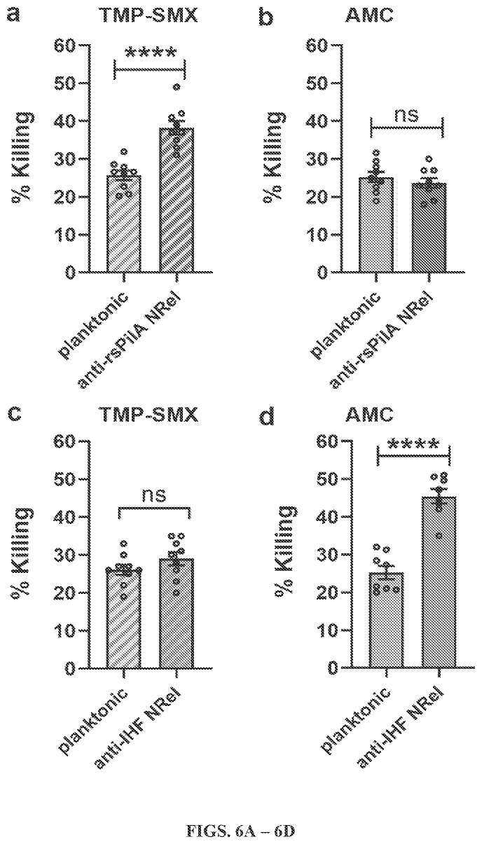

With the observed significant differences in relative expression of distinct proteins between the two NRel populations demonstrated, Applicant next examined how these differences altered phenotypic character. As bacteria newly released from a biofilm are typically more sensitive to killing by antibiotics than their planktonic counterparts (Brockson et al., 2014; Chambers et al., 2017; Goodwine et al., 2019; Mokrzan et al., 2018), Applicant assessed the sensitivity of NRel NTHI to TMP-SMX or to AMC, as these represent antibiotics commonly prescribed for NTHI-induced OM and respiratory infections (Harrison et al., 2009; K et al., 2017; Pelton, 2020; Sethi and Murphy, 2008; Tristram et al., 2007; Wald and DeMuri, 2018; Wasserman and Gerber, 2017). Applicant compared the anti-rsPilA NRel and anti-IHF NRel susceptibilities to killing by TMP-SMX or AMC, to that of both biofilm-resident NTHI (canonically highly resistant) and to planktonic NTHI grown to mid-log phase of growth (canonically sensitive and representative of the population commonly used to determine MIC values in clinical microbiology laboratories) (FIG. 5a). To control for differences in the numbers of NTHI released by exposure to anti-IHF IgG for 15 min or by anti-rsPilA IgG for 6 h (see FIG. 1), Applicant adjusted the density of the planktonic NTHI in each experiment to match that of either the anti-IHF or anti-rsPilA NRel population. After Applicant identified the concentrations of AMC or TMP-SMX needed to reproducibly kill ˜25% of planktonic NTHI at each bacterial density, Applicant then used these concentrations to assess relative killing of the corresponding NRel or biofilm-resident NTHI.

As expected, anti-rsPilA NRel were significantly more sensitive than biofilm-resident NTHI to killing by either TMP-SMX or AMC (P≤0.0001) (FIGS. 5b& 5c). Notably however, sensitivity of anti-rsPilA NRel to killing by TMP-SMX was significantly greater compared to that for planktonic NTHI (P≤0.001) after only 2 h of antibiotic exposure (FIG. 5b). In contrast, anti-rsPilA NRel were only equally as sensitive as their planktonic counterparts to killing by the β-lactam antibiotic AMC (FIG. 5c). Applicant then similarly evaluated anti-IHF NRel and found that they too were significantly more sensitive than biofilm-resident NTHI to killing by either TMP-SMX or AMC (P≤0.0001), again as expected (FIGS. 5d& 5e). Intriguingly however, and in direct contrast to anti-rsPilA NRel, the anti-IHF NRel population was only equally as sensitive to TMP-SMX mediated killing as their planktonic counterparts (FIG. 5d), but significantly more sensitive to AMC (P≤0.0001) (FIG. 5e).

Bacterial sensitivity to antibiotic killing is the result of multiple processes that include drug uptake, efflux, and degradation, as well as the direct mechanism of antibiotic action (Kapoor et al., 2017). To identify possible mechanisms for the selectively enhanced antibiotic sensitivities of anti-IHF or anti-rsPilA NRel, Applicant used qRT-PCR to examine the relative expression of several genes likely to play a role in susceptibility or resistance to TMP-SMX or AMC. The protein targets of TMP and SMX, dihydrofolate reductase and dihydropteroate synthetase, are encoded by folA and folP, respectively; overproduction of FolA and FolP is associated with resistance to TMP-SMX (Huovinen, 2001). Accordingly, Applicant speculated that anti-rsPilA NRel NTHI would likely demonstrate less relative expression of folA and/or folP than anti-IHF NRels at the selected time points. Applicant's results confirmed this hypothesis, as folA and folP expression were both significantly reduced in anti-rsPilA vs. anti-IHF NRel (FIG. 4b, P<0.0001).

Efflux pumps enable bacteria to decrease the concentration of intracellular antibiotic. The EmrE efflux system transports β-lactam antibiotics in Neisseria gonorrhoeae (Du et al., 2018), and in E. coli, TMP-SMX exposure stimulates expression of the EmrAB efflux pump (Barrero et al., 2014), which suggested that the EmrAB efflux pump could influence NRel sensitivity to TMP-SMX. Applicant found that relative expression of emrA and emrB by anti-rsPilA NRel was significantly less that by anti-IHF NRel, consistent with the greater sensitivity of anti-rsPilA NRel to TMP-SMX (FIG. 4c, P<0.001). In contrast, the ArcAB efflux pump can transport β-lactam antibiotics and is under the control of the transcriptional repressor acrR (Anes et al., 2015). While enhanced AcrAB expression via acrR mutations has been linked to amoxicillin resistance in Haemophilus (aac.asm.org/content/51/7/2564.long), Applicant argued that enhanced expression of acrR would likely have the opposite effect, enhanced susceptibility of NTHI to AMC. As Applicant anticipated, relative acrR expression was significantly greater by anti-IHF vs. anti-rsPilA NRel, consistent with the heightened sensitivity to AMC observed for anti-IHF NRel (FIG. 4c, P<0.0001).

Taken together, the antibiotic sensitivity and transcript abundance data suggested that anti-rsPilA and anti-IHF NRel differed significantly in their relative antibiotic sensitivities due to the mechanism by which they were released from biofilm residence. However, Applicant were concerned that the difference in time required for maximal disruption (minutes) vs. dispersal (hours) might have also played a role in the observed phenotype. Thereby, Applicant repeated the analysis of relative antibiotic sensitivities on time-matched NRel populations recovered after incubation with either anti-rsPilA or anti-IHF IgG for 2 h, a timepoint approximately midway between the times of maximal release for both antibodies. Similar to the results shown in FIG. 5, anti-rsPilA NRel were significantly more susceptible to killing by TMP-SMX (P≤0.0001) (FIG. 6a), and equally susceptible to killing by AMC, as planktonic NTHI (FIG. 6b). Moreover, time-matched anti-IHF NRel were significantly more susceptible to killing by AMC (P≤0.0001) (FIG. 6c), and equally susceptible to killing by TMP-SMX (FIG. 6d), compared to planktonic NTHI. These results provided further support for the hypothesis that the distinct antibiotic sensitivity phenotypes shown resulted from the two different mechanisms of release, dispersal vs. disruption.

Collectively, these data added additional phenotypic distinctions between anti-rsPilA and anti-IHF NRel, wherein these two genetically identical populations exhibited not only distinctive proteomic and targeted transcriptomic expression profiles but also revealed that they were released from biofilm residence in distinct phases of growth, release as either aggregates vs. as individual cells and demonstrated significantly increased but different susceptibilities to killing by either a sulfonamide or a β-lactam antibiotic when compared to planktonic NTHI, both of which are first-line antibiotics recommended for treatment of NTHI-induced diseases.

### DISCUSSION

Historically, the development paths for vaccines and those for antibiotics have proceeded in parallel, with one focused on prevention and the other on treatment (Tagliabue and Rappuoli, 2018). Whereas this strategy has indeed been successful for many diseases, there is still the issue of multiple chronic and recurrent infections for which neither path has yet achieved overall success. There are widely acknowledged obstacles to progress in this regard, none the least of which is the typically inherently slow vaccine development process (Koff and Schenkelberg, 2019). Similarly, there are tremendous challenges to the antibiotic development pathway, with no clinically approved truly new class of drug introduced for >30 years, despite extensive effort necessitated by a worrisome rapid increase in the rise of multi-antibiotic resistant bacteria worldwide (2017a; Ribeiro da Cunha et al., 2019). These obstacles to progress are understandable given the complex and difficult-to-treat nature of chronic diseases, which has confounded both discovery pathways. Such persistent and recurrent infections are attributable to causative agents that form biofilms wherein the resident bacteria have a unique transcriptome and a highly recalcitrant phenotype that renders them resistant to antibiotics and host immune effectors that readily kill their planktonic counterparts (Ahearn et al., 2017; Hall and Mah, 2017; Pang et al., 2012; Sharma et al., 2019; Silva and Sillankorva, 2019; Stewart, 2002). The advances in the recognition and understanding of the NRel phenotype now provide us with the opportunity to merge aspects of these development pathways to consider the use of therapeutic antibodies to release bacteria from the recalcitrant biofilm-residence into a state that is now markedly more vulnerable to killing.

In the previous studies, Applicant showed that established NTHI biofilms concurrently exposed to both antiserum against a bacterial DNABII protein and an antibiotic were significantly more sensitive to killing by three antibiotics commonly used to treat OM (e.g. ampicillin, AMC and cefdinir) at concentrations ≥4-fold below the MIC, compared to their planktonic counterparts (Brockson et al., 2014). Applicant also showed that both NRel NTHI and NRel M. catarrhalis are more susceptible to TMP-SMX or clarithromycin, respectively, when released from a dual-species biofilm by incubation with anti-rsPilA, than those from growth on agar (Mokrzan et al., 2018). Given Applicant's previous observations, here Applicant aimed to begin to understand the relative phenotypes of the two NRel populations as induced by the specifically targeted anti-rsPilA and anti-IHF sera, which release NTHI from biofilm residence by discrete mechanisms as this could have a notable influence on relative clinical approach to best mediate eradication of the resultant NRel population.

In this study, Applicant characterized anti-rsPilA and anti-IHF NRel and showed that their relative proteomic expression profiles, relative expression of targeted genes and sensitivity to killing by antibiotics were distinct not only from planktonically grown NTHI, but importantly, also from each other. Although the kinetics of release by anti-IHF-mediated disruption vs. anti-rsPilA-mediated dispersal are clearly different, Applicant's results strongly suggest that the observed differences in NRel phenotype were dependent on the specific antibody that mediated release from biofilm residence. The proteomic expression profiles provided a snapshot in time of the total released NRel populations. The anti-rsPilA NRel population proteomic profile was defined by an adaptive state of energy metabolism & conversion and amino acid transport & metabolism in response to their being induced to actively disperse from the biofilm. This adaptive metabolic state is likely somewhat more heterogenous than anti-IHF NRel NTHI due to the more gradual release of cells as they actively disperse from the biofilm due to expression of both AI-2 and the Type IV twitching pilus over the 6 h incubation period. Nonetheless, the adaptive metabolic state of this anti-rsPilA NRel population was very similar to that described for other genera (e.g. S. pneumoniae, K. pneumoniae, and P. aeruginosa) in response to release from a biofilm and thereby does not appear to be atypical (Guilhen et al., 2016; Pettigrew et al., 2014; Rumbaugh and Sauer, 2020).

In analysis of the anti-IHF NRel population, Applicant found that it was defined by an increased production of ribosomal proteins with a concurrent decrease in LOS modification, cell membrane maintenance and lipid metabolism proteins. The enrichment of ribosomal proteins suggested that the anti-IHF NRel population was poised for protein synthesis with an altered membrane composition in response to rapid passive release en masse into the surrounding milieu. As such, anti-IHF NRel might be expected to still largely resemble biofilm-resident NTHI bacteria. Indeed, targeted transcriptomics indicated that anti-IHF NRel were in a state of growth similar to lag phase. Nonetheless, the anti-IHF NRel phenotype was also clearly different from that of biofilm-resident NTHI, as revealed by their significantly greater killing by both sulfonamide and β-lactam antibiotics compared to biofilm-resident NTHI.

As expected from Applicant's previous work (Brockson et al., 2014; Mokrzan et al., 2018), NRel NTHI were highly sensitive to two first-line antibiotics prescribed to treat NTHI-induced diseases. However, here Applicant showed for the first time that these NRel populations displayed unique enhanced sensitivity to killing by a different class of antibiotic dependent on whether they had been released from biofilm residence by either dispersal (e.g. via anti-rsPilA) or disruption (e.g. via anti-IHF). These differences cannot be explained by a direct effect of either anti-rsPilA or anti-IHF on NRel NTHI, since neither NTHI viability or susceptibility to killing by either of these antibiotics is affected by incubation with either antibody (Brockson et al., 2014; Mokrzan et al., 2018). Collectively, the two resultant NRel populations showed significant differences in relative proteomic expression profiles, targeted transcriptomic profiles, character of release from biofilm residence (both growth phase and as single cells vs. aggregates), and antibiotic sensitivities.

The anti-rsPilA NRel adaptive amino acid transport & metabolism state provided insight into the mechanism of this population's uniquely increased susceptibility to TMP-SMX, because the sulfonamide class of antibiotic targets the folic acid synthesis pathway involved in amino acid synthesis (Fernandez-Villa et al., 2019). Also, compared to anti-IHF NRel, the lower relative expression of folA and folP, which encode the protein targets for TMP and SMX, respectively, together with lower expression of emrA and emrB, which encode subunits of the EmrAB efflux pump that transports TMP-SMX out of the cell, also supported the greater sensitivity to TMP-SMX of anti-rsPilA vs. anti-IHF NRels. Similarly, the differences noted in the anti-IHF NRel lipid metabolism and cell membrane composition proteins supported the observed increased sensitivity to AMC, wherein the modified membrane content could have altered membrane permeability to allow greater access of the β-lactam antibiotic to the periplasm where they could bind to the penicillin binding proteins to prevent peptidoglycan crosslinking (Delcour, 2009). Additional insight into the mechanism of unique sensitivity to the β-lactam antibiotic in the anti-IHF NRel population was provided by the increased abundance of the peptidoglycan synthesis protein MurB, which suggested that the anti-IHF NRel were actively synthesizing peptidoglycan which would support their greater susceptibility to the action of a β-lactam antibiotic (Delcour, 2009; Zapun et al., 2008). The upregulation of acrR, which represses expression of AcrAB efflux pump, likely results in increased AMC concentration within the anti-IHF NRel and enhanced killing. The upregulation of fis in the anti-IHF NRel population presents yet another possible mechanism for increased sensitivity to AMC, since both P. aeruginosa and E. coli mutants with a nonfunctional fis gene showed enhanced resistance to a β-lactam antibiotic (Grkovic et al., 2001; Long et al., 2020; Martin and Rosner, 1997). In addition to the possible mechanisms discussed above, many other factors, such as accessibility to the bacteria released from the biofilm matrix into the surrounding milieu, combined with overall changes in metabolic activity and/or alterations in membrane content and permeability could all have likely contributed to the observed susceptibilities to the specific class of antibiotic shown, as well as perhaps additional classes of antibiotics not yet tested. This premise is currently under investigation as Applicant continues to further define the phenotypes of anti-rsPilA and anti-IHF NRel NTHI.

Taken together, Applicant's data suggested that the NRel phenotype is not ‘generic,’ but rather highly distinct and dependent on the antibody-mediated mechanism of release of NTHI from biofilm residence. Given that Applicant has already shown that NRel NTHI are rapidly eradicated in vivo by either immune effectors alone (Novotny et al., 2019; Novotny et al., 2015) or when needed, in combination with co-delivered antibiotics (but now at a reduced dose) (Novotny et al., 2016), it is clear that while there are phenotypic distinctions, NRel NTHI and other NRel bacterial species (Brockson et al., 2014; Chambers et al., 2017; Goodwine et al., 2019; Mokrzan et al., 2018; Petrova and Sauer, 2016), appear to be in a highly vulnerable state wherein they can be much more effectively eliminated. Further investigation to characterize the likely manifold distinctions between NRel NTHI populations will include examination of environmental conditions under which biofilms are formed, maturation status and character of biofilms formed by diverse strains of NTHI as well as other genera of bacteria. Furthermore, since the two NRel populations described here represent an adaptive state, the NRel phenotype is likely dynamic over time. Indeed, dissecting the contribution of release kinetics and means to disperse or disrupt biofilms is a focus of investigations to fully characterize the onset and duration of the distinct antibiotic-sensitive phenotypes.

In a world ‘running out of antibiotics’ (2017b) there is a push to identify new antimicrobials, institute antibiotic stewardship and educate the public as to the dangers of inappropriate antibiotic use (Piltcher et al., 2018). This situation has inspired many to attack this problem in novel and creative ways. In a Nature commentary, Rappuoli, Bloom and Black (Rappuoli et al., 2017) suggested Applicant combines the power of vaccine-induced antibodies with a more appropriate use of antibiotics as Applicant's “last hope against multi-drug resistant bacteria and persistent disease”. Whereas their focus was on antibodies that reduce carriage, and thus transmission of antibiotic-resistant bacteria (Rappuoli et al., 2017), Applicant envisions use of specifically induced antibodies to release biofilm-resident NTHI from these highly resistant communities so they can be killed by host immune effectors and when necessary, traditional antibiotics, with the latter now used at a markedly reduced dose and for a shorter course due to the highly sensitive phenotype of NRel NTHI. An additional potential benefit of a less frequent antibiotic treatment regimen is reduction of off-target side effects and other undesirable sequelae of oral antibiotic use; which includes development of antibiotic resistance (Patini et al., 2020; Tagliabue and Rappuoli, 2018) and/or disruption of the gut microbiome (Bailey et al., 2020; Gillies et al., 2015; Kuehn et al., 2015; MacPherson et al., 2018; Pallav et al., 2014).

Herein, Applicant provided proof-of-principal for this strategy to treat biofilm-associated diseases caused by NTHI via use of NRel-inducing antibodies directed against unique biofilm associated targets of this important human pathogen. Moreover, use of the species independent anti-DNABII approach broadens the potential use of this combination strategy for treatment of many other diseases caused by diverse human pathogens wherein a biofilm similarly contributes significantly to pathogenesis, chronicity, recurrence and recalcitrance to treatment. Recent humanization and demonstrated efficacy of NRel-inducing monoclonal antibodies directed against a DNABII protein both in vitro and in vivo is expected to expedite transition to human clinical trials (D'Andrea and Lau, 2020; Novotny et al., 2020).

### Experiment No. 2—Material and Methods

### Collection and Quantitation of NRel NTHI

Nontypeable Haemophilus influenzae strain 86-028NP is a clinical isolate recovered from the nasopharynx of a child undergoing tympanostomy tube insertion due to chronic OM (Harrison et al., 2005; Sirakova et al., 1994) and has been maintained frozen at a low passage number. NTHI biofilms were established in brain heart infusion broth supplemented (sBHI) with 2 μg each of β-nicotinamide adenine dinucleotide (β-NAD) and heme per ml for 16 h in 8-well chambered coverglass slides as described (Jurcisek et al., 2011). After 16 h, biofilms were washed with 200 μl of equilibrated (37° C.) Dulbecco's phosphate buffered saline without calcium or magnesium (DPBS).

To collect and enumerate NRel, 16 h NTHI biofilms were gently washed, then incubated with 13.6 μg rabbit polyclonal IgG derived from anti-rsPilA antiserum (generated against rsPilA expressed by NTHI strain 86-028NP) (Novotny et al., 2009) per cm² of biofilm surface area for 6 h (‘anti-rsPilA NRel’) or 6.2 μg rabbit polyclonal IgG derived from anti-native IHF antiserum (generated against native IHF expressed by NTHI strain 86-028NP) (Novotny et al., 2019) per cm² of biofilm surface area for 15 min (‘anti-IHF NRel’), antibodies were diluted in pre-warmed equilibrated (37° C., 5% CO₂) sBHI. These amounts of IgG match the IgG concentration present within a 1:50 dilution of each respective rabbit hyperimmune serum Applicant used in previous studies (Brockson et al., 2014; Mokrzan et al., 2018). The incubation times used coincide with the time wherein maximal release of NTHI from biofilm residence is achieved (Mokrzan et al., 2018; Novotny et al., 2019). Rabbit polyclonal IgG derived from anti-NTHI OMP P5 or that isolated from naive rabbit serum served as negative controls and were used at equivalent concentrations to the NRel-inducing antisera (e.g. either 11 μg when compared to anti-rsPilA or 5 μg when compared to anti-IHF). Rabbit polyclonal IgG was generated by passage of whole serum or antiserum through rProtein A Protein G GraviTrap columns per manufacturer's instructions (GE Healthcare Life Sciences). After incubation for either 6 h (anti-rsPilA NRel) or 15 min (anti-IHF NRel), 190 μl of supernatant above the biofilm was gently collected from each well, sonicated for 2 min in a water bath sonicator to break up any aggregated NTHI, then serially diluted and plated on chocolate agar to quantitate CFU NTHI.

### Sample Preparation for LC-MS/MS

Anti-rsPilA NRel and anti-IHF NTHI were collected as described. Planktonic NTHI were incubated statically in sBHI until mid-log phase of growth. Samples were centrifuged for 4 min at 13,200×g, resuspended in 1 ml DPBS and centrifuged again. Pellets were flash frozen and stored at −80° C. All further processing was done by MS Bioworks, LLC (Ann Arbor, MI) as described next.

Cell pellets were suspended in buffer (2% sodium dodecyl sulfate, 150 mM NaCl, 50 mM Tris pH 8), lysed with a sonic probe (Q Sonica, Newtown, CT) and heated at 100° C. for 10 min. Protein concentration of the extract was determined by Qubit fluorometry, and 10 μg of each sample was processed by SDS-PAGE using a 10% Bis Tris NuPage mini-gel (Invitrogen) in the MES buffer system. The migration windows (1 cm gel lane) were excised and digested in-gel with trypsin using a ProGest robot (DigiLab, Hopkinton, MA) with the following protocol: 1) wash with 25 mM ammonium bicarbonate followed by acetonitrile; 2) reduce with 10 mM dithiothreitol at 60° C. followed by alkylation with 50 mM iodoacetamide at room temperature; 3) digest with trypsin (Promega, Madison, WI) at 37° C. for 4h; 4) quench with formic acid. Supernatants were analyzed directly without further processing.

Mass Spectrometry

Half of each pooled fraction was analyzed by nano LC-MS/MS with a Waters M-Class HPLC system interfaced to a ThermoFisher Fusion Lumos mass spectrometer. Peptides were loaded on a trapping column and eluted over a 75 m analytical column at 350 nL/min; both columns were packed with Luna C18 resin (Phenomenex, Torrance, CA). The mass spectrometer was operated in data-dependent mode, with MS and MS-MS performed in the Orbitrap at 60,000 FWHM (full width at half maximum) resolution and 15,000 FWHM resolution, respectively. The instrument was run with a 3 s cycle for MS and MS/MS. Two hours of instrument time was employed for the analysis of each sample.

Mass Spectrometry Data Processing

Data were searched using a local copy of Mascot (Matrix Science) with the following parameters: enzyme, trypsin/P; database, www.ncbi.nlm.nih.gov/nuccore/CP000057.2 (concatenated forward and reverse plus common contaminants); fixed modifications, carbamidomethyl (C); variable modifications: acetyl (N-term), deamidation (N,Q), oxidation (M), pyro-glu (N-term Q); mass values, monoisotopic; peptide mass tolerance, 10 ppm; fragment mass tolerance, 0.02 Da; maximum missed cleavages, 2. Mascot DAT files were parsed into Scaffold (Proteome Software) for validation, filtering and to create a non-redundant list per sample. Data were filtered using a 1% protein and peptide false discovery rate (FDR), requiring at least two unique peptides per protein. Applicant used the normalized spectral counts for downstream analysis. To evaluate the variation and reproducibility in the replicates, Applicant generated the PCA plot with 95% confidence ellipses surrounding each population (using the FactoMineR and ggplot2 packages in R), with the normalized spectral counts for each protein identified by mass spectrometry in the three samples of planktonic, anti-rsPilA NRel, and anti-IHF NRel groups (Lê et al., 2008; Wickham et al., 2016).

Differential Expression Analysis

Normalized spectral counts from three biological replicates of the three sample groups (planktonic, anti-rsPilA NRel and anti-IHF NRel), were used for pairwise comparisons to determine the differential expression of each protein using a two-tailed t-test. The P-values, Benjamini-Hochberg adjusted P-values, and fold changes are provided (Supplementary data set 1). Proteins with >1.5-fold increase or decrease and with an associated P<0.05 were considered to be significantly different in expression. NTHI strain 86-028NP proteins were annotated by Clusters of Orthologous Groups of proteins (Aziz et al., 2019; Tatusov et al., 2000).

Antibiotic Sensitivity of Biofilm-Resident, Planktonic or NRel NTHI

As Applicant had hypothesized that NRel would be more sensitive than planktonic NTHI to certain antibiotics, Applicant set the baseline for planktonic killing to 25% so that Applicant would be able to demonstrate a dynamic range in NRel killing by a given antibiotic. To avoid excessive manipulation or dilution of NRel NTHI, and to permit direct comparison of equal numbers of planktonic and NRel, Applicant adjusted the planktonic cell density to the same CFU per ml as the NRel population in each experiment as follows: NTHI were incubated statically to mid log phase growth then diluted to either 3×10⁸ CFU/ml for comparison with anti-rsPilA NRel collected at 6 h, or to 2×10⁸ CFU/ml for comparison with anti-IHF NRel collected at 15 min (see FIG. 1). Applicant then determined the concentrations of amoxicillin (Sigma-Aldrich, St. Louis, MO) and clavulanate (U.S. Pharmacopeia, Rockville, MD), or of trimethoprim (Sigma-Aldrich) and sulfamethoxazole (Santa Cruz Biotech, Dallas, TX) that killed approximately 25% of planktonic NTHI at each density. The same antibiotic concentrations were used for the anti-IHF or anti-rsPilA NRel, the density-matched (same CFU/ml) planktonic NTHI, and the adherent biofilm for each relevant experiment. Likewise, for anti-IHF or anti-rsPilA NRel collected at 2 h, Applicant first quantitated NRel, and found ˜2.0×10⁸ or 4.0×10⁸ CFU/ml released by exposure of biofilms to anti-rsPilA or anti-IHF IgG, respectively. Applicant then assessed killing of anti-IHF or anti-rsPilA NRel by concentrations of amoxicillin plus clavulanic acid (“augmentin”, AMC) or trimethoprim plus sulfamethoxazole (TMP-SMX) that killed 25% of the planktonic NTHI at the same density.

Anti-rsPilA or anti-IHF NRel were collected and sonicated as described above. After sonication for 2 min in a water bath sonicator to break up any NTHI aggregates, NRel or planktonic NTHI were incubated with the indicated antibiotics at 37° C. for 2 h, then serially diluted and plated on chocolate agar to quantify viable NTHI. To assay biofilm-resident NTHI, biofilms were established for 16 h at 37° C. as described above, washed twice with DPBS and incubated in sBHI supplemented with the indicated antibiotics at 37° C. After 2 h, biofilm-resident NTHI were collected by forceful pipetting, sonicated for 2 min and enumerated as described above. All experiments were performed at least three times on separate days with two or three technical replicates for each treatment and control group.

Flow Cytometry

As described above, NTHI biofilms were established in 8-well chambered coverglass slides. After 16 h, medium was aspirated from each well and biofilms incubated with 5 μg IgG from rabbit polyclonal IHF for 15 min or 11 μg IgG from polyclonal rabbit anti-rsPilA for 6 h at 37° C., 5% CO₂. At each respective timepoint, 190 μl of supernatant above each biofilm was collected, transferred into 1 μM FM1-43FX (Invitrogen) in Hank's Balanced Salt Solution and incubated static for 15 min at room temperature. NTHI scraped from a chocolate agar plate into buffer served as a ‘clumped’ bacterial control. NTHI suspended in buffer by gentle pipetting followed by sonication for 5 min served as a ‘non-clumped’ bacterial control. Forward scatter and side scatter profiles of fluorescently stained NTHI were examined with a BD LSR II flow cytometer and FloJo software, 10,000 events were collected for each sample.

RNA Isolation and qRT-PCR Assay

For RNA isolation, Applicant seeded 6 ml of NTHI at 2×10⁵ CFU/ml into a T-25 tissue culture flask. After 16 h incubation at 37° C., 5% CO₂, the flask was gently inverted and the medium poured off. With the flask upside down, 6 ml prewarmed DPBS was added then the flask was slowly inverted to gently wash the biofilm. To remove the DPBS wash, the flask was inverted again and DPBS poured off. Antibody diluted in sBHI was added with the flask still upside down, to deliver the same concentration of antibody/cm² as used in chamberslide assays (51.5 μg anti-IHF IgG per ml or 113 μg anti-rsPilA IgG per ml). The flask was returned to the incubator, inverted gently so that the medium again covered the biofilm. After 3 min for anti-IHF, or 3 h for anti-rsPilA the flask was inverted and the medium poured off to collect the NRel NTHI, and centrifuged for 1 min at 16.1×g. The supernatant was aspirated and the bacterial pellet was immediately added 1 ml TRIzol™ Reagent (ThermoFisher, Waltham, MA). Samples were stored at −80° C.

RNA was purified with a Qiagen RNeasy kit (Qiagen, Germantown, MD). Residual DNA was removed by treatment with DNase I (NEB), per manufacturer's instructions for 45 min at 37° C. in the presence of 20 U SUPERase In RNase inhibitor (Ambion). Relative gene expression was assessed by quantitative reverse transcription-PCR (qRT-PCR) with a Superscript III Platinum SYBR Green One-Step qRT-PCR kit (ThermoFisher) per manufacturer. Gene expression was normalized to 16S, and relative expression was calculated by the comparative (ΔΔC_{T}) method, with fold change in gene expression expressed as 2^{(-ΔΔCT)}. Results represent the mean of 3 biological samples, each assayed in triplicate. A 2-fold change in gene expression was considered biologically significant. Primers used are listed in Table 3.

TABLE 3
Primers used in this study

| Primer | Sequence | SEQ ID NO: |
|---|---|---|
| acrR-forward | CGGCGATAAATTTAGCCTCTGA | |
| acrR-reverse | TGAATCGCACGCCAAGAG | |
| artM-forward | GTCTTATCCAATGCGTGGTTCT | |
| artM-reverse | GGATGCTAATGCCGTTCCTTTA | |
| deaD-forward | TGTGGTGAACTACGACATTCC | |
| deaD-reverse | GATCCTGATCGGCTGTGAATAA | |
| emrA-forward | CCGCAAATACAGAATGCGATAAA | |
| emrA-reverse | ATTACGACGCGCCACATAG | |
| emrB-forward | CGGTAACTTTCGAGCCATCA | |
| emrB-reverse | GCCAGACAGCGTTATTGTAGTA | |
| fis-forward | TAATCCTGCCGATGCCTTAAC | |
| fis-reverse | CGGGTTTGATTACCACGAGTAT | |
| folA-forward | TTGGTCGTCCACTACCTAAAC | |
| folA-reverse | GACCGCACTTTCAAAGCTATC | |
| folP-forward | TGCTGGATTTCTGTCGATTCTT | |
| folP-reverse | GCTCTTGCAAAGCACGAATATC | |
| 16S-forward | AAAGGAGACTGCCAGTGATAAA | |
| 16S-reverse | CCCTCTGTATACGCCATTGTAG | |

Statistical Analyses

Data are expressed as mean±SEM of at least three biological replicates performed on separate days with two or three technical replicates per sample. Statistical analyses were performed with GraphPad (Prism) software version 8.2. Multiple comparisons were made by one-way analysis of variance with the Holm-Sidak correction. All other comparisons were made with student's t-test. Comparisons of fold changes in normalized spectral counts of proteins identified by mass spectrometry and generation principal component analysis were performed in R (Bioconductor). Flow cytometry data were analyzed by the Kolmogorov-Smirnov test to compare the cumulative distribution of anti-rsPilA NRel versus anti-IHF NRel for forward scatter and side scatter profiles.

Data Availability

All data generated or analyzed during this study can be found in Table 1.

### Experiment No. 3—Synergistic Effects of Anti-rsPilA and Anti-DNABII Tip Chimer Antibody

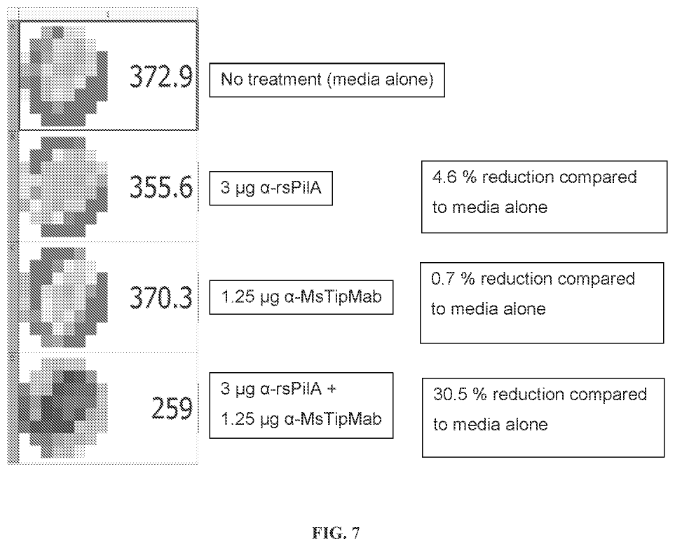

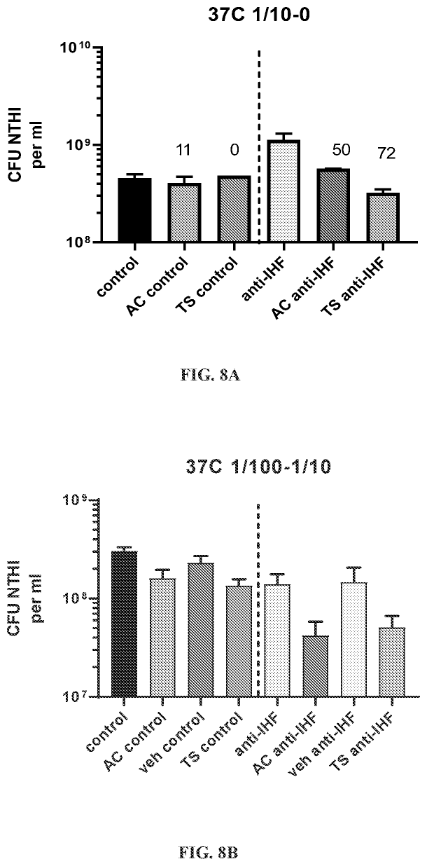

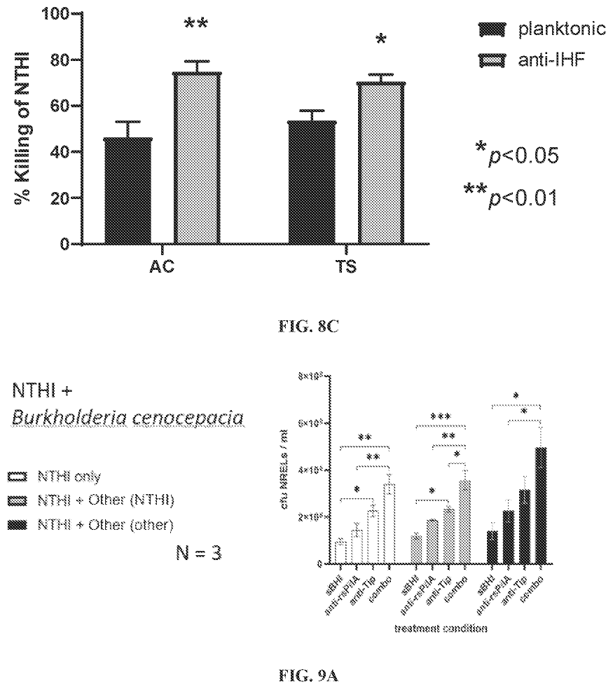

NTHI+Streptococcus pneumoniae+Staphylococcus aureas were grown in a 1:1:1 ratio for 16 hours at 37° C. and 5% CO₂ and allowed to form a biofilm prior to a 2 hour treatment. Four treatment groups are included, such as media alone as a negative control, 3 μg anti-rsPilA antibodies, 1.25 μg anti-MsTipMab (i.e., mouse monoclonal antibody against DNABII tip chimer), and 3 μg anti-rsPilA antibodies plus 1.25 μg anti-MsTipMab. Bacteria were labeled with FM1-43 FX and relative fluorescence was calculated per well. As shown in FIG. 7, a 30.5% reduction of biofilm was observed in the combinatorial treatment, while the monotreatments showed 4.6% and 0.7% reductions, suggesting that a clear synergistic outcome was achieved via combinatorial treatment with anti-rsPilA plus anti-tip chimer monoclonal antibody.

### Experiment No. 4—Leveraging Two Unique Mechanisms to Release Bacteria from Biofilms to an NRel State to Achieve an Extended Therapeutic Window of Opportunity

Applicant determined that the exact mechanism by which one releases a bacterium from biofilm residence has a profound influence on the phenotype of that newly released (NRel) population. See, for example, Experiment No. 1. By combining these two methods that target distinct determinants Applicant can overcome several important bottlenecks to treatment and/or prevention.

Whereas it has been shown that bacteria released from biofilms via a variety of mechanisms are different from those that are either resident within that biofilm or when planktonically grown (as commonly done in the laboratory), Applicant determined that despite genetic identity, populations of NRel bacteria are unique due to the exact manner by which they were released from the biofilm. The phenotype of these NRel bacterial populations can be leveraged in a unique way to overcome multiple bottlenecks to both treatment and/or prevention of chronic and recurrent bacterial diseases wherein biofilms contribute significantly to pathogenesis and the disease course.

In view of this unanticipated development, Applicant further observed a synergistic protective/therapeutic outcome combining anti-DNABII and anti-PilA, and immunizes with both a DNABII protein AND PilA to repeat such synergistic effects. See, Novotny et al 2015 and Novotny et al 2017 Clinical Vaccine Immunology 24:e00563-16 for more details relating to methods and others. Applicant also had demonstrated that antibodies to PilA or those to IHF would release bacteria from a biofilm and into a state that was highly sensitive to the action of different antibiotics than were those in the biofilm (see: Brockson, M E et al. 2014; Mokrzan, E M et al. 2018). Prior to those results, Applicant did not expect that the antibodies induced by these two immunogens would result in two genetically identical but phenotypically distinct populations of bacteria that had different proteomes, transcriptomes and susceptibilities to antibiotics. Susceptibility to other immune effectors is under investigation.

Chronic and recurrent bacterial diseases present a substantial global health burden. They are by nature, exceptionally difficult to treat and highly resistant to traditional antibiotics. Applicant envisioned being able to combine immunization induced antibodies and/or therapeutic delivery of antibodies that induce the distinct described NRel states in a manner that fosters a sustained window of opportunity for treatment and/or a synergistic clinical outcome.

In the literature, it has been demonstrated that bacteria released from biofilm-residence via any number of ways are different from both when they were part of the biofilm community and/or when they are grown planktonically, as typically done in the laboratory. Applicant was intrigued by this observation and wondered if either an approach which was directed specifically against the type IV twitching pilus of NTHI or that which was species-agnostic and directed specifically against protective epitopes within either of the two DNABII proteins, both of which released NTHI and other bacteria from biofilm-residence, did indeed also render these newly released (NRel) bacteria highly sensitive to killing by antibiotics. This was a clinically relevant question as bacteria are never truly planktonic in a disease state, they prefer to exist within a protective biofilm which is notoriously difficult to treat. Applicant hypothesized that if Applicant could release bacteria from a biofilm with either anti-rsPilA or anti-DNABII antisera into a highly vulnerable state (to either traditional antibiotics and/or immune effectors), perhaps this could be leveraged clinically. Applicant was indeed able to demonstrate this enhanced killing by antibiotics for both NRel NTHI and NRel M. catarrhalis, however what Applicant could not have anticipated was that for at least NTHI, the populations of NRel that resulted after release from biofilm residence by either anti-rsPilA or anti-DNABII sera were phenotypically distinct, despite being genetically identical. This distinction was evidenced by proteomics, transcriptomics and distinct statistically significant increased killing by a given antibiotic. Without wishing to be bound by the theory, Applicant hypothesizes that in states of mixed bacterial biofilm infection Applicant can leverage this understanding to capitalize on the power of anti-rsPilA to induce sustained but slower top-down release of NTHI from an established biofilm due to programmed expression of both LuxS and type IV pili as well as prevent the formation of any new NTHI biofilm as a result of active immunization but also, via either active immunization and/or therapeutic delivery of anti-DNABII antibody directed specifically against the protective epitopes, rapidly disrupt biofilms formed by multiple bacterial species via a non-programmed mechanism. Given that both of these antisera induce a distinct NRel phenotype, this provides a window of opportunity for antibiotic treatment, if needed, and for host mediated clearance that is sustained and amplified beyond that which either would likely be able to induce alone. This hypothesis is predicated on the highly unexpected result wherein the induction of the NRel state was distinct and attributable to the exact mechanism of release, despite the genetic identity of the NRel populations. In diseases wherein the biofilms have been in place for an extended period of time and are comprised of multiple bacteria, Applicant hypothesizes that anti-DNABII antibodies debulks these existing biofilms in a species agnostic manner and generate multiple NRel populations susceptible to the killing action of specific antibiotics and likely immune effectors whereas anti-rsPilA antisera acts both preventatively and therapeutically to specifically target NTHI which plays a roles in diseases throughout the mammalian airway by inducing an NRel population with its own unique susceptibilities to antibiotics, if needed, and immune effectors. The more ways one can induce an effective anti-biofilm strategy, the greater the anticipated power and predicted positive clinical outcome. Ideally, for therapeutic applications, Applicant also uses humanized monoclonal antibodies directed against the specific protective epitopes of both rsPilA and the DNABII proteins which Applicant identified and characterized. For active immunization applications, Applicant similarly immunizes with vaccine candidates that Applicant designed and tested extensively preclinically for their relative effectiveness.

Applicant uses the high throughput 96-well biofilm plate assay developed to build both diverse single species and mixed species biofilms by multiple high priority pathogens then treat these biofilms with either anti-rsPilA alone, anti-DNABII alone or a combination of both to determine their relative ability to disrupt/disperse an existing biofilm. Bacteria to be tested include: NTHI, M. catarrhalis, Staphylococcus aureus, Pseudomonas aeruginosa, Streptococcus pneumonia, Burkholderia cenocepacia, the ESKAPE pathogens and others.

Further, Applicant uses the high throughput assay to generate/recover NRel bacteria to test for relative antibiotic sensitivity when compared to planktonic counterparts (as used in clinical microbiology laboratories to determine MIC values and treatment regimens for patients) testing both single species and mixed species biofilms as above.

In addition, Applicant uses any of the three animal models of biofilm diseases (chinchilla model of OM; rat model of periimplantitis or murine model of lung disease) to test the combined active immunization+therapeutic approach as hypothesized above.

### Experiment No. 5—2-Species Biofilm Disruption/Dispersal Synergy Assay

Bacteria were grown on agar for 18-20 hrs, then diluted in supplemented BHI broth (sBHI) to achieve a specific target concentration that was pre-determined to allow both microorganisms to survive being cultured together and allowed us to be able to recover NTHI at similar concentrations to those achievable when NTHI was in a single-species biofilm of the same age when being treated. 100 μl of each of the two bacterial suspensions was added to the appropriate wells of an 8-well covered chamber glass slide. However, for those wells in which only NTHI was seeded, 100 μl of sBHI was added so that all wells contained 200 μl total volume of culture medium. The target concentrations for each bacterial pairing are listed below (Note that the ratios of NTHI:Second bacterial species was unique to each pairing).

TABLE 4

| | cfu/well | | cfu/well |
|---|---|---|---|
| NTHI | 2.00E+04 | B. cenocepacia | 5.00E+03 |
| NTHI | 2.00E+04 | S. aureus | 2.50E+03 |
| NTHI | 1.00E+08 | S. pneumoniae | 5.00E+04 |
| NTHI | 2.00E+04 | M. catarrhalis | 6.00E+04 |

Chambered coverglass slides were then incubated at 37° C., 5% CO₂ for 24 hours. Wells were gently washed once with 200 μl pre-warmed sBHI to remove any bacteria not within the biofilm, then 200 μl of either sterile sBHI (negative control), anti-rsPilA monoclonal antibody, anti-tip chimer monoclonal antibody (the A5-mB4 tip chimer antibody), or a cocktail of anti-rsPilA+anti-tip chimer monoclonal antibodies (‘combo’, all diluted in sBHI) was added to the well. Chambered coverglass slides were then incubated for 2 hours at 37° C., 5% CO2.

After incubation, 150 μl of medium was removed from each well and placed into a sterile Eppendorf tube. 200 μl sterile DPBS was then carefully added to each well so as not to disturb any remaining biofilm and 150 μl of this was collected and added to the Eppendorf tube and repeated for each well. These pooled fluids contained bacteria that had either been newly-released from biofilm residence (NRel) by the action of the monoclonal antibody or antibody cocktail applied or in the case of those biofilms ‘treated’ with the negative control (sBHI), represented bacteria simply present in the culture fluid as they had either come off the biofilm or had never associated with the biofilm and were simply growing in the culture fluid as a normal part of “biofilm remodeling.”

Because Applicant had already determined that NRel produced via the action of antisera that target the DNABII proteins induce release of bacteria from biofilm residence in small aggregates (whereas those produced by the action of anti-rsPilA are single cells), all tubes were gently sonicated for 2 minutes before serially diluting and plating bacteria onto the appropriate agar in order to assure the most accurate count.

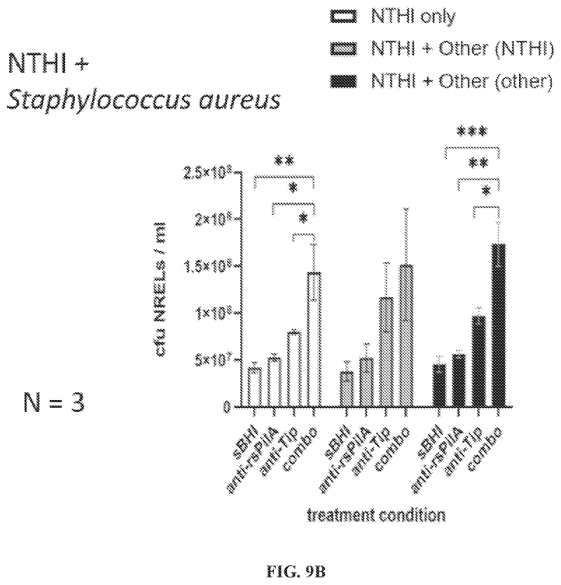

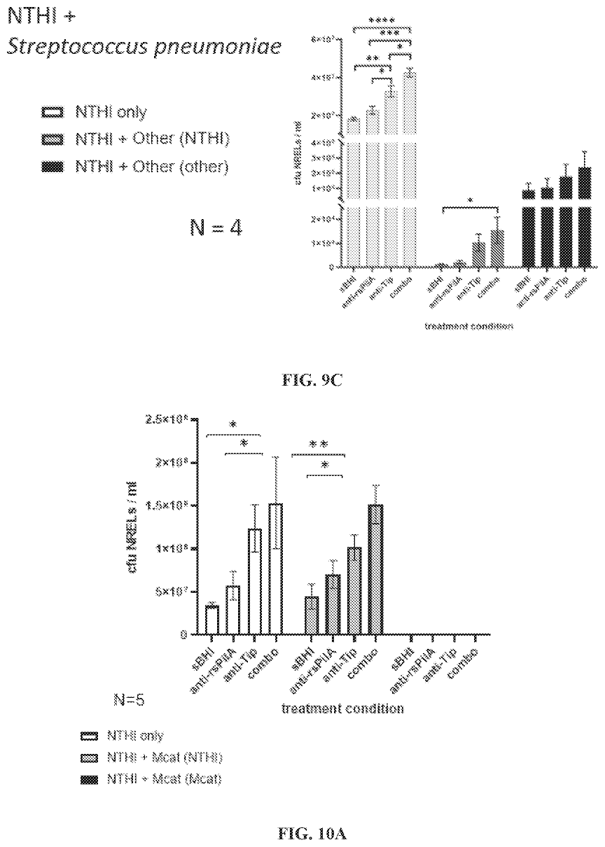

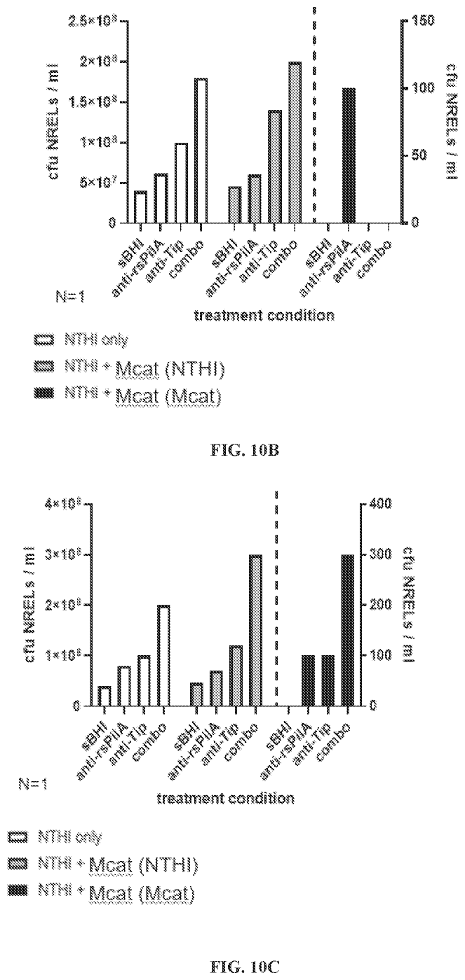

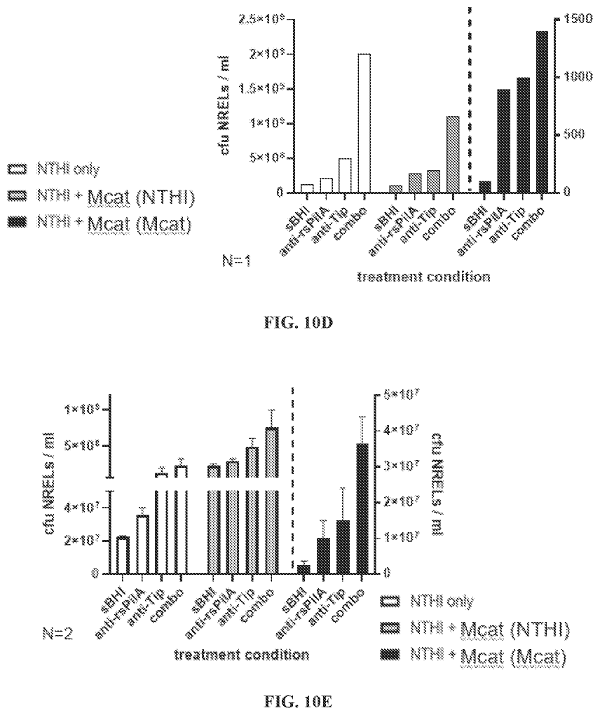

In this study, Applicant looked at biofilms formed by NTHI and another human respiratory tract pathogen that is commonly found with NTHI in many human diseases wherein biofilms contribute to pathogenesis. NTHI+Burkholderia cenocepacia is highly problematic and particularly so in individuals with cystic fibrosis (CF); NTHI+Staphylococcus aureus is a problematic combo throughout the airway and NTHI+Streptococcus pneumoniae is problematic throughout the airway. Each plot in FIG. 9 shows many bacteria (NRel) are newly released by media alone (sBHI) which is the negative control, by anti-rsPilA, by anti-TipMab or by the antiserum cocktail. The open bars depict what happens to NTHI when it builds a biofilm alone (see FIG. 7 wherein it is shown that sBHI does not release bacteria from a biofilm but that both anti-rsPilA antibody and anti-TipMab do and the cocktail is synergistic and thus induces the greatest release)—open bars. They gray shaded and solid black bars to show that when NTHI forms a biofilm with another bacterial species and treated with sBHI, one of the two antisera or a cocktail of both antisera. See FIG. 9. FIG. 10 shows the results of treatment of NTHI and M cat. biofilms.

### Experiment No. 6—Otitis Media

Middle ear infection (or otitis media, OM) is a highly prevalent disease worldwide, with the most severe form (called chronic suppurative OM or CSOM) afflicting 50-330 million children globally each year. The socioeconomic burden of OM is also great, with cost estimates between $5-6 billion in the United States alone annually. All three of the predominant bacterial pathogens of OM are known to form biofilms both in vitro and in vivo and recently, clinicians have come to appreciate that the chronicity and recurrence of OM is due, at least in part, to the formation of bacterial biofilms within the middle ear cavity.

In fact, results of labeling of otorrhea solids from pediatric patients with tympanostomy tubes and persistent otorrhea for eDNA and IHF in combination with microbiological culture indicate that biofilms play a role in chronic otorrhea. Specifically, of 15 pediatric otorrhea samples analyzed, 9 (60%) contained solids positive for labeling IHF in association with a lattice of eDNA (labeled using rabbit anti-IHF, detected with goat anti-rabbit IgG conjugated to AlexaFlour 594) and 75% yielded positive bacterial cultures. Bacterial culture results demonstrated the presence of H. influenzae, Staphylococcus aureus (MRSA), S. pneumonia, M. catarrhalis, and P. aeruginosa. These data suggest that DNABII proteins may serve as a therapeutic target in post-tympanostomy tube otorrhea among other otic disease.

In one chinchilla model of OM, juvenile chinchillas are first given a viral “cold” followed a week later by their being challenged intranasally with an inoculum viable bacteria. Similar to the human condition wherein “my child has a cold and a week later gets an ear infection” chinchillas also develops a bacterial OM approximately one week after a challenge, and while experiencing the viral upper respiratory tract infection. Once bacteria gain access to the middle ear (either via ascension of the Eustachian tube or following direct challenge to the middle ear space), they form a robust biofilm. Applicants thus contemplate and indeed have already used chinchilla models as reported herein to demonstrate the protective efficacy of IHF immunization which results in rapid resolution of existing biofilms. This model is also useful for therapeutic approaches via either passive delivery of anti-DNABII antibody or via delivery of a small molecule or other agent known to bind to IHF or other DNABII family members.

In order to determine the efficacy of the combination and/or method as disclosed herein, NTHI bacteria were injected into the middle ear space of the chinchillas and allowed to form a biofilm. In particular, adult chinchillas (Chinchilla lanigera) with no evidence of middle ear disease were procured (Rauscher's Chinchilla Ranch, LLC) and rested for 7 days prior to transbullar challenge with 1000 colony-forming units (CFU) of nontypeable Haemophilus influenzae #86-028NP per bulla diluted in sterile, pyrogen-free saline (Day zero (0)). Then, either on days 4 and 5 or on days 4, 5, and 6, a composition as disclosed herein is infused into each middle ear space (100 μl per bulla). For chinchillas administered on days 4 and 5, 3 chinchillas per cohort are sacrificed either on day 6 or day 12 (two dose experiments). For those administered on day 4, 5, and 6, 3 chinchillas per cohort are sacrificed on day 13 (three dose experiments). Following sacrifice, chinchillas are imaged, middle ear mucosa is collected, adherent biofilm is assessed, middle ear fluids are collected, quantitation of bacteria is performed, and middle ear fluids are assessed using a cytokine multiplex assay.

Middle ear fluids are collected and an aliquot serially diluted and plated on to chocolate agar to quantitate the relative planktonic bacterial load. Remaining fluids are centrifuged at 1000×g for 5 min, supernatants separated and both cellular pellet and fluid fractions snap-frozen prior to storage at −80° C. The middle ear mucosa and adherent bacterial biomass are digitally imaged, collected into pre-weighed microcentrifuge tubes and homogenized in 1.0 ml sterile 0.9% sodium chloride. Homogenates are also serially diluted and plated, as before, to quantitate the population of bacteria adherent within the middle ear space per mg tissue/biomass. Remaining sample is snap-frozen prior to storage at −80° C. Culture plates are incubated for 24 h at 37° C. in a humidified atmosphere prior to enumeration of bacterial colonies via Protocol2 instrument (Synbiosis).

Video otoscopy using a 0-degree, 3-inch probe connected to a digital camera system (MedRx, Largo, FL) is utilized to monitor signs of OM (e.g. tympanic membrane inflammation and/or presence of fluid in the middle ear space). Tympanometry is performed with a MADSEN Otoflex tympanometer and data analyzed with OTOsuite software (Otometrics, Schaumburg, IL). Overall signs of OM are blindly rated on an established 0 to 4+ scale and middle ears with a score of ≥2.0 are considered positive for OM if middle ear fluid is visible behind the tympanic membrane. If the tympanic membrane cannot be visualized due to an obstruction within the ear canal (i.e. due to cerumen accumulation), that ear is excluded from the day's count. Per established protocol, each middle ear is considered independent, and for each cohort, the percentage of middle ears with OM is calculated.

To rank the residual biofilm within the middle ear space, middle ear images are scrambled and reviewed blindly by 7 individuals. Using an established rubric, a score of 0 to 4 is assigned to each image, whereby 0: no biofilm visible, 1: biofilm fills ≤25% of middle ear space, 2: biofilm fills >25% to ≤50% of middle ear space, 3: biofilm fills >50% to ≤75% middle ear space, 4: biofilm fills >75% to ≤100% middle ear space.

This ranking/scoring scale is described in detail in the Table below, indicating the relative amount of biomass remaining within the middle ear of each animal.

TABLE 5

| Score | Criteria |
|---|---|
| 0 | No evidence of biomass. |
| 1+ | Biomass fills ≤25% of middle ear space. |
| | Junction of the bony septa to inferior bulla is visible. |
| 2+ | Biomass fills >25% to ≤50% of middle ear space. |
| | Unable to visualize where the bony septa meet the inferior bulla. |
| 3+ | Biomass fills >50% to ≤75% of middle ear space. |
| | Biomass covers >50% of the length of bony septa. |
| 4+ | Biomass fills >75% to ≤100% of middle ear space. |
| | Bony septa not visible; obscured by biomass. |

The relative quantity of a panel of pro- and anti-inflammatory cytokines in clarified middle ear fluids (IL-1β, IL-6, IL-8, IL-12p70, IL-17A, TNF, IFNγ, IL-4, IL-10, and IL-13) is determined using BD Cytometric Bead array (BD Biosciences) according to manufacturer's instructions and samples are assessed on a BD Accuri C6 cytometer. Data are analyzed with FloJo V_10 software.

### Experiment No. 7—Treatment of Oral Disease

A number of oral bacteria (e.g., Aggregatibacter actinomycetemcomitans, Porphyromonas gingivalis) have been implicated in the pathogenesis of inflammatory diseases such as periodontitis and peri-implantitis, which destroy alveolar bone and gingiva. Investigations of the pathogenesis of these bacteria are hampered by lack of effective animal models. One of the challenges of investigating the pathogenicity of specific bacteria is the difficulty of establishing a biofilm when exogenous bacteria are introduced into the oral cavity of animals. Though animal models of periodontitis have been developed, cultivable bacteria are rarely recovered from the oral cavity of inoculated animals. Developing an effective animal model which can assess the pathogenicity of specific bacteria greatly aids in elucidating their pathogenic mechanisms.

The surface of machined titanium dental implants (1.2×4.5 mm) can be modified by grit blasting with A103 (100 m) and HCl etching (pH 7.8 for 20 min at 80° C.). Machined and nano-textured implants are incubated in TSB medium inoculated with D7S clinical strain of Aggregatibacter actinomycetemcomitans (Aa) for 1 to 3 days at 37° C. The bacterial biofilm on the implants are analyzed by SEM, as well as by confocal laser scanning microscopy following staining with LIVE/DEAD® BacLight™. Implants with and without established Aa biofilm are transmucosally placed into the alveolar bone of female rats between premolar and incisor region of the maxillae. To detect the presence of Aa biofilm on the implants placed in vivo, bacterial samples are collected from saliva and the oral surfaces of implants after 2 days. Aa can be detected by culture, as well as by PCR analysis. Micro-CT and histological analysis of peri-implant bone and mucosal tissues can be performed at various time points, e.g., six weeks after implantation.

Further, the oral cavity contains an array of bacterial species that coexist within biofilm communities. These communities contain both commensal bacteria, such as some oral streptococci, and other bacterial species, such as the periodontal pathogen Porphyromonas gingivalis. The ability of P. gingivalis to enter, persist, and expand within a preexisting biofilm depends on its ability to interact with the commensal bacteria present within the biofilm. One well-characterized binding partner of P. gingivalis is Streptococcus gordonii. Understanding how P. gingivalis interacts with S. gordonii and interfering with those interactions may allow exclusion or removal of this important periodontal pathogen from native biofilm communities within the oral cavity. Confocal microscopy can be utilized to analyze the biofilm structure, and differential plating and immunofluorescence microscopy can be used to determine the composition of the bacterial species present within the biofilms.

The methods and compositions disclosed herein are contemplated to develop both therapeutic as well as preventative strategies for reduction and/or elimination of these biofilms. A decrease in redness, inflammation, and bleeding compared to infected controls would indicate biofilm reduction and/or elimination. In addition, reduced or absent inflammatory or proinflammatory histology and maintenance of torque removal force for the implant screw compared to infected controls would indicate biofilm reduction and/or elimination.

### Experiment No. 8—Cystic Fibrosis

This experiment provides a porcine model for pre-clinical testing of interfering agents to treat cystic fibrosis. See Stoltz et al. (2010) Science Translational Medicine 2(29):29-31. Cystic fibrosis is an autosomal recessive disease due to mutations in a gene that encodes the CF transmembrane conductance regulator (called CFTR) anion channel. In this model, pigs which have been specifically bred to carry a defect in the genes called “CFTR” and called CF pigs spontaneously develop hallmark features of CF lung disease that includes infection of the lower airway by multiple bacterial species. The methods and compositions disclosed herein are contemplated to develop both therapeutic as well as preventative strategies for reduction and/or elimination of these biofilms and/or for amelioration of the signs of disease and associated pathologies.

### Experiment No. 9—PilA Vaccine

Methods described herein may be used to elicit immune responses against PilA in humans and animals. Immunogenic compositions may be administered to human and animal subjects in the presence of adjuvants such as but not limited to aluminum salts and liposomes. Those skilled in the art will understand that any number of pharmaceutically acceptable adjuvants can also be used. Immunogenic compositions may be administered to a human or animal subjects intramuscularly, subdermally, intranasally, or through any other suitable route. Immunogenic compositions may be prepared in a manner consistent with the selected mode of administration. Immunogenic compositions may take the form of polypeptides, nucleic acids, or a combination thereof, and may comprise full-length or partial antigens. Additionally, or alternatively, immunogenic compositions may take the form of APCs pulsed with a particular antigen, or APCs transfected with one or more polynucleotides encoding a particular antigen. Administration may comprise a single dose of an immunogenic composition, or an initial administration, followed by one or more booster doses. Booster doses may be provided a day, two days, three days, a week, two weeks, three weeks, one, two, three, six or twelve months, or at any other time point after an initial dose. A booster dose may be administered after an evaluation of the subject's antibody titer.

### Experiment No. 10—Passive Immunity

Methods described herein may be used to confer passive immunity on a non-immune subject against PilA and/or DNABII as disclosed herein. Passive immunity against a given antigen may be conferred through the transfer of antibodies or antigen binding fragments that specifically recognize or bind to a particular antigen. Antibody donors and recipients may be human or non-human subjects. Additionally, or alternatively, the antibody composition may comprise an isolated or recombinant polynucleotide encoding an antibody or antigen binding fragment that specifically recognizes or binds to a particular antigen.

Passive immunity may be conferred in cases where the administration of immunogenic compositions poses a risk for the recipient subject, the recipient subject is immuno-compromised, or the recipient subject requires immediate immunity. Immunogenic compositions may be prepared in a manner consistent with the selected mode of administration. Compositions may comprise whole antibodies, antigen binding fragments, polyclonal antibodies, monoclonal antibodies, antibodies generated in vivo, antibodies generated in vitro, purified or partially purified antibodies, or whole serum. Administration may comprise a single dose of an antibody composition, or an initial administration followed by one or more booster doses. Booster doses may be provided a day, two days, three days, a week, two weeks, three weeks, one, two, three, six or twelve months, or at any other time point after an initial dose. A booster dose may be administered after an evaluation of the subject's antibody titer.

### EQUIVALENTS

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

It should be understood that although the present disclosure has been specifically disclosed by specific embodiments and optional features, modification, improvement and variation of the embodiments therein herein disclosed may be resorted to by those skilled in the art, and that such modifications, improvements and variations are considered to be within the scope of this disclosure. The materials, methods, and examples provided here are representative of particular embodiments, are exemplary, and are not intended as limitations on the scope of the disclosure.

The scoped of the disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

SEQUENCE LISTING
SEQ ID NO: 1
(H10210 (1F8.F1 Humanized HC1 (anti-tip chimer antibody))
Bold font indicates an exemplified variable region
while the bold, italic and underlined font
indicates exemplified CDRs.
MDPKGSLSWRILLFLSLAFELSYGEVKLVESGGGLVQPGGSLRLSCAASGFTFRTY
AMSWVRQAPGKGLEWVATIGSDRRHTYYPDSVKGRFTISRDNAKNTLYLQMNS
LRAEDTAVYYCVGPYDGYYGEFDYWGQGTLVTVSSASTKGPSVFPLAPSSKSTSGG
TAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMI
SRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGN
VFSCSVMHEALHNHYTQKSLSLSPG**
SEQ ID NO: 2
(H10211 (1F8.F1 Humanized HC2, anti-tip chimer antibody)
Bold font indicates an exemplified variable region
while the bold, italic and underlined font
indicates exemplified CDRs.
MDPKGSLSWRILLFLSLAFELSYGEVQLVESGGGLVQPGGSLRLSCAASGFTFRTY
AMSWVRQAPGKGLEWVATIGSDRRHTYYPDSVKGRFTISRDNSKNTLYLQMNS
LRAEDTAVYYCVGPYDGYYGEFDYWGQGTLVTVSSASTKGPSVFPLAPSSKSTSGG
TAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMI
SRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGN
VFSCSVMHEALHNHYTQKSLSLSPG
SEQ ID NO: 3
(H10212 (1F8.F1 Humanized HC3, anti-tip chimer antibody))
Bold font indicates an exemplified variable region
while the bold, italic and underlined font
indicates exemplified CDRs.
MDPKGSLSWRILLFLSLAFELSYGEVKLVQSGAEVKKPGASVKVSCKASGFTFRTY
AMSWVRQAPGQRLEWVATIGSDRRHTYYPDKFQGRVTITRDNAKNTLYMELSSLR
SEDTAVYYCVGPYDGYYGEFDYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQG
NVFSCSVMHEALHNHYTQKSLSLSPG**
SEQ ID NO: 4
(H10213 (11E7.C7))
Bold font indicates an exemplified variable region
while the bold, italic and underlined font
indicates exemplified CDRs.
MDPKGSLSWRILLFLSLAFELSYGEVQLVESGGGLVKPGGSLRLSCAASGFTFSRY
GMSWVRQAPGKGLEWVATISSGGSYTYYTDSVKGRFTISRDNAKNSLYLQMNSL
RAEDTAVYYCERHGGDGYWYFDVWGQGTMVTVSSASTKGPSVFPLAPSSKSTSG
GTAALGCLVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTKNQV
SLTCLVKGFYPSDIAVEWESNGQPENNYKTTPPVLDSDGSFFLYSKLTVDKSRWQQG
NVFSCSVMHEALHNHYTQKSLSLSPG**
SEQ ID NO: 5
(H10214 (11E7.C7 Humanized HC2))
Bold font indicates an exemplified variable region
while the bold, italic and underlined font
indicates exemplified CDRs.
MDPKGSLSWRILLFLSLAFELSYGEVQLVESGGGLVKPGGSLRLSCAASGFTFSR
YGMSWVRQAPGKGLEWVSTISSGGSYTYYTDSVKGRFTISRDNAKNSLYLQMNSL
RAEDTAVYYCERHGGDGYWYFDVWGQGTMVTVSSASTKGPSVFPLAPSSKSTSGG
TAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSKLTVD
KSRWQQGNVFSCSVMHEALHNHYTQKSLSLSPG**
SEQ ID NO: 6
(H10215 (11E7.C7 Humanized HC3, anti-tip chimer antibody))
Bold font indicates an exemplified variable region
while the bold, italic and underlined font
indicates exemplified CDRs.
MDPKGSLSWRILLFLSLAFELSYGEVQLVESGGGLVQPGRSLRLSCTASGFTESR
YGMSWVRQAPGKGLEWVATISSGGSYTYYTDSVKGRFTISRDNAKNILYLQMNSL
KTEDTAVYYCERHGGDGYWYFDVWGQGTMVTVSSASTKGPSVFPLAPSSKSTSGGT
AALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTKNQV
SLTCLVKGFYPSDIA VEWESNGQPENNYKTTPPVLDSDGSFFLYSKLTVDKSRWQ
QGNVFSCSVMHEALHNHYTQKSLSLSPG**
SEQ ID NO: 7
(L10210 (1F8.F1 Humanized LC1, anti-tip chimer antibody)
Bold font indicates an exemplified variable region
while the bold, italic and underlined font
indicates exemplified CDRs.
METDTLLLWVLLLWVPGSTGDVVMTQSPLSLPVTLGQPASISCRSSQSLLDSDGKT
FLNWLQQRPGQSPRRLIYLVSKLDSGVPDRFSGSGSGTDFTLKISRVEAEDVGVYY
CWQGTHEPYTFGQGTKLEIK RTVAAPSVFIFPPSDEQLKSGTASVVCLLNNFYPR
EAKVQWKVDNALQSGNSQESVTEQDSKDSTYSLSSTLTLSKADYEKHKVYACEVTH
QGLSSPVTKSFNRGEC*
SEQ ID NO: 8
(L10211 (1F8.F1 Humanized LC2, anti-tip chimer antibody)
Bold font indicates an exemplified variable region
while the bold, italic and underlined font
indicates exemplified CDRs.
METDTLLLWVLLLWVPGSTGDVVMTQSPLSLPVTLGQPASISCRSSQSLLDSDGKT
FLNWLQQRPGQSPRRLIYLVSKRDSGVPDRFSGSGSGTDFTLKISRVEAEDVGVYY
CWQGTHFPYTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKVYACEVTH
QGLSSPVTKSFNRGEC*
SEQ ID NO: 9
(L10212 (1F8.F1 Humanized LC3, anti-tip chimer antibody))
Bold font indicates an exemplified variable region
while the bold, italic and underlined font
indicates exemplified CDRs.
METDTLLLWVLLLWVPGSTGDVVMTQSPDSLAVSLGERATINCKSSQSLLDSDGK
TFLNWLQQKPGQPPKRLIYLVSKLDSGVPDRFSGSGSGTDFTLTISSLQAEDVAV
YYCWQGTHEPYTFGQGTKLEIKRTVAAPSVFIFPPSDEQLKSGTASVVCLLNNFY
PREAKVQWKVDNALQSGNSQESVTEQDSKDSTYSLSSTLTLSKADYEKHKVYACE
VTHQGLSSPVTKSFNRGEC*
SEQ ID NO: 10
(L10213 (11E7.C7 Humanized LC1))
Bold font indicates an exemplified variable region
while the bold, italic and underlined font
indicates exemplified CDRs.
METDTLLLWVLLLWVPGSTGDIQMTQSPSSLSASVGDRVTITCRASQDISNYLNW
YQQKPGKAVKLLIYYTSRLHSGVPSRFSGSGSGTDYTLTISSLQPEDFATYFCQQ
GNPLRTFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGL
SSPVTKSFNRGEC*
SEQ ID NO: 11
(L10214 (11E7.C7 Humanized LC2))
Bold font indicates an exemplified variable region
while the bold, italic and underlined font
indicates exemplified CDRs.
METDTLLLWVLLLWVPGSTGDIQMTQSPSSLSASVGDRVTITCRASQDISNYLN
WYQQKPGKAVKLLIYYTSRLHSGVPSRFSGSGSGTDYTLTISSLQPEDFATYYCQ
QGNPLRTFGGGTKVEIKRTVAAPSVFIFPPSDEQLKSGTASVVCLLNNFYPREAK
VQWKVDNALQSGNSQESVTEQDSKDSTYSLSSTLTLSKADYEKHKVYACEVTHQG
LSSPVTKSFNRGEC*
SEQ ID NO: 12
(L10215 (11E7.C7 Humanized LC3))
Bold font indicates an exemplified variable region
while the bold, italic and underlined font
indicates exemplified CDRs.
METDTLLLWVLLLWVPGSTGDIVMTQSPATLSLSPGERATLSCRASQDISNYLNW
YQQKPGQAVRLLIYYTSRLHSGIPARFSGSGSGTDYTLTISSLEPEDFAVYFCQQ
GNPLRTFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGL
SSPVTKSFNRGEC*
SEQ ID NO: 13
(Heavy Chain Consensus Sequence, anti-tip chimer antibody)
MDPKGSLSWR ILLFLSLAFE LSYGEVqLVe SGgglvXPGg SlrlSCaASG 50
FTFXXYXMSW VRQAPGkgLE WVaTIXSXXX XTYYXDsvkG RfTIsRDNaK 100
NtLYlqmnSL raEDTAVYYC XXXXXXXYXX FDXWGQGTXV TVSSASTKGP 150
SVFPLAPSSK STSGGTAALG CLVKDYFPEP VTVSWNSGAL TSGVHTFPAV 200
LQSSGLYSLS SVVTVPSSSL GTQTYICNVN HKPSNTKVDK KVEPKSCDKT 250
HTCPPCPAPE LLGGPSVFLF PPKPKDTLMI SRTPEVTCVV VDVSHEDPEV 300
KFNWYVDGVE VHNAKTKPRE EQYNSTYRVV SVLTVLHQDW LNGKEYKCKV 350
SNKALPAPIE KTISKAKGQP REPQVYTLPP SREEMTKNQV SLTCLVKGFY 400
PSDIAVEWES NGQPENNYKT TPPVLDSDGS FFLYSKLTVD KSRWQQGNVF 450
SCSVMHEALH NHYTQKSLSL SPG** 475
Wherein X and a small letter can be substituted with
any amino acid or alternatively, with an
amino acid selected from SEQ ID NOs: 1-6, in the
corresponding position. In one
embodiment, X may also indicate absence of an
amino acid residue.
SEQ ID NO: 14
(Light Chain Consensus Sequence, anti-tip chimer antibody)
METDTLLLWV LLLWVPGSTG DXvMTQSPXs LsvslGXrat isCrXSQXXX 50
XXXXXXXLNW XQQkPGqaXX XLIYXXSXIX SGvPdRFSGS GSGTDXTLtI 100
SslXXEDXav YyCXQGXXXX XTFGXGTKXE IKRTVAAPSV FIFPPSDEQL 150
KSGTASVVCL LNNFYPREAK VQWKVDNALQ SGNSQESVTE QDSKDSTYSL 200
SSTLTLSKAD YEKHKVYACE VTHQGLSSPV TKSFNRGEC* 240
Wherein X and a small letter can be substituted with
any amino acid or alternatively, with an
amino acid selected from SEQ ID NOs: 7-12, in the
corresponding position. In one
embodiment, X may also indicate absence of an amino
acid residue.
SEQ ID NO: 15
Human IgD constant region, Uniprot: P01880
APTKAPDVFPIISGCRHPKDNSPVVLACLITGYHPTSVTVTWYMGTQSQPQRTFPEIQ
RRDSYYMTSSQLSTPLQQWRQGEYKCVVQHTASKSKKEIFRWPESPKAQASSVPTA
QPQAEGSLAKATTAPATTRNTGRGGEEKKKEKEKEEQEERETKTPECPSHTQPLGVY
LLTPAVQDLWLRDKATFTCFVVGSDLKDAHLTWEVAGKVPTGGVEEGLLERHSNG
SQSQHSRLTLPRSLWNAGTSVTCTLNHPSLPPQRLMALREPAAQAPVKLSLNLLASS
DPPEAASWLLCEVSGFSPPNILLMWLEDQREVNTSGFAPARPPPQPGSTTFWAWSVL
RVPAPPSPQPATYTCVVSHEDSRTLLNASRSLEVSYVTDHGPMK
SEQ ID NO: 16
Human IgG1 constant region, Uniprot: P01857
ASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAP
ELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPR
EPQVYTLPPSRDELTKNQVSLTCLVKGFYPSDIA VEWESNGQPENNYKTTPPVLDSD
GSFFLYSKLTVDKSRWQQGNVFSCSVMHEALHNHYTQKSLSLSPGK
SEQ ID NO: 17
Human IgG2 constant region, Uniprot: P01859
ASTKGPSVFPLAPCSRSTSESTAALGCLVKDYFPEPVTVSWNSGALTSGVHTFPAVLQ
SSGLYSLSSVVTVPSSNFGTQTYTCNVDHKPSNTKVDKTVERKCCVECPPCPAPPVA
GPSVFLFPPKPKDTLMISRTPEVTCVVVDVSHEDPEVQFNWYVDGVEVHNAKTKPR
EEQFNSTFRVVSVLTVVHQDWLNGKEYKCKVSNKGLPAPIEKTISKTKGQPREPQVY
TLPPSREEMTKNQVSLTCLVKGFYPSDISVEWESNGQPENNYKTTPPMLDSDGSFFL
YSKLTVDKSRWQQGNVFSCSVMHEALHNHYTQKSLSLSPGK
SEQ ID NO: 18
Human IgG3 constant region, Uniprot: P01860
ASTKGPSVFPLAPCSRSTSGGTAALGCLVKDYFPEPVTVSWNSGALTSGVHTFPAVL
QSSGLYSLSSVVTVPSSSLGTQTYTCNVNHKPSNTKVDKRVELKTPLGDTTHTCPRC
PEPKSCDTPPPCPRCPEPKSCDTPPPCPRCPEPKSCDTPPPCPRCPAPELLGGPSVFL
FPPKPKDTLMISRTPEVTCVVVDVSHEDPEVQFKWYVDGVEVHNAKTKPREEQYNSTF
RVVSVLTVLHQDWLNGKEYKCKVSNKALPAPIEKTISKTKGQPREPQVYTLPPSREEM
TKNQVSLTCLVKGFYPSDIAVEWESSGQPENNYNTTPPMLDSDGSFFLYSKLTVDKS
RWQQGNIFSCSVMHEALHNRFTQKSLSLSPGK
SEQ ID NO: 19
Human IgM constant region, Uniprot: P01871
GSASAPTLFPLVSCENSPSDTSSVAVGCLAQDFLPDSITLSWKYKNNSDISSTRGFPS
VLRGGKYAATSQVLLPSKDVMQGTDEHVVCKVQHPNGNKEKNVPLPVIAELPPKVSV
FVPPRDGFFGNPRKSKLICQATGFSPRQIQVSWLREGKQVGSGVTTDQVQAEAKESG
PTTYKVTSTLTIKESDWLGQSMFTCRVDHRGLTFQQNASSMCVPDQDTAIRVFAIPPS
FASIFLTKSTKLTCLVTDLTTYDSVTISWTRQNGEAVKTHTNISESHPNATFSAVGEAS
ICEDDWNSGERFTCTVTHTDLPSPLKQTISRPKGVALHRPDVYLLPPAREQLNLRESA
TITCLVTGFSPADVFVQWMQRGQPLSPEKYVTSAPMPEPQAPGRYFAHSILTVSEEE
WNTGETYTCVAHEALPNRVTERTVDKSTGKPTLYNVSLVMSDTAGTCY
SEQ ID NO: 20
Human IgG4 constant region, Uniprot: P01861
ASTKGPSVFPLAPCSRSTSESTAALGCLVKDYFPEPVTVSWNSGALTSGVHTFPAVLQ
SSGLYSLSSVVTVPSSSLGTKTYTCNVDHKPSNTKVDKRVESKYGPPCPSCPAPEFLG
GPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLDSDGSFFL
YSRLTVDKSRWQEGNVFSCSVMHEALHNHYTQKSLSLSLGK
SEQ ID NO: 21
Human IgA1 constant region, Uniprot: P01876
ASPTSPKVFPLSLCSTQPDGNVVIACLVQGFFPQEPLSVTWSESGQGVTARNFPPSQD
ASGDLYTTSSQLTLPATQCLAGKSVTCHVKHYTNPSQDVTVPCPVPSTPPTPSPSTPP
TPSPSCCHPRLSLHRPALEDLLLGSEANLTCTLTGLRDASGVTFTWTPSSGKSAVQGP
PERDLCGCYSVSSVLPGCAEPWNHGKTFTCTAAYPESKTPLTATLSKSGNTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKPTHVNVSVV
MAEVDGTCY
SEQ ID NO: 22
Human IgA2 constant region, Uniprot: P01877
ASPTSPKVFPLSLDSTPQDGNVVVACLVQGFFPQEPLSVTWSESGQNVTARNFPPSQD
ASGDLYTTSSQLTLPATQCPDGKSVTCHVKHYTNPSQDVTVPCPVPPPPPCCHPRLSL
HRPALEDLLLGSEANLTCTLTGLRDASGATFTWTPSSGKSAVQGPPERDLCGCYSVS
SVLPGCAQPWNHGETFTCTAAHPELKTPLTANITKSGNTFRPEVHLLPPPSEELALNE
LVTLTCLARGFSPKDVLVRWLQGSQELPREKYLTWASRQEPSQGTTTFAVTSILRVA
AEDWKKGDTFSCMVGHEALPLAFTQKTIDRMAGKPTHVNVSVVMAEVDGTCY
SEQ ID NO: 23
Human Ig kappa constant region, Uniprot: P01834
TVAAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
SEQ ID NO: 24
(Tip Heavy Chain Consensus Sequence)
MDPKGSLSWR ILLFLSLAFE LSYGEVKLVe SGgglvqPGg SlrlSCaASG 50
FTFRTYAMSW VRQAPGkgLE WVATIGSDRR HTYYPDsvkG RfTIsRDNaK 100
NTLYlqmnSL RaEDTAVYYC VGPYDGYYGE FDYWGQGTLV TVSSASTKGP 150
SVEPLAPSSK STSGGTAALG CLVKDYFPEP VTVSWNSGAL TSGVHTFPAV 200
LQSSGLYSLS SVVTVPSSSL GTQTYICNVN HKPSNTKVDK KVEPKSCDKT 250
HTCPPCPAPE LLGGPSVFLF PPKPKDTLMI SRTPEVTCVV VDVSHEDPEV 300
KFNWYVDGVE VHNAKTKPRE EQYNSTYRVV SVLTVLHQDW LNGKEYKCKV 350
SNKALPAPIE KTISKAKGQP REPQVYTLPP SREEMTKNQV SLTCLVKGFY 400
PSDIAVEWES NGQPENNYKT TPPVLDSDGS FFLYSKLTVD KSRWQQGNVF 450
SCSVMHEALH NHYTQKSLSL SPG** 475
Wherein a small letter can be substituted with an
amino acid selected from SEQ ID NOs: 1-3
in the corresponding position.
SEQ ID NO: 25
(Tip Light Chain Consensus Sequence)
METDTLLLWV LLLWVPGSTG DVVMTQSPIS LpVtLGqpAs IsCrSSQSLL 50
DSDGKTFLNW LQQrPGQsPr RLIYLVSKID SGVPDRFSGS GSGTDFTLKI 100
SrveAEDVgV YYCWQGTHFP YTFGQGTKLE IKRTVAAPSV FIFPPSDEQL 150
KSGTASVVCL LNNFYPREAK VQWKVDNALQ SGNSQESVTE QDSKDSTYSL 200
SSTLTLSKAD YEKHKVYACE VTHQGLSSPV TKSENRGEC** 240
Wherein a small letter can be substituted with an
amino acid selected from SEQ ID NOs: 7-9
in the corresponding position.
SEQ ID NO: 26
(Tail Heavy Chain Consensus Sequence)
MDPKGSLSWR ILLFLSLAFE LSYGEVQLVE SGGGLVKPGg SLRLSCaASG 50
FTFSRYGMSW VRQAPGKGLE WVaTISSGGS YTYYTDSVKG RFTISRDNAK 100
NsLYLQMNSL raEDTAVYYC ERHGGDGYWY FDVWGQGTMV TVSSASTKGP 150
SVFPLAPSSK STSGGTAALG CLVKDYFPEP VTVSWNSGAL TSGVHTFPAV 200
LQSSGLYSLS SVVTVPSSSL GTQTYICNVN HKPSNTKVDK KVEPKSCDKT 250
HTCPPCPAPE LLGGPSVFLF PPKPKDTLMI SRTPEVTCVV VDVSHEDPEV 300
KFNWYVDGVE VHNAKTKPRE EQYNSTYRVV SVLTVLHQDW LNGKEYKCKV 350
SNKALPAPIE KTISKAKGQP REPQVYTLPP SREEMTKNQV SLTCLVKGFY 400
PSDIAVEWES NGQPENNYKT TPPVLDSDGS FFLYSKLTVD KSRWQQGNVF 450
SCSVMHEALH NHYTQKSLSL SPG** 475
Wherein a small letter can be substituted with an
amino acid selected from SEQ ID NOs: 4-6
in the corresponding position.
SEQ ID NO: 27
(Tail Light Chain Consensus Sequence)
METDTLLLWV LLLWVPGSTG DIqMTQSPss LSaSvGdRvT itCRASQDIS 50
NYLNWYQQKP GKAVKLLIYY TSRLHSGvPs RFSGSGSGTD YTLTISSLqP 100
EDFAtYfCQQ GNPLRTFGGG TKVEIKRTVA APSVFIFPPS DEQLKSGTAS 150
VVCLLNNFYP REAKVQWKVD NALQSGNSQE SVTEQDSKDS TYSLSSTLTL 200
SKADYEKHKV YACEVTHQGL SSPVTKSFNR GEC* 234
Wherein a small letter can be substituted with an
amino acid selected from SEQ ID NOs: 10-12
in the corresponding position.
SEQ ID NO: 28
(the consensus sequence that IHF binds)
WATCAANNNNTTR
Wherein W is A or T and R is a purine.
SEQ ID NO. 29 (E. coli hupA, Genbank accession
No.: AP_003818, Last accessed
Mar 21, 2011)
MNKTQLIDVIAEKAELSKTQAKAALESTLAAITESLKEGDAVQLVGFGTFK
VNHRAERTGRNPQTGKEIKIAAANVPAFVSGKALKDAVK
SEQ ID NO. 30
(E. coli hupB, Genbank accession
No.: AP_001090.1, Last accessed
March 21, 2011)
MNKSQLIDKIAAGADISKAAAGRALDAIIASVTESLKEGDDVALVGFG
TFAVKERAARTGRNPQTGKEITIAAAKVPSFRAGKALKDAVN
SEQ ID NO: 31
(IhfA, A tip fragment)
NFELRDKSSRPGRNPKTGDVV
SEQ ID NO: 32
(IhfB, B tip fragment)
SLHHR QPRLGRNPKTGDSVNL
SEQ ID NO: 33
(a peptide linker)
Gly-Pro-Ser-Leu-Lys-Leu
SEQ ID NO: 34
(a peptide linker)
Gly-Pro-Ser-Leu
SEQ ID NO: 35
(a peptide linker)
Pro-Ser-Leu-Lys
SEQ ID NO: 36
(a peptide linker)
Gly-Pro-Ser-Leu-Lys
SEQ ID NO: 37
(a peptide linker)
Ser-Leu-Lys-Leu
SEQ ID NO: 38
(tip-chimeric peptide IhfA5-mIhfB4NTHI)
RPGRNPX₁TGDVVPVSARRVV-X-FSLHHRQPRLGRNPX₁TGDSV
wherein “X” is an optional amino acid linker
sequence, optionally comprising, or consisting
essentially of, or yet further consisting of
between 1 to 20 amino acids
wherein “X₁” is any amino acid or alternatively
“X₁” is selected from the amino acids Q, R,
K, S, or T.
SEQ ID NO: 39
(tip-chimeric peptide IhfA5-mIhfB4NTHI)
RPGRNPKTGDVVPVSARRVV-X-FSLHHRQPRLGRNPKTGDSV
wherein “X” is an optional amino acid linker
sequence optionally comprising between 1 to 20
amino acids.
SEQ ID NO: 40
(tip-chimeric peptide IhfA5-mIhfB4NTHI,
in some embodiments,
which is referred to herein as the tip chimer)
RPGRNPKTGDVVPVSARRVVGPSLFSLHHRQPRLGRNPKTGDSV
SEQ ID NO: 41
(tail-chimeric peptide IhfA3-IhfB2NTHI)
FLEEIRLSLESGQDVKLSGF-X-TLSAKEIENMVKDILEFISQ
SEQ ID NO. 42:
Non-limiting exemplary linker: GGSGGS
SEQ ID NO. 43:
Non-limiting exemplary linker: GPSLKL.
SEQ ID NO. 44:
Non-limiting exemplary linker: GGG.
SEQ ID NO. 45:
Non-limiting exemplary linker: GPSL.
SEQ ID NO. 46:
Non-limiting exemplary linker: GPS.
SEQ ID NO. 47:
Non-limiting exemplary linker: PSLK.
SEQ ID NO. 48:
Non-limiting exemplary linker: GPSLK.
SEQ ID NO. 49:
Non-limiting exemplary linker: SLKL.
SEQ ID NO: 50
(tail-chimeric peptide IhfA3-IhfB2NTHI)
FLEEIRLSLESGQDVKLSGFGPSLTLSAKEIENMVKDILEFISQ

### REFERENCES

2017a. Antibacterial agents in clinical development: an analysis of the antibacterial clinical development pipeline, including tuberculosis. Geneva: World Health Organization. apps.who.int/iris/bitstream/handle/10665/258965/WHO-EMP-IAU-2017.11-eng.pdf;jsessionid=4C4B74B963CED463A94DD2DE9CB5EACA?sequence=1
2017b. The world is running out of antibiotics, WHO report confirms. Geneva, World Health Organization. www.who.int/news-room/detail/20-09-2017-the-world-is-running-out-of-antibiotics-who-report-confirms
Ahearn, C. P., Gallo, M. C., Murphy, T. F., 2017. Insights on persistent airway infection by non-typeable Haemophilus influenzae in chronic obstructive pulmonary disease. Pathog Dis 75. doi: 10.1093/femspd/ftx042
Andre, F. E., Booy, R., Bock, H. L., Clemens, J., Datta, S. K., John, T. J., Lee, B. W., Lolekha, S., Peltola, H., Ruff, T. A., Santosham, M., Schmitt, H. J., 2008. Vaccination greatly reduces disease, disability, death and inequity worldwide. Bull World Health Organ 86, 140-146. doi: 10.2471/blt.07.040089
Anes, J., McCusker, M. P., Fanning, S., Martins, M., 2015. The ins and outs of RND efflux pumps in Escherichia coli. Front Microbiol 6, 587. doi: 10.3389/fmicb.2015.00587
Armbruster, C. E., Hong, W., Pang, B., Dew, K. E., Juneau, R. A., Byrd, M. S., Love, C. F., Kock, N. D., Swords, W. E., 2009. LuxS promotes biofilm maturation and persistence of nontypeable Haemophilus influenzae in vivo via modulation of lipooligosaccharides on the bacterial surface. Infect Immun 77, 4081-4091. doi: 10.1128/IAI.00320-09
Armbruster, C. E., Hong, W., Pang, B., Weimer, K. E., Juneau, R. A., Turner, J., Swords, W. E., 2010. Indirect pathogenicity of Haemophilus influenzae and Moraxella catarrhalis in polymicrobial otitis media occurs via interspecies quorum signaling. mBio 1, e00102-00110. doi: 10.1128/mBio.00102-10
Armbruster, C. E., Pang, B., Murrah, K., Juneau, R. A., Perez, A. C., Weimer, K. E., Swords, W. E., 2011. RbsB (NTHI_0632) mediates quorum signal uptake in nontypeable Haemophilus influenzae strain 86-028NP. Mol Microbiol 82, 836-850. doi: 10.1111/j.1365-2958.2011.07831.x
Autio, T. J., Tapiainen, T., Koskenkorva, T., Narkio, M., Lappalainen, M., Nikkari, S., Hemmila, H., Koskela, K. A., Koskela, M., Koivunen, P., Alho, O. P., 2015. The role of microbes in the pathogenesis of acute rhinosinusitis in young adults. Laryngoscope 125, El-7. doi: 10.1002/lary.24862
Aziz, A., Sarovich, D. S., Nosworthy, E., Beissbarth, J., Chang, A. B., Smith-Vaughan, H., Price, E. P., Harris, T. M., 2019. Molecular signatures of non-typeable Haemophilus influenzae lung adaptation in pediatric chronic lung disease. Front Microbiol 10, 1622. doi: 10.3389/fmicb.2019.01622
Bailey, M. T., Lauber, C. L., Novotny, L. A., Goodman, S. D., Bakaletz, L. O., 2020. Immunization with a biofilm-disrupting nontypeable Haemophilus influenzae vaccine antigen did not alter the gut microbiome in chinchillas, unlike oral delivery of a broad-spectrum antibiotic commonly used for otitis media. mSphere 5, e00296-00220. doi: 10.1128/mSphere.00296-20
Bakaletz, L. O., Baker, B. D., Jurcisek, J. A., Harrison, A., Novotny, L. A., Bookwalter, J. E., Mungur, R., Munson, R. S., Jr., 2005. Demonstration of type IV pilus expression and a twitching phenotype by Haemophilus influenzae. Infect Immun 73, 1635-1643. doi: 10.1128/IAI.73.3.1635-1643.2005
Barkai, G., Leibovitz, E., Givon-Lavi, N., Dagan, R., 2009. Potential contribution by nontypable Haemophilus influenzae in protracted and recurrent acute otitis media. Pediatr Infect Dis J 28, 466-471. doi: 10.1097/inf.0b013e3181950c74
Barrero, M. A., Pietralonga, P. A., Schwarz, D. G., Silva, A., Jr., Paula, S. O., Moreira, M. A., 2014. Effect of the inhibitors phenylalanine arginyl ss-naphthylamide (PAssN) and 1-(1-naphthylmethyl)-piperazine (NMP) on expression of genes in multidrug efflux systems of Escherichia co/i isolates from bovine mastitis. Res Vet Sci 97, 176-181. doi: 10.1016/j.rvsc.2014.05.013
Beekmann, S. E., Heilmann, K. P., Richter, S. S., Garcia-de-Lomas, J., Doern, G. V., Group, G. S., 2005. Antimicrobial resistance in Streptococcus pneumoniae, Haemophilus influenzae, Moraxella catarrhalis and group A beta-haemolytic streptococci in 2002-2003. Results of the multinational GRASP Surveillance Program. Int J Antimicrob Agents 25, 148-156. doi: 10.1016/j.ijantimicag.2004.09.016
Belfield, K., Bayston, R., Birchall, J. P., Daniel, M., 2015. Do orally administered antibiotics reach concentrations in the middle ear sufficient to eradicate planktonic and biofilm bacteria?A review. Int J Pediatr Otorhinolaryngol 79, 296-300. doi: 10.1016/j.ijporl.2015.01.003
Brockson, M. E., Novotny, L. A., Mokrzan, E. M., Malhotra, S., Jurcisek, J. A., Akbar, R., Devaraj, A., Goodman, S. D., Bakaletz, L. O., 2014. Evaluation of the kinetics and mechanism of action of anti-integration host factor-mediated disruption of bacterial biofilms. Mol Microbiol 93, 1246-1258. doi: 10.1111/mmi.12735
Cardines, R., Giufre, M., Pompilio, A., Fiscarelli, E., Ricciotti, G., Di Bonaventura, G., Cerquetti, M., 2012. Haemophilus influenzae in children with cystic fibrosis: antimicrobial susceptibility, molecular epidemiology, distribution of adhesins and biofilm formation. Int J Med Microbiol 302, 45-52. doi: 10.1016/j.ijmm.2011.08.003
Carruthers, M. D., Tracy, E. N., Dickson, A. C., Ganser, K. B., Munson, R. S., Jr., Bakaletz, L. O., 2012. Biological roles of nontypeable Haemophilus influenzae type IV pilus proteins encoded by the pil and com operons. J Bacteriol 194, 1927-1933. doi: 10.1128/JB.06540-11
Chambers, J. R., Cherny, K. E., Sauer, K., 2017. Susceptibility of Pseudomonas aeruginosa dispersed cells to antimicrobial agents is dependent on the dispersion cue and class of the antimicrobial agent used. Antimicrob Agents Chemother 61, e00846-00817. doi: 10.1128/AAC.00846-17
Chua, S. L., Liu, Y., Yam, J. K., Chen, Y., Vejborg, R. M., Tan, B. G., Kjelleberg, S., Tolker-Nielsen, T., Givskov, M., Yang, L., 2014. Dispersed cells represent a distinct stage in the transition from bacterial biofilm to planktonic lifestyles. Nat Commun 5, 4462. doi: 10.1038/ncomms5462
Cleary, D., Devine, V., Morris, D., Osman, K., Gladstone, R., Bentley, S., Faust, S., Clarke, S., 2018. Pneumococcal vaccine impacts on the population genomics of non-typeable Haemophilus influenzae. Microb Genom 4, e000209. doi: 10.1099/mgen.0.000209
Costerton, J. W., Stewart, P. S., Greenberg, E. P., 1999. Bacterial biofilms: a common cause of persistent infections. Science 284, 1318-1322. doi: 10.1126/science.284.5418.1318
D'Andrea, M. M., Lau, G. W., 2020. DNABII targeting antibodies as vaccines against biofilm diseases. EBioMedicine 58, 102921. doi: 10.1016/j.ebiom.2020.102921
Daines, D. A., Bothwell, M., Furrer, J., Unrath, W., Nelson, K., Jarisch, J., Melrose, N., Greiner, L., Apicella, M., Smith, A. L., 2005. Haemophilus influenzae luxS mutants form a biofilm and have increased virulence. Microb Pathog 39, 87-96. doi: 10.1016/j.micpath.2005.06.003
Das, J., Mokrzan, E., Lakhani, V., Rosas, L., Jurcisek, J. A., Ray, W. C., Bakaletz, L. O., 2017. Extracellular DNA and type IV pilus expression regulate the structure and kinetics of biofilm formation by nontypeable Haemophilus influenzae. mBio 8, e01466-01417. doi: 10.1128/mBio.01466-17
Delcour, A. H., 2009. Outer membrane permeability and antibiotic resistance. Biochim Biophys Acta 1794, 808-816. doi: 10.1016/j.bbapap.2008.11.005
Devaraj, A., Buzzo, J., Rocco, C. J., Bakaletz, L. O., Goodman, S. D., 2018. The DNABII family of proteins is comprised of the only nucleoid associated proteins required for nontypeable Haemophilus influenzae biofilm structure. Microbiology Open 7, e00563. doi: 10.1002/mbo3.563
Devaraj, A., Justice, S. S., Bakaletz, L. O., Goodman, S. D., 2015. DNABII proteins play a central role in UPEC biofilm structure. Mol Microbiol 96, 1119-1135. doi: 10.1111/mmi.12994
Dongari-Bagtzoglou, A., 2008. Pathogenesis of mucosal biofilm infections: challenges and progress. Expert Rev Anti Infect Ther 6, 201-208. doi: 10.1586/14787210.6.2.201
Du, D., Wang-Kan, X., Neuberger, A., van Veen, H. W., Pos, K. M., Piddock, L. J. V., Luisi, B. F., 2018. Multidrug efflux pumps: structure, function and regulation. Nat Rev Microbiol 16, 523-539. doi: 10.1038/s41579-018-0048-6
Fernandez-Villa, D., Aguilar, M. R., Rojo, L., 2019. Folic acid antagonists: Antimicrobial and immunomodulating mechanisms and applications. Int J Mol Sci 20, 4996. doi: 10.3390/ijms20204996
Flemming, H. C., Wingender, J., 2010. The biofilm matrix. Nat Rev Microbiol 8, 623-633. doi: 10.1038/nrmicro2415
Freire, M. O., Devaraj, A., Young, A., Navarro, J. B., Downey, J. S., Chen, C., Bakaletz, L. O., Zadeh, H. H., Goodman, S. D., 2017. A bacterial-biofilm-induced oral osteolytic infection can be successfully treated by immuno-targeting an extracellular nucleoid-associated protein. Mol Oral Microbiol 32, 74-88. doi: 10.1111/omi.12155
Gilbert, J. A., Blaser, M. J., Caporaso, J. G., Jansson, J. K., Lynch, S. V., Knight, R., 2018. Current understanding of the human microbiome. Nat Med 24, 392-400. doi: 10.1038/nm.4517
Gillies, M., Ranakusuma, A., Hoffmann, T., Thorning, S., McGuire, T., Glasziou, P., Del Mar, C., 2015. Common harms from amoxicillin: a systematic review and meta-analysis of randomized placebo-controlled trials for any indication. CMAJ 187, E21-E31. doi: 10.1503/cmaj.140848
Goodman, S. D., Obergfell, K. P., Jurcisek, J. A., Novotny, L. A., Downey, J. S., Ayala, E. A., Tjokro, N., Li, B., Justice, S. S., Bakaletz, L. O., 2011. Biofilms can be dispersed by focusing the immune system on a common family of bacterial nucleoid-associated proteins. Mucosal Immunol 4, 625-637. doi: 10.1038/mi.2011.27
Goodwine, J., Gil, J., Doiron, A., Valdes, J., Solis, M., Higa, A., Davis, S., Sauer, K., 2019. Pyruvate-depleting conditions induce biofilm dispersion and enhance the efficacy of antibiotics in killing biofilms in vitro and in vivo. Sci Rep 9, 3763. doi: 10.1038/s41598-019-40378-z
Grevers, G., Wiedemann, S., Bohn, J. C., Blasius, R. W., Harder, T., Kroeniger, W., Vetter, V., Pircon, J. Y., Marano, C., 2012. Identification and characterization of the bacterial etiology of clinically problematic acute otitis media after tympanocentesis or spontaneous otorrhea in German children. BMC Infect Dis 12, 312. doi: 10.1186/1471-2334-12-312
Grkovic, S., Brown, M. H., Skurray, R. A., 2001. Transcriptional regulation of multidrug efflux pumps in bacteria. Semin Cell Dev Biol 12, 225-237. doi: 10.1006/scdb.2000.0248
Guilhen, C., Charbonnel, N., Parisot, N., Gueguen, N., Iltis, A., Forestier, C., Balestrino, D., 2016. Transcriptional profiling of Klebsiellapneumoniae defines signatures for planktonic, sessile and biofilm-dispersed cells. BMC Genomics 17, 237. doi: 10.1186/s12864-016-2557-x
Hall, C. W., Mah, T. F., 2017. Molecular mechanisms of biofilm-based antibiotic resistance and tolerance in pathogenic bacteria. FEMS Microbiol Rev 41, 276-301. doi: 10.1093/femsre/fux010
Harrison, A., Dyer, D. W., Gillaspy, A., Ray, W. C., Mungur, R., Carson, M. B., Zhong, H., Gipson, J., Gipson, M., Johnson, L. S., Lewis, L., Bakaletz, L. O., Munson, R. S., Jr., 2005. Genomic sequence of an otitis media isolate of nontypeable Haemophilus influenzae: comparative study with H. influenzae serotype d, strain KW20. J Bacteriol 187, 4627-4636. doi: 10.1128/JB.187.13.4627-4636.2005
Harrison, C. J., Woods, C., Stout, G., Martin, B., Selvarangan, R., 2009. Susceptibilities of Haemophilus influenzae, Streptococcus pneumoniae, including serotype 19A, and Moraxella catarrhalis paediatric isolates from 2005 to 2007 to commonly used antibiotics. J Antimicrob Chemother 63, 511-519. doi: 10.1093/jac/dkn538 Hassan, F., 2013. Molecular mechanisms of Moraxella catarrhalis-induced otitis media. Curr Allergy Asthma Rep 13, 512-517. doi: 10.1007/s11882-013-0374-8
Huovinen, P., 2001. Resistance to trimethoprim-sulfamethoxazole. Clin Infect Dis 32, 1608-1614. doi: 10.1086/320532
Jurcisek, J. A., Bakaletz, L. O., 2007. Biofilms formed by nontypeable Haemophilus influenzae in vivo contain both double-stranded DNA and type IV pilin protein. J Bacteriol 189, 3868-3875. doi: 10.1128/JB.01935-06
Jurcisek, J. A., Bookwalter, J. E., Baker, B. D., Fernandez, S., Novotny, L. A., Munson, R. S., Jr., Bakaletz, L. O., 2007. The PilA protein of non-typeable Haemophilus influenzae plays a role in biofilm formation, adherence to epithelial cells and colonization of the mammalian upper respiratory tract. Mol Microbiol 65, 1288-1299. doi: 10.1111/j.1365-2958.2007.05864.x
Jurcisek, J. A., Dickson, A. C., Bruggeman, M. E., Bakaletz, L. O., 2011. In vitro biofilm formation in an 8-well chamber slide. J Vis Exp 20, 2481. doi: 10.3791/2481
K, L. C. H., Akyol, S., Parkan, O. M., Dinc, G., Sav, H., Aydemir, G., 2017. Molecular characterization and antibiotic susceptibility of Haemophilus influenzae clinical isolates. Infez Med 25, 27-32.
Kaplan, E., Greene, N. P., Crow, A., Koronakis, V., 2018. Insights into bacterial lipoprotein trafficking from a structure of LolA bound to the LolC periplasmic domain. Proc Natl Acad Sci USA 115, E7389-E7397. doi: 10.1073/pnas.1806822115
Kapoor, G., Saigal, S., Elongavan, A., 2017. Action and resistance mechanisms of antibiotics: A guide for clinicians. J Anaesthesiol Clin Pharmacol 33, 300-305. doi: 10.4103/joacp.JOACP_349_15
Koff, W. C., Schenkelberg, T., 2019. The future of vaccine development. Vaccine. doi: 10.1016/j.vaccine.2019.07.101
Kuehn, J., Ismael, Z., Long, P. F., Barker, C. I., Sharland, M., 2015. Reported rates of diarrhea following oral penicillin therapy in pediatric clinical trials. J Pediatr Pharmacol Ther 20, 90-104. doi: 10.5863/1551-6776-20.2.90
Lamont, R. F., Moller Luef, B., Stener Jorgensen, J., 2020. Childhood inflammatory and metabolic disease following exposure to antibiotics in pregnancy, antenatally, intrapartum and neonatally. F1000Res 9, F1000 Faculty Rev-1144. doi: 10.12688/fl000research.19954.1
Lê, S., Josse, J., Husson, F., 2008. FactoMineR: A package for multivariate analysis. J. Stat. Softw. 25, 1-18. doi: 10.18637/jss.v025.i01
Leibovitz, E., Broides, A., Greenberg, D., Newman, N., 2010. Current management of pediatric acute otitis media. Expert Rev Anti Infect Ther 8, 151-161. doi: 10.1586/eri.09.112
Long, Y., Fu, W., Wang, S., Deng, X., Jin, Y., Bai, F., Cheng, Z., Wu, W., 2020. Fis Contributes to resistance of Pseudomonas aeruginosa to ciprofloxacin by regulating pyocin synthesis. J Bacteriol 202. doi: 10.1128/JB.00064-20
MacPherson, C. W., Mathieu, O., Tremblay, J., Champagne, J., Nantel, A., Girard, S. A., Tompkins, T. A., 2018. Gut bacterial microbiota and its resistome rapidly recover to basal state levels after short-term amoxicillin-clavulanic acid treatment in healthy adults. Sci Rep 8, 11192. doi: 10.1038/s41598-018-29229-5
Marks, L. R., Davidson, B. A., Knight, P. R., Hakansson, A. P., 2013. Interkingdom signaling induces Streptococcuspneumoniae biofilm dispersion and transition from asymptomatic colonization to disease. mBio 4, e00438-00413. doi: 10.1128/mBio.00438-13
Martin, R. G., Rosner, J. L., 1997. Fis, an accessorial factor for transcriptional activation of the mar (multiple antibiotic resistance) promoter of Escherichia coli in the presence of the activator MarA, SoxS, or Rob. J Bacteriol 179, 7410⁻⁷⁴¹⁹. doi: 10.1128/jb.179.23.7410-7419.1997
Martinez-Antonio, A., Collado-Vides, J., 2003. Identifying global regulators in transcriptional regulatory networks in bacteria. Curr Opin Microbiol 6, 482-489. doi: 10.1016/j.mib.2003.09.002
Mittal, R., Parrish, J. M., Soni, M., Mittal, J., Mathee, K., 2018. Microbial otitis media: recent advancements in treatment, current challenges and opportunities. J Med Microbiol 67, 1417-1425. doi: 10.1099/jmm.0.000810
Mokrzan, E. M., Johnson, T. J., Bakaletz, L. O., 2019. Expression of the nontypeable Haemophilus influenzae type IV pilus is stimulated by coculture with host respiratory tract epithelial cells. Infect Immun 87, e00704-00719. doi: 10.1128/IAI.00704-19
Mokrzan, E. M., Novotny, L. A., Brockman, K. L., Bakaletz, L. O., 2018. Antibodies against the majority subunit (PilA) of the type IV pilus of nontypeable Haemophilus influenzae disperse Moraxella catarrhalis from a dual-species biofilm. mBio 9, e02423-02418. doi: 10.1128/mBio.02423-18
Mokrzan, E. M., Ward, M. O., Bakaletz, L. O., 2016. Type IV pilus expression is upregulated in nontypeable Haemophilus influenzae biofilms formed at the temperature of the human nasopharynx. J Bacteriol 198, 2619-2630. doi: 10.1128/JB.01022-15
Murphy, T. F., Kirkham, C., Lesse, A. J., 2006. Construction of a mutant and characterization of the role of the vaccine antigen P6 in outer membrane integrity of nontypeable Haemophilus influenzae. Infect Immun 74, 5169-5176. doi: 10.1128/IAI.00692-06
Nikolaidis, I., Favini-Stabile, S., Dessen, A., 2014. Resistance to antibiotics targeted to the bacterial cell wall. Protein Sci 23, 243-259. doi: 10.1002/pro.2414
Novotny, L. A., Adams, L. D., Kang, D. R., Wiet, G. J., Cai, X., Sethi, S., Murphy, T. F., Bakaletz, L. O., 2009. Epitope mapping immunodominant regions of the PilA protein of nontypeable Haemophilus influenzae (NTHI) to facilitate the design of two novel chimeric vaccine candidates. Vaccine 28, 279-289. doi: 10.1016/j.vaccine.2009.08.017
Novotny, L. A., Amer, A. O., Brockson, M. E., Goodman, S. D., Bakaletz, L. O., 2013a. Structural stability of Burkholderia cenocepacia biofilms is reliant on eDNA structure and presence of a bacterial nucleic acid binding protein. PLoS One 8, e67629. doi: 10.1371/journal.pone.0067629
Novotny, L. A., Clements, J. D., Bakaletz, L. O., 2013b. Kinetic analysis and evaluation of the mechanisms involved in the resolution of experimental nontypeable Haemophilus influenzae-induced otitis media after transcutaneous immunization. Vaccine 31, 3417-3426. doi: 10.1016/j.vaccine.2012.10.033
Novotny, L. A., Goodman, S. D., Bakaletz, L. O., 2019. Redirecting the immune response towards immunoprotective domains of a DNABII protein resolves experimental otitis media. NPJ Vaccines 4, 43. doi: 10.1038/s41541-019-0137-1
Novotny, L. A., Goodman, S. D., Bakaletz, L. O., 2020. Targeting a bacterial DNABII protein with a chimeric peptide immunogen or humanised monoclonal antibody to prevent or treat recalcitrant biofilm-mediated infections. EBioMedicine. doi: 10.1016/j.ebiom.2020.102867
Novotny, L. A., Jurcisek, J. A., Goodman, S. D., Bakaletz, L. O., 2016. Monoclonal antibodies against DNA-binding tips of DNABII proteins disrupt biofilms in vitro and induce bacterial clearance in vivo. EBioMedicine 10, 33-44. doi: 10.1016/j.ebiom.2016.06.022
Novotny, L. A., Jurcisek, J. A., Ward, M. O., Jr., Jordan, Z. B., Goodman, S. D., Bakaletz, L. O., 2015. Antibodies against the majority subunit of type IV Pili disperse nontypeable Haemophilus influenzae biofilms in a LuxS-dependent manner and confer therapeutic resolution of experimental otitis media. Mol Microbiol 96, 276-292. doi: 10.1111/mmi.12934
Pallav, K., Dowd, S. E., Villafuerte, J., Yang, X., Kabbani, T., Hansen, J., Dennis, M., Leffler, D. A., Newburg, D. S., Kelly, C. P., 2014. Effects of polysaccharopeptide from Trametes versicolor and amoxicillin on the gut microbiome of healthy volunteers: a randomized clinical trial. Gut Microbes 5, 458-467. doi: 10.4161/gmic.29558
Pang, B., Armbruster, C. E., Foster, G., Learman, B. S., Gandhi, U., Swords, W. E., 2018. Autoinducer 2 (AI-2) production by nontypeable Haemophilus influenzae 86-028NP promotes expression of a predicted glycosyltransferase that is a determinant of biofilm maturation, prevention of dispersal, and persistence in vivo. Infect Immun 86. doi: 10.1128/IAI.00506-18
Pang, B., Hong, W., Kock, N. D., Swords, W. E., 2012. Dps promotes survival of nontypeable Haemophilus influenzae in biofilm communities in vitro and resistance to clearance in vivo. Front Cell Infect Microbiol 2, 58. doi: 10.3389/fcimb.2012.00058
Parsons, J. B., Rock, C. O., 2013. Bacterial lipids: metabolism and membrane homeostasis. Prog Lipid Res 52, 249-276. doi: 10.1016/j.plipres.2013.02.002
Patini, R., Mangino, G., Martellacci, L., Quaranta, G., Masucci, L., Gallenzi, P., 2020. The effect of different antibiotic regimens on bacterial resistance: A systematic review. Antibiotics (Basel) 9, 22. doi: 10.3390/antibiotics9010022
Pelton, S., 2020. Acute otitis media in children: Treatment, in: Post, T. (Ed.), UpToDate, Waltham, MA.
Petrova, O. E., Sauer, K., 2016. Escaping the biofilm in more than one way: desorption, detachment or dispersion. Curr Opin Microbiol 30, 67-78. doi: 10.1016/j.mib.2016.01.004
Pettigrew, M. M., Marks, L. R., Kong, Y., Gent, J. F., Roche-Hakansson, H., Hakansson, A. P., 2014. Dynamic changes in the Streptococcus pneumoniae transcriptome during transition from biofilm formation to invasive disease upon influenza A virus infection. Infect Immun 82, 4607-4619. doi: 10.1128/IAI.02225-14
Piltcher, O. B., Kosugi, E. M., Sakano, E., Mion, O., Testa, J. R. G., Romano, F. R., Santos, M. C. J., Di Francesco, R. C., Mitre, E. I., Bezerra, T. F. P., Roithmann, R., Padua, F. G., Valera, F. C. P., LubiancaNeto, J. F., Sa, L. C. B., Pignatari, S. S. N., Avelino, M. A. G., Caixeta, J. A. S., Anselmo-Lima, W. T., Tamashiro, E., 2018. How to avoid the inappropriate use of antibiotics in upper respiratory tract infections? A position statement from an expert panel. Braz J Otorhinolaryngol 84, 265-279. doi: 10.1016/j.bjorl.2018.02.001
Poole, J., Foster, E., Chaloner, K., Hunt, J., Jennings, M. P., Bair, T., Knudtson, K., Christensen, E., Munson, R. S., Jr., Winokur, P. L., Apicella, M. A., 2013. Analysis of nontypeable Haemophilus influenzae phase-variable genes during experimental human nasopharyngeal colonization. J Infect Dis 208, 720-727. doi: 10.1093/infdis/jit240
Rappuoli, R., Bloom, D. E., Black, S., 2017. Deploy vaccines to fight superbugs. Nature 552, 165-167. doi: 10.1038/d41586-017-08323-0
Ribeiro da Cunha, B., Fonseca, L. P., Calado, C. R. C., 2019. Antibiotic discovery: Where have we come from, where do we go? Antibiotics (Basel) 8, 45. doi: 10.3390/antibiotics8020045
Rolfe, M. D., Rice, C. J., Lucchini, S., Pin, C., Thompson, A., Cameron, A. D., Alston, M., Stringer, M. F., Betts, R. P., Baranyi, J., Peck, M. W., Hinton, J. C., 2012. Lag phase is a distinct growth phase that prepares bacteria for exponential growth and involves transient metal accumulation. J Bacteriol 194, 686-701. doi: 10.1128/JB.06112-11
Rumbaugh, K. P., Sauer, K., 2020. Biofilm dispersion. Nat Rev Microbiol. doi: 10.1038/s41579-020-0385-0
Sauer, K., Cullen, M. C., Rickard, A. H., Zeef, L. A., Davies, D. G., Gilbert, P., 2004. Characterization of nutrient-induced dispersion in Pseudomonas aeruginosa PAO1 biofilm. J Bacteriol 186, 7312-7326. doi: 10.1128/JB.186.21.7312-7326.2004
Sethi, S., Murphy, T. F., 2008. Infection in the pathogenesis and course of chronic obstructive pulmonary disease. N Engl J Med 359, 2355-2365. doi: 10.1056/NEJMra0800353
Sharma, D., Misba, L., Khan, A. U., 2019. Antibiotics versus biofilm: an emerging battleground in microbial communities. Antimicrob Resist Infect Control 8, 76. doi: 10.1186/s13756-019-0533-3
Sikkema, D. J., Murphy, T. F., 1992. Molecular analysis of the P2 porin protein of nontypeable Haemophilus influenzae. Infect Immun 60, 5204-5211. doi: 10.1128/IAI.60.12.5204-5211.1992
Silva, M. D., Sillankorva, S., 2019. Otitis media pathogens—A life entrapped in biofilm communities. Crit Rev Microbiol 45, 595-612. doi: 10.1080/1040841X.2019.1660616
Sirakova, T., Kolattukudy, P. E., Murwin, D., Billy, J., Leake, E., Lim, D., DeMaria, T., Bakaletz, L., 1994. Role of fimbriae expressed by nontypeable Haemophilus influenzae in pathogenesis of and protection against otitis media and relatedness of the fimbrin subunit to outer membrane protein A. Infect Immun 62, 2002-2020. doi:
Song, J. H., Dagan, R., Klugman, K. P., Fritzell, B., 2012. The relationship between pneumococcal serotypes and antibiotic resistance. Vaccine 30, 2728-2737. doi: 10.1016/j.vaccine.2012.01.091
Stewart, P. S., 2002. Mechanisms of antibiotic resistance in bacterial biofilms. Int J Med Microbiol 292, 107-113. doi: 10.1078/1438-4221-00196
Surette, M. G., Miller, M. B., Bassler, B. L., 1999. Quorum sensing in Escherichia coli, Salmonella typhimurium, and Vibrio harveyi: a new family of genes responsible for autoinducer production. Proc Natl Acad Sci USA 96, 1639-1644. doi: 10.1073/pnas.96.4.1639
Swinger, K. K., Rice, P. A., 2004. IHF and HU: flexible architects of bent DNA. Curr Opin Struct Biol 14, 28-35. doi: 10.1016/j.sbi.2003.12.003
Swords, W. E., Jones, P. A., Apicella, M. A., 2003. The lipo-oligosaccharides of Haemophilus influenzae: an interesting array of characters. J Endotoxin Res 9, 131-144. doi: 10.1179/096805103125001531
Tagliabue, A., Rappuoli, R., 2018. Changing priorities in vaccinology: Antibiotic resistance moving to the top. Front Immunol 9, 1068. doi: 10.3389/fimmu.2018.01068
Tatusov, R. L., Galperin, M. Y., Natale, D. A., Koonin, E. V., 2000. The COG database: a tool for genome-scale analysis of protein functions and evolution. Nucleic Acids Res 28, 33-36. doi: 10.1093/nar/28.1.33
Toone, S. L., Ratkiewicz, M., Novotny, L. A., Phong, B. L., Bakaletz, L. O., 2020. Nontypeable Haemophilus influenzae Type IV pilus mediates augmented adherence to rhinovirus-infected human airway epithelial cells. Infect Immun. doi: 10.1128/IAI.00248-20
Tristram, S., Jacobs, M. R., Appelbaum, P. C., 2007. Antimicrobial resistance in Haemophilus influenzae. Clin Microbiol Rev 20, 368-389. doi: 10.1128/CMR.00040-06
Vergison, A., 2008. Microbiology of otitis media: a moving target. Vaccine 26 Suppl 7, G5-10. doi: 10.1016/j.vaccine.2008.11.006
Wald, E. R., DeMuri, G. P., 2018. Antibiotic recommendations for acute otitis media and acute bacterial sinusitis: Conundrum no more. Pediatr Infect Dis J 37, 1255-1257. doi: 10.1097/INF.0000000000002009
Wasserman, R. C., Gerber, J. S., 2017. Acute otitis media in the 21st century: What now? Pediatrics 140. doi: 10.1542/peds.2017-1966
Whitchurch, C. B., Tolker-Nielsen, T., Ragas, P. C., Mattick, J. S., 2002. Extracellular DNA required for bacterial biofilm formation. Science 295, 1487. doi: 10.1126/science.295.5559.1487
Wickham, H., Chang, W., Henry, L., Pedersen, T. L., Takahashi, K., Wilke, C., Woo, K., Yutani, H., Dunnington, D., 2016. ggplot2: Elegant Graphics for Data Analysis. Springer-Verlag, New York.
Wiertsema, S. P., Kirkham, L. A., Corscadden, K. J., Mowe, E. N., Bowman, J. M., Jacoby, P., Francis, R., Vijayasekaran, S., Coates, H. L., Riley, T. V., Richmond, P., 2011. Predominance of nontypeable Haemophilus influenzae in children with otitis media following introduction of a 3+0 pneumococcal conjugate vaccine schedule. Vaccine 29, 5163-5170. doi: 10.1016/j.vaccine.2011.05.035
Ysebaert, C., Denoel, P., Weynants, V., Bakaletz, L. O., Novotny, L. A., Godfroid, F., Hermand, P., 2019. A protein E-PilA fusion protein shows vaccine potential against nontypeable Haemophilus influenzae in mice and chinchillas. Infect Immun 87, e00345-00319. doi: 10.1128/IAI.00345-19
Zapun, A., Contreras-Martel, C., Vernet, T., 2008. Penicillin-binding proteins and beta-lactam resistance. FEMS Microbiol Rev 32, 361-385. doi: 10.1111/j.1574-6976.2007.00095.x
Zhang, L., Xie, J., Patel, M., Bakhtyar, A., Ehrlich, G. D., Ahmed, A., Earl, J., Marrs, C. F., Clemans, D., Murphy, T. F., Gilsdorf, J. R., 2012. Nontypeable Haemophilus influenzae genetic islands associated with chronic pulmonary infection. PLoS One 7, e44730. doi: 10.1371/journal.pone.0044730
Clark, K., Karsch-Mizrachi, I., Lipman, D. J., Ostell, J., & Sayers, E. W. (2016). GenBank. Nucleic acids research, 44(D1), D67-D72. doi.org/10.1093/nar/gkv1276
Aziz, A., Sarovich, D. S., Nosworthy, E., Beissbarth, J., Chang, A. B., Smith-Vaughan, H., Price, E. P., & Harris, T. M. (2019). Molecular Signatures of Non-typeable Haemophilus influenzae Lung Adaptation in Pediatric Chronic Lung Disease. Frontiers in microbiology, 10, 1622. doi.org/10.3389/fmnicb.2019.01622
Tatusov, R. L., Galperin, M. Y., Natale, D. A., & Koonin, E. V. (2000). The COG database: a tool for genome-scale analysis of protein functions and evolution. Nucleic acids research, 28(1), 33-36. doi.org/10.1093/nar/28.1.33
Greiner L L, Watanabe H, Phillips N J, Shao J, Morgan A, Zaleski A, Gibson B W, Apicella M A. Nontypeable Haemophilus influenzae strain 2019 produces a biofilm containing N-acetylneuraminic acid that may mimic sialylated O-linked glycans. Infect Immun. 2004 July; 72(7):4249-60. doi: 10.1128/IAI.72.7.4249-4260.2004. PMID: 152.13170; PMCID: PMC4271468.

1. A method for one or more of: preventing, inhibiting, disrupting, dispersing or treating a polymicrobial biofilm or a disease related to a polymicrobial biofilm that is caused by, comprises or will be caused by a Haemophilus bacteria in a subject in need thereof, comprising administering to the subject: (i) an anti-DNABII antibody or a biologically active fragment thereof and an anti-PilA antibody or biologically active fragment thereof; or (ii) an anti-DNABII antibody or a biologically active fragment thereof and a PilA polypeptide or a biologically active fragment thereof.

2. (canceled)

3. (canceled)

4. The method of claim 1, wherein the subject was administered an antibiotic comprising a β-lactam antibiotic and/or a sulfonamide antibiotic.

5. A method for one or more of: preventing, inhibiting, disrupting, dispersing or treating a polymicrobial biofilm that is caused by, comprises or will be caused by a Haemophilus bacteria, comprising contacting the polymicrobial biofilm with: (i) an anti-DNA binding and bending protein (DNABII) antibody or a biologically active fragment thereof, and (ii) an anti-majority subunit (PilA) of type IV pilus (T4P) antibody or a biologically active fragment thereof.

6. (canceled)

7. (canceled)

8. A method for sensitizing a polymicrobial biofilm comprising a Haemophilus bacteria, optionally in a subject in need thereof, for an antibiotic therapy or inducing bacteria that forms the polymicrobial biofilm to a newly released (NRel) state, comprising contacting the biofilm with: (i) an anti-DNABII antibody or a biologically active fragment thereof, and (ii) an anti-PilA antibody or a biologically active fragment thereof or a PilA polypeptide or a biologically active fragment thereof.

9. (canceled)

10. (canceled)

11. (canceled)

12. The method of claim 1, wherein the DNABII is selected from an integration host factor (IHF) or a histone-like protein (HU) protein.

13. The method of claim 1, wherein the anti-DNABII antibody or biologically active fragment thereof recognizes and binds to one or more of: a DNABII A5 peptide, a DNABII mB4 peptide, a tip peptide, a tip chimer peptide, or a recombinant tip chimer polypeptide comprising the A5 and the mB4 peptides.

14. The method of claim 1, wherein the PilA is a recombinant and soluble PilA (rsPilA).

15. (canceled)

16. (canceled)

17. (canceled)

18. (canceled)

19. The method of claim 4, wherein the antibiotic is contacted with the biofilm or administrated to the subject concurrently with or after (i) and (ii).

20. The method of claim 1, wherein the Haemophilus bacteria comprises Haemophilus influenza or Nontypeable Haemophilus influenzae (NTHI).

21. The method of claim 1, wherein the polymicrobial biofilm comprises a Haemophilus bacteria and at least one of: Moraxella catarrhalis, Staphylococcus aureus, Pseudomonas aeruginosa, Streptococcus pneumoniae, Burkholderia cenocepacia, an ESKAPE pathogen (selected from Enterococcus faecium, Klebsiella pneumoniae, Acinetobacter baumannii, Pseudomonas aeruginosa, and Enterobacter spp.), Aggregatibacter actinomycetemcomitans.

22. (canceled)

23. The method of claim 1, wherein the polymicrobial biofilm is recurrent, or recalcitrant to an antibiotic monotherapy.

24. The method of claim 1 wherein the bacteria in a sample of the subject or the polymicrobial biofilm was identified prior to the contacting or administering step.

25. The method of claim 1, whereby the polymicrobial biofilm is one or more of: prevented, inhibited, disrupted, dispersed or treated, in a synergistic manner compared to contacting or administering one of an anti-DNABII antibody or a biologically active fragment thereof, an anti-PilA antibody or a biologically active fragment thereof, a PilA polypeptide or a biologically active fragment thereof, or an antibiotic alone.

26. The method of claim 8, wherein the newly released (NRel) state of the bacteria released from the biofilm comprises one or more of the following:
an altered gene expression of one or more of the following genes: deaD, artM, fis, folA, folP, emrA, emrB, or acrR;
an altered level of one or more of the following proteins: AsnC, CyaA, GlpC, NrfA, TehB, TrpB, TrpC, TrpD, TruA, or TrxA;
a low level of one or more of the following proteins: AbgA, AroE, AroK, ArtP, AtpH, BioB, BioF, CcmA, CitD, CitT, CmK, CoaD, DcD, DjlA, DksA, DnaE, DnaQ, DsbE, FabG, FdhX, FtnB, FtsE, FtsI, FumC, Fur, GlpA, GlpB, GmK, GpsA, GreA, Hfq, HisI, HugZ, InfA, InfB, IspD, IspF, Lic2A, LicC, LicD, LolA, LpsA, menC, MenC, NapA, NrfB, NrfC, NTHI0053, NTHI0249, NTHI0291, NTHI0319, NTHI0349, NTHI0436, NTHI0487, NTHI0490, NTHI0555, NTHI0732, NTHI0779, NTHI0820, NTHI1025, NTHI1085, NTHI1199, NTHI1437, NTHI1439, NTHI1503, NTHI1590, NTHI1748, OppF, PanF, PdxH, PepT, PhoB, PlsC, PolA, PpC, PpiB, PrfB, PurU, RadA, RelB, RibA, RplW, RpoE, RpoZ, RseA, SecF, Sxy, TesB, ThrB, TolA, UnG, UreG, UuaP, or UvrB;
a high level of one or more of the following proteins: ClpB, CydD, DeaD, DlD, DmsB, ExbD, GroES, HfeA, HfeB, HgpB, HisJ, HisJ, HitA, HktE, HxuC, LctP, LldD, LpxC, MurB, NdhA, NifS2, NTHI0043, NTHI0052, NTHI0175, NTHI0364, NTHI1208, NTHI1214, NTHI1369, NTHI1703, OmpP2, OrfG, Pal, PdgX, RbfA, RecN, RplO, RplU, RpmE, RpsE, RpsJ, RpsL, RpsQ, RpsT, SodA, Tbpl, TolB, TonB, TrpA, TrxA, or ZnuA.

27. The method of claim 26, wherein the high, low, altered expression or level is compared to the same bacteria but grown planktonically.

28. A kit for use in the method of claim 1, comprising at least two of:
(i) an anti-DNABII antibody or a biologically active fragment thereof,
(ii) one or both of: an anti-PilA antibody or a biologically active fragment thereof, or a PilA polypeptide or a biologically active fragment thereof, or
(iii) an antibiotic optionally comprising a β-lactam antibiotic or a sulfonamide antibiotic, and optionally instructions for use.

29. A composition comprising at least two of the following:
(i) an anti-DNABII antibody or a biologically active fragment thereof,
(ii) one or both of: an anti-PilA antibody or a biologically active fragment thereof, or a PilA polypeptide or a biologically active fragment thereof, or
(iii) an antibiotic optionally comprising a β-lactam antibiotic or a sulfonamide antibiotic, and a carrier optionally a pharmaceutically acceptable carrier.

30. (canceled)

31. The composition of claim 30, wherein the anti-DNABII antibody or biologically active fragment thereof recognizes and binds to one or more of: a DNABII A5 peptide, a DNABII mB4 peptide, or a recombinant polypeptide comprising the A5 and the mB4 peptides.

32. (canceled)

33. (canceled)

34. (canceled)

35. (canceled)

36. (canceled)

37. A method for selecting a polymicrobial biofilm suitable for one or more of the following: prevention, inhibition, disruption, dispersion, treatment or sensitization by the method of claim 1, comprising
(a) contacting the polymicrobial biofilm, or a polymicrobial biofilm isolated and grown therefrom, with one or both of (a) an anti-DNABII antibody or a biologically active fragment thereof or (b) an anti-PilA antibody or biologically active fragment thereof or a PilA polypeptide or a biologically active fragment thereof; and
(b) assaying released bacteria from the biofilm of (a) for expression of one or more of the following genes: deaD, artM, fis, folA, folP, emrA, emrB, or acrR and level of one or more of proteins: AsnC, CyaA, GlpC, NrfA, TehB, TrpB, TrpC, TrpD, TruA, TrxA; AbgA, AroE, AroK, ArtP, AtpH, BioB, BioF, CcmA, CitD, CitT, CmK, CoaD, DcD, DjlA, DksA, DnaE, DnaQ, DsbE, FabG, FdhX, FtnB, FtsE, FtsI, FumC, Fur, GlpA, GlpB, GmK, GpsA, GreA, Hfq, HisI, HugZ, InfA, InfB, IspD, IspF, Lic2A, LicC, LicD, LolA, LpsA, menC, MenC, NapA, NrfB, NrfC, NTHI0053, NTHI0249, NTHI0291, NTHI0319, NTHI0349, NTHI0436, NTHI0487, NTHI0490, NTHI0555, NTHI0732, NTHI0779, NTHI0820, NTHI1025, NTHI1085, NTHI1199, NTHI1437, NTHI1439, NTHI1503, NTHI1590, NTHI1748, OppF, PanF, PdxH, PepT, PhoB, PlsC, PolA, PpC, PpiB, PrfB, PurU, RadA, RelB, RibA, RplW, RpoE, RpoZ, RseA, SecF, Sxy, TesB, ThrB, TolA, UnG, UreG, UuaP, UvrB; ClpB, CydD, DeaD, DlD, DmsB, ExbD, GroES, HfeA, HfeB, HgpB, HisJ, HisJ, HitA, HktE, HxuC, LctP, LldD, LpxC, MurB, NdhA, NifS2, NTHI0043, NTHI0052, NTHI0175, NTHI0364, NTHI1208, NTHI1214, NTHI1369, NTHI1703, OmpP2, OrfG, Pal, PdgX, RbfA, RecN, RplO, RplU, RpmE, RpsE, RpsJ, RpsL, RpsQ, RpsT, SodA, Tbpl, TolB, TonB, TrpA, TrxA, or ZnuA,
wherein one or more of the following indicates the biofilm is suitable for the method of claim 1,
an altered gene expression of one or more of the following genes: deaD, artM, fis, folA, folP, emrA, emrB, or acrR;
an altered level of one or more of the following proteins: AsnC, CyaA, GlpC, NrfA, TehB, TrpB, TrpC, TrpD, TruA, or TrxA;
a low level of one or more of the following proteins: AbgA, AroE, AroK, ArtP, AtpH, BioB, BioF, CcmA, CitD, CitT, CmK, CoaD, DcD, DjlA, DksA, DnaE, DnaQ, DsbE, FabG, FdhX, FtnB, FtsE, FtsI, FumC, Fur, GlpA, GlpB, GmK, GpsA, GreA, Hfq, HisI, HugZ, InfA, InfB, IspD, IspF, Lic2A, LicC, LicD, LolA, LpsA, menC, MenC, NapA, NrfB, NrfC, NTHI0053, NTHI0249, NTHI0291, NTHI0319, NTHI0349, NTHI0436, NTHI0487, NTHI0490, NTHI0555, NTHI0732, NTHI0779, NTHI0820, NTHI1025, NTHI1085, NTHI1199, NTHI1437, NTHI1439, NTHI1503, NTHI1590, NTHI1748, OppF, PanF, PdxH, PepT, PhoB, PlsC, PolA, PpC, PpiB, PrfB, PurU, RadA, RelB, RibA, RplW, RpoE, RpoZ, RseA, SecF, Sxy, TesB, ThrB, TolA, UnG, UreG, UuaP, or UvrB;
a high level of one or more of the following proteins: ClpB, CydD, DeaD, DlD, DmsB, ExbD, GroES, HfeA, HfeB, HgpB, HisJ, HisJ, HitA, HktE, HxuC, LctP, LldD, LpxC, MurB, NdhA, NifS2, NTHI0043, NTHI0052, NTHI0175, NTHI0364, NTHI1208, NTHI1214, NTHI1369, NTHI1703, OmpP2, OrfG, Pal, PdgX, RbfA, RecN, RplO, RplU, RpmE, RpsE, RpsJ, RpsL, RpsQ, RpsT, SodA, Tbpl, TolB, TonB, TrpA, TrxA, or ZnuA.

38. (canceled)

39. The method of claim 37, wherein the selected polymicrobial biofilm was contacted with:
(i) an anti-DNABII antibody or a biologically active fragment thereof,
(ii) an anti-PilA antibody or a biologically active fragment thereof or a PilA polypeptide or a biologically active fragment thereof; and
(iii) an optional antibiotic optionally comprising a β-lactam antibiotic or a sulfonamide antibiotic.

40. (canceled)
